(19) United States
(12) Patent Application Publication
(10) Pub. No.: US 2025/0283167 A1
(43) Pub. Date: Sep. 11, 2025

(54) SYSTEMS AND METHODS FOR EVALUATING TUMOR FRACTION

(71) Applicant: Foundation Medicine, Inc., Cambridge, MA (US)

(72) Inventors: Russell MADISON, Encinitas, CA (US); Dean PAVLICK, Cambridge, MA (US); Bernard FENDLER, Auburndale, MA (US); Brady FORCIER, Dorchester, MA (US); Daniel LIEBER, Needham, MA (US); Mark KENNEDY, Cambridge, MA (US)

(73) Assignee: Foundation Medicine, Inc., Cambridge, MA (US)

(21) Appl. No.: 18/570,600

(22) PCT Filed: Jun. 22, 2021
(86) PCT No.: PCT/US2021/038547

§ 371 Date: Dec. 14, 2023

(51) Int. Cl. C12Q 1/6874; C12Q 1/6844; C12Q 1/6886; G16B 20/20; G16B 30/00; G16B 40/20

(52) U.S. Cl. CPC C12Q 1/6874; C12Q 1/6844; C12Q 1/6886; G16B 20/20; G16B 30/00; G16B 40/20; C12Q 2600/156

(57) ABSTRACT

Disclosed herein are methods of determining a tumor fraction of a sample from a subject. The methods can include, for example, acquiring a value for a target variable associated with a subgenomic interval m the sample; determining, from the target variable, a certainty metric: accessing a determined relationship between a stored certainty metric and a stored tumor fraction; and determining, with reference to the certainty metric and the determined relationship, the tumor fraction of the sample. In some instances, the method can further include determining a tumor fraction of the sample using a determination of somatic allele frequency when the estimate of tumor fraction calculated from the certainty metric falls below a cut-off threshold.

### CROSS REFERENCE TO RELATED APPLICATIONS

This application is a national stage application under 35 U.S.C. § 371 of International Application No. PCT/US2021/038547, filed internationally on Jun. 22, 2021, which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 197102006500SEQLIST.txt, date recorded: Nov. 7, 2023, size: 1,292 bytes).

### BACKGROUND

Cancer can be caused by genomic mutations, and cancer cells may accumulate mutations during cancer development and progression. These mutations may be the consequence of intrinsic malfunction of DNA repair, replication, or modification mechanisms, or may be a consequence of exposure to external mutagens. Certain mutations confer growth advantages on cancer cells and are positively selected in the microenvironment of the tissue in which the cancer arises. Detection of these mutations in patient samples using next generation sequencing (NGS) or other genomic analysis techniques can provide valuable insights with respect to diagnosis, prognosis, and treatment of cancer. However, translating the results of genomic studies into routine clinical practice remains expensive, time intensive, and technically challenging.

Liquid biopsy has become a promising tool for applying the results of genomics studies to practical clinical application. One of the challenges of detecting mutations related to cancer in liquid biopsy samples is the low abundance of circulating tumor DNA (ctDNA) shed by cancerous tissue into the bloodstream relative to the total amount of cell-free DNA (cfDNA) present, as well as the often very low allele frequencies of the mutations of interest. Existing techniques for determining circulating tumor fraction (i.e., the amount of circulating tumor DNA (ctDNA) relative to the total amount of cell-free DNA (cfDNA) present in the sample), such as those based solely on determining somatic allele frequencies, or on cfDNA fragment size, may suffer from performance limitations at low ctDNA levels. Therefore, there is a need for novel methods that provide more accurate determinations of circulating tumor fraction, which in turn may provide more accurate determinations of the presence or absence of cancer-related biomarkers in patient samples.

### BRIEF SUMMARY OF THE INVENTION

Disclosed herein are methods and systems that utilize comprehensive genomic profiling (CGP) for determining tumor fraction (e.g., the amount of circulating tumor DNA (ctDNA) relative to the total amount of cell-free DNA (cfDNA)) in a liquid biopsy sample). In some embodiments, the disclosed methods provide a determination of a composite tumor fraction that takes into account both variant allele frequencies (VAF) and the tumor fraction of the sample rather than tumor fraction alone. The methods comprise the combined use of two complementary processes that, in combination, provide a more accurate, composite determination of circulating tumor fraction over a broader range of ctDNA concentrations. The first stage of the combined process comprises the determination of a certainty metric for the sample which is indicative of the dispersion in, for example, allele fraction data for a specified set of genomic loci. The certainty metric for the sample is compared to a reference curve that relates certainty metric values to the circulating tumor fraction for a series of samples comprising known percentages of circulating tumor DNA to determine a tumor fraction estimate for the sample. If the estimate of circulating tumor fraction, or a parameter related thereto, returned by the first stage of the combined process is less than or equal to a first threshold value, a second stage of the combined process. i.e., for estimating circulating tumor fraction based on an allele frequency determination, may be utilized.

Disclosed herein are methods of identifying a genomic sequence of interest as germline or somatic, the method comprising: providing a plurality of nucleic acid molecules obtained from a sample from a subject, wherein the plurality of nucleic acid molecules comprises a mixture of tumor nucleic acid molecules and non-tumor nucleic acid molecules; optionally, ligating one or more adapters onto one or more nucleic acid molecules from the plurality of nucleic acid molecules; amplifying nucleic acid molecules from the plurality of nucleic acid molecules; capturing nucleic acid molecules from the amplified nucleic acid molecules, wherein the captured nucleic acid molecules are captured from the amplified nucleic acid molecules by hybridization to one or more bait molecules; sequencing, by a sequencer, the captured nucleic acid molecules to obtain a plurality of sequence reads corresponding to one or more genomic loci within a subgenomic interval in the sample; receiving, at one or more processors, a plurality of values, each value indicative of an allele fraction at a corresponding locus within the subgenomic interval in the sample; determining, by the one or more processors, a certainty metric value indicative of a dispersion of the plurality of values; determining, by the one or more processors, a first estimate of the tumor fraction of the sample, the first estimate based on the certainty metric value for the sample and a predetermined relationship between one or more stored certainty metric values and one or more stored tumor fraction values; determining, by the one or more processors, whether a value associated with the first estimate is greater than a first threshold; based on a determination that the value associated with the first estimate is greater than the first threshold, outputting, by the one or more processors, the first estimate as the tumor fraction of the sample; and based on a determination that the value associated with the first estimate is less than or equal to the first threshold: determining, by the one or more processors, a second estimate of the tumor fraction of the sample based on an allele frequency determination; and outputting, by the one or more processors, the second estimate as the tumor fraction of the sample.

In some embodiments, determining the second estimate of the tumor fraction of the sample based on the allele frequency determination comprises: determining whether a quality metric for the plurality of values is greater than a second threshold; based on a determination that the quality metric for the plurality of values is greater than the second threshold, determining the second estimate for the tumor fraction of the sample based on a first determination of somatic allele frequency, and based on a determination that the quality metric for the plurality of values is less than or equal to the second threshold, determining the second estimate for the tumor fraction of the sample based on a second determination of somatic allele frequency. In some embodiments, the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected. In some embodiments, the second determination of somatic allele frequency comprises a determination of variant allele frequencies for all variant alleles associated with the plurality of values, and the second estimate of the tumor fraction of the sample is set equal to a maximum variant allele frequency detected. In some embodiments, the subject is a cancer patient. In some embodiments, the method further comprises obtaining the sample from the subject. In some embodiments, the sample comprises a tissue biopsy sample, a liquid biopsy sample, a circulating tumor cell (CTC) sample, a cell-free DNA (cfDNA) sample, or a normal control. In some embodiments, the sample is a liquid biopsy sample and comprises blood, plasma, cerebrospinal fluid, sputum, stool, urine, or saliva. In some embodiments, the tumor nucleic acid molecules are derived from a tumor portion of a heterogeneous tissue biopsy sample, and the non-tumor nucleic acid molecules are derived from a normal portion of the heterogeneous tissue biopsy sample. In some embodiments, the tumor nucleic acid molecules are derived from a circulating tumor DNA (ctDNA) fraction of a cell-free DNA sample, and the non-tumor nucleic acid molecules are derived from a non-tumor fraction of the cell-free DNA sample. In some embodiments, the one or more adapters comprise amplification primers or sequencing adapters. In some embodiments, the one or more bait molecules comprise one or more nucleic acid molecules, each comprising a region that is complementary to a region of a captured nucleic acid molecule. In some embodiments, amplifying nucleic acid molecules comprises performing a polymerase chain reaction (PCR) or isothermal amplification technique. In some embodiments, the sequencing comprises use of a next generation sequencing (NGS) technique. In some embodiments, the sequencer comprises a next generation sequencer. In some embodiments, the method further comprises generating, by the one or more processors, a report indicating the tumor fraction of the sample. In some embodiments, the method comprises transmitting the report to a healthcare provider. In some embodiments, the report is transmitted via a computer network or a peer-to-peer connection.

Disclosed herein are methods for determining a tumor fraction of a sample from a subject, comprising: receiving, at one or more processors, a plurality of values, each value indicative of an allele fraction at a corresponding locus within a subgenomic interval in the sample; determining, by the one or more processors, a certainty metric value indicative of a dispersion of the plurality of values; determining, by the one or more processors, a first estimate of the tumor fraction of the sample, the first estimate based on the certainty metric value for the sample and a predetermined relationship between one or more stored certainty metric values and one or more stored tumor fraction values; determining, by the one or more processors, whether a value associated with the first estimate is greater than a first threshold; based on a determination that the value associated with the first estimate is greater than the first threshold, outputting, by the one or more processors, the first estimate as the tumor fraction of the sample; and based on a determination that the value associated with the first estimate is less than or equal to the first threshold: determining, by the one or more processors, a second estimate of the tumor fraction of the sample based on an allele frequency determination; and outputting, by the one or more processors, the second estimate as the tumor fraction of the sample.

In some embodiments, the tumor fraction is a value indicative of a ratio of circulating tumor DNA (ctDNA) to total cell-free DNA (cfDNA) in the sample. In some embodiments, the first threshold is indicative of a minimum detectable quantity for the tumor fraction of the sample. In some embodiments, determining whether the value associated with the first estimate of the tumor fraction of the sample is greater than the first threshold comprises determining whether the first estimate is greater than a predefined tumor fraction threshold. In some embodiments, determining whether the value associated with the first estimate of the tumor fraction of the sample is greater than a first threshold comprises determining whether a statistical lower bound associated with the first estimate is greater than 0.

In some embodiments, determining the second estimate of the tumor fraction of the sample based on the allele frequency determination comprises: determining whether a quality metric for the plurality of values is greater than a second threshold; based on a determination that the quality metric for the plurality of values is greater than the second threshold, determining the second estimate for the tumor fraction of the sample based on a first determination of somatic allele frequency, and based on a determination that the quality metric for the plurality of values is less than or equal to the second threshold, determining the second estimate for the tumor fraction of the sample based on a second determination of somatic allele frequency.

In some embodiments, the quality metric for the plurality of values is indicative of an average sequence coverage for the sample, an allele coverage at each of the loci corresponding to the plurality of values, a degree of nucleic acid contamination in the sample, a number of single nucleotide polymorphism (SNP) loci within the loci corresponding to the plurality of values, or any combination thereof. In some embodiments, the quality metric for the plurality of values is indicative of a minimum average sequence coverage for the sample, a minimum allele coverage at each of the loci corresponding to the plurality of values, a maximum degree of nucleic acid contamination in the sample, a minimum number of single nucleotide polymorphism (SNP) loci within the loci corresponding to the plurality of values, or any combination thereof.

In some embodiments, the second threshold comprises a specified lower limit of the quality metric. In some embodiments, the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected. In some embodiments, the second determination of somatic allele frequency comprises a determination of variant allele frequencies for all variant alleles associated with the plurality of values, and the second estimate of the tumor fraction of the sample is set equal to a maximum variant allele frequency detected. In some embodiments, the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample. In some embodiments, the first determination of somatic allele frequency and the second determination of somatic allele frequency further comprise using a variant allele frequency for a rearrangement as the second estimate of the tumor fraction of the sample if rearrangements are detected in the sample.

In some embodiments, each value within the plurality of values is an allele fraction. In some embodiments, each value within the plurality of values comprises a ratio of the difference in abundance between a maternal allele and a paternal allele relative to abundance of the maternal allele or the paternal allele at the corresponding locus. In some embodiments, the certainty metric value for the sample is indicative of a deviation of each of the plurality of values from a corresponding expected value. In some embodiments, the expected value is a locus-specific expected value. In some embodiments, the certainty metric for the sample is a root mean squared deviation of the plurality of values from their corresponding expected values. In some embodiments, the expected value is an expected allele frequency for a non-tumorous sample. In some embodiments, each value within the plurality of values is an allele fraction, and the expected value is 0.5. In some embodiments, each value within the plurality of values is a ratio of the difference in abundance between a maternal allele and a paternal allele, relative to an abundance of the maternal allele or the paternal allele at the corresponding locus, and the expected value comprises the expected ratio of the difference in abundance between a maternal allele and a paternal allele, relative to an abundance of the maternal allele or the paternal allele, wherein the expected value is the expected ratio for a non-tumorous sample. In some embodiments, the expected value is 0. In some embodiments, the plurality of values comprises a plurality of allele coverages. In some embodiments, the method further comprises determining a probability distribution function for the plurality of values; wherein the certainty metric value for the sample is determined using the probability distribution function. In some embodiments, the certainty metric value for the sample is an entropy of the probability distribution function. In some embodiments, the corresponding loci comprise one or more loci having a different maternal allele and paternal allele. In some embodiments, the corresponding loci consist of loci having a different maternal allele and paternal allele. In some embodiments, the corresponding loci comprise one or more loci having the same maternal allele and paternal allele.

Disclosed herein are methods of determining a tumor fraction of a sample from a subject, comprising: receiving, at one or more processors, a plurality of values, each value indicative of a difference between an allele coverage of a locus in a tumor sample and an allele coverage of the same locus in a non-tumor sample at a plurality of loci within a sub genomic interval; determining, by the one or more processors, a certainty metric value indicative of a dispersion of the plurality of values; determining, by the one or more processors, a first estimate of the tumor fraction of the sample, the first estimate based on the certainty metric value for the sample and a predetermined relationship between one or more stored certainty metric values and one or more stored tumor fraction values; determining, by the one or more processors, whether a value associated with the first estimate is greater than a first threshold; based on a determination that the value associated with the first estimate is greater than the first threshold, outputting, by the one or more processors, the first estimate as the tumor fraction of the sample; and based on a determination that the value associated with the first estimate of the tumor fraction is less than or equal to the first threshold: determining, by the one or more processors, a second estimate of the tumor fraction of the sample based on an allele frequency determination; and outputting, by the one or more processors, the second estimate as the tumor fraction of the sample.

In some embodiments, the tumor fraction is a value indicative of the ratio of circulating tumor DNA (ctDNA) to total cell-free DNA (cfDNA) in the sample. In some embodiments, the first threshold is indicative of a minimum detectable quantity for the tumor fraction of the sample. In some embodiments, determining whether the value associated with the first estimate of the tumor fraction of the sample is greater than the first threshold comprises determining whether the first estimate is greater than a predefined tumor fraction threshold. In some embodiments, determining whether the value associated with the first estimate of the tumor fraction of the sample is greater than a first threshold comprises determining whether a statistical lower bound associated with the first estimate is greater than 0.

In some embodiments, determining the second estimate of the tumor fraction of the sample comprises: determining whether a quality metric for the plurality of values is greater than a second threshold; based on a determination that the quality metric for the plurality of values is greater than the second threshold, determining the second estimate for the tumor fraction of the sample based on a first determination of somatic allele frequency, and based on a determination that the quality metric for the plurality of values is less than or equal to the second threshold, determining the second estimate for the tumor fraction of the sample based on a second determination of somatic allele frequency.

In some embodiments, the quality metric for the plurality of values is indicative of a predetermined average sequence coverage for the sample, a predetermined allele coverage at each of the corresponding loci, a predetermined degree of nucleic acid contamination in the sample, a predetermined number of single nucleotide polymorphism (SNP) loci within the plurality of corresponding loci, or any combination thereof. In some embodiments, the quality metric for the plurality of values is indicative of a minimum average sequence coverage for the sample, a minimum allele coverage at each of the corresponding loci, a maximum degree of nucleic acid contamination in the sample, a minimum number of single nucleotide polymorphism (SNP) loci within the plurality of corresponding loci, or any combination thereof. In some embodiments, the second threshold comprises a specified lower limit of the quality metric.

In some embodiments, the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected. In some embodiments, the second determination of somatic allele frequency comprises a determination of variant allele frequencies for all variant alleles associated with the plurality of values, and the second estimate of the tumor fraction of the sample is set equal to a maximum variant allele frequency detected. In some embodiments, the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample. In some embodiments, the first determination of somatic allele frequency and the second determination of somatic allele frequency further comprise using a variant allele frequency for a rearrangement as the second estimate of the tumor fraction of the sample if rearrangements are detected in the sample.

In some embodiments, each value within the plurality of values comprises a ratio of an allele coverage of a locus in the tumor sample compared to an allele coverage of the same locus in the non-tumor sample. In some embodiments, each value within the plurality of values comprises a log ratio of an allele coverage of a locus in the tumor sample compared to an allele coverage of the same locus in the non-tumor sample. In some embodiments, the log ratio is a log 2 ratio. In some embodiments, each value within the plurality of values comprises a ratio of the difference in an allele coverage the locus in the tumor sample and same locus in the non-tumor sample, relative to an allele coverage of the same locus in the non-tumor sample. In some embodiments, the certainty metric value for the sample is indicative of a deviation of each value within the plurality of values from an expected value across the corresponding loci, wherein the expected value is the value that would be expected if the tumor sample were a non-tumor sample.

In some embodiments, each value comprises a ratio of an allele coverage of a locus in the tumor sample compared to an allele coverage of the same locus in the non-tumor sample, and the expected value is 1; each value comprises a log ratio of an allele coverage of a locus in the tumor sample compared to an allele coverage of the same locus in the non-tumor sample, and the expected value is 0; or each value comprises a ratio of the difference in an allele coverage the locus in the tumor sample and same locus in the non-tumor sample, relative to an allele coverage of the same locus in the non-tumor sample, and the expected value is 0.

In some embodiments, the certainty metric value for the sample is a root mean squared deviation from the expected value. In some embodiments, the method further comprises determining a probability distribution function for the plurality of values; wherein the certainty metric value for the sample is determined using the probability distribution function. In some embodiments, the certainty metric value for the sample is an entropy of the probability distribution function. In some embodiments, the allele coverage comprises an allele coverage of a maternal allele and a paternal allele. In some embodiments, the allele coverage consists of an allele coverage of a maternal allele and a paternal allele. In some embodiments, the plurality of loci comprises at least one nucleotide associated with a single nucleotide polymorphism (SNP). In some embodiments, the plurality of loci comprises two or more nucleotides each associated with a single nucleotide polymorphism (SNP). In some embodiments, the SNP is associated with a cancer. In some embodiments, at least a portion of the plurality of loci is associated with a copy number variation (CNV). In some embodiments, the CNV is associated with a cancer.

In some embodiments, the method further comprises obtaining the sample from the subject. In some embodiments, the method further comprises extracting nucleic acid molecules from the sample. In some embodiments, the nucleic acid molecules comprise deoxyribonucleic acid (DNA) molecules. In some embodiments, the deoxyribonucleic acid (DNA) molecules comprise cell-free DNA (cfDNA) molecules or circulating tumor DNA (ctDNA) molecules. In some embodiments, the method further comprises ligating adapters to the nucleic acid molecules extracted from the sample. In some embodiments, the method further comprises sequencing the nucleic acid molecules extracted from the sample, to determine an allele abundance or coverage at each locus. In some embodiments, the method further comprises performing array hybridization on the nucleic acid molecules extracted from the sample to determine an allele abundance or coverage at each locus. In some embodiments, the method further comprises: obtaining a training dataset comprising a plurality of training certainty metric values and associated training tumor fractions values; training a machine learning model based on the training dataset; and using the trained machine learning model to determine a tumor fraction value from the certainty metric value for the sample. In some embodiments, the method further comprises a report comprising information identifying the subject and the determined tumor fraction. In some embodiments, the method further comprises displaying the report on a display device. In some embodiments, the method further comprises providing the report to the subject or a healthcare provider. In some embodiments, the method further comprises formatting the report for an electronic health record. In some embodiments, the method further comprises treating the subject for cancer based on the determined tumor fraction.

Also disclosed herein are methods of treating a tumor in a subject, comprising: based on a determined tumor fraction, administering an effective amount of a selected tumor therapy to the subject, wherein the tumor fraction is determined according to any of the methods for determining a tumor fraction or circulating tumor fraction disclosed herein.

In some embodiments, the method further comprises determining, based on the determined tumor fraction, the presence of the tumor in the patient. In some embodiments, the tumor therapy comprises chemotherapy, radiation therapy, targeted immunotherapy, or surgery. In some embodiments, the tumor therapy comprises a targeted immunotherapy selected based on a result of a genomic profiling assay. In some embodiments, the genomic profiling assay comprises a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profiling test comprises a nucleic acid sequencing-based test.

Disclosed herein are methods of monitoring tumor progression or recurrence in a subject, comprising: determining a first tumor fraction of a first sample obtained from the subject at a first time point according to any of the methods for determining a tumor fraction or circulating tumor fraction disclosed herein; determining a second tumor fraction of a second sample obtained from the subject at a second time point; and comparing the first tumor fraction to the second tumor fraction, thereby monitoring the tumor progression.

In some embodiments, determining the second tumor fraction comprises: receiving, at one or more processors, a second plurality of values, each value indicative of an allele fraction at a corresponding locus within a subgenomic interval in the second sample, wherein the subgenomic interval in the second sample is the same as or different than the subgenomic interval in the first sample; determining, by the one or more processors, a second certainty metric value indicative of a dispersion of the second plurality of values; determining, by the one or more processors, a third estimate of the second tumor fraction of the second sample, the third estimate based on the second certainty metric value for the second sample and the predetermined relationship between one or more stored certainty metric values and one or more stored tumor fraction values; determining, by the one or more processors, whether a value associated with the third estimate is greater than the first threshold; based on a determination that the value associated with the third estimate is greater than the first threshold, outputting, by the one or more processors, the third estimate as the second tumor fraction of the second sample; and based on a determination that the value associated with the third estimate is less than or equal to the first threshold: determining, by the one or more processors, a fourth estimate of the tumor fraction of the sample based on an allele frequency determination; and outputting, by the one or more processors, the fourth estimate as the second tumor fraction of the second sample.

In some embodiments, the method further comprises adjusting a tumor therapy in response to the tumor progression. In some embodiments, the method further comprises adjusting a dosage of the tumor therapy or selecting a different tumor therapy in response to the tumor progression. In some embodiments, the method further comprises administering the adjusted tumor therapy to the subject. In some embodiments, the tumor therapy comprises chemotherapy, radiation therapy, targeted immunotherapy, or surgery. In some embodiments, the tumor therapy comprises a targeted immunotherapy selected based on a result of a genomic profiling assay. In some embodiments, the genomic profiling assay comprises a comprehensive genomic profiling (CGP) test, a gene expression profiling test, a cancer hotspot panel test, a DNA methylation test, a DNA fragmentation test, an RNA fragmentation test, or any combination thereof. In some embodiments, the genomic profiling test comprises a nucleic acid sequencing-based test. In some embodiments, the first time point is before the subject has been administered a tumor therapy, and wherein the second time point is after the subject has been administered the tumor therapy. In some embodiments, the subject has a cancer, is at risk of having a cancer, or is suspected of having a cancer. In some embodiments, the cancer is a solid tumor. In some embodiments, the cancer is a hematological cancer. In some embodiments, the sample is a liquid sample. In some embodiments, the sample comprises cell-free DNA (cfDNA) or circulating tumor DNA (ctDNA). In some embodiments, the one or more stored certainty metric values comprise a plurality of stored certainty metric values, and the one or more stored tumor fraction values comprise a plurality of stored tumor fraction values.

Also disclosed herein are computer systems comprising: a processor; and a memory communicatively coupled to the processor, configured to store: a predetermined relationship between one or more stored certainty metric values and one or more associated stored tumor fraction values; and instructions that, when executed by the processor cause the processor to: receive a plurality of values, each value indicative of: (i) an allele fraction at a corresponding locus within a subgenomic interval in a sample, or (ii) a difference between an allele coverage of a locus in the sample and an allele coverage of the same locus in a non-tumor sample at a plurality of loci within a subgenomic interval; calculate a certainty metric value indicative of a dispersion for the plurality of values; calculate a first estimate of the tumor fraction of the sample, the first estimate based on the certainty metric value and the stored predetermined relationship; determine whether a value associated with the first estimate is greater than a first threshold; based on a determination that the value associated with the first estimate is greater than the first threshold, output the first estimate as the tumor fraction of the sample; and based on a determination that the value associated with the first estimate is less than or equal to the first threshold: calculate a second estimate of the tumor fraction of the sample based on an allele frequency determination; and output the second estimate as the tumor fraction of the sample.

In some embodiments, the tumor fraction is a value indicative of the ratio of circulating tumor DNA (ctDNA) to total cell-free DNA (cfDNA) in the sample. In some embodiments, the first threshold is indicative of a minimum detectable quantity for the tumor fraction of the sample. In some embodiments, determining whether the value associated with the first estimate of the tumor fraction of the sample is greater than the first threshold comprises determining whether the first estimate is greater than a predefined tumor fraction threshold. In some embodiments, determining whether the value associated with the first estimate of the tumor fraction of the sample is greater than a first threshold comprises determining whether a statistical lower bound associated with the first estimate is greater than 0.

In some embodiments, determining the second estimate of the tumor fraction of the sample comprises: determining whether a quality metric for the plurality of values is greater than a second threshold; based on a determination that the quality metric for the plurality of values is greater than the second threshold, determining the second estimate for the tumor fraction of the sample based on a first determination of somatic allele frequency, and based on a determination that the quality metric for the plurality of values is less than or equal to the second threshold, determining the second estimate for the tumor fraction of the sample based on a second determination of somatic allele frequency.

In some embodiments, the quality metric for the plurality of values is indicative of an average sequence coverage for the sample, an allele coverage at each of the loci corresponding to the plurality of values, a degree of nucleic acid contamination in the sample, a number of single nucleotide polymorphism (SNP) loci within the loci corresponding to the plurality of values, or any combination thereof. In some embodiments, the quality metric for the plurality of values is indicative of a minimum average sequence coverage for the sample, a minimum allele coverage at each of the loci corresponding to the plurality of values, a maximum degree of nucleic acid contamination in the sample, a minimum number of single nucleotide polymorphism (SNP) loci within the loci corresponding to the plurality of values, or any combination thereof. In some embodiments, the second threshold comprises a specified lower limit of the quality metric. In some embodiments, the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected. In some embodiments, the second determination of somatic allele frequency comprises a determination of variant allele frequencies for all variant alleles associated with the plurality of values, and the second estimate of the tumor fraction of the sample is set equal to a maximum variant allele frequency detected. In some embodiments, the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample. In some embodiments, the first determination of somatic allele frequency and the second determination of somatic allele frequency further comprise using a variant allele frequency for a rearrangement as the second estimate of the tumor fraction of the sample if rearrangements are detected in the sample.

In some embodiments, the memory further comprises instructions that, when executed by the processor, cause the processor to: obtaining a training dataset comprising a plurality of training certainty metric values and associated training tumor fraction values; training a machine learning model based on the training dataset; and using the trained machine learning model to determine a tumor fraction value from the certainty metric value for the sample.

Also disclosed herein are computer systems comprising: a processor; and a memory communicatively coupled to the processor, configured to store instructions that, when executed by the processor, cause the processor to perform any of the methods for determining tumor fraction or circulating tumor fraction disclosed herein.

Also disclosed herein are non-transitory computer-readable storage media storing one or more programs, the one or more programs comprising instructions which, when executed by one or more processors, cause a system comprising the one or more processors to: receive a plurality of values, each value indicative of: (i) an allele fraction at a corresponding locus within a subgenomic interval in a sample, or (ii) a difference between an allele coverage of a locus in the sample and an allele coverage of the same locus in a non-tumor sample at a plurality of loci within a subgenomic interval; calculate a certainty metric value indicative of a dispersion for the plurality of values; calculate a first estimate of the tumor fraction of the sample, the first estimate based on the certainty metric value and the stored predetermined relationship; determine whether a value associated with the first estimate is greater than a first threshold; based on a determination that the value associated with the first estimate is greater than the first threshold, output the first estimate as the tumor fraction of the sample; and based on a determination that the value associated with the first estimate is less than or equal to the first threshold: calculate a second estimate of the tumor fraction of the sample based on an allele frequency determination; and output the second estimate as the tumor fraction of the sample.

Methods and systems are also described herein that more generally allow for the evaluation of tumor fraction levels in a sample, a biopsy or a subject. Typically, tumor fraction is expressed or measured as the level or proportion of tumor-derived DNA in a sample relative to a reference, e.g., non-tumor DNA or all DNA, in the sample. In the methods described herein, a value for a certainty metric for the sample is obtained and that value can be evaluated in terms of a reference, e.g., by being compared with a reference. A certainty metric can itself be a function of a target variable that reflects the level of an allele at a subgenomic interval. Target variables may include variables that are a function of allele fraction, as well as variables that are a function of reads of subgenomic intervals.

In some embodiments, a value for the target variable is acquired, e.g., directly acquired, from the sample. Typically, the reference against which the certainty metric for the sample is compared is a certainty metric value (or a plurality of certainty metric values) that is associated with, e.g., correlated to, a level of tumor fraction. Certainty metric values that are incorporated into the reference can be based, e.g., on entities or relationships within the sample (e.g., 0.5 for an allele at a heterologous subgenomic interval) or external to the sample (e.g., a standard curve made from one or more other subjects).

In some examples, the target variable may be an allele fraction at one or more subgenomic intervals. Other examples of target variable include variables like log 2ratio, which is a function of the number of reads at one or more subgenomic intervals. Typically, a plurality of subgenomic intervals (e.g., 10, 20, 30, 40, 50, 60, 70, 80, 90, 100, 150, 200, 250, 300, or more subgenomic intervals) are analyzed to determine tumor faction. The plurality of subgenomic intervals may be present on the same chromosome or on different chromosomes (e.g., distributed among 2, 3, 4, 5, 6, 7, 8, 9, 10, 11, 12, 13, 14, 15, 16, 17, 18, 19, 20, 21, 22, or more chromosomes). In an embodiment, at least a portion of the plurality of subgenomic intervals are heterozygous (in terms of the alleles at the subgenomic interval).

In an embodiment, a certainty metric for a sample from a subject is compared with a curve that relates certainty metric to tumor fraction, and a value for sample tumor fraction is obtained.

In an embodiment, the certainty metric is a function of a target variable, e.g., allele fraction. By way of example, the certainty metric can be related to the degree by which the observed allele fraction deviates from a reference, e.g., an expected allele fraction or log 2ratio, and compared with a reference associated with a level of tumor fraction. In other examples, the certainty metric may measure the relative certainty of the target variable, e.g., an entropy metric described herein.

Thus, methods described herein include methods of evaluating, e.g., estimating, the tumor fraction of a sample. Such methods include, for example: obtaining a value for a target variable of a sample; obtaining a value for a reference, e.g., certainty metric as a function of the target variable; and comparing the sample value with the reference value to obtain a value for the tumor fraction of the sample.

In some embodiments, a method of determining a tumor fraction of a sample from a subject comprises acquiring a plurality of values, each value indicative of an allele fraction at a corresponding locus within a subgenomic interval in the sample; determining a certainty metric indicative of a dispersion of the plurality of values; accessing a predetermined relationship between one or more stored certainty metric and one or more stored tumor fraction; and determining, from the certainty metric and the predetermined relationship, the tumor fraction of the sample.

In some embodiments, each value within the plurality of values is an allele fraction. In some embodiments, each value within the plurality of values comprises a ratio of the difference in abundance between a maternal allele and a paternal allele relative to abundance of the maternal allele or the paternal allele at the corresponding locus. In some embodiments, the certainty metric is indicative of a deviation of each of the plurality of values from an expected value. In some embodiments, the expected value is a locus-specific expected value.

In some embodiments, the certainty metric is a root mean squared deviation from the expected value. In some embodiments, the expected value is an expected allele frequency for a non-tumorous. In some embodiments, each value within the plurality of values is and allele fraction, and the expected value is 0.5.

In some embodiments, each value within the plurality of values is a ratio of the difference in abundance between a maternal allele and a paternal allele, relative to abundance of the maternal allele or the paternal allele at the corresponding locus, and the expected value comprises the expected ratio of the difference in abundance between a maternal allele and a paternal allele relative, to abundance of the maternal allele or the paternal allele, wherein the expected value is the expected ratio for a non-tumorous sample. In some embodiments, the expected value is 0.

In some embodiments, the plurality of values comprises a plurality of allele coverages.

In some embodiments, the method further comprising determining a probability distribution function for the plurality of values; wherein the certainty metric is determined using the probability distribution function. In some embodiments, the certainty metric is an entropy of the probability distribution function.

In some embodiments, the corresponding loci comprise one or more loci having a different maternal allele and paternal allele. In some embodiments, the corresponding loci consist of loci having a different maternal allele and paternal allele. In some embodiments, the corresponding loci comprise one or more loci having the same maternal allele and paternal allele.

In some embodiments, a method of determining a tumor fraction of a sample from a subject, comprises: acquiring a plurality of values, each value indicative of a difference between an allele coverage of a locus in a tumor sample and an allele coverage of the same locus in a non-tumor sample at a plurality of loci within a subgenomic interval; determining a certainty metric indicative of a dispersion of the plurality of values; accessing a predetermined relationship between one or more stored certainty metric and one or more stored tumor fraction; and determining, from the certainty metric and the predetermined relationship, the tumor fraction of the sample.

In some embodiments, each value within the plurality of values comprises a ratio of an allele coverage of a locus in the tumor sample compared to an allele coverage of the same locus in the non-tumor sample.

In some embodiments, each value within the plurality of values comprises a log ratio of an allele coverage of a locus in the tumor sample compared to an allele coverage of the same locus in the non-tumor sample. In some embodiments, the log ratio is a log 2 ratio.

In some embodiments, each value within the plurality of values comprises a ratio of the difference in an allele coverage the locus in the tumor sample and same locus in the non-tumor sample, relative to an allele coverage of the same locus in the non-tumor sample.

In some embodiments, the certainty metric is indicative of a deviation of each value within the plurality of values from an expected value across the corresponding loci, wherein the expected value is the value that would be expected if the tumor sample were a non-tumor sample.

In some embodiments, each value comprises a ratio of an allele coverage of a locus in the tumor sample compared to an allele coverage of the same locus in the non-tumor sample, and the expected value is 1; each value comprises a log ratio of an allele coverage of a locus in the tumor sample compared to an allele coverage of the same locus in the non-tumor sample, and the expected value is 0; or each value comprises a ratio of the difference in an allele coverage the locus in the tumor sample and same locus in the non-tumor sample, relative to an allele coverage of the same locus in the non-tumor sample, and the expected value is 0.

In some embodiments, the certainty metric is a root mean squared deviation from the expected value.

In some embodiments, the method further comprising determining a probability distribution function for the plurality of values; wherein the certainty metric is determined using the probability distribution function. In some embodiments, the certainty metric is an entropy of the probability distribution function.

In some embodiments, the allele coverage comprises an allele coverage of a maternal allele and a paternal allele.

In some embodiments, the allele coverage consists of an allele coverage of a maternal allele and a paternal allele.

In some embodiments of the above methods, the plurality of loci comprises at least one nucleotide associated with a single nucleotide polymorphism (SNP). In some embodiments, the plurality of loci comprises two or more nucleotides each associated with a single nucleotide polymorphism (SNP). In some embodiments, the SNP is associated with a cancer.

In some embodiments of the above methods, at least a portion of the plurality of loci is associated with a copy number variation (CNV). In some embodiments, the CNV is associated with a cancer.

In some embodiments of the above methods, the method further comprises sequencing the sample, to determine an allele abundance or coverage at each locus.

In some embodiments of the above methods, the method further comprises performing array hybridization on the sample to determine an allele abundance or coverage at each locus.

In some embodiments of the above methods, the method further comprises accessing a training dataset comprising a plurality of relationships between a plurality of training certainty metrics and associated training tumor fractions; and applying a machine learning process to the training dataset to determine the predetermined relationship between the training certainty metrics and the training tumor fractions.

In some embodiments of the above methods, the method further comprises generating a report comprising information identifying the subject and the determined tumor fraction. In some embodiments, the method further comprises providing the report to the subject or a healthcare provider. In some embodiments, the method further comprises formatting the report for an electronic health record.

In some embodiments, a method of treating a tumor in a subject comprises, responsive to a determined tumor fraction, administering an effective amount of a tumor therapy to the subject, wherein the tumor fraction is determined according to any one of the methods described above. In some embodiments, the method comprises determining, based on the determined tumor fraction, the presence of the tumor in the patient. In some embodiments, the tumor therapy comprises chemotherapy, radiation therapy, targeted immunotherapy, or surgery.

In some embodiments, a method of monitoring tumor progression or recurrence in a subject comprises (a) determining a first tumor fraction of a first sample obtained from the subject at a first time point according to any one of the methods described above; (b) determining a second tumor fraction of a second sample obtained from the subject at a second time point; and (c) comparing the first tumor fraction to the second tumor fraction, thereby monitoring the tumor progression.

In some embodiments of the method of monitoring tumor progression or recurrence, determining the second tumor fraction comprises acquiring a second plurality of values, each value indicative of an allele fraction at a corresponding locus within a subgenomic interval in the second tumor sample, wherein the subgenomic interval in the second sample is the same or different than the subgenomic interval in the first sample; determining a second certainty metric indicative of a dispersion of the second plurality of values; accessing the predetermined relationship between one or more stored certainty metrics and one or more stored tumor fractions; and determining, from the second certainty metric and the predetermined relationship, the second tumor fraction of the second sample.

In some embodiments of the method of monitoring tumor progression or recurrence, determining the second tumor fraction comprises acquiring a second plurality of values, each value indicative of a difference between an allele coverage of a locus in the second tumor sample and an allele coverage of the same locus in a non-tumor sample at a plurality of loci within a subgenomic interval in the sample, wherein the subgenomic interval used to determine the second tumor fraction is the same or different than the subgenomic interval used to determine the first tumor fraction; determining a second certainty metric indicative of a dispersion of the second plurality of values; accessing the predetermined relationship between one or more stored certainty metrics and one or more stored tumor fractions; and determining, from the second certainty metric and the predetermined relationship, the second tumor fraction of the second tumor sample.

In some embodiments of the method of monitoring tumor progression or recurrence, the method further comprises adjusting a tumor therapy in response to the tumor progression. In some embodiments, the method comprises adjusting a dosage of the tumor therapy or selecting a different tumor therapy in response to the tumor progression. In some embodiments, the method comprises administering the adjusted tumor therapy to the subject.

In some embodiments of the method of monitoring tumor progression or recurrence the method comprises the first time point is before the subject has been administered a tumor therapy, and wherein the second time point is after the subject has been administered the tumor therapy.

In some embodiments of any of the methods described above, the subject has a cancer, is at risk of having a cancer, or is suspected of having a cancer. In some embodiments, the cancer is a solid tumor. In some embodiments, the cancer is a hematological cancer.

In some embodiments of any of the methods described above, the sample is a liquid sample.

In some embodiments of any of the methods described above, the sample is a solid sample.

In some embodiments of any of the methods described above, the sample comprises cell-free DNA (cfDNA) or circulating tumor DNA (ctDNA).

In some embodiments of any of the methods described above, the one or more stored certainty metrics comprises a plurality of stored certainty metrics, and the one or more stored tumor fractions comprises plurality of stored tumor fractions.

Also described herein is a computer system comprising: a processor; and a memory communicatively coupled to the processor, configured to store: a predetermined relationship between a one or more stored certainty metric and one or more associated stored tumor fraction; and instructions that, when executed by the processor cause the processor to: (a) (i) acquire a plurality of values, each value indicative of an allele fraction at a corresponding locus within a subgenomic interval in the sample, or (ii) acquire plurality of values, each value indicative of a difference between an allele coverage of a locus in a tumor sample and an allele coverage of the same locus in a non-tumor sample at a plurality of loci within a subgenomic interval; (b) determine a certainty metric indicative of a dispersion for the plurality of values; (c) access the stored predetermined relationship; and (d) determine, from the certainty metric and the predetermined relationship, the tumor fraction of the sample.

In some embodiments, of the computer system, the memory further comprises instructions that, when executed by the processor, cause the processor to: access a training dataset comprising a plurality of relationships between a plurality of training certainty metrics and associated training tumor fractions; and apply a machine learning process to the training dataset to determine the predetermined relationship between the training certainty metrics and the training tumor fractions.127. The method of any of claims 1-94, further comprising: determining, identifying, or applying the outputted tumor fraction of the sample as a diagnostic value associated with the sample.

In some embodiments, the method further comprises generating a genomic profile for the subject based on the outputted tumor fraction.

In some embodiments, the method further comprises administering an anti-cancer agent or applying an anti-cancer treatment to the subject based on the generated genomic profile.

In some embodiments, the outputted tumor fraction of the sample is used in generating a genomic profile for the subject.

In some embodiments, the outputted tumor fraction of the sample is used in making suggested treatment decisions for the subject.

In some embodiments, the outputted tumor fraction of the sample is used in applying or administering a treatment to the subject.

In some embodiments of the computer system, the instructions, when executed by the processor, cause the processor to perform any one of the methods described above.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts a process for determining a tumor fraction in a sample according to one embodiment of the present disclosure.

FIG. 2 provides a non-limiting example of a process for determining tumor fraction in a sample according to another embodiment of the present disclosure.

FIG. 3 provides a non-limiting example of a process for determining tumor fraction in a sample according to embodiments of the present disclosure.

FIG. 4 provides a non-limiting example of a process for determining tumor fraction in a sample according to a first determination of maximum somatic allele frequency.

FIG. 5 provides a non-limiting example of a process for determining tumor fraction in a sample according to a second determination of maximum somatic allele frequency.

FIG. 6 depicts and exemplary device, in accordance with some embodiment described herein.

FIG. 7 depicts an exemplary computer system, in accordance with some embodiments described herein.

FIG. 8 shows an exemplary relationship between entropy of a probability distribution function for SNP allele fractions in a sample with an associated tumor fraction (as represented by maximum somatic allele frequency), as determined using several serially diluted cancer samples.

FIG. 9 provides a non-limiting example of simulation data for determining tumor fraction according to examples of the present disclosure.

FIGS. 10A-D provide non-limiting examples of box-whisker plots of circulating cell-free DNA (cfDNA) yield based on patient's sex, age, stage, and tumor type. FIG. 10λ is a plot of cfDNA yield versus sex. FIG. 10B is a plot of cfDNA yield versus age. FIG. 10C is a plot of cfDNA yield versus disease stage. FIG. 10D is a plot of cfDNA yield versus tumor type.

FIGS. 11A-F provide non-limiting examples of box-whisker plots of circulating cell-free DNA (cfDNA) yield based on subgroups of select tumor types. FIG. 11A is a plot of cfDNA yield versys hematologic subgroup. FIG. 11B is a plot of cfDNA yield versus neuroendocrine subgroup. FIG. 11C is a plot of cfDNA yield versus skin subgroup. FIG. 11D is a plot of cfDNA yield for unknown/unclassified/low count subgroups. FIG. 11E is a plot of cfDNA yield versus non-small cell lung cancer (NSCLC) subgroup. FIG. 11F is a plot of cfDNA yield versus gastrointestinal subgroup.

FIGS. 12A-B provide non-limiting examples of box-whisker plots of circulating tumor DNA (ctDNA) fraction and estimated ctDNA quantity based on tumor type. FIG. 12A is a plot of ctDNA fraction versus tumor type. FIG. 12B is a plot of ctDNA yield versus tumor type. In each of FIGS. 10A-12B, the order of the items in the legend from top to bottom matches the order of the items (e.g., bars) in the corresponding plot from left to right.

### DETAILED DESCRIPTION

Described herein are methods and systems for determining a composite tumor fraction of a sample, e.g., a liquid biopsy sample, from a subject that takes into account both variant allele frequencies (VAF) and the tumor fraction of the sample and provides a more accurate value than conventional methods based on tumor fraction alone. In some embodiments, the disclosed methods provide a determination of a composite tumor fraction that takes into account both variant allele frequencies (VAF) and the tumor fraction of the sample rather than tumor fraction alone. Also described are methods of treating a tumor in a subject in response to a determined tumor fraction, and methods and systems for monitoring tumor progression or recurrence in a subject that include determining a tumor fraction in samples obtained from the subject at two or more time points. Quick and accurate tumor fraction determination, particularly at low tumor fraction levels, can substantially enhance tumor therapy by ensuring the subject receives effective therapy during early stages of the tumor or tumor recurrence. Other uses for tumor fraction are also contemplated and further discussed herein. For example, the tumor fraction may be used to analyze a tumor biopsy in some embodiments. In some embodiments, the tumor fraction is used to characterize a variant (for example as somatic or germline, or as homozygous, heterozygous, or sub-clonal), for example using a somatic-germline-zygosity (SGZ) algorithm. The methods and systems described herein provide accurate tumor fraction determination, even at low tumor fraction levels.

Liquid biopsy has become a promising tool for applying the results of genomics studies to practical clinical application. While the detection of mutations that function as predictive biomarkers for solid tumors typically uses a tissue biopsy as a sample type, an adequate tissue sample is sometimes difficult to obtain (e.g., for patients whose health status doesn't allow them to undergo a tissue biopsy, or because a biopsy is not technically feasible for the type of tissue to be sampled (e.g., bone)). For these patients, liquid biopsy (a test performed on a bodily fluid sample, e.g., blood or blood plasma, cerebrospinal fluid, sputum, stool, urine, or saliva, etc.) is an attractive, non-invasive alternative to tissue biopsy. In addition, liquid biopsy has the potential to become the method of choice for early detection and surveillance of disease. Studies have shown circulating tumor fraction to have prognostic value (Stover, et al. (2018) “Association of Cell-Free DNA Tumor Fraction and Somatic Copy Number Alterations With Survival in Metastatic Triple-Negative Breast Cancer”, J Clin Oncol 36:543-553) and the potential for monitoring disease progression, tumor burden, and response to therapy (Radovich, et al. (2020) “Association of Circulating Tumor DNA and Circulating Tumor Cells After Neoadjuvant Chemotherapy With Disease Recurrence in Patients With Triple-Negative Breast Cancer: Preplanned Secondary Analysis of the BRE12-158 Randomized Clinical Trial”, JAMA Oncol. 6(9):1410-1415).

One of the challenges of detecting mutations related to cancer in liquid biopsy samples is the low abundance of circulating tumor DNA (ctDNA) shed by cancerous tissue into the bloodstream relative to the total amount of cell-free DNA (cfDNA) present. A necessary first step for any liquid biopsy-based genomic analysis is therefore to obtain an accurate estimate of the circulating tumor fraction for the sample, i.e., the fraction of total cell-free DNA that comprises tumor-derived DNA. Existing processes for determining circulating tumor fraction, such as those based solely on somatic allele frequencies, or on cfDNA fragment size, suffer from significant performance limitations at low ctDNA levels. Therefore, there is a need for techniques that more accurately determine circulating tumor fraction.

In some instances, the methods and systems disclosed herein may be used to determine circulating tumor fraction (e.g., the amount of circulating tumor DNA (ctDNA) relative to the total amount of cell-free DNA (cfDNA)) in a sample. In some instances, the methods comprise determining a certainty metric which is indicative of the dispersion in, e.g., allele fraction across a plurality of analyzed loci. In some instances, the methods comprise the combined use of two different processes that, in combination, provide a more accurate, composite determination of circulating tumor fraction over a broader range of ctDNA concentrations. The first stage of the combined process comprises the determination of a certainty metric for the sample, as previously indicated. The certainty metric for the sample is compared to a reference curve that relates certainty metric values to the circulating tumor fraction for a series of samples comprising known percentages of circulating tumor DNA to determine a tumor fraction estimate for the sample. If the estimate of circulating tumor fraction, or a parameter related thereto, returned by the first stage of the combined process is less than or equal to a threshold value, a second stage of the combined process, i.e., for estimating circulating tumor fraction based on determining a somatic allele frequency, may be utilized, as will be discussed in more detail below.

Tumor fraction is closely associated with allele fraction dispersion across a plurality of analyzed loci. The dispersion can be referred to as a “certainty metric.” A relationship between one or more certainty metrics and one or more corresponding tumor fractions can be used to determine the tumor fraction of the sample from the determined certainty metric of the sample from the subject. The relationship receives the determined certainty metric as an input, and outputs the tumor fraction for the sample. This relationship can be applied to determine a tumor fraction of a sample from a subject, which can allow for effective tumor therapy, monitoring of the subject for tumor progression or recurrence, and/or analysis of a tumor sample.

In some embodiments, the tumor fraction of sample is determined for a tumor sample using the tumor sample and a non-tumor sample (e.g., a healthy tissue sample). The tumor sample and the non-tumor sample may be obtained from the same individual (i.e., a matched normal control) or different individuals. The certainty metric can be a dispersion for a plurality of values wherein each of the values are indicative of a difference between coverage of a locus in the tumor sample and coverage of the same locus in the non-tumor sample at a plurality of loci. As above, a relationship between certainty metrics and tumor fractions can be used to determine the tumor fraction of the sample from the determined certainty metric of the sample from the subject. The relationship receives the determined certainty metric as an input, and outputs the tumor fraction for the sample. This relationship can be applied to determine a tumor fraction of a sample from a subject, which can allow for effective tumor therapy, monitoring of the subject for tumor progression or recurrence, and/or analysis of a tumor sample.

In some embodiments, e.g., if the tumor fraction estimated by the certainty metric process is below a specified cut-off threshold or confidence level (e.g., a minimum detectable quantity for the tumor fraction of the sample, or a statistical lower bound for the tumor fraction estimate), an alternative process based on, e.g., determining an allele frequency (such as a maximum somatic allele frequency, a second highest somatic allele frequency, or the highest allele frequency that is less than some specified percentage or value) may be used to determine the tumor fraction (e.g., a circulating tumor fraction in a liquid biopsy sample). Thus, in some instances, the method for determining a tumor fraction of a sample from a subject may comprise: receiving a plurality of values, each value indicative of an allele fraction at a corresponding locus within a subgenomic interval in the sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; and outputting the second estimate of the tumor fraction of the sample. In some instances, this composite process may expand the lower bound limit of detection for determining tumor fraction while minimizing the possibility of overestimation of tumor fraction due to germline and clonal hematopoiesis (CH) variants.

### Tumor Fraction Determination

An important indicator in monitoring for, diagnosing, and treating cancer is tumor fraction. In some embodiments, tumor fraction is a measure of tumor genomic content, for example in a sample (e.g., biopsy), in proportion to the total genomic content regardless of cell origin. In general, it is advantageous to determine (e.g., estimate) tumor content, or a change in tumor content, from a sample, since this can aid in both reporting alterations and informing on disease presence or progression. For example, liquid biopsies, which typically utilize blood samples from cancer patients, can be useful when solid biopsies are not possible or recommended. The methods described herein can be used to determine tumor fraction in various types of samples, for example, in solid and liquid samples. In some embodiments, the methods described herein are used for solid samples, e.g., as an alternative, or in combination with, visual screening methods. In other embodiments, the methods described herein are used for liquid samples, e.g., when visual screening methods are not effective or available.

In some embodiments, tumor fraction in a cell-free sample comprises a measure of the tumor DNA that has shed into the vasculature or lymphatics from a primary tumor relative to the amount of total DNA (e.g., tumor and normal) shed into the blood stream, and is being carried around the body in the blood circulation. Tumor fraction can be used to monitor a patient at risk for cancer (with or without current diagnosis); as a factor used in diagnosing cancer; or to determine if a current treatment regimen is having an effect, e.g., a beneficial effect.

Traditional processes for measuring tumor fraction typically require that both purity and ploidy, modeled parameters, be inferred from either log ratio and allele frequency measurements or both, or from pathology review. In some embodiments, tumor fraction can be considered as a modeled parameter of the fraction of cancer cells in a heterogeneous tumor sample and can take into account tumor purity or other measures. In some embodiments, tumor cell ploidy can refer to the average weighted copy number of all chromosomes (or portions thereof). The ploidy observed in a sample can be impacted by the varying degrees of aneuploidy of tumor cells, the heterogeneity of the sample (e.g., different ratios of tumor cells to normal cells), or both.

Traditional processes for predicting tumor fraction can be highly unreliable for low tumor content due to poorly fit models. In some embodiments, the methods described herein can overcome certain drawbacks of the traditional processes, for example, by determining tumor fraction (and associated confidence levels) based on the effects of tumor cell aneuploidy, e.g., as measured by the allele coverage or allele fraction at one or more subgenomic intervals in a sample. In some embodiments, the subgenomic interval comprises a heterozygous single nucleotide polymorphism (SNP) site. In other embodiments, the subgenomic interval comprises more than one nucleotide positions.

The term “allele coverage,” or simply “coverage” or “Cvg” as used herein, refers to the number of reads (e.g., unique reads) generated from DNA sequencing of a subgenomic interval in a sample. The term “allele intensity,” or simply “intensity,” as used herein, refers to the number of signals (e.g., unique signals) generated from a genomic hybridization at a subgenomic interval in a sample. It will be appreciated that “reads” or “signal” is intended to encompass situations in which there may exist duplicates of the same “unique read” or “unique signal” (i.e., duplicates are not removed prior to performing the methods described herein), but any ratios calculated using the described methods will yield a value very similar to “unique” read or signal ratios, since the duplicates will be represented in both the numerator and denominator.

The term “allele fraction,” as used herein, refers to the relative level (e.g., abundance) of an allele at a subgenomic interval in a sample. Allele fraction can be expressed as a fraction or percentage. For example, allele fraction can be expressed as the ratio of the number of one particular allele (e.g., A, T, C, or G) at a subgenomic interval relative to the number of all different alleles at that subgenomic interval. In some embodiments, allele fraction is measured by determining the ratio of the coverage or intensity from one particular allele (e.g., A, T, C, or G) to the total coverage or intensity from all different alleles at a given subgenomic interval. Sometimes, the terms “allele fraction” and “allele frequency” are used interchangeably herein. As used herein, a log ratio is typically measured by log 2 (T/R), where T is the level (e.g., abundance) of one or more alleles associated with a subgenomic interval in a sample, and R is the level (e.g., abundance) of the one or more alleles associated with the subgenomic interval in a reference sample. The term, “allele,” as used herein, refers to one of the two or more alternative forms of a genomic sequence (e.g., a gene or any portion thereof). For example, if a “C” to “T” SNP is associated with a subgenomic interval, then the subgenomic interval can be described as being associated with alleles “C” and “T” with respect to the SNP.

In some embodiments, there are two or more different alleles associated with a subgenomic interval. If the two or more different alleles are present in a sample, the subgenomic interval is considered as heterozygous for the sample. If the subgenomic interval is not heterozygous for the sample, it can, in some embodiments, be homozygous, semizygous, or hemizygous.

The term, “abundance,” as used herein, refers to the amount, number, or quantity of an object. For example, the abundance of an allele associated with a subgenomic interval can mean the amount, number, or quantity of an allele associated with a subgenomic interval in a sample, for example, as determined by sequencing or array-based comprehensive genomic hybridization (aCGH). For example, if there are two alleles, “A” and “G,” associated with a particular subgenomic interval, and there are 10 copies of allele “A” and 20 copies of allele “G” in a sample, the abundance of allele “A” can be considered as 10 and the abundance of allele “G” can be considered as 20. In some embodiments, the abundance of an allele is measured by allele coverage or allele intensity. For example, the number of unique reads for allele “A” or “G” reflects how many copies of allele “A” or “G” are present in the sample.

The term “certainty metric,” as used herein, refers to a metric derived from a measure or value of a target variable. In some embodiments, the target variable may represent an abundance of a subgenomic interval, or an allele associated with the subgenomic interval, in a sample. In some examples, the certainty metric may be a deviation of an allele fraction from an expected allele fraction. In other examples, the certainty metric may be a measure of allele intensity. These examples are intended to be illustrative, and other certainty metrics may be used.

As an example, for a heterozygous SNP, an allele fraction value of 0.50 can indicate a typical diploid subgenomic interval; and an allele fraction that deviates from an expected value of 0.50 indicates aneuploidy at that site. In these examples, this deviation of allele coverage can be correlated with tumor fraction in a training set in order to build a model that determines (e.g., predicts or estimates) tumor fraction based on allele coverage. In some embodiments, the methods described herein correlate deviation of allele fraction or log ratio with tumor fraction, thereby eliminating the need to model tumor purity and ploidy. In some embodiments, the methods described herein allow for more accurate determination of tumor fraction of low level, e.g., less than 30%. In an embodiment, the allele fraction or log ratio is determined by a method comprising sequencing, e.g., next generation sequencing (NGS). It will be appreciated that the methods for determining allele fraction or log ratio are not limited to sequencing. Any method that measures, for example, SNP coverage or relative level (e.g., abundance) of SNPs, as well as, any method that measures coverage from larger genomic regions can be used. In an embodiment, the allele fraction or log ratio is determined by a method other than sequencing, e.g., is determined by an array-based comprehensive genomic hybridization (aCGH). In an embodiment, the tumor fraction is, or is expected to be, less than or equal to 0.25, less than or equal to 0.2, less than or equal to 0.15, or less than or equal to 0.1, e.g., between 0.1 and 0.3, between 0.1 and 0.2, between 0.2 and 0.3, or between 0.15 and 0.25.

While in some embodiments the methods described herein use allele fraction or log ratio to indicate expected coverage proportions, it will be appreciated that the present disclosure is generally intended to describe the correlation of tumor fraction to expected coverage deviations, without limitation to allele fraction, log ratio, or any other specific metric.

As used herein, a “single-nucleotide polymorphism,” or SNP, refers to an alteration of a single nucleotide that occurs at a specific position in the genome. In some embodiments, such alteration is present to some appreciable degree within a population (e.g., >1%). Typically, a SNP is a germline alteration and is not a somatic single-nucleotide variant (SNV).

In an embodiment, the tumor fraction is a numerical representation (e.g., fraction or percentage) indicating the amount of DNA from tumor cells versus the total amount of DNA (e.g., tumor and non-tumor DNA) in the sample. In an embodiment, the sample is a liquid biopsy. In an embodiment, the sample is a solid tissue sample. In an embodiment, the tumor is a solid tumor. In an embodiment, the tumor is a hematological cancer. In an embodiment, the tumor fraction in a liquid biopsy indicates the presence or level of detectable tumor in the body.

It should be understood by those skilled in that the outputs described herein may include, for example, determining, identifying or applying the value as a diagnostic value associated with the sample. In particular, the output may be used in generating a genomic profile for the subject and making suggested treatment decisions for the subject that may be applied or administered based on the outputs.

An exemplary method of determining a tumor fraction of a sample from a subject includes: acquiring a plurality of values, each value indicative of an allele fraction at a corresponding locus within a subgenomic interval in the sample; determining a certainty metric indicative of a dispersion of the plurality of values; accessing a predetermined relationship between a stored certainty metric and a stored tumor fraction; and determining, from the certainty metric and the predetermined relationship, the tumor fraction of the sample

A value indicative of an allele fraction can be determined for each corresponding locus. The loci include may include one or more nucleotide. In some embodiments, the corresponding loci comprise one or more loci having a different maternal allele and paternal allele. In some embodiments, the corresponding loci consist of loci having a different maternal allele and paternal allele. In some embodiments, the corresponding loci comprise one or more loci having the same maternal allele and paternal allele.

In some embodiments, the plurality of values indicative of an allele fraction at a plurality of corresponding loci in the sample is a plurality of allele fractions at the plurality of corresponding loci in the sample. The allele fraction at each of the corresponding loci may be determined, for example, by sequencing nucleic acid molecules in the tumor sample and assigning an allele coverage for each allele at each locus. For example, the allele fraction at locus i (afᵢ) may be determined by:

$${af}_{i} = \frac{{Cvg}_{i , a}}{{Cvg}_{i , a} + {Cvg}_{i , b}}$$

wherein Cvg_{i,a} is the coverage of allele a at locus i, and Cvg_{i,b} is the coverage of allele b at locus i. In some embodiments, allele a and allele b are assigned such that Cvg_{i,a}≤Cvg_{i,b}, such that afᵢ≤0.5.

In some embodiments, the expected allele fraction is the allele fraction expected in a healthy individual or healthy sample (i.e., a non-tumor sample). For example, the allele fraction at a heterozygous locus (that is, having a different maternal allele and paternal allele) is expected to be 0.5, and the allele fraction at a homozygous locus (that is, wherein the maternal allele and the paternal allele are the same) is expected to be 1.0.

Allele fraction is an exemplary value for determining tumor fraction according to the methods described herein, although other values indicative of allele fraction may be used in some embodiments. In some embodiments, the value indicative of the allele fraction is a relative difference in allele frequency. For example, the value indicative of the allele fraction may be ratio of the difference in the abundance (e.g., a coverage or sequencing depth) between a maternal allele and a paternal allele relative to the abundance of the maternal allele or the paternal allele. That is, in some embodiments, the value can a relative_difference as de

$$relative\_difference = \frac{{Cvg}_{i , a} - {Cvg}_{i , b}}{{Cvg}_{i , b}}$$

wherein Cvg_{i,a} is the coverage of allele a at locus i, and Cvg_{i,b} is the coverage of allele b at locus i. In a healthy individual or healthy sample, the difference between the allele frequency, as well as the relative difference, is expected to be 0. In some embodiments, a probability distribution function is determined for the plurality of values indicative of allele fraction. For example, in some embodiments, the probability distribution function is determined for the plurality of allele fractions at the plurality of corresponding loci in the sample. In some embodiments, the probability distribution function for the plurality of allele fractions is defined by:

$$P ( af ) = P ( \frac{{Cvg}_{i , a}}{{Cvg}_{i , a} + {Cvg}_{i , b}} )$$

wherein Cvg_{i,a} is the coverage of allele a at locus i, and Cvg_{i,b} is the coverage of allele b at locus i.

The dispersion (or certainty metric) can be, for example, a deviation from the expected allele fraction (or value indicative of expected allele fraction) across the plurality of loci. In some embodiments, the certainty metric is a root mean squared deviation from the expected allele fraction (or value indicative thereof). For example, in some embodiments, the certainty metric is a root mean squared deviation (RMSD) defined by:

$$R M S D = {[ \frac{1}{N} {\sum }_{i = 0}^{N} {( {af}_{i} - {af}_{i , expected} )}^{2} ]}^{( 1 / 2 )}$$

wherein afᵢ is the allele frequency (or value indicative of the allele frequency, such as a relative difference ratio) at locus i, af_{expected} is the expected allele frequency at locus i, and N is the number of loci in the plurality of corresponding loci. For example, for some loci, af_{expected} may be 0.5, and at other loci af_{expected} may be 1. In some embodiments, the loci include only those loci having a different maternal allele and paternal allele. Thus, the af_{expected} may be defined as 0.5 across all loci, and the RMSD can be defined as:

$$RMSD = {[ \frac{1}{N} {\sum }_{i = 1}^{N} {( {af}_{i} - 0.5 )}^{2} ]}^{( 1 / 2 )}$$

In some embodiments, the value indicative of the allele fraction may be ratio of the difference in abundance (e.g., a coverage or sequencing depth) between a maternal allele and a paternal allele, relative to the abundance of the maternal allele or the paternal allele, and the af_{expected} may be defined as 0. Thus, the RMSD can be defined as:

$$RMSD = {[ \frac{1}{N} {\sum }_{i = 0}^{N} {( \frac{{Cvg}_{i , a} - {Cvg}_{i , b}}{{Cvg}_{i , b}} )}^{2} ]}^{( 1 / 2 )}$$

wherein Cvg_{i,a} is the coverage of allele a at locus i, and Cvg_{i,b} is the coverage of allele b at locus i.

In some embodiments, a probability distribution (e.g., a probability distribution function) can be determined for allele fractions across a plurality of loci. The certainty metric (e.g., a dispersion) can be a metric of the probability distribution, such as an entropy of the probability distribution. For example, in some embodiments, the entropy of an allele fraction probability distribution function (S[P(af)]) may be defined as:

$$S [ P ( af ) ] = {\sum }_{af = 0}^{0.5} P ( af ) {log}_{n} ( P ( af ) )$$

wherein P(af) is the allele fraction probability distribution function, and n is the log base. In some embodiments, the log base is 2 (i.e., log₂). Accordingly, in some embodiments, the entropy of an allele fraction probability distribution function (S[P(af)]) may be defined as:

$$S [ P ( af ) ] = {\sum }_{af = 0}^{0.5} P ( af ) {log}_{n} ( P ( af ) )$$

In some embodiments, a method of determining a tumor fraction of a sample from a subject, the method comprising: acquiring a plurality of values, each value indicative of a difference between an allele coverage of a locus in a tumor sample and an allele coverage of the same locus in a non-tumor sample at a plurality of loci within a subgenomic interval; determining a certainty metric indicative of a dispersion of the plurality of values; accessing a predetermined relationship between a stored certainty metric and a stored tumor fraction; and determining, from the certainty metric and the predetermined relationship, the tumor fraction of the sample. In some embodiments, the tumor sample and the non-tumor sample are obtained from the same individual (i.e., a matched normal control). In some embodiments, the tumor sample and the non-tumor sample are obtained from different individuals. The coverage may be a raw coverage (for example, a raw number of sequencing reads), a normalized coverage (for example, normalized to a mean or median sequencing depth), and/or otherwise bias-corrected coverage (for example, a GC-bias corrected coverage depth). In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele).

In some embodiments, each value indicative of the difference between an allele coverage of the locus in a tumor sample and an allele coverage of the same locus in the non-tumor sample comprises a ratio of the allele coverage of a locus in the tumor sample compared to the allele coverage of the same locus in the non-tumor sample. In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, in some embodiments, the ratio may be defined as:

$$ratio = \frac{( {Cvg}_{i , a}^{cancer} + {Cvg}_{i , b}^{cancer} )}{( {Cvg}_{i , a}^{normal} + {Cvg}_{i , b}^{normal} )}$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

In some embodiments, each value indicative of the difference between an allele coverage of the locus in a tumor sample and an allele coverage of the same locus in the non-tumor sample is a log ratio (such as a log 2 ratio) of the allele coverage of a locus in the tumor sample compared to the allele coverage of the same locus in the non-tumor sample. In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, the log ratio may be defined, in some embodiments, as:

$${log}_{n} ratio = {log}_{n} \frac{( {Cvg}_{i , a}^{cancer} + {Cvg}_{i , b}^{cancer} )}{( {Cvg}_{i , a}^{normal} + {Cvg}_{i , b}^{normal} )}$$

wherein logₙ is the log at base n, Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample. For example, the log ratio may be a log 2 ratio. In some embodiments, the log ratio is defined as:

$${log}_{2} ratio = {log}_{2} \frac{( {Cvg}_{i , a}^{cancer} + {Cvg}_{i , b}^{cancer} )}{( {Cvg}_{i , a}^{normal} + {Cvg}_{i , b}^{normal} )}$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample

In some embodiments, each value indicative of the difference between an allele coverage of the locus in a tumor sample and an allele coverage of the same locus in the non-tumor sample comprises a ratio of the difference between the allele coverage of a locus in the tumor sample compared to the allele coverage of the same locus in the non-tumor sample, relative to the allele coverage of the same locus in the non-tumor sample. In some embodiments, the allele coverage comprise the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, in some embodiments, the ratio is defined as:

$$\frac{( {Cvg}_{i , a}^{cancer} + {Cvg}_{i , b}^{cancer} ) - ( {Cvg}_{i , a}^{normal} + {Cvg}_{i , b}^{normal} )}{( {Cvg}_{i , a}^{normal} + {Cvg}_{i , b}^{normal} )}$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

In some embodiments, a probability distribution function is determined for the plurality of values indicative of the difference between an allele coverage of the locus in a tumor sample and an allele coverage of the same locus in the non-tumor sample. In some embodiments, the allele coverage comprises the coverage of a maternal allele and a coverage of a paternal allele (such as a sum of the coverage of the maternal allele and the coverage of the paternal allele). In some embodiments, the allele coverage consists the coverage of a maternal allele and a coverage of a paternal allele (such as a sum of the coverage of the maternal allele and the coverage of the paternal allele). For example, in some embodiments, the probability distribution function is determined for the plurality of ratios of the allele coverage of a locus in the tumor sample compared to the allele coverage of the same locus in the non-tumor sample (such as a log ratio, for example a log 2 ratio). In some embodiments, the probability distribution function for the plurality of allele fractions is defined by:

$$P ( {log}_{n} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein logₙ is the log at base n, Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample. In some embodiment, log ratio is a log 2 ratio. For example, in some embodiments, the probability distribution function for the plurality of allele fractions is defined by:

$$P ( {log}_{2} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

The dispersion (or certainty metric) can be, for example, a deviation of each value within the plurality of values from an expected value across the corresponding loci. The expected value is the value that would be expected if the tumor sample were non-tumor (e.g., a healthy) sample. In some embodiments, the certainty metric is a root mean squared deviation from the expected value. For example, in some embodiments, the certainty metric is a root mean squared deviation (RMSD) defined by:

$$RMSD = \sqrt{\frac{1}{N} {\sum }_{i}^{N} [ ( {log}_{2} \frac{( {Cvg}_{i , a}^{cancer} + {Cvg}_{i , b}^{cancer} )}{( {Cvg}_{i , a}^{normal} + {Cvg}_{i , b}^{normal} )} ) - {0}^{2}}$$

In some embodiments, the value indicative of the allele fraction is a ratio of the difference between the allele coverage of a locus in the tumor sample compared to the allele coverage of the same locus in the non-tumor sample, relative to the allele coverage of the same locus in the non-tumor sample. Thus, the RMSD can be defined as:

$$RMSD = {[ \frac{1}{N} {\sum }_{i = 0}^{N} {( \frac{( {Cvg}_{i , a}^{cancer} + {Cvg}_{i , b}^{cancer} ) - ( {Cvg}_{i , a}^{normal} + {Cvg}_{i , b}^{normal} )}{( {Cvg}_{i , a}^{normal} + {Cvg}_{i , b}^{normal} )} )}^{2} ]}^{( 1 / 2 )}$$

In some embodiments, a probability distribution (e.g., a probability distribution function) can be determined for the plurality of values indicative of the difference between an allele coverage of the locus in a tumor sample and an allele coverage of the same locus in the non-tumor sample. The certainty metric (e.g., a dispersion) can be a metric of the probability distribution, such as an entropy of the probability distribution. For example, in some embodiments, the entropy of an allele fraction probability distribution function (S[P(af)]) may be defined as:

$$S [ P ( lnr ) ] = {\sum }_{l n r = 0} P ( lnr ) {log}_{n} ( P ( lnr ) )$$

wherein:

$$P ( lnr ) = P ( {log}_{n} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein logₙ is a log having base n. Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample. In some embodiments, the log base is 2 (i.e., log 2). Accordingly, in some embodiments, the entropy of an allele fraction probability distribution function (S[P(af)]) may be defined as:

$$s [ P ( l 2 r ) ] = {\sum }_{lnr = 0} P ( l 2 r ) {log}_{2} ( P ( l 2 r ) )$$

wherein:

$$P ( l 2 r ) = P ( {log}_{2} ( \frac{{Cvg}_{i , a}^{cancer} + {Cvg}_{i , b}^{cancer}}{{Cvg}_{i , a}^{normal} + {Cvg}_{i , b}^{normal}} ) )$$

wherein Cvg_{i,a}^{Cancer} is the coverage of the maternal allele at the locus i within the tumor sample, Cvg_{i,b}^{Cancer} is the coverage of the paternal allele at the locus i within the tumor sample, Cvg_{i,a}^{Normal} is the coverage of the maternal allele at the locus i within the non-tumor sample, and Cvg_{i,b}^{Normal} is the coverage of the paternal allele at the locus i within the non-tumor sample.

A relationship between one or more stored certainty metrics and one or more stored tumor fractions can be used to determine the tumor fraction based on the determined certainty metrics. In some embodiments, a model is trained to using a training dataset that includes training certainty metrics and associated tumor fractions to determine the relationship between the certainty metrics and the tumor fractions. The training dataset may be determined, for example, using a plurality of clinical samples with known (i.e., training) tumor fractions (for example, as determined by maximum somatic allele frequency (MSAF), which filters germline variant calls from all calls in a tumor sample and compares residual variants (i.e., the maximum somatic variants) to the total variants (maximum somatic variants plus germline variants) to determine the maximum somatic allele frequency). Nucleic acid molecules in the clinical samples can be sequenced to determine allele frequency across a plurality of loci (or a value indicative of an allele frequency), as well as an associated training certainty metric. The training certainty metrics can be correlated with the training tumor fractions to determine the relationship between certainty metric and tumor fraction. In another method, serial dilutions may be made from one or more clinical samples to obtain a plurality of different tumor fractions, which can be correlated with the certainty metric for the serially diluted samples to determine the relationship.

In some embodiments, to determine (e.g., estimate) the tumor fraction, a training subprocess is first performed. A dataset can be constructed from clinical specimens. Using the training set and in-silico dilutions of the training set, tumor fraction can be correlated to variation in allele fractions or log ratios corresponding to aneuploidy typically observed in tumors. In other examples, cell-line/clinical sample dilutions can be performed.

In some embodiments, the certainty metric may be functions of the coverage at particular SNP bins for particular alleles and/or an allele frequency (e.g., in the range of 0 to 0.5). In some examples, the training data uses as input a deviation metric (e.g., allele fraction deviation or log ratio deviation) and returns the estimated tumor fraction, along with lower and upper bounds. Values that deviate from (i.e., fall between) 0 and 1 and not 0.5 (exclusive) may be thought of as “noise,” and the averaged noise may be correlated with an expected or estimated tumor fraction. In other examples, the training data provides as input a log ratio deviation metric, or in general, any metric which quantifies coverage deviations from expectations. In either case, the allele coverage deviation metric or the log ratio deviation metric may be a measure of the tumor fraction.

Utilizing these correlations derived during training, a tumor fraction of a patient can be estimated or evaluated with upper and lower bounds. Coverage metrics, such as SNP allele coverage variation metrics, may be used in generating the correlation.

The methods described herein can, for example, improve the ability to identify whether tumor is present in biological samples and provide tumor fraction determination (e.g., estimates) with known estimate bounds; provide a systematic and orthogonal process to assess somatic variants; and provide the framework for a new inexpensive tumor-tracking/identifying assay.

In some embodiments the methods described herein also offer advantages in the particular case of liquid biopsies (though this disclosure is not limited to liquid biopsies). Solid tumors have multiple different means for estimating tumor content, including pathology review, somatic allele frequencies (MSAF), and analytical copy number alteration (CNA) modeling. Liquid biopsies, however, are typically not suitable for these methods or require significant re-adjustment. Since cell-free DNA is free floating in the blood, its presence is nanoscopic, and thus, cannot be reviewed by a pathologist. Further, the amount of DNA the tumor tends to shed into the blood stream may be minuscule in comparison to normal DNA. As such, analytical CNA modeling may fail due to low tumor content.

The methods described herein typically do not require pathology review; are sufficiently sensitive and free of analytical equations, such that analytical CNA modeling is not needed to identify tumor presence or content; are independent of short variant calling, providing an orthogonal assessment of short variants; and are improved (e.g., not confounded) when there are CNA events.

The methods described herein allow for the development of a new inexpensive tumor-tracking (e.g., monitoring) assay. For example, if a patient presents tumor content in an assay (e.g., a comprehensive genomic profiling assay) that covers a sufficient number of subgenomic intervals (e.g., subgenomic intervals that include one or more SNPs), the tumor progression can be tracked over-time on a second assay for considerably less cost since this method can be based solely on SNP variation. In some embodiments, the first assay covers more subgenomic intervals than the second assay. In other embodiments, the first assay covers fewer subgenomic intervals than the second assay. In certain embodiments, the first assay and the second assay cover essentially the same number of subgenomic intervals.

The gene panels included in the first and second assays may have the same or different sizes. For example, an assay that includes a panel of at least about 100, 150, 200, 250, 300, 350, 400, 450, 500, or more genes may be considered as a large panel, and an assay that includes fewer than about 100, 90, 80, 70, 60, 50, 40, 30, 20, or 10 genes may be considered as a small panel. The “large” and “small” panel sizes are typically determined by the purposes of the assays and should not be limited to the exemplary sizes above. In some embodiments, the first assay includes a large panel and the second assay includes the same or a different large panel. In other embodiments, the first assay includes a small panel and the second assay includes the same or a different small panel. In certain embodiments, the first assay includes a large panel and the second assay includes a small panel, or vice versa. The first and second assays need not be the same assay type. For example, the first assay can be based on sequencing (e.g., NGS) and the second assay can be based on genomic hybridization, or vice versa.

In some embodiments, the subgenomic intervals covered by the second assay may be a subset of the subgenomic intervals covered by the first assay. In some embodiments, the subgenomic intervals covered by the first assay may be a subset of the subgenomic intervals covered by the second assay. In other embodiments, the subgenomic intervals covered by the second assay overlap with the subgenomic intervals covered by the first assay, but are not the same. In certain embodiments, the first assay covers one or more subgenomic intervals that are not covered by the second assay. In certain embodiments, the second assay covers one or more subgenomic intervals that are not covered by the first assay.

In some embodiments, even though the estimated tumor fraction may have wide margins of error across patients, any intra-patient comparison will provide small margins of error, leading to the ability to track the progression of the tumor originally identified in the comprehensive genomic profiling assay. Since the second assay could be much less expensive than the comprehensive genomic profiling assay, it can be used as a standard screening technique for at least a subset of patients, such as at-risk patients, to answer the question whether the patient has cancer.

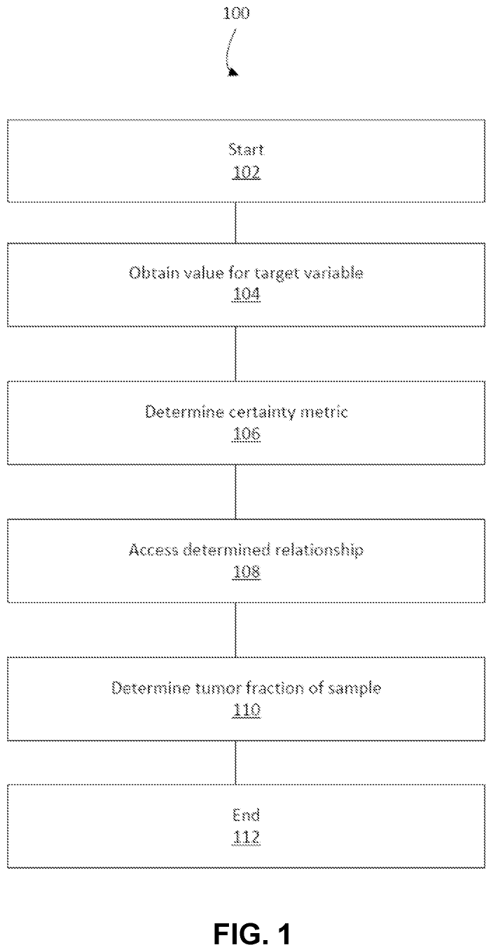

FIG. 1 shows a method 100 of estimating a tumor fraction from a sample. The method 100 begins at step 102. At step 104, a value for a target variable associated with a subgenomic interval is obtained, e.g., directly obtained, from a sample from a subject. The target variable may be, for example, an allele fraction. The sample may be, e.g., a liquid sample or a solid sample.

In some examples, a patient allele fraction for at least one heterozygous single nucleotide polymorphism (SNP) site is determined from a biopsy taken from a patient. In one example, the biopsy may be a liquid biopsy, i.e., a sample of non-solid biological tissue, for example, blood. The disclosure is not so limited, however, and is intended to cover any solid or liquid assays or biopsies without limitation. In an embodiment, the liquid biopsy comprises a blood sample. In an embodiment, the liquid biopsy comprises cell free DNA (cfDNA). In an embodiment, the liquid biopsy comprises circulating tumor DNA (ctDNA). In an embodiment, the liquid biopsy comprises DNA shed from a tumor. In an embodiment, the liquid biopsy comprises nucleic acids other than DNA, e.g., RNA. In an embodiment, the liquid biopsy comprises circulating tumor cells (CTCs). Other types of liquid biopsies are described, e.g., in Crowley et al. Nat Rev Clin Oncol. 2013; 10(8):472-484, the content of which is incorporated by reference in its entirety.

At step 106, a certainty metric may be determined from the target variable, and at step 108, a determined relationship is accessed between a stored certainty metric and a stored tumor fraction. The determined relationship may include historical sample data (collected from patients or other test subjects) relating a certainty metric (e.g., a sampled allele fraction deviation) for at least one heterozygous SNP site to a corresponding sampled tumor fraction. In some examples, the sampled allele coverage deviation is a “noise” metric, reflecting the degree to which an allele fraction varies from an expected value. In some examples, the number of data points correlating tumor fraction to noise metrics calculated from the allele fraction may exceed one hundred (100), one thousand (1,000), ten thousand (10,000), or more.

In one example, the determined relationship may be derived from an in silico process, and the analysis may be performed by a machine learning process. The process may perform a sample dilution (e.g., using a matched normal) starting at a particular tumor fraction in order to correlate one or more coverage deviation metrics (e.g., allele fraction values) across one or more subgenomic intervals (e.g., SNPs, SNP bins, and/or chromosomes). The metric may be a measure of the frequency and degree to which tumor fraction falls in between the values of 0 or 1. Averaged “noise” metrics between 0 and 1 (exclusive) may be correlated with an expected or estimated tumor fraction. In some embodiments, the disclosed methods may comprise: obtaining a training dataset comprising a plurality of relationships between a plurality of training certainty metric values and associated training tumor fraction values; training a machine learning model based on the training dataset; and using the trained machine learning model to determine a tumor fraction value from the certainty metric value for the sample.

The number of elements associated with subgenomic intervals that contribute to the determination of the certainty metric value, which is correlated to tumor fraction, may be on the order of ten (10), one hundred (100), one thousand (1,000), ten thousand (10,000), or more.

Due to the large number of elements associated with subgenomic intervals that contribute to the certainty metric determination in the correlation, the elements may be “binned” or aggregated by subgenomic interval position or other characteristics in some examples. Binning may avoid a single (or small set of) element(s) disproportionately weighting a correlation in the certainty metric, adversely affecting the estimated tumor fraction. For example, if one element at a single subgenomic interval represents a copy variant with 5,000 copies, it may result in an estimated tumor fraction that is inaccurately high. Therefore, in some examples, elements that contribute to a certainty metric are averaged or otherwise aggregated by chromosome, for example, for each of 22 relevant chromosomes. Those 22 aggregate chromosome values can then be used to calculate the certainty metric which is then correlated with tumor fraction, ensuring that a single subgenomic interval (e.g., SNP site) does not disproportionately affect the correlation. Other methods can be utilized to limit the effect of extreme copy-number events, such as, but not limited to, excluding outlier values from the certainty metric determinations.

In some examples, the correlation may be a mean (i.e., average) correlation, with upper bound correlations and lower bound correlations also calculated. In this way, the mean correlation is bounded by a 95% confidence interval.

The subgenomic interval may comprise one or several subgenomic intervals, and in some examples may be at least one heterozygous SNP site. Subgenomic intervals may be selected based on various criteria. For example, subgenomic intervals may be selected based on how polymorphic the subgenomic interval is in a general healthy population, as well as, healthy subpopulations (including different genders, ages or ethnic backgrounds). It may be advantageous that the subgenomic intervals vary considerably in the healthy population. The sequencing characteristics of the subgenomic intervals may also be selected on the basis of being “well-behaved,” i.e., near expected allele-frequencies, such as 0, 0.5, and 1.0. Furthermore, the regions may be selected on the basis of being “well covered,” i.e., having typical coverage across populations for the site. Subgenomic intervals may be excluded if they occur in simple repeats of gene families or in any generally repeating sequence of DNA, since this characteristic can challenge alignment methodologies. In an embodiment, subgenomic intervals may be located in a genomic region that is free, or essentially free, of high homology, simple repeats, or gene families.

In an embodiment, the subgenomic interval comprises a minor allele. As used herein, a “minor allele” is an allele other than the most common allele (e.g., the second most common allele or the least common allele) associated with a particular subgenomic interval in a given population. In an embodiment, at least 10, 20, 50, 100, 150, 200, 300, 400, 500, 600, 700, 800, 900, 1000, 1200, 1400, 1600, 1800, 2000, or 10000 heterozygous subgenomic intervals are selected. In one example, no more than 10, 20, 50, 100, 150, 200, 300, 400, 500, 600, 700, 800, 900, 1000, 1200, 1400, 1600, 1800, 2000, or 10000 heterozygous SNP sites are selected.

In one example, the selected subgenomic intervals and/or correlation may be universal, i.e., across all disease ontologies, in order to provide a broad screening technique. In other examples, subgenomic intervals may be selected, and the correlation tuned, based on disease ontology (e.g., tumor type).

One or more certainty metrics may be used in correlating a target variable (e.g., allele coverage deviation and/or allele fraction variation) to tumor fraction. For example, metrics relating to allele fraction may be applied. In one example, an allele frequency entropy metric or root mean squared deviation (RMSD) metric may be used:

$$Allele Frequency Entropy :$$

$$S [ P ( af ) ] = {\sum }_{af = 0}^{0.5} P ( af ) {log}_{2} ( P ( af ) )$$

$$Root Mean Squared Deviation :$$

$$RMSD = {[ \frac{1}{N} {\sum }_{i = 0}^{N} {( {af}^{i} - 0.5 )}^{2} ]}^{( 1 / 2 )}$$

where i=SNP bin and af=allele frequency on the range of 0 to 0.5. Folded SNP allele frequencies are used here by convention (e.g., as described in Nielsen. Hum Genomics. 2004; 1(3): 218-224 and Marth, et al. Genetics. 2004; 6(1): 351-372), but the methodology holds if the full range of 0 to 1 is utilized. Other metrics may also be used, such as metrics based off the log 2 ratio. Any of these metrics may incorporate factors such as coverage at a particular SNP bin, where the “bin” can be defined to be 1 or more base-pairs. In some embodiments, the certainty metric may be written as a function of coverage, such that certainty_metric=f(Cvg). Further, any mathematical transformation or operation acting on the certainty_metric, may also be considered a certainty_metric.

In some examples, the certainty metric may be a deviation from the expected log₂ ratio for at least one subgenomic interval. In other examples, the certainty metric may be a deviation from expected allele fraction in a healthy population for at least one subgenomic interval (e.g., a SNP) that is known to be heterozygous. In other examples, the certainty metric may be a deviation from expected allele coverage in the healthy population for at least one subgenomic interval (e.g., a SNP) that is known to be heterozygous.

Table 1 shows exemplary certainty metrics that may be used, including any p-moment or combination thereof:

TABLE 1

| Conditions to Relate | | |
|---|---|---|
| Metric to Tumor | Metric Calculating Relative Certainty | |
| Fraction | of Variable | Comments |
| INTRA-SAMPLE- | | |
| Comparisons | | |
| • All coverage under consideration is from the cancer sample • af is a metric which compares coverages from the maternal and paternal chromosomes • Cvgₐ is maternal or paternal, Cvg_{b} is the | variable = af $af = \frac{{Cvg}_{a}}{{Cvg}_{a} + {Cvg}_{b}} {af}_{expectation} = 0.5$ $certainty\_metric = \sqrt{\frac{1}{N} {\sum }_{i}^{N} {( af - {af}_{expectation} )}^{2}} = \sqrt{\frac{1}{N} {\sum }_{i}^{N} {( \frac{{Cvg}_{i , a}}{{Cvg}_{i , a} + {Cvg}_{i , b}} - 0.5 )}^{2}}$ | • Intra-sample comparison • Cvgₐ < Cvg_{b}, so that af ≤ 0.5 |
| other allele. | | |
| • N is a number of | | |
| subgenomic intervals, | | |
| and i is an index of N. | | |
| • Any loci may be used | | |
| such that the germline | | |
| maternal and paternal | | |
| chromosomes differ at | | |
| the same genomic | | |
| location; could be a SNP | | |
| or something larger. | | |
| • All coverage under | variable = Cvg | • Intra-sample comparison |
| consideration is from the cancer sample | $relative\_difference = \frac{{Cvg}_{a} - {Cvg}_{b}}{{Cvg}_{b}}$ | • Cvg. > Cvgb |
| • Cvgₐ is maternal or | relative_difference_{expectation} = 0 | • Previous choice is chosen |
| paternal, Cvg_{b} is the other allele. • Any loci may be used such that the germline | $certainty\_metric = \sqrt{\frac{1}{N} {\sum }_{i}^{N} {( \frac{{Cvg}_{i , a} - {Cvg}_{ib}}{{Cvg}_{i , b}} )}^{2}}$ | because we assume the allele is amplified and thus the “b” allele would be the normal. In general, it should not |
| maternal and paternal | | matter since this will switch |
| chromosomes differ at | | for deletions. Thus, Cvgₐ |
| the same genomic | | could be < Cvg_{b}, or one |
| location; could be a | | could completely ignore |
| SNP or something | | the convention |
| larger. | | |
| • All coverage under consideration is from the cancer sample • af is a metric which compares coverages from the maternal and paternal chromosomes | variable = af $probability of allele frequency = P ( af ) = P ( \frac{{Cvg}_{i , a}}{{Cvg}_{i , a} + {Cvg}_{i , b}} )$ certainty_metric = S[P(af)] | • Intra-sample comparison • S = Entropy is a metric that inherently measures relative certainty of the variable |
| • Cvgₐ is maternal or | | |
| paternal, Cvg_{b} is the | | |
| other allele. | | |
| • Any loci may be used | | |
| such that the germline | | |
| maternal and paternal | | |
| chromosomes differ at | | |
| the same genomic | | |
| location; could be a SNP | | |
| or something larger. | | |
| INTER- SAMPLE- | | |
| Comparisons | | |
| • All coverage under | variable = log2ratio = l2r | • Inter-sample comparison |
| consideration is from both the cancer and reference sample | $ratio = \frac{( {Cvg}_{a}^{cancer} + {Cvg}_{b}^{cancer} )}{( {Cvg}_{a}^{normal} + {Cvg}_{b}^{normal} )}$ | |
| • ratio is determined | 12r = log₂ (ratio) | |
| from the total coverage | 12r_{expectation} = 0 | |
| from maternal and paternal from cancer sample to the maternal and paternal from the | $certainty\_metric = \sqrt{\frac{1}{N} {\sum }_{i}^{N} {[ l 2 r - l 2 {r}_{expectation} ]}^{2}} =$ | |
| reference. | $\sqrt{\frac{1}{N} {\sum }_{i}^{N} {[ ( {log}_{2} \frac{( {Cvg}_{i , a}^{cancer} + {Cvg}_{i , b}^{cancer} )}{( {Cvg}_{i , a}^{normal} + Cvgi {,}_{b}^{normal} )} ) - 0 ]}^{2}}$ | |
| • Any loci of the genome | | |
| from 1 to an infinite set | | |
| of bases can be used. | | |
| • All coverage under | variable = Cvg | • Inter-sample comparison |
| consideration is from | Cvg_{cancer} = (Cvgₐ^{cancer} + Cvg_{b}^{cancer}) | |
| both the cancer and | Cvgₙₒᵣₘₐₗ = (Cvgₐ^{normal} + Cvg_{b}^{normal}) | |
| reference sample • ratio is determined from the total coverage | $relative\_difference = \frac{{Cvg}_{cancer} - {Cvg}_{normal}}{{Cvg}_{normal}}$ | |
| from maternal and | relative_difference_{expectation} = 0 | |
| paternal from cancer | | |
| sample to the maternal | | |
| and paternal from the | | |
| reference. | | |
| • Any loci of the genome from 1 to an infinite set of bases can be used. | $certainty\_metric = \sqrt{\frac{1}{N} {\sum }_{i}^{N} {( \frac{{Cvg}_{i , cancer} - {Cvg}_{i , normal}}{{Cvg}_{i , normal}} )}^{2}}$ | |
| • Total coverage from | variable = log2ratio = l2r | |
| maternal and paternal from cancer sample is compared to the maternal | $probability of log 2 ratio = P ( l2r ) = P ( {log}_{2} ( \frac{{Cvg}_{i , a}^{cancer} + {Cvg}_{i , b}^{cancer}}{{Cvg}_{i , a}^{normal} + {Cvg}_{i , b}^{normal}} ) )$ | • Inter-sample comparison • S = Entropy is a metric that does not calculate a |
| and paternal from the | certainty metric = S[P(l2r)] | deviation from expectation. |
| reference. | | It is a metric that inherently |
| • Any loci of the genome | | measures relative certainty |
| from 1 to an infinite set | | of the variable |
| of bases can be used | | |

At step 110, the tumor fraction of the sample is determined (e.g., estimated) with reference to the certainty metric and the determined relationship. In some examples, the coefficients of the determined relationship are applied to the certainty metric determined from the patient sample, and the products summed to arrive at an evaluated (e.g., estimated) tumor fraction. It will be appreciated that other functions may be performed to yield a final estimated tumor fraction. For example, the estimated tumor fraction may be scaled, normalized, or otherwise adjusted from an initial or raw estimated tumor fraction measure.

At step 112, method 100 ends.

The estimated tumor fraction may be used by a medical practitioner in a number of ways. For example, the estimated tumor fraction may be used to monitor a patient at risk for one or more types of cancer. The estimated tumor fraction may also be used to diagnose cancer, or to determine if a treatment of cancer is successfully affecting the tumor.

The estimated tumor fraction may also be used in connection with other screening techniques to confirm or validate test results. For example, a CNA screening may yield multiple possible combinations of purity and ploidy for a patient, particularly in a patient having a low tumor fraction (e.g., less than 30%). The present technique can be used to disambiguate such results,

In some embodiments, a report may be generated comprising the estimated tumor fraction. In an embodiment, the report further comprises a treatment option based on the estimated tumor fraction. In an embodiment, the report further comprises prognosis based on the estimated tumor fraction.

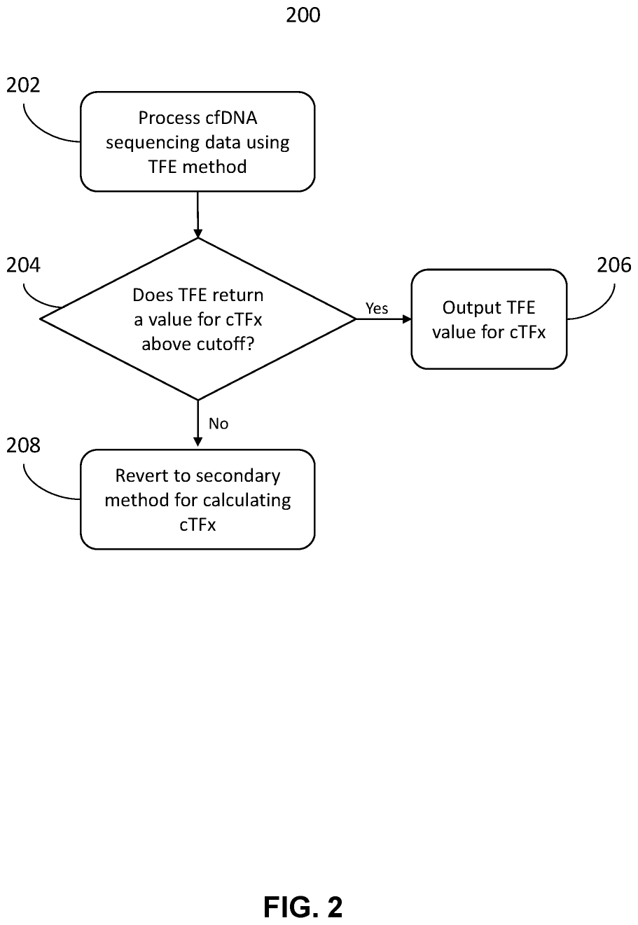

FIG. 2 provides another non-limiting example of a process 200 for determining the tumor fraction in a sample according to embodiments of the present disclosure. Sequencing data (e.g., cell-free DNA (cfDNA) sequencing data obtained using a CGP assay) representing values for, e.g., allele fraction, at a plurality of loci within a genome or subgenomic interval of a subject may be processed according to a first tumor fraction estimation process 202 (e.g., the tumor fraction estimator (TFE) process 100 described in FIG. 1). In some instances, the values derived from the sequencing data may represent, e.g., a difference between an allele coverage of a locus in a tumor sample and an allele coverage of the same locus in a non-tumor sample at the plurality of loci within the genome or subgenomic interval of the subject. The estimate of tumor fraction (e.g., a circulating tumor fraction) for the sample returned by the first stage determination is compared to a first threshold, 204. The first threshold may be, for example, a limit-of-detection (LoD) or specified confidence level for determining tumor fraction using the first stage determination. If the estimated tumor fraction returned by the first stage determination is greater than the first threshold, the estimate is output as the determined value of the tumor fraction for the sample, 206. If the estimated tumor fraction returned by the first stage determination is less than or equal to the first threshold, a secondary process may be used to calculate tumor fraction for the sample, 208. In some instances, the secondary method 208 may comprise the use of, for example, a maximum somatic allele frequency (MSAF) determination to estimate tumor fraction of the sample. In some instances, the use of two complementary processes in a composite methodology for determining tumor fraction provides for more accurate determinations of tumor fraction over a larger range of DNA concentrations (e.g., circulating tumor DNA (ctDNA) concentrations).

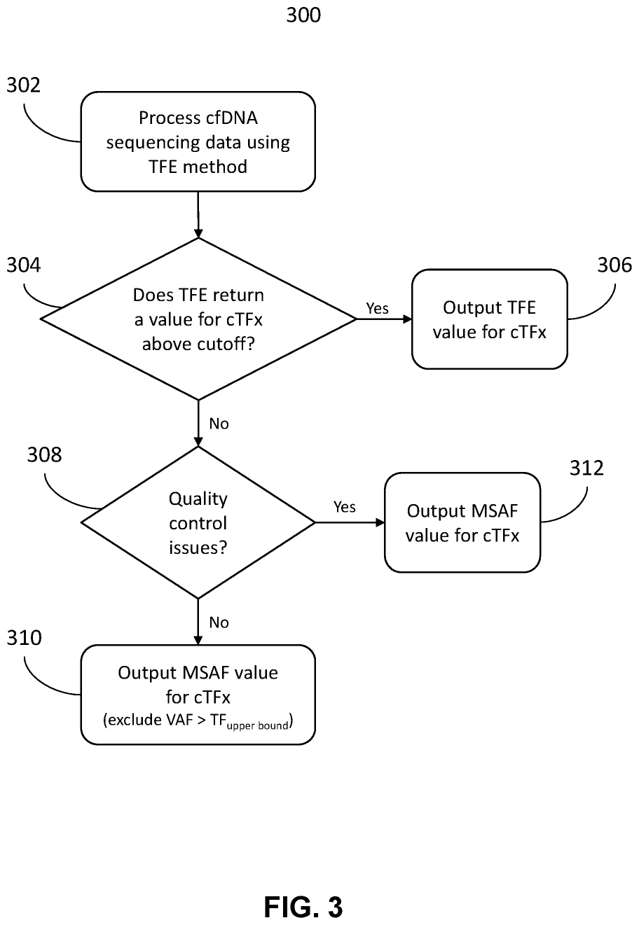

FIG. 3 provides another non-limiting example of a process 300 for determining tumor fraction according to embodiments of the present disclosure. Sequencing data (e.g., cell-free DNA (cfDNA) sequencing data) representing values for, e.g., allele fraction or a difference between an allele coverage of a locus in a tumor sample and an allele coverage of the same locus in a non-tumor sample, at a plurality of loci within a genome or subgenomic interval of a subject may be processed according to a first tumor fraction estimation process 302 (e.g., the tumor fraction estimator (TFE) process 100 described in FIG. 1). The estimate of tumor fraction (e.g., a circulating tumor fraction) for the sample returned by the first stage determination is compared to a first threshold, 304. The first threshold may be, for example, a limit-of-detection (LoD) or specified confidence level for determining tumor fraction using the first stage determination. If the estimated tumor fraction returned by the first stage determination is greater than the first threshold, the estimate is output as the determined value of the tumor fraction for the sample, 306. If the estimated tumor fraction returned by the first stage determination is less than or equal to the first threshold, the sequencing data may be examined for quality control issues, 308. For example, a quality metric may be calculated for the sequencing data and compared to a quality control threshold (e.g., a second threshold). Examples of quality control parameters that may be examined, used as a quality metric, and/or used to calculate a quality metric for the sequencing data include, but are not limited to, an average sequence coverage for the sample, a minimum average sequence coverage for the sample, an allele coverage at each of the corresponding loci in the plurality of loci, a minimum allele coverage at each of the corresponding loci in the plurality of loci, a degree of nucleic acid contamination in the sample (determined, e.g., by quantifying aberrations in SNP allele frequencies), a maximum degree of nucleic acid contamination in the sample, a number of single nucleotide polymorphism (SNP) loci within the plurality of loci examined a minimum number of single nucleotide polymorphism (SNP) loci within the plurality of loci examined, or any combination thereof. In some instances, the quality control threshold (or second threshold) may comprise a specified lower limit of the quality metric.

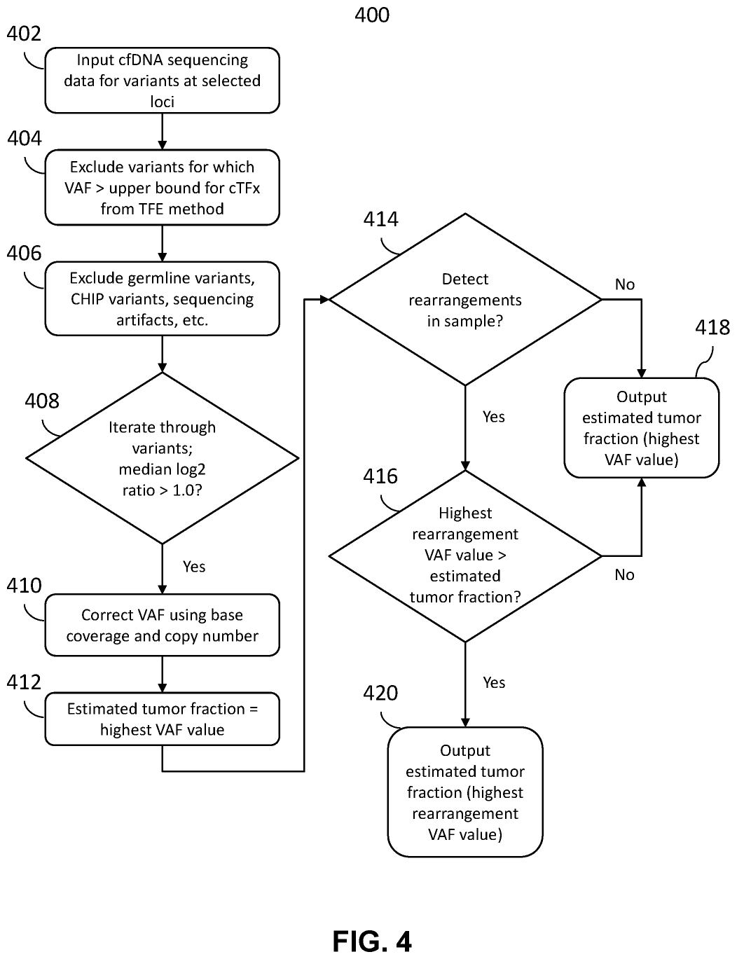

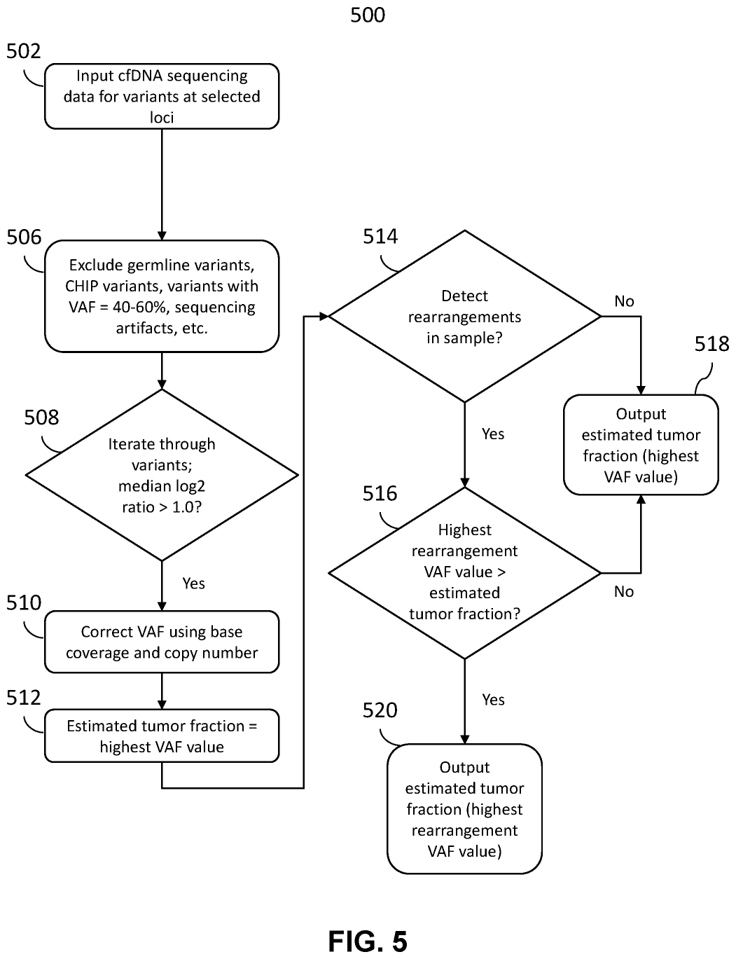

In the case that no quality control issues are identified for the sequencing data (e.g., the quality metric for the sequencing data is greater than a specified quality threshold), a first version of a secondary method 310 may be used to calculate tumor fraction for the sample. In some instances, the secondary method 310 may comprise, for example, a first determination of an allele frequency (e.g., a maximum somatic allele frequency (MSAF1)) to estimate tumor fraction of the sample. In the case that quality control issues are identified for the sequencing data (e.g., the quality metric for the sequencing data is less than or equal to a specified quality threshold), a second version of the secondary method 312 may be used to calculate tumor fraction for the sample. In some instances, the secondary method 312 may comprise, for examples, a second determination of an allele frequency (e.g., a maximum somatic allele frequency (MSAF2)) to estimate tumor fraction for the sample. The differences between the first and second determinations of maximum somatic allele frequency will be described in more detail with respect to FIG. 4 and FIG. 5. The choice of which of the two different versions of MSAF to use is based on the level of trust in the TFE estimate of tumor fraction. If the TFE was based on sequencing data that exhibited quality control issues, the upper bound estimation returned by TFE is not reliable and cannot be used as a gating filter for variant inclusion.

The process for determining tumor fraction illustrated in FIG. 3 again utilizes two (or more) complementary processes in a composite methodology for determining tumor fraction to provide for more accurate determinations of tumor fraction (e.g., circulating tumor fraction) over a larger range of DNA concentrations (e.g., circulating tumor DNA (ctDNA) concentrations).

FIG. 4 provides a non-limiting example of a process 400 for determining tumor fraction according to a first determination of maximum somatic allele frequency (MSAF1) according to embodiments of the present disclosure. Referring back to FIG. 3, in the case that the value for tumor fraction (e.g., circulating tumor fraction) returned by the first process (e.g., the TFE process) is less than a specified cutoff threshold 304, and there are no quality control issues identified for the input sequencing data 308, the tumor fraction for the sample may be calculated according to a first determination of maximum somatic allele frequency (MSAF1). Returning to FIG. 4, the first determination of maximum somatic allele frequency begins with the input of the sequencing data for the sample at step 402 (e.g., sequence data for variant sequences at the plurality of loci selected for analysis). In some instances, the sequencing data may be, e.g., sequencing data for cell-free DNA (cfDNA) in the sample (e.g., a liquid biopsy sample). Variant alleles for which the variant allele frequency (VAF) is greater than an upper bound for estimated tumor fraction determined using the TFE method are excluded from the determination at step 404. The upper bound determined by THE gives a 95% confidence limit for the ctDNA fraction in a sample. Variants with VAF above this threshold are excluded to limit potential overestimations of ctDNA content due to germline variants or variants with elevated VAF due to amplification. Known germline variant sequences (e.g., variants with consensus SGZ germline status), variant sequences associated with clonal hematopoiesis of indeterminate potential (CHIP variants), sequencing artifacts, and the like, are also excluded from the determination at step 406 by, e.g., comparing variant sequences against one or more sequence databases. The remaining variant sequences are then iteratively examined to identify variants which occur on amplified alleles (median log 2 ratio>1.0, where the log 2 ratio is the logarithm base 2 of the ratio of sample coverage and matched coverage across the targeted region for a specific allele), 408. For these variants, base coverage and copy number are used to correct their VAF, 410, by reverse engineering what the VAF would be if the variant occurred on a non-amplified allele. The value of the highest VAF observed for all remaining coding variants is assigned as the output value for the estimated tumor fraction of the sample, 412. The sequencing data is also examined for the presence of rearrangements, 414. If no rearrangements are detected, the highest value of VAF is output as the final determination of tumor fraction in the sample, 418. If rearrangements are detected, but the rearrangement VAF is less than the estimated tumor fraction based on the previously determined highest value of VAF, 416, the highest value of VAF is kept as the final determination of tumor fraction in the sample, 418. If rearrangements are detected, and the rearrangement VAF is greater than or equal to the estimated tumor fraction based on the previously determined highest value of VAF, 416, the highest observed value for rearrangement VAF is output as the final determination of tumor fraction for the sample, 420.

FIG. 5 provides a non-limiting example of a process 500 for determining tumor fraction according to a second determination of maximum somatic allele frequency (MSAF2) according to embodiments of the present disclosure. Referring back to FIG. 3, in the case that the value for tumor fraction (e.g., circulating tumor fraction) returned by the first process (e.g., the TFE process) is less than a specified cutoff threshold 304, and quality control issues are identified for the input sequencing data 308, the tumor fraction for the sample may be calculated according to a second determination of maximum somatic allele frequency (MSAF2). Returning to FIG. 5, the first determination of maximum somatic allele frequency begins with the input of the sequencing data for the sample, 502 (e.g., sequence data for variant sequences at the plurality of loci selected for analysis). In some instances, the sequencing data may be, e.g., sequencing data for cell-free DNA (cfDNA) in the sample (e.g., a liquid biopsy sample). Known germline variant sequences (e.g., variants with consensus SGZ germline status), variant sequences associated with clonal hematopoiesis of indeterminate potential (CHIP variants), variant alleles having a VAF ranging from 40-60% (in this version of MSAF there is no upper bound to use as a filter, so 40-60% represents a broad range that should capture most germline variants), sequencing artifacts, and the like, are excluded from the determination, 506. The remaining variant sequences are then iteratively examined to identify variants with occur on amplified alleles (median log 2 ratio>1.0), 508. For these variants, base coverage and copy number are used to correct their VAF, 510. The value of the highest VAF observed for all remaining coding variants is assigned as the output value for the estimated tumor fraction of the sample, 512. The sequencing data is also examined for the presence of rearrangements, 514. If no rearrangements are detected, the highest value of VAF is output as the final determination of tumor fraction in the sample, 518. If rearrangements are detected, but the rearrangement VAF is less than the estimated tumor fraction based on the previously determined highest value of VAF, 516, the highest value of VAF is kept as the final determination of tumor fraction in the sample, 518. If rearrangements are detected, and the rearrangement VAF is greater than or equal to the estimated tumor fraction based on the previously determined highest value of VAF, 516, the highest observed value for rearrangement VAF is output as the final determination of tumor fraction for the sample, 520.

If the output value for the tumor fraction of the sample is zero (according to the process illustrated in FIG. 5), the original sequencing data is checked for detection of amplifications (i.e. copy number gains for one or more loci being analyzed) by the sequencing data analysis pipeline used to input the variant sequence data, and a first override is returned if amplifications have been detected. If the output value for the tumor fraction of the sample is zero (according to the process illustrated in FIG. 5), the original sequencing data is also checked for deflections in SNP allele frequencies (i.e., deflections from an expected value of 0.5), and a second override is returned if an average minor allele frequency>0.47 was observed.

In some instances, a limit-of-detection (LoD) for accurately determining tumor fraction using the process illustrated in FIG. 1 (e.g., the TFE process for determining tumor fraction used as a first process in the composite processes illustrated in FIG. 2 and FIG. 3) may range from about 0.01% to about 5%. In some instances, the limit-of-detection for accurately determining tumor fraction may be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 1%, at least 1.5%, at least 2%, at least 2.5%, at least 3%, at least 3.5%, at least 4%, at least 4.5%, or at least 5%. In some instances, the limit-of-detection for accurately determining tumor fraction according to the TFE method illustrated in FIG. 1 may be any value within the preceding range of values.

In some instances, a limit-of-detection (LoD) for accurately determining tumor fraction using the first determination of maximum somatic allele frequency (MSAF1; illustrated in FIG. 4) and/or the second determination of maximum somatic allele frequency (MSAF2; illustrated in FIG. 5) may range from about 0.01% to about 2.5%. In some instances, the limit-of-detection for accurately determining tumor fraction may be at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 1%, at least 1.5%, at least 2%, at least 2.5%, at least 3%, at least 3.5%, at least 4%, at least 4.5%, or at least 5%. In some instances, the limit-of-detection for accurately determining tumor fraction according to the MSAF1 and/or MSAF2 methods (illustrated in FIG. 4 and FIG. 5 respectively) may be any value within the preceding range of values.

In some instances, the certainty metric-based methods disclosed herein for determining a tumor fraction in a sample (e.g., the tumor fraction estimator (TFE) method illustrated in FIG. 1) may provide accurate determinations of tumor fraction over a wide range of tumor DNA concentration. In some instances, the accuracy for determining the tumor fraction in a sample may range from within about ±0.2% to within about #10% of the tumor fraction determined by a reference method for samples containing a tumor fraction ranging from about 1% to about 50%. In some instances, the accuracy for determining the tumor fraction in a sample be within about ±10%, ±9%, ±8%, ±7%, ±6%, ±5%, ±4%, ±3%, 2%, ±1%, ±0.9%, ±0.8%, ±0.7%, ±0.6%, ±0.5%, ±0.4%, ±0.3%, or ±0.2% (or any value within this range) of the value determined by a reference method for samples comprising a tumor fraction ranging from about 1%, 1.5%, 2%, 2.5%, 5%, 10%, 15%, 20%, 25%, 30%, 35%, 40%, 45%, or 50% to about 1%, 1.5%, 2%, 2.5%, 5%, 10%, 15%, 20%, 25%, 30%, 35%, 40%, 45%, or 50% (or a tumor fraction ranging between any pair of increasing values within this range).

In some instances, the composite methods disclosed herein for determining a tumor fraction in a sample (e.g., those illustrated in FIG. 2 and FIG. 3) may provide improved accuracy in determining tumor fraction over a wider range of tumor DNA concentration. In some instances, the accuracy for determining the tumor fraction in a sample may range from within about #0.1% to within about ±10% of the tumor fraction determined by a reference method for samples containing a tumor fraction ranging from about 0.1% to about 50%. In some instances, the accuracy for determining the tumor fraction in a sample be within about #10%, ±9%, ±8%, ±7%, ±6%, ±5%, ±4%, ±3%, ±2%, ±1%, ±0.9%, ±0.8%, ±0.7%, ±0.6%, 0.5%, ±0.4%, ±0.3%, ±0.2%, or ±0.1% (or any value within this range) of the value determined by a reference method for samples comprising a tumor fraction ranging from about 0.1%, 0.2%, 0.3%, 0.4%, 0.5%, 1%, 1.5%, 2%, 2.5%, 5%, 10%, 15%, 20%, 25%, 30%, 35%, 40%, 45%, or 50% to about 0.1%, 0.2%, 0.3%, 0.4%, 0.5%, 1%, 1.5%, 2%, 2.5%, 5%, 10%, 15%, 20%, 25%, 30%, 35%, 40%, 45%, or 50% (or a tumor fraction ranging between any pair of increasing values within this range).

In some instances, the disclosed methods for determination of tumor fraction may be used for the analysis of samples comprising a quantity of cell-free DNA (cfDNA) and/or circulating tumor DNA (ctDNA) ranging from about 25 nanograms to about 1,000 nanograms. In some instances, the quantity of cfDNA and/or ctDNA in the sample may be at least 25 nanograms, at least 50 nanograms, at least 75 nanograms, at least 100 nanograms, at least 200 nanograms, at least 300 nanograms, at least 400 nanograms, at least 500 nanograms, at least 600 nanograms, at least 700 nanograms, at least 800 nanograms, at least 900 nanograms, or at least 1,000 nanograms. In some instances, the quantity of cfDNA and/or ctDNA in the sample may be at most 1,000 nanograms, at most 900 nanograms, at most 800 nanograms, at most 700 nanograms, at most 600 nanograms, at most 500 nanograms, at most 400 nanograms, at most 300 nanograms, at most 200 nanograms, at most 100 nanograms, at most 75 nanograms, at most 50 nanograms, or at most 25 nanograms. Any of the lower and upper values described in this paragraph may be combined to form a range included within the present disclosure, for example, in some instances, the quantity of cfDNA and/or ctDNA in the sample may range from about 100 nanograms to about 700 nanograms. Those of skill in the art will recognize that quantity of cfDNA and/or ctDNA in the sample may have any value within this range, e.g., about 232 nanograms.

### Tumor Treatment and Monitoring Methods

Methods of treating a disease in a subject are also disclosed. The methods include, responsive to a determination (e.g., an estimation) of tumor fraction (e.g., determined in accordance with a method described herein), administering an effective amount of a therapy to the subject, thereby treating the disease, wherein the estimation of tumor fraction comprises acquiring a value for a target variable associated with a subgenomic interval in the sample; determining, from the target variable, a certainty metric; accessing a determined relationship between a stored certainty metric and a stored tumor fraction; and determining, with reference to the certainty metric and the determined relationship, the tumor fraction of the sample.

Also disclosed are methods for treating a disease in a subject that comprise: responsive to a determination of tumor fraction, administering an effective amount of a tumor therapy to the subject, wherein the tumor fraction is determined by receiving a plurality of values, each value indicative of: (i) an allele fraction at a corresponding locus within a subgenomic interval in the sample, or (ii) a difference between an allele coverage of a locus in a tumor sample and an allele coverage of the same locus in a non-tumor sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; and outputting the second estimate of the tumor fraction of the sample.

In some embodiments, an effective amount of a tumor therapy to be administered to the subject may be directly related to the tumor fraction determined in a sample, or to a difference in the tumor fraction determined for successive samples, from the subject using any of the methods disclosed herein. For example, in some instances, the dose of the tumor therapy to be administered may be linearly related, non-linearly related, or otherwise proportional to the determined tumor fraction or to a change in tumor fraction. In some instances, the dose of the tumor therapy to be administered may be varied over time depending on the tumor fraction determined for a series of samples collected from the subject over time.

In an embodiment, the methods further comprises administering a second therapy to the subject. In an embodiment, the methods further comprises discontinuing a second therapy to the subject. In an embodiment, the methods further comprises determining the presence of a somatic alteration (e.g., a somatic alteration associated with the disease) in the subject.

In an embodiment, the allele fraction is determined by a method comprising sequencing, e.g., next-generation sequencing (NGS). In an embodiment, the allele fraction is determined by a method further comprising target selection, e.g., by solution hybridization. In other embodiments, other methodologies used for detecting DNA (e.g., cfDNA, ctDNA, etc.) can be employed, such as microarrays.

Methods of evaluating (or monitoring) a disease in a subject are also described, wherein the determination (e.g., estimation) of tumor fraction is determined in accordance with a method described herein. In some instances, the method comprises acquiring a value for a target variable associated with a subgenomic interval in the sample; determining, from the target variable, a certainty metric; accessing a determined relationship between a stored certainty metric and a stored tumor fraction; and determining, with reference to the certainty metric and the determined relationship, the tumor fraction of the sample, thereby evaluating the disease. In some instances, the method comprises receiving a plurality of values, each value indicative of: (i) an allele fraction at a corresponding locus within a subgenomic interval in the sample, or (ii) a difference between an allele coverage of a locus in a tumor sample and an allele coverage of the same locus in a non-tumor sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; and outputting the second estimate of the tumor fraction of the sample, thereby allowing one to evaluate the disease based on the determination of tumor fraction.

In an embodiment, the allele fraction is determined by a method comprising sequencing, e.g., NGS. In an embodiment, the allele fraction is determined by a method further comprising target selection, e.g., by solution hybridization. In other embodiments, other methodologies used for detecting DNA (e.g., cfDNA, ctDNA, etc.) can be employed, such as microarrays. In an embodiment, the disclosed methods further comprise selecting a therapy for the disease. In an embodiment, the methods further comprise discontinuing a therapy to the subject. In an embodiment, the methods further comprise selecting the subject for a clinical trial. In an embodiment, the methods further comprise determining a disease status, e.g., remission, stable, relapse, etc. In an embodiment, the disease is evaluated periodically, e.g., every month, every two months, every three months, every six months, or every year. In an embodiment, the methods further comprise determining the presence of a somatic alteration (e.g., a somatic alteration associated with the disease) in the subject.

A method of evaluating a subject is described, wherein the determination (e.g., estimation) of tumor fraction (e.g., determined in accordance with a method described herein) comprises acquiring a value for a target variable associated with a subgenomic interval in the sample; determining, from the target variable, a certainty metric; accessing a determined relationship between a stored certainty metric and a stored tumor fraction; and determining, with reference to the certainty metric and the determined relationship, the tumor fraction of the sample, thereby evaluating the subject. In some instances, the determination of tumor fraction comprises receiving a plurality of values, each value indicative of: (i) an allele fraction at a corresponding locus within a subgenomic interval in the sample, or (ii) a difference between an allele coverage of a locus in a tumor sample and an allele coverage of the same locus in a non-tumor sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; and outputting the second estimate of the tumor fraction of the sample, thereby allowing one to evaluate the subject based on the determination of tumor fraction.

In an embodiment, the allele fraction is determined by a method comprising sequencing, e.g., NGS. In an embodiment, the allele fraction is determined by a method further comprising target selection, e.g., by solution hybridization. In other embodiments, other methodologies used for detecting DNA (e.g., cfDNA, ctDNA, etc.) can be employed, such as microarrays.

In an embodiment, the disclosed methods further comprise selecting the subject for a therapy. In an embodiment, the methods further comprise discontinuing a therapy to the subject. In an embodiment, the methods further comprise selecting the subject for a clinical trial.

In an embodiment, the subject is evaluated periodically, e.g., every month, every two months, every three months, every six months, or every year.

In an embodiment, the disclosed methods further comprise determining the presence of a somatic alteration (e.g., a somatic alteration associated with the disease) in the subject.

In an embodiment, the target variable (e.g., allele fraction) is determined by a method comprising sequencing, e.g., NGS. In an embodiment, the allele fraction is determined by a method further comprising target selection, e.g., by solution hybridization. In other embodiments, other methodologies used for detecting DNA (e.g., cfDNA, ctDNA, etc.) can be employed, such as microarrays,

A method of evaluating a therapy is described, wherein the determination (e.g., estimation) of tumor fraction (e.g., determined in accordance with a method described herein) comprises acquiring a value for a target variable associated with a subgenomic interval in the sample; determining, from the target variable, a certainty metric; accessing a determined relationship between a stored certainty metric and a stored tumor fraction; and determining, with reference to the certainty metric and the determined relationship, the tumor fraction of the sample, thereby evaluating the therapy. In some instances, the determination of the tumor fraction of the sample comprises receiving a plurality of values, each value indicative of: (i) an allele fraction at a corresponding locus within a subgenomic interval in the sample, or (ii) a difference between an allele coverage of a locus in a tumor sample and an allele coverage of the same locus in a non-tumor sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; and outputting the second estimate of the tumor fraction of the sample, thereby allowing one to evaluate the therapy based on the determination of tumor fraction.

In an embodiment, the target variable (e.g., allele fraction) is determined by a method comprising sequencing, e.g., NGS. In an embodiment, the allele fraction is determined by a method further comprising target selection, e.g., by solution hybridization. In other embodiments, other methodologies used for detecting DNA (e.g., cfDNA, ctDNA, etc.) can be employed, such as microarrays,

In an embodiment, the disclosed methods further comprise selecting the therapy for the subject.

In an embodiment, the therapy is evaluated periodically, e.g., every month, every two months, every three months, every six months, or every year,

A method of providing a report (e.g., to report tumor fraction determined in accordance with a method described herein) is described. The method includes acquiring a value for a target variable associated with a subgenomic interval in the sample; determining, from the target variable, a certainty metric; accessing a determined relationship between a stored certainty metric and a stored tumor fraction; and determining, with reference to the certainty metric and the determined relationship, the tumor fraction of the sample; and recording the estimated tumor fraction in a report, thereby providing the report. In some instances, the method includes receiving a plurality of values, each value indicative of: (i) an allele fraction at a corresponding locus within a subgenomic interval in the sample, or (ii) a difference between an allele coverage of a locus in a tumor sample and an allele coverage of the same locus in a non-tumor sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; outputting the second estimate of the tumor fraction of the sample; and recording the estimated tumor fraction in a report, thereby providing the report.

In an embodiment, the allele fraction is determined by a method comprising sequencing, e.g., NGS. In an embodiment, the allele fraction is determined by a method further comprising target selection, e.g., by solution hybridization. In other embodiments, other methodologies used for detecting DNA (e.g., cfDNA, ctDNA, etc.) can be employed, such as microarrays.

In an embodiment, the disclosed methods further comprise transmitting the report to the subject or a third party. In an embodiment, the report further comprises a treatment option based on the estimated tumor fraction.

In an embodiment, the reporting further comprises a genomic profile (e.g., a genomic profile associated with the disease) of the subject.

A method of evaluating a biopsy (e.g., comprising determining tumor fraction in accordance with a method described herein) from a subject is described. In some instances, the method includes acquiring a value for a target variable associated with a subgenomic interval in a sample from the biopsy; determining, from the target variable, a certainty metric; accessing a determined relationship between a stored certainty metric and a stored tumor fraction; and determining, with reference to the certainty metric and the determined relationship, the tumor fraction of the sample, thereby evaluating the biopsy. In some instances, the method includes receiving a plurality of values, each value indicative of: (i) an allele fraction at a corresponding locus within a subgenomic interval in the sample, or (ii) a difference between an allele coverage of a locus in a tumor sample and an allele coverage of the same locus in a non-tumor sample; determining a certainty metric value indicative of a dispersion of the plurality of values; determining, based on the certainty metric value for the sample and a predetermined relationship between one or more stored certainty metric values and one or more stored tumor fraction values, a first estimate of the tumor fraction of the sample; determining whether a value associated with the first estimate of the tumor fraction of the sample is greater than a first threshold; based on a determination that the value associated with the first estimate of the tumor fraction of the sample is greater than the first threshold, outputting the first estimate of the tumor fraction of the sample; and based on a determination that the value associated with the first estimate of the tumor fraction is less than or equal to the first threshold: determining a second estimate of the tumor fraction of the sample based on maximum somatic allele frequency; outputting the second estimate of the tumor fraction of the sample; thereby evaluating the biopsy.

In an embodiment, an estimated tumor fraction above a threshold value is indicative that the biopsy is suitable for genomic profiling.

### Exemplary Computer Implementations

Processes described above are merely illustrative embodiments of methods and systems that may be used to estimate tumor fraction. Such illustrative embodiments are not intended to limit the scope of the present disclosure. None of the embodiments and claims set forth herein are intended to be limited to any particular implementation, unless such claim includes a limitation explicitly reciting a particular implementation.

Processes and methods associated with various embodiments, acts thereof and various embodiments and variations of these methods and acts, individually or in combination, may be defined by computer-readable signals tangibly embodied on a computer-readable medium, for example, a non-volatile recording medium, an integrated circuit memory element, or a combination thereof. According to one embodiment, the computer-readable medium may be non-transitory in that the computer-executable instructions may be stored permanently or semi-permanently on the medium. Such signals may define instructions, for example, as part of one or more programs that, as a result of being executed by a computer, instruct the computer to perform one or more of the methods or acts described herein, and/or various embodiments, variations and combinations thereof. Such instructions may be written in any of a plurality of programming languages, for example, Java, Visual Basic, C, C#, or C++, Fortran, Pascal, Eiffel, Basic, COBOL, etc., or any of a variety of combinations thereof. The computer-readable medium on which such instructions are stored may reside on one or more of the components of a general-purpose computer described above, and may be distributed across one or more of such components.

The computer-readable medium may be transportable such that the instructions stored thereon can be loaded onto any computer system resource to implement the aspects of the present disclosure discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement the above-discussed aspects of the present disclosure,

Various embodiments according to the disclosure may be implemented on one or more computer systems. These computer systems may be, for example, general-purpose computers such as those based on Intel PENTIUM-type processor, Motorola PowerPC, Sun UltraSPARC, Hewlett-Packard PA-RISC processors, ARM Cortex processor, Qualcomm Scorpion processor, or any other type of processor. It should be appreciated that one or more of any type computer system may be used to partially or fully automate extending offers to users and redeeming offers according to various embodiments of the disclosure. Further, the software design system may be located on a single computer or may be distributed among a plurality of computers attached by a communications network.

The computer system may include specially-programmed, special-purpose hardware, for example, an application-specific integrated circuit (ASIC). Aspects of the disclosure may be implemented in software, hardware or firmware, or any combination thereof. Further, such methods, acts, systems, system elements and components thereof may be implemented as part of the computer system described above or as an independent component.

A computer system may be a general-purpose computer system that is programmable using a high-level computer programming language. A computer system may be also implemented using specially programmed, special purpose hardware. In a computer system there may be a processor that is typically a commercially available processor such as the well-known Pentium class processor available from the Intel Corporation. Many other processors are available. Such a processor usually executes an operating system which may be, for example, the Windows NT, Windows 2000 (Windows ME), Windows XP, Windows Vista or Windows 7 operating systems available from the Microsoft Corporation, MAC OS X Snow Leopard, MAC OS X Lion operating systems available from Apple Computer, the Solaris Operating System available from Oracle Corporation, iOS, Blackberry OS, Windows 7 Mobile or Android OS operating systems, or UNIX available from various sources. Many other operating systems may be used.

Some aspects of the disclosure may be implemented as distributed application components that may be executed on a number of different types of systems coupled over a computer network. Some components may be located and executed on mobile devices, servers, tablets, or other system types. Other components of a distributed system may also be used, such as databases or other component types.

The processor and operating system together define a computer platform for which application programs in high-level programming languages are written. It should be understood that the disclosure is not limited to a particular computer system platform, processor, operating system, computational set of algorithms, code, or network. Further, it should be appreciated that multiple computer platform types may be used in a distributed computer system that implement various aspects of the present disclosure. Also, it should be apparent to those skilled in the art that the present disclosure is not limited to a specific programming language, computational set of algorithms, code or computer system. Further, it should be appreciated that other appropriate programming languages and other appropriate computer systems could also be used.

One or more portions of the computer system may be distributed across one or more computer systems coupled to a communications network. These computer systems also may be general-purpose computer systems. For example, various aspects of the disclosure may be distributed among one or more computer systems configured to provide a service (e.g., servers) to one or more client computers, or to perform an overall task as part of a distributed system. For example, various aspects of the disclosure may be performed on a client-server system that includes components distributed among one or more server systems that perform various functions according to various embodiments of the disclosure. These components may be executable, intermediate (e.g., IL) or interpreted (e.g., Java) code which communicate over a communication network (e.g., the Internet) using a communication protocol (e.g., TCP/IP). Certain aspects of the present disclosure may also be implemented on a cloud-based computer system (e.g., the EC2 cloud-based computing platform provided by Amazon.com), a distributed computer network including clients and servers, or any combination of systems.

It should be appreciated that the disclosure is not limited to executing on any particular system or group of systems. Also, it should be appreciated that the disclosure is not limited to any particular distributed architecture, network, or communication protocol.

Various embodiments of the present disclosure may be programmed using an object-oriented programming language, such as SmallTalk, Java, C++, Ada, or C#(C-Sharp). Other object-oriented programming languages may also be used. Alternatively, functional, scripting, and/or logical programming languages may be used. Various aspects of the disclosure may be implemented in a non-programmed environment (e.g., documents created in HTML, XML or other format that, when viewed in a window of a browser program, render aspects of a graphical-user interface (GUI) or perform other functions). Various aspects of the disclosure may be implemented as programmed or non-programmed elements, or any combination thereof.

Further, on each of the one or more computer systems that include one or more components of the device, each of the components may reside in one or more locations on the system. For example, different portions of the components of the device may reside in different areas of memory (e.g., RAM, ROM, disk, etc.) on one or more computer systems. Each of such one or more computer systems may include, among other components, a plurality of known components such as one or more processors, a memory system, a disk storage system, one or more network interfaces, and one or more busses or other internal communication links interconnecting the various components,

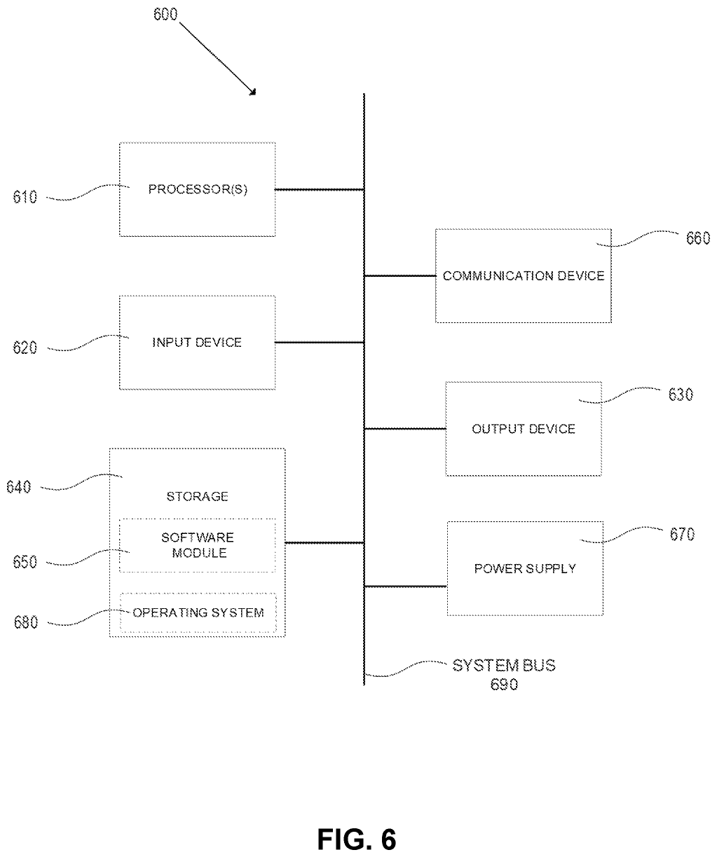

FIG. 6 illustrates an example of a computing device in accordance with one embodiment. Device 600 can be a host computer connected to a network. Device 600 can be a client computer or a server. As shown in FIG. 6, device 600 can be any suitable type of microprocessor-based device, such as a personal computer, workstation, server or handheld computing device (portable electronic device) such as a phone or tablet. The device can include, for example, one or more of processor(s) 610, input device 620, output device 630, storage 640, communication device 660, power supply 670, operating system 680, and system bus 690. Input device 620 and output device 630 can generally correspond to those described herein, and can either be connectable or integrated with the computer.

Input device 620 can be any suitable device that provides input, such as a touch screen, keyboard or keypad, mouse, or voice-recognition device. Output device 630 can be any suitable device that provides output, such as a touch screen, haptics device, or speaker,

Storage 640 can be any suitable device that provides storage (e.g., an electrical, magnetic or optical memory including a RAM (volatile and non-volatile), cache, hard drive, or removable storage disk). Communication device 660 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 6, the components are connected by system bus 690.

Software module 650, which can be stored as executable instructions in storage 640 and executed by processor(s) 610, can include, for example, the processes that embody the functionality of the methods of the present disclosure (e.g., as embodied in the devices as described herein).

Software module 650 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 640, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 650 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

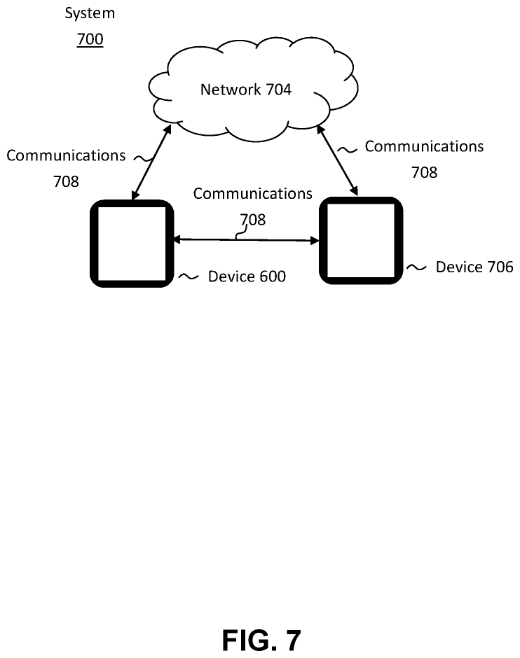

Device 600 may be connected to a network (e.g., network 704, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, TI or T3 lines, cable networks, DSL, or telephone lines.

Device 600 can implement any operating system (e.g., operating system 680) suitable for operating on the network. Software module 650 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, operating system 680 is executed by one or more processors, e.g., processor(s) 610.

Device 600 can further include power supply 670, which can be any suitable power supply.

FIG. 7 illustrates an example of a computing system in accordance with one embodiment. In system 700, device 600 (e.g., as described above and illustrated in FIG. 6) is connected to network 704, which is also connected to device 706. In some embodiments, device 706 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio RS system. Devices 600 and 706 may communicate, e.g., using suitable communication interfaces via network 704, such as a Local Area Network (LAN), Virtual Private Network (VPN), or the Internet. In some embodiments, network 704 can be, for example, the Internet, an intranet, a virtual private network, a cloud network, a wired network, or a wireless network. Devices 600 and 706 may communicate, in part or in whole, via wireless or hardwired communications, such as Ethernet, IEEE 802.11b wireless, or the like. Additionally, devices 600 and 706 may communicate, e.g., using suitable communication interfaces, via a second network, such as a mobile/cellular network. Communication between devices 600 and 706 may further include or communicate with various servers such as a mail server, mobile server, media server, telephone server, and the like. In some embodiments, Devices 600 and 706 can communicate directly (instead of, or in addition to, communicating via network 704), e.g., via wireless or hardwired communications, such as Ethernet, IEEE 802.11b wireless, or the like. In some embodiments, devices 600 and 706 communicate via communications 708, which can be a direct connection or can occur via a network (e.g., network 704).

One or all of devices 600 and 706 generally include logic (e.g., http web server logic) or is programmed to format data, accessed from local or remote databases or other sources of data and content, for providing and/or receiving information via network 704 according to various examples described herein.

### Definitions

Certain terms are defined. Additional terms are defined throughout the specification.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” or “acquiring a read” as the term is used herein, refers to obtaining possession of a nucleotide sequence or amino acid sequence, by “directly acquiring” or “indirectly acquiring” the sequence or read. “Directly acquiring” a sequence or read means performing a process (e.g., performing a synthetic or analytical method) to obtain the sequence, such as performing a sequencing method (e.g., a Next-generation Sequencing (NGS) method). “Indirectly acquiring” a sequence or read refers to receiving information or knowledge of, or receiving, the sequence from another party or source (e.g., a third-party laboratory that directly acquired the sequence). The sequence or read acquired need not be a full sequence, e.g., sequencing of at least one nucleotide, or obtaining information or knowledge, that identifies one or more of the alterations disclosed herein as being present in a sample, biopsy or subject constitutes acquiring a sequence.

Directly acquiring a sequence or read includes performing a process that includes a physical change in a physical substance, e.g., a starting material, such as a sample described herein. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above. The size of the fragment (e.g., the average size of the fragments) can be 2500 bp or less, 2000 bp or less, 1500 bp or less, 1000 bp or less, 800 bp or less, 600 bp or less, 400 bp or less, or 200 bp or less. In some embodiments, the size of the fragment (e.g., cfDNA) is between about 150 bp and about 200 bp (e.g., between about 160 bp and about 170 bp). In some embodiments, the size of the fragment (e.g., DNA fragments from FFPE samples) is between about 150 bp and about 250 bp. In some embodiments, the size of the fragment (e.g., cDNA fragments obtained from RNA in FFPE samples) is between about 100 bp and about 150 bp.

“Acquiring a sample” as the term is used herein, refers to obtaining possession of a sample, e.g., a sample described herein, by “directly acquiring” or “indirectly acquiring” the sample. “Directly acquiring a sample” means performing a process (e.g., performing a physical method such as a surgery or extraction) to obtain the sample. “Indirectly acquiring a sample” refers to receiving the sample from another party or source (e.g., a third-party laboratory that directly acquired the sample). Directly acquiring a sample from a subject includes performing a process that includes a physical change in a physical substance, e.g., a starting material, such as a tissue, e.g., a tissue in a human patient or a tissue that has was previously isolated from a patient. Exemplary changes include making a physical entity from a starting material, dissecting or scraping a tissue; separating or purifying a substance (e.g., a sample tissue or a nucleic acid sample); combining two or more separate entities into a mixture; performing a chemical reaction that includes breaking or forming a covalent or non-covalent bond. Directly acquiring a sample includes performing a process that includes a physical change in a sample or another substance, e.g., as described above.

“Alteration” or “altered structure” as used herein, of a gene or gene product (e.g., a marker gene or gene product) refers to the presence of a mutation or mutations within the gene or gene product, e.g., a mutation, which affects integrity, sequence, structure, amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell, as compared to a normal, healthy tissue or cell. Exemplary mutations include, but are not limited to, point mutations (e.g., silent, missense, or nonsense), deletions, insertions, inversions, duplications, amplification, translocations, inter- and intra-chromosomal rearrangements. Mutations can be present in the coding or non-coding region of the gene. In certain embodiments, the alteration(s) is detected as a rearrangement, e.g., a genomic rearrangement comprising one or more introns or fragments thereof (e.g., one or more rearrangements in 5′- and/or 3′-UTR). In certain embodiments, the alterations are associated (or not associated) with a phenotype, e.g., a cancerous phenotype (e.g., one or more of cancer risk, cancer progression, cancer treatment or resistance to cancer treatment). In one embodiment, the alteration (or tumor mutational burden) is associated with one or more of; a genetic risk factor for cancer, a positive treatment response predictor, a negative treatment response predictor, a positive prognostic factor, a negative prognostic factor, or a diagnostic factor.

As used herein, the term “indel” refers to an insertion, a deletion, or both, of one or more nucleotides in a nucleic acid of a cell. In certain embodiments, an indel includes both an insertion and a deletion of one or more nucleotides, where both the insertion and the deletion are nearby on the nucleic acid. In certain embodiments, the indel results in a net change in the total number of nucleotides. In certain embodiments, the indel results in a net change of about 1 to about 50 nucleotides.

“Clonal profile”, as that term is used herein, refers to the occurrence, identity, variability, distribution, expression (the occurrence or level of transcribed copies of a subgenomic signature), or abundance, e.g., the relative abundance, of one or more sequences, e.g., an allele or signature, of a subject interval (or of a cell comprising the same). In an embodiment, the clonal profile is a value for the relative abundance for one sequence, allele, or signature, for a subject interval (or of a cell comprising the same) when a plurality of sequences, alleles, or signatures for that subject interval are present in a sample. E.g., in an embodiment, a clonal profile comprises a value for the relative abundance, of one or more of a plurality of VDJ or VJ combinations for a subject interval. In an embodiment, a clonal profile comprises a value for the relative abundance of a selected V segment for a subject interval. In an embodiment, a clonal profile comprises a value for the diversity, e.g., as arises from somatic hypermutation, within the sequences of a subject interval. In an embodiment, a clonal profile comprises a value for the occurrence or level of expression of a sequence, allele, or signature, e.g., as evidenced by the occurrence or level of an expressed subgenomic interval comprising the sequence, allele or signature.

“Expressed subgenomic interval”, as that term is used herein, refers to the transcribed sequence of a subgenomic interval. In an embodiment, the sequence of the expressed subgenomic interval will differ from the subgenomic interval from which it is transcribed, e.g., as some sequence may not be transcribed.

“Mutant allele frequency” (MAF) as that term is used herein, refers to the relative frequency of a mutant allele at a particular locus, e.g., in a sample. In some embodiments, a mutant allele frequency is expressed as a fraction or percentage.

“Signature”, as that term is used herein, refers to a sequence of a subject interval. A signature can be diagnostic of the occurrence of one of a plurality of possibilities at a subject interval, e.g., a signature can be diagnostic of: the occurrence of a selected V segment in a rearranged heavy or light chain variable region gene; the occurrence of a selected VJ junction, e.g., the occurrence of a selected V and a selected J segment in a rearranged heavy chain variable region gene. In an embodiment, a signature comprises a plurality of a specific nucleic acid sequences. Thus, a signature is not limited to a specific nucleic acid sequence, but rather is sufficiently unique that it can distinguish between a first group of sequences or possibilities at a subject interval and a second group of possibilities at a subject interval, e.g., it can distinguish between a first V segment and a second V segment, allowing e.g., evaluation of the usage of various V segments. The term signature comprises the term specific signature, which is a specific nucleic acid sequence. In an embodiment the signature is indicative of, or is the product of, a specific event, e.g., a rearrangement event.

“Subgenomic interval” as that term is used herein, refers to a portion of genomic sequence. In an embodiment, a subgenomic interval can be a single nucleotide position, e.g., a variant at the position is associated (positively or negatively) with a tumor phenotype. In an embodiment, a subgenomic interval comprises more than one nucleotide position. Such embodiments include sequences of at least 2, 5, 10, 50, 100, 150, or 250 nucleotide positions in length. Subgenomic intervals can comprise an entire gene, or a portion thereof, e.g., the coding region (or portions thereof), an intron (or portion thereof) or exon (or portion thereof). A subgenomic interval can comprise all or a part of a fragment of a naturally occurring, e.g., genomic DNA, nucleic acid. E.g., a subgenomic interval can correspond to a fragment of genomic DNA which is subjected to a sequencing reaction. In an embodiment, a subgenomic interval is continuous sequence from a genomic source. In an embodiment, a subgenomic interval includes sequences that are not contiguous in the genome, e.g., subgenomic intervals in cDNA can include exon-exon junctions formed as a result of splicing. In an embodiment, the subgenomic interval comprises a tumor nucleic acid molecule. In an embodiment, the subgenomic interval comprises a non-tumor nucleic acid molecule.

In an embodiment, a subgenomic interval corresponds to a rearranged sequence, e.g., a sequence in a B or T cell that arises as a result of the joining of, a V segment to a D segment, a D segment to a J segment, a V segment to a J segment, or a J segment to a class segment.

In an embodiment, the subgenomic interval is represented by one sequence. In an embodiment, the subgenomic interval is represented by more than one sequence, e.g., the subgenomic interval that covers a VD sequence can be represented by more than one signature.

In an embodiment, a subgenomic interval comprises or consists of: a single nucleotide position; an intragenic region or an intergenic region; an exon or an intron, or a fragment thereof, typically an exon sequence or a fragment thereof; a coding region or a non-coding region, e.g., a promoter, an enhancer, a 5′ untranslated region (5′ UTR), or a 3′ untranslated region (3′ UTR), or a fragment thereof; a cDNA or a fragment thereof; an SNP; a somatic mutation, a germline mutation or both; an alteration, e.g., a point or a single mutation; a deletion mutation (e.g., an in-frame deletion, an intragenic deletion, a full gene deletion); an insertion mutation (e.g., intragenic insertion); an inversion mutation (e.g., an intra-chromosomal inversion); an inverted duplication mutation; a tandem duplication (e.g., an intrachromosomal tandem duplication); a translocation (e.g., a chromosomal translocation, a non-reciprocal translocation); a rearrangement (e.g., a genomic rearrangement (e.g., a rearrangement of one or more introns, a rearrangement of one or more exons, or a combination and/or a fragment thereof; a rearranged intron can include a 5′- and/or 3′-UTR)); a change in gene copy number; a change in gene expression; a change in RNA levels; or a combination thereof. The “copy number of a gene” refers to the number of DNA sequences in a cell encoding a particular gene product. Generally, for a given gene, a mammal has two copies of each gene. The copy number can be increased, e.g., by gene amplification or duplication, or reduced by deletion.

“Subject interval”, as that term is used herein, refers to a subgenomic interval or an expressed subgenomic interval. In an embodiment, a subgenomic interval and an expressed subgenomic interval correspond, meaning that the expressed subgenomic interval comprises sequence expressed from the corresponding subgenomic interval. In an embodiment, a subgenomic interval and an expressed subgenomic interval are non-corresponding, meaning that the expressed subgenomic interval does not comprise sequence expressed from the non-corresponding subgenomic interval, but rather corresponds to a different subgenomic interval. In an embodiment, a subgenomic interval and an expressed subgenomic interval partially correspond, meaning that the expressed subgenomic interval comprises sequence expressed from the corresponding subgenomic interval and sequence expressed from a different corresponding subgenomic interval.

As used herein, the term “library” refers to a collection of nucleic acid molecules. In one embodiment, the library includes a collection of nucleic acid nucleic acid molecules, e.g., a collection of whole genomic, subgenomic fragments, cDNA, cDNA fragments, RNA, e.g., mRNA, RNA fragments, or a combination thereof. Typically, a nucleic acid molecule is a DNA molecule, e.g., genomic DNA or cDNA. A nucleic acid molecule can be fragmented, e.g., sheared or enzymatically prepared, genomic DNA. Nucleic acid molecules comprise sequence from a subject and can also comprise sequence not derived from the subject, e.g., an adapter sequence, a primer sequence, or other sequences that allow for identification, e.g., “barcode” sequences. In one embodiment, a portion or all of the library nucleic acid molecules comprises an adapter sequence. The adapter sequence can be located at one or both ends. The adapter sequence can be useful, e.g., for a sequencing method (e.g., an NGS method), for amplification, for reverse transcription, or for cloning into a vector. The library can comprise a collection of nucleic acid molecules, e.g., a target nucleic acid molecule (e.g., a tumor nucleic acid molecule, a reference nucleic acid molecule, or a combination thereof). The nucleic acid molecules of the library can be from a single individual. In embodiments, a library can comprise nucleic acid molecules from more than one subject (e.g., 2, 3, 4, 5, 6, 7, 8, 9, 10, 20, 30 or more subjects), e.g., two or more libraries from different subjects can be combined to form a library comprising nucleic acid molecules from more than one subject. In one embodiment, the subject is a human having, or at risk of having, a cancer or tumor.

“Library catch” refers to a subset of a library, e.g., a subset enriched for subject intervals, e.g., product captured by hybridization with target capture reagents.

“Target Capture Reagent,” as used herein, refers to a molecule capable of capturing a target. A target capture reagent (e.g., a bait or a target capture oligonucleotide) can comprise a nucleic acid molecule, e.g., a DNA or RNA molecule, which can hybridize to (e.g., be complementary to), and thereby allow capture of a target nucleic acid. In one embodiment, a target capture reagent comprises a DNA molecule (e.g., a naturally-occurring or modified DNA molecule), an RNA molecule (e.g., a naturally-occurring or modified RNA molecule), or a combination thereof. In one embodiment, a target capture reagent is suitable for solution phase hybridization.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Likely to” or “increased likelihood,” as used herein, refers to an increased probability that an item, object, thing or person will occur. Thus, in one example, a subject that is likely to respond to treatment has an increased probability of responding to treatment relative to a reference subject or group of subjects.

“Unlikely to” refers to a decreased probability that an event, item, object, thing or person will occur with respect to a reference. Thus, a subject that is unlikely to respond to treatment has a decreased probability of responding to treatment relative to a reference subject or group of subjects.

“Control nucleic acid molecule” refers to a nucleic acid molecule having sequence from a non-tumor cell.

“Next-generation sequencing” or “NGS” or “NG sequencing” as used herein, refers to any sequencing method that determines the nucleotide sequence of either individual nucleic acid molecules (e.g., in single molecule sequencing) or clonally expanded proxies for individual nucleic acid molecules in a high throughput fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment, Next-generation sequencing methods are known in the art, and are described, e.g., in Metzker, M. (2010) Nature Biotechnology Reviews 11:31-46, incorporated herein by reference. Next-generation sequencing can detect a variant present in less than 5% or less than 1% of the nucleic acids in a sample.

“Nucleotide value” as referred herein, represents the identity of the nucleotide(s) occupying or assigned to a nucleotide position. Typical nucleotide values include: missing (e.g., deleted); additional (e.g., an insertion of one or more nucleotides, the identity of which may or may not be included); or present (occupied); A; T; C; or G. Other values can be, e.g., not Y, wherein Y is A, T, G, or C; A or X, wherein X is one or two of T, G, or C; T or X, wherein X is one or two of A, G, or C; G or X, wherein X is one or two of T, A, or C; C or X, wherein X is one or two of T, G, or A; a pyrimidine nucleotide; or a purine nucleotide. A nucleotide value can be a frequency for 1 or more, e.g., 2, 3, or 4, bases (or other value described herein, e.g., missing or additional) at a nucleotide position. E.g., a nucleotide value can comprise a frequency for A, and a frequency for G, at a nucleotide position.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

“Primary control” refers to a non-tumor tissue other than a normal adjacent tissue (NAT) tissue in a sample. Blood is a typical primary control.

“Sample,” as used herein, refers to a biological sample obtained or derived from a source of interest, as described herein. In some embodiments, a source of interest comprises an organism, such as an animal or human. The source of the sample can be solid tissue as from a fresh, frozen and/or preserved organ, tissue sample, biopsy, resection, smear, or aspirate; blood or any blood constituents; bodily fluids such as cerebral spinal fluid, amniotic fluid, peritoneal fluid or interstitial fluid; or cells from any time in gestation or development of the subject. In some embodiments, the source of the sample is blood or blood constituents.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA). In some embodiments, cfDNA is DNA from cells undergoing apoptosis or necrotic cells. Typically, cfDNA is bound by protein (e.g., histone) and protected by nucleases. CfDNA can be used as a biomarker for non-invasive prenatal testing (NIPT), organ transplant, cardiomyopathy, microbiome, and cancer. In another embodiment, the sample comprises circulating tumor DNA (ctDNA). In some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). In some embodiments, CTCs are cells shed from a primary or metastatic tumor into the circulation. In some embodiments, CTC apoptosis and are a source of ctDNA in the blood/lymph.

In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or bronchoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained.

In some embodiments, a sample is a “primary sample” obtained directly from a source of interest by any appropriate means. For example, in some embodiments, a primary biological sample is obtained by a method chosen from biopsy (e.g., fine needle aspiration or tissue biopsy), surgery, collection of body fluid (e.g., blood, lymph, or feces), etc. In some embodiments, as will be clear from context, the term “sample” refers to a preparation that is obtained by processing (e.g., by removing one or more components of and/or by adding one or more agents to) a primary sample, e.g., filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

In an embodiment, the sample is a cell associated with a tumor, e.g., a tumor cell or a tumor-infiltrating lymphocyte (TIL). In one embodiment, the sample includes one or more premalignant or malignant cells. In an embodiment, the sample is acquired from a hematologic malignancy (or premaligancy), e.g., a hematologic malignancy (or premaligancy) described herein. In certain embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In an embodiment, the sample is a cell not associated with a tumor, e.g., a non-tumor cell or a peripheral blood lymphocyte.

“Sensitivity,” as used herein, is a measure of the ability of a method to detect a sequence variant in a heterogeneous population of sequences. A method has a sensitivity of ST % for variants of F % if, given a sample in which the sequence variant is present as at least F % of the sequences in the sample, the method can detect the sequence at a confidence of C %, ST % of the time. By way of example, a method has a sensitivity of 90% for variants of 5% if, given a sample in which the variant sequence is present as at least 5% of the sequences in the sample, the method can detect the sequence at a confidence of 99%, 9 out of 10 times (F=5%; C=99%; ST=90%). Exemplary sensitivities include those of ST-90%, 95%, 99% for sequence variants at F=1%, 5%, 10%, 20%, 50%, 100% at confidence levels of C=90%, 95%, 99%, and 99.9%.

“Specificity,” as used herein, is a measure of the ability of a method to distinguish a truly occurring sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or nucleic acid molecules of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

A “control nucleic acid” or “reference nucleic acid” “ ” as used herein, refers to nucleic acid molecules from a control or reference sample. Typically, it is DNA, e.g., genomic DNA, or cDNA derived from RNA, not containing the alteration or variation in the gene or gene product. In certain embodiments, the reference or control nucleic acid sample is a wild-type or a non-mutated sequence. In certain embodiments, the reference nucleic acid sample is purified or isolated (e.g., it is removed from its natural state). In other embodiments, the reference nucleic acid sample is from a blood control, a normal adjacent tissue (NAT), or any other non-cancerous sample from the same or a different subject. In some embodiments, the reference nucleic acid sample comprises normal DNA mixtures. In some embodiments, the normal DNA mixture is a process matched control. In some embodiments, the reference nucleic acid sample has germline variants. In some embodiments, the reference nucleic acid sample does not have somatic alterations, e.g., serves as a negative control.

“Sequencing” a nucleic acid molecule requires determining the identity of at least 1 nucleotide in the molecule (e.g., a DNA molecule, an RNA molecule, or a cDNA molecule derived from an RNA molecule). In embodiments the identity of less than all of the nucleotides in a molecule are determined. In other embodiments, the identity of a majority or all of the nucleotides in the molecule is determined.

“Threshold value,” as used herein, is a value that is a function of the number of reads required to be present to assign a nucleotide value to a subject interval (e.g., a subgenomic interval or an expressed subgenomic interval). E.g., it is a function of the number of reads having a specific nucleotide value, e.g., “A,” at a nucleotide position, required to assign that nucleotide value to that nucleotide position in the subgenomic interval. The threshold value can, e.g., be expressed as (or as a function of) a number of reads, e.g., an integer, or as a proportion of reads having the value. By way of example, if the threshold value is X, and X+1 reads having the nucleotide value of “A” are present, then the value of “A” is assigned to the position in the subject interval (e.g., subgenomic interval or expressed subgenomic interval). The threshold value can also be expressed as a function of a mutation or variant expectation, mutation frequency, or of Bayesian prior. In an embodiment, a mutation frequency would require a number or proportion of reads having a nucleotide value, e.g., A or G, at a position, to call that nucleotide value. In embodiments the threshold value can be a function of mutation expectation, e.g., mutation frequency, and tumor type. E.g., a variant at a nucleotide position could have a first threshold value if the patient has a first tumor type and a second threshold value if the patient has a second tumor type.

As used herein, “target nucleic acid molecule” refers to a nucleic acid molecule that one desires to isolate from the nucleic acid library. In one embodiment, the target nucleic acid molecules can be a tumor nucleic acid molecule, a reference nucleic acid molecule, or a control nucleic acid molecule, as described herein.

“Tumor nucleic acid molecule,” or other similar term (e.g., a “tumor or cancer-associated nucleic acid molecule”), as used herein refers to a nucleic acid molecule having sequence from a tumor cell. The terms “tumor nucleic acid molecule” and “tumor nucleic acid” may sometimes be used interchangeably herein. In one embodiment, the tumor nucleic acid molecule includes a subject interval having a sequence (e.g., a nucleotide sequence) that has an alteration (e.g., a mutation) associated with a cancerous phenotype. In other embodiments, the tumor nucleic acid molecule includes a subject interval having a wild-type sequence (e.g., a wild-type nucleotide sequence). For example, a subject interval from a heterozygous or homozygous wild-type allele present in a cancer cell. A tumor nucleic acid molecule can include a reference nucleic acid molecule. Typically, it is DNA, e.g., genomic DNA, or cDNA derived from RNA, from a sample. In certain embodiments, the sample is purified or isolated (e.g., it is removed from its natural state). In some embodiments, the tumor nucleic acid molecule is a cfDNA. In some embodiments, the tumor nucleic acid molecule is a ctDNA. In some embodiments, the tumor nucleic acid molecule is DNA from a CTC.

“Reference nucleic acid molecule,” or other similar term (e.g., a “control nucleic acid molecule”), as used herein, refers to a nucleic acid molecule that comprises a subject interval having a sequence (e.g., a nucleotide sequence) that is not associated with the cancerous phenotype. In one embodiment, the reference nucleic acid molecule includes a wild-type or a non-mutated nucleotide sequence of a gene or gene product that when mutated is associated with the cancerous phenotype. The reference nucleic acid molecule can be present in a cancer cell or non-cancer cell,

“Variant,” as used herein, refers to a structure that can be present at a subgenomic interval that can have more than one structure, e.g., an allele at a polymorphic locus.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 KB or less than about 0.1 kB of nucleotide sequences which naturally flank the nucleic acid molecule in genomic DNA of the cell from which the nucleic acid is derived. Moreover, an “isolated” nucleic acid molecule, such as an RNA molecule or a cDNA molecule, can be substantially free of other cellular material or culture medium, e.g., when produced by recombinant techniques, or substantially free of chemical precursors or other chemicals, e.g., when chemically synthesized.

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

As used herein, “X is a function of Y” means, e.g., one variable X is associated with another variable Y. The association between X and Y can be direct or indirect. In one embodiment, if X is a function of Y, a causal relationship between X and Y may be implied, but does not necessarily exist.

Headings, e.g., (a), (b), (i) etc., are presented merely for ease of reading the specification and claims. The use of headings in the specification or claims does not require the steps or elements to be performed in alphabetical or numerical order or the order in which they are presented. The use of headings in the specification or claims also does not require performance of all of the steps or elements.

### Multigene Analysis

The methods described herein can be used in combination with, or as part of, a method for evaluating a set of subject intervals, e.g., from a set of genes or gene products described herein.

In certain embodiments, the set of genes comprises a plurality of genes, which in mutant form, are associated with an effect on cell division, growth or survival, or are associated with a cancer, e.g., a cancer described herein.

In certain embodiments, the set of genes comprises at least about 50 or more, about 100 or more, about 150 or more, about 200 or more, about 250 or more, about 300 or more, about 350 or more, about 400 or more, about 450 or more, about 500 or more, about 550 or more, about 600 or more, about 650 or more, about 700 or more, about 750 or more, or about 800 or more genes, e.g., as described herein. In some embodiments, the set of genes comprises at least about 50 or more, about 100 or more, about 150 or more, about 200 or more, about 250 or more, about 300 or more, or all of the genes chosen described in Tables 2A-5B.

In certain embodiments, the method comprises acquiring a library comprising a plurality of tumor nucleic acid molecules from the sample. In certain embodiments, the method further comprises contacting a library with target capture reagents to provide selected tumor nucleic acid molecules, wherein said target capture reagents hybridize with a tumor nucleic acid molecule from the library, thereby providing a library catch. In certain embodiments, the method further comprises acquiring a read for a subject interval comprising an alteration (e.g., somatic alteration) from a tumor nucleic acid molecule from a library or library catch, thereby acquiring a read for the subject interval, e.g., by a next-generation sequencing method. In certain embodiments, the method further comprises aligning a read for the subject interval by an alignment method, e.g., an alignment method described herein. In certain embodiments, the method further comprises assigning a nucleotide value for a nucleotide position from a read for the subject interval, e.g., by a mutation calling method described herein.

In certain embodiments, the method comprises one, two, three, four, or all of:
(a) acquiring a library comprising a plurality of tumor nucleic acid molecules from a sample;
(b) contacting the library with a plurality of target capture reagents to provide selected tumor nucleic acid molecules, wherein said plurality of target capture reagents hybridize with the tumor nucleic acid molecules, thereby providing a library catch;
(c) acquiring a read for a subject interval comprising the alteration (e.g., somatic alteration) from a tumor nucleic acid molecule from said library catch, thereby acquiring a read for the subject interval, e.g., by a next-generation sequencing method;
(d) aligning said read by an alignment method, e.g., an alignment method described herein; or
(e) assigning a nucleotide value from said read for a nucleotide position, e.g., by a mutation calling method described herein.

In certain embodiments, acquiring a read for the subject interval comprises sequencing a subject interval from at least about 50 or more, about 100 or more, about 150 or more, about 200 or more, about 250 or more, about 300 or more, about 350 or more, about 400 or more, about 450 or more, about 500 or more, about 550 or more, about 600 or more, about 650 or more, about 700 or more, about 750 or more, or about 800 or more genes. In certain embodiments, acquiring a read for the subject interval comprises sequencing a subject interval from at least about 50 or more, about 100 or more, about 150 or more, about 200 or more, about 250 or more, about 300 or more, or all of the genes described in Tables 2A-5B.

In certain embodiments, acquiring a read for the subject interval comprises sequencing with 100× or more average depth. In certain embodiments, acquiring a read for the subject interval comprises sequencing with about 250× or more average depth. In other embodiments, acquiring a read for the subject interval comprises sequencing with about 500× or more average depth. In certain embodiments, acquiring a read for the subject interval comprises sequencing with about 800× or more average depth. In other embodiments, acquiring a read for the subject interval comprises sequencing with about 1,000× or more average depth. In other embodiments, acquiring a read for the subject interval comprises sequencing with about 1,500× or more average depth. In other embodiments, acquiring a read for the subject interval comprises sequencing with about 2,000× or more average depth. In other embodiments, acquiring a read for the subject interval comprises sequencing with about 2,500× or more average depth. In certain embodiments, acquiring a read for the subject interval comprises sequencing with about 3,000× or more average depth. In certain embodiments, acquiring a read for the subject interval comprises sequencing with about 3,500× or more average depth. In certain embodiments, acquiring a read for the subject interval comprises sequencing with about 4,000× or more average depth. In certain embodiments, acquiring a read for the subject interval comprises sequencing with about 4,500× or more average depth. In certain embodiments, acquiring a read for the subject interval comprises sequencing with about 5,000× or more average depth. In certain embodiments, acquiring a read for the subject interval comprises sequencing with about 5,500× or more average depth. In certain embodiments, acquiring a read for the subject interval comprises sequencing with about 6,000× or more average depth.

In certain embodiments, acquiring a read for the subject interval comprises sequencing with about 100× or more average depth, at greater than about 99% of genes (e.g., exons) sequenced. In certain embodiments, acquiring a read for the subject interval comprises sequencing with about 250× or more average depth, at greater than about 99% of genes (e.g., exons) sequenced. In other embodiments, acquiring a read for the subject interval comprises sequencing with about 500× or more average depth, at greater than about 95% of genes (e.g., exons) sequenced. In other embodiments, acquiring a read for the subject interval comprises sequencing with about 800× or more average depth, at greater than about 95% of genes (e.g., exons) sequenced. In other embodiments, acquiring a read for the subject interval comprises sequencing with greater than about 1,000× average depth, at greater than about 90% of genes (e.g., exons) sequenced. In other embodiments, acquiring a read for the subject interval comprises sequencing with about 2,000× or more average depth, at greater than about 90% of genes (e.g., exons) sequenced. In other embodiments, acquiring a read for the subject interval comprises sequencing with about 3,000× or more average depth, at greater than about 90% of genes (e.g., exons) sequenced. In other embodiments, acquiring a read for the subject interval comprises sequencing with about 3,500× or more average depth, at greater than about 90% of genes (e.g., exons) sequenced. In other embodiments, acquiring a read for the subject interval comprises sequencing with about 4,000× or more average depth, at greater than about 90% of genes (e.g., exons) sequenced. In other embodiments, acquiring a read for the subject interval comprises sequencing with about 4,500× or more average depth, at greater than about 90% of genes (e.g., exons) sequenced. In other embodiments, acquiring a read for the subject interval comprises sequencing with about 5,000× or more average depth, at greater than about 90% of genes (e.g., exons) sequenced. In other embodiments, acquiring a read for the subject interval comprises sequencing with about 5,500× or more average depth, at greater than about 90% of genes (e.g., exons) sequenced. In other embodiments, acquiring a read for the subject interval comprises sequencing with about 6,000× or more average depth, at greater than about 90% of genes (e.g., exons) sequenced. In certain embodiments, acquiring a read for the subject interval comprises sequencing with about 100× or more, about 250× or more, about 500× or more, about 1,000× or more, about 1,500× or more, about 2,000× or more, about 2,500× or more, about 3,000× or more, about 3,500× or more, about 4,000× or more, about 4,500× or more, about 5,000× or more, about 5,500× or more, or about 6,000× or more average depth, at greater than about 99% of genes (e.g., exons) sequenced.

In certain embodiments, the sequence, e.g., a nucleotide sequence, of a set of subject intervals (e.g., coding subject intervals), described herein, is provided by a method described herein. In certain embodiments, the sequence is provided without using a method that includes a matched normal control (e.g., a wild-type control), a matched tumor control (e.g., primary versus. metastatic), or both.

### Gene Selection

Subject intervals, e.g., subgenomic intervals, expressed subgenomic intervals, or both, for analysis, e.g., a group or set of subgenomic intervals for sets or groups of genes and other regions, are described herein.

In some embodiments, the method comprises sequencing, e.g., by a next-generation sequencing method, a subject interval from at least 5, 6, 7, 8, 9, 10, 15, 20, 25, 30, 40, 50, 60, 70, 80, 90, 100, 200, 300, 400, 500, or more genes or gene products from the acquired nucleic acid sample, wherein the genes are chosen from Tables 2A-5B.

In some embodiments, the method comprises sequencing, e.g., by a next-generation sequencing method, a subject interval from at least 5, 6, 7, 8, 9, 10, 15, 20, 25, 30, 40, 50, 60, 70, 80, 90, 100, 200, 300, 400, 500, or more genes or gene products from the sample, wherein the genes are chosen from Tables 2A-5B.

In another embodiment, subject intervals of one of the following sets or groups are analyzed. E.g., subject intervals associated with a tumor or cancer gene or gene product and a reference (e.g., a wild-type) gene or gene product can provide a group or set of subgenomic intervals from the sample.

In an embodiment, the method acquires a read, e.g., sequences, a set of subject intervals from the sample, wherein the subject intervals are chosen from at least 1, 2, 3, 4, 5, 6, 7 or all of the following:
A) at least 5, 6, 7, 8, 9, 10, 15, 20, 25, 30, 40, 50, 60, 70, 80, 90, 100, 200, 300, 400, 500, or more subject intervals, e.g., subgenomic intervals, or expressed subgenomic intervals, or both, from a mutated or wild-type gene according to Tables 2A-5B;
B) at least 5, 6, 7, 8, 9, 10, 15, 20, 25, 30, 40, 50, 60, 70, 80, 90, 100, 200, 300, 400, 500, or more subject intervals from a gene or gene product that is associated with a tumor or cancer (e.g., is a positive or negative treatment response predictor, is a positive or negative prognostic factor for, or enables differential diagnosis of a tumor or cancer, e.g., a gene according to Tables 2A-5B); C) at least 5, 6, 7, 8, 9, 10, 15, 20, 25, 30, 40, 50, 60, 70, 80, 90, 100, 200, 300, 400, 500, or more of subject intervals from a mutated or wild-type gene or gene product (e.g., single nucleotide polymorphism (SNP)) of a subgenomic interval that is present in a gene chosen from Tables 2A-5B;
D) at least 5, 6, 7, 8, 9, 10, 15, 20, 25, 30, 40, 50, 60, 70, 80, 90, 100, 200, 300, 400, 500, or more of subject intervals from a mutated or wild-type gene (e.g., single nucleotide polymorphism (SNP)) of a subject interval that is present in a gene chosen from Tables 2A-5B associated with one or more of: (i) better survival of a cancer patient treated with a drug (e.g., better survival of a breast cancer patient treated with paclitaxel); (ii) paclitaxel metabolism; (iii) toxicity to a drug; or (iv) a side effect to a drug;
E) a plurality of translocation alterations involving at least 5, 6, 7, 8, 9, 10, 15, 20, 25, 30, 40, 50, 60, 70, 80, 90, 100, 200, 300, 400, 500, or more genes or gene products according to Tables 2A-5B;
F) at least five genes selected from Tables 2A-5B, wherein an allelic variation, e.g., at a position, is associated with a type of tumor and wherein said allelic variation is present in less than 5% of the cells in said tumor type;
G) at least five genes selected from Tables 2A-5B, which are embedded in a GC-rich region; or
H) at least five genes indicative of a genetic (e.g., a germline risk) factor for developing cancer (e.g., the gene or gene product is chosen from Tables 2A-5B).

In yet another embodiment, the method acquires reads, e.g., sequences, for a set of subject intervals from the sample, wherein the subject intervals are chosen from 5, 6, 7, 8, 9, 10, 15, 20, 25, 30, 40, 50, 60, 70, 80, 90, 100, 200, 300, 400, or all of the genes described in Tables 2A-2C.

In yet another embodiment, the method acquires reads, e.g., sequences, for a set of subject intervals from the sample, wherein the subject intervals are chosen from 5, 6, 7, 8, 9, 10, 15, 20, 25, 30, or all of the genes described in Tables 3A-3B.

In yet another embodiment, the method acquires reads, e.g., sequences, for a set of subject intervals from the sample, wherein the subject intervals are chosen from 5, 6, 7, 8, 9, 10, 15, 20, 25, 30, 40, 50, 60, 70, 80, 90, 100, 200, 300, or all of the genes described in Tables 4A-4C.

In yet another embodiment, the method acquires reads, e.g., sequences, for a set of subject intervals from the sample, wherein the subject intervals are chosen from 5, 6, 7, 8, 9, 10, 15, 20, 25, 30, 40, 50, 60, 70, 80, or all of the genes described in Tables 5A-5B.

The selected genes or gene products (also referred to herein as the “target genes or gene products”) can include subject intervals comprising intragenic regions or intergenic regions. For example, the subject intervals can include an exon or an intron, or a fragment thereof, typically an exon sequence or a fragment thereof. The subject interval can include a coding region or a non-coding region, e.g., a promoter, an enhancer, a 5′ untranslated region (5′ UTR), or a 3′ untranslated region (3′ UTR), or a fragment thereof. In other embodiments, the subject interval includes a cDNA or a fragment thereof. In other embodiments, the subject interval includes an SNP, e.g., as described herein.

In other embodiments, the subject intervals include substantially all exons in a genome, e.g., one or more of the subject intervals as described herein (e.g., exons from selected genes or gene products of interest (e.g., genes or gene products associated with a cancerous phenotype as described herein)). In one embodiment, the subject interval includes a somatic mutation, a germline mutation or both. In one embodiment, the subject interval includes an alteration, e.g., a point or a single mutation, a deletion mutation (e.g., an in-frame deletion, an intragenic deletion, a full gene deletion), an insertion mutation (e.g., intragenic insertion), an inversion mutation (e.g., an intra-chromosomal inversion), a linking mutation, a linked insertion mutation, an inverted duplication mutation, a tandem duplication (e.g., an intrachromosomal tandem duplication), a translocation (e.g., a chromosomal translocation, a non-reciprocal translocation), a rearrangement, a change in gene copy number, or a combination thereof. In certain embodiments, the subject interval constitutes less than 5%, 1%, 0.5%, 0.1%, 0.05%, 0.01%, 0.005%, or 0.001% of the coding region of the genome of the tumor cells in a sample. In other embodiments, the subject intervals are not involved in a disease, e.g., are not associated with a cancerous phenotype as described herein.

In one embodiment, the target gene or gene product is a biomarker. As used herein, a “biomarker” or “marker” is a gene, mRNA, or protein which can be altered, wherein said alteration is associated with cancer. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, a marker associated with cancer, or predictive of responsiveness to anti-cancer therapeutics, can have an altered nucleotide sequence, amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell as compared to a normal, healthy tissue or cell. Furthermore, a “marker” includes a molecule whose structure is altered, e.g., mutated (contains a mutation), e.g., differs from the wild-type sequence at the nucleotide or amino acid level, e.g., by substitution, deletion, or insertion, when present in a tissue or cell associated with a disease state, such as cancer.

In one embodiment, the target gene or gene product includes a single nucleotide polymorphism (SNP). In another embodiment, the gene or gene product has a small deletion, e.g., a small intragenic deletion (e.g., an in-frame or frame-shift deletion). In yet another embodiment, the target sequence results from the deletion of an entire gene. In still another embodiment, the target sequence has a small insertion, e.g., a small intragenic insertion. In one embodiment, the target sequence results from an inversion, e.g., an intrachromosomal inversion. In another embodiment, the target sequence results from an interchromosal translocation. In yet another embodiment, the target sequence has a tandem duplication. In one embodiment, the target sequence has an undesirable feature (e.g., high GC content or repeat element). In another embodiment, the target sequence has a portion of nucleotide sequence that cannot itself be successfully targeted, e.g., because of its repetitive nature. In one embodiment, the target sequence results from alternative splicing. In another embodiment, the target sequence is chosen from a gene or gene product, or a fragment thereof according to Tables 2A-5B.

In an embodiment, the target gene or gene product, or a fragment thereof, is an antibody gene or gene product, an immunoglobulin superfamily receptor (e.g., B-cell receptor (BCR) or T-cell receptor (TCR)) gene or gene product, or a fragment thereof.

Human antibody molecules (and B cell receptors) are composed of heavy and light chains with both constant (C) and variable (V) regions that are encoded by genes on at least the following three loci.
1. Immunoglobulin heavy locus (IGH@) on chromosome 14, containing gene segments for the immunoglobulin heavy chain;
2. Immunoglobulin kappa (κ) locus (IGK@) on chromosome 2, containing gene segments for the immunoglobulin light chain;
3. Immunoglobulin lambda (λ) locus (IGL@) on chromosome 22, containing gene segments for the immunoglobulin light chain.

Each heavy chain and light chain gene contains multiple copies of three different types of gene segments for the variable regions of the antibody proteins. For example, the immunoglobulin heavy chain region can contain one of five different classes γ, δ, α, μ and ε, 44 Variable (V) gene segments, 27 Diversity (D) gene segments, and 6 Joining (J) gene segments. The light chains can also possess numerous V and J gene segments, but do not have D gene segments. The lambda light chain has 7 possible C regions and the kappa light chain has 1.

Immunoglobulin heavy locus (IGH@) is a region on human chromosome 14 that contains genes for the heavy chains of human antibodies (or immunoglobulins). For example, the IGH locus includes IGHV (variable), IGHD (diversity), IGHJ (joining), and IGHC (constant) genes. Exemplary genes encoding the immunoglobulin heavy chains include, but are not limited to IGHV1-2, IGHV1-3, IGHV1-8, IGHV1-12, IGHV1-14, IGHV1-17, IGHV1-18, IGHV1-24, IGHV1-45, IGHV1-46, IGHV1-58, IGHV1-67, IGHV1-68, IGHV1-69, IGHV1-38-4, IGHV1-69-2, IGHV2-5, IGHV2-10, IGHV2-26, IGHV2-70, IGHV3-6, IGHV3-7, IGHV3-9, IGHV3-11, IGHV3-13, IGHV3-15, IGHV3-16, IGHV3-19, IGHV3-20, IGHV3-21, IGHV3-22, IGHV3-23, IGHV3-25, IGHV3-29, IGHV3-30, IGHV3-30-2, IGHV3-30-3, IGHV3-30-5, IGHV3-32, IGHV3-33, IGHV3-33-2, IGHV3-35, IGHV3-36, IGHV3-37, IGHV3-38, IGHV3-41, IGHV3-42, IGHV3-43, IGHV3-47, IGHV3-48, IGHV3-49, IGHV3-50, IGHV3-52, IGHV3-53, IGHV3-54, IGHV3-57, IGHV3-60, IGHV3-62, IGHV3-63, IGHV3-64, IGHV3-65, IGHV3-66, IGHV3-71, IGHV3-72, IGHV3-73, IGHV3-74, IGHV3-75, IGHV3-76, IGHV3-79, IGHV3-38-3, IGHV3-69-1, IGHV4-4, IGHV4-28, IGHV4-30-1, IGHV4-30-2, IGHV4-30-4, IGHV4-31, IGHV4-34, IGHV4-39, IGHV4-55, IGHV4-59, IGHV4-61, IGHV4-80, IGHV4-38-2, IGHV5-51, IGHV5-78, IGHV5-10-1, IGHV6-1, IGHV7-4-1, IGHV7-27, IGHV7-34-1, IGHV7-40, IGHV7-56, IGHV7-81, IGHVII-1-1, IGHVII-15-1, IGHVII-20-1, IGHVII-22-1, IGHVII-26-2, IGHVII-28-1, IGHVII-30-1, IGHVII-31-1, IGHVII-33-1, IGHVII-40-1, IGHVII-43-1, IGHVII-44-2, IGHVII-46-1, IGHVII-49-1, IGHVII-51-2, IGHVII-53-1, IGHVII-60-1, IGHVII-62-1, IGHVII-65-1, IGHVII-67-1, IGHVII-74-1, IGHVII-78-1, IGHVIII-2-1, IGHVIII-5-1, IGHVIII-5-2, IGHVIII-11-1, IGHVIII-13-1, IGHVIII-16-1, IGHVIII-22-2, IGHVIII-25-1, IGHVIII-26-1, IGHVIII-38-1, IGHVIII-44, IGHVIII-47-1, IGHVIII-51-1, IGHVIII-67-2, IGHVIII-67-3, IGHVIII-67-4, IGHVIII-76-1, IGHVIII-82, IGHVIV-44-1, IGHD1-1, IGHD1-7, IGHD1-14, IGHD1-20, IGHD1-26, IGHD2-2, IGHD2-8, IGHD2-15, IGHD2-21, IGHD3-3, IGHD3-9, IGHD3-10, IGHD3-16, IGHD3-22, IGHD4-4, IGHD4-11, IGHD4-17, IGHD4-23, IGHD5-5, IGHD5-12, IGHD5-18, IGHD5-24, IGHD6-6, IGHD6-13, IGHD6-19, IGHD6-25, IGHD7-27, IGHJ1, IGHJ1P, IGHJ2, IGHJ2P, IGHJ3, IGHJ3P, IGHJ4, IGHJ5, IGHJ6, IGHA1, IGHA2, IGHG1, IGHG2, IGHG3, IGHG4, IGHGP, IGHD, IGHE, IGHEP1, IGHM, and IGHV1-69D. Immunoglobulin kappa locus (IGK@) is a region on human chromosome 2 that contains genes for the kappa (κ) light chains of antibodies (or immunoglobulins). For example, the IGK locus includes IGKV (variable), IGKJ (joining), and IGKC (constant) genes. Exemplary genes encoding the immunoglobulin kappa light chains include, but are not limited to, IGKV1-5, IGKV1-6, IGKV1-8, IGKV1-9, IGKV1-12, IGKV1-13, IGKV1-16, IGKV1-17, IGKV1-22, IGKV1-27, IGKV1-32, IGKV1-33, IGKV1-35, IGKV1-37, IGKV1-39, IGKV1D-8, IGKV1D-12, IGKV1D-13, IGKV1D-16 IGKV1D-17, IGKV1D-22, IGKV1D-27, IGKV1D-32, IGKV1D-33, IGKV1D-35, IGKV1D-37, IGKV1D-39, IGKV1D-42, IGKV1D-43, IGKV2-4, IGKV2-10, IGKV2-14, IGKV2-18, IGKV2-19, IGKV2-23, IGKV2-24, IGKV2-26, IGKV2-28, IGKV2-29, IGKV2-30, IGKV2-36, IGKV2-38, IGKV2-40, IGKV2D-10, IGKV2D-14, IGKV2D-18, IGKV2D-19, IGKV2D-23, IGKV2D-24, IGKV2D-26, IGKV2D-28, IGKV2D-29, IGKV2D-30, IGKV2D-36, IGKV2D-38, IGKV2D-40, IGKV3-7, IGKV3-11, IGKV3-15, IGKV3-20, IGKV3-25, IGKV3-31, IGKV3-34, IGKV3D-7, IGKV3D-11, IGKV3D-15, IGKV3D-20, IGKV3D-25, IGKV3D-31. IGKV3D-34, IGKV4-1, IGKV5-2, IGKV6-21, IGKV6D-21, IGKV6D-41, IGKV7-3, IGKJ1, IGKJ2, IGKJ3, IGKJ4, IGKJ5, and IGKC.

Immunoglobulin lambda locus (IGL@) is a region on human chromosome 22 that contains genes for the lambda light chains of antibody (or immunoglobulins). For example, the IGL locus includes IGLV (variable), IGLJ (joining), and IGLC (constant) genes. Exemplary genes encoding the immunoglobulin lambda light chains include, but are not limited to, IGLV1-36, IGLV1-40, IGLV1-41, IGLV1-44, IGLV1-47, IGLV1-50, IGLV1-51, IGLV1-62, IGLV2-5, IGLV2-8, IGLV2-11, IGLV2-14, IGLV2-18, IGLV2-23, IGLV2-28, IGLV2-33, IGLV2-34, IGLV3-1, IGLV3-2, IGLV3-4, IGLV3-6, IGLV3-7, IGLV3-9, IGLV3-10, IGLV3-12, IGL V3-13, IGLV3-15, IGLV3-16, IGLV3-17, IGLV3-19, IGLV3-21, IGLV3-22, IGLV3-24, IGL V3-25, IGLV3-26, IGLV3-27, IGLV3-29, IGLV3-30, IGLV3-31, IGLV3-32, IGLV4-3, IGLV4-60, IGLV4-69, IGLV5-37, IGLV5-39, IGLV5-45, IGLV5-48, IGLV5-52, IGLV6-57, IGLV7-35, IGLV7-43, IGLV7-46, IGLV8-61, IGLV9-49, IGLV10-54, IGLV10-67, IGLV11-55, IGLVI-20, IGLVI-38, IGLVI-42, IGLVI-56, IGLVI-63, IGLVI-68, IGL VI-70, IGLVIV-53, IGLVIV-59, IGLVIV-64, IGLVIV-65, IGLVIV-66-1, IGLVV-58, IGLVV-66, IGLVVI-22-1, IGLVVI-25-1, IGLVVII-41-1, IGLJ1, IGLJ2, IGLJ3, IGLJ4, IGLJ5, IGLJ6, IGLJ7, IGLC1, IGLC2, IGLC3, IGLC4, IGLC5, IGLC6, and IGLC7.

The B-cell receptor (BCR) is composed of two parts: i) a membrane-bound immunoglobulin molecule of one isotype (e.g., IgD or IgM). With the exception of the presence of an integral membrane domain, these can be identical to their secreted forms and ii) a signal transduction moiety: a heterodimer called Ig-α/Ig-β (CD79), bound together by disulfide bridges. Each nucleic acid molecule of the dimer spans the plasma membrane and has a cytoplasmic tail bearing an immunoreceptor tyrosine-based activation motif (ITAM).

The T-cell receptor (TCR) is composed of two different protein chains (i.e., a heterodimer). In 95% of T cells, this consists of an alpha (α) and beta (β) chain, whereas in 5% of T cells this consists of gamma (γ) and delta (δ) chains. This ratio can change during ontogeny and in diseased states. The T cell receptor genes are similar to immunoglobulin genes in that they too contain multiple V, D and J gene segments in their beta and delta chains (and V and J gene segments in their alpha and gamma chains) that are rearranged during the development of the lymphocyte to provide each cell with a unique antigen receptor.

T-cell receptor alpha locus (TRA) is a region on human chromosome 14 that contains genes for the TCR alpha chains. For example, the TRA locus includes, e.g., TRAV (variable), TRAJ (joining), and TRAC (constant) genes. Exemplary genes encoding the T-cell receptor alpha chains include, but are not limited to, TRAV1-1, TRAV1-2, TRAV2, TRAV3, TRAV4, TRAV5, TRAV6, TRAV7, TRAV8-1, TRAV8-2, TRAV8-3, TRAV8-4, TRAV8-5, TRAV8-6, TRAV8-7, TRAV9-1, TRAV9-2, TRAV10, TRAV11, TRAV12-1, TRAV12-2, TRAV12-3, TRAV13-1, TRAV13-2, TRAV14DV4, TRAV15, TRAV16, TRAV17, TRAV18, TRAV19, TRAV20, TRAV21, TRAV22, TRAV23DV6, TRAV24, TRAV25, TRAV26-1, TRAV26-2, TRAV27, TRAV28, TRAV29DV5, TRAV30, TRAV31, TRAV32, TRAV33, TRAV34, TRAV35, TRAV36DV7, TRAV37, TRAV38-1, TRAV38-2DV8, TRAV39, TRAV40, TRAV41, TRAJ1, TRAJ2, TRAJ3, TRAJ4, TRAJ5, TRAJ6, TRAJ7, TRAJ8, TRAJ9, TRAJ10, TRAJ11, TRAJ12, TRAJ13, TRAJ14, TRAJ15, TRAJ16, TRAJ17, TRAJ18, TRAJ19, TRAJ20, TRAJ21, TRAJ22, TRAJ23, TRAJ24, TRAJ25, TRAJ26, TRAJ27, TRAJ28, TRAJ29, TRAJ30, TRAJ31, TRAJ32, TRAJ33, TRAJ34, TRAJ35, TRAJ36, TRAJ37, TRAJ38, TRAJ39, TRAJ40, TRAJ41, TRAJ42, TRAJ43, TRAJ44, TRAJ45, TRAJ46, TRAJ47, TRAJ48, TRAJ49, TRAJ50, TRAJ51, TRAJ52, TRAJ53, TRAJ54, TRAJ55, TRAJ56, TRAJ57, TRAJ58, TRAJ59, TRAJ60, TRAJ61, and TRAC.

T-cell receptor beta locus (TRB) is a region on human chromosome 7 that contains genes for the TCR beta chains. For example, the TRB locus includes, e.g., TRBV (variable), TRBD (diversity), TRBJ (joining), and TRBC (constant) genes. Exemplary genes encoding the T-cell receptor beta chains include, but are not limited to, TRBV1, TRBV2, TRBV3-1, TRBV3-2, TRBV4-1, TRBV4-2, TRBV4-3, TRBV5-1, TRBV5-2, TRBV5-3, TRBV5-4, TRBV5-5, TRBV5-6, TRBV5-7, TRBV6-2, TRBV6-3, TRBV6-4, TRBV6-5, TRBV6-6, TRBV6-7, TRBV6-8, TRBV6-9, TRBV7-1, TRBV7-2, TRBV7-3, TRBV7-4, TRBV7-5, TRBV7-6, TRBV7-7, TRBV7-8, TRBV7-9, TRBV8-1, TRBV8-2, TRBV9, TRBV10-1, TRBV10-2, TRBV10-3, TRBV11-1, TRBV11-2, TRBV11-3, TRBV12-1, TRBV12-2, TRBV12-3, TRBV12-4, TRBV12-5, TRBV13, TRBV14, TRBV15, TRBV16, TRBV17, TRBV18, TRBV19, TRBV20-1, TRBV21-1, TRBV22-1, TRBV23-1, TRBV24-1, TRBV25-1, TRBV26, TRBV27, TRBV28, TRBV29-1, TRBV30, TRBVA, TRBVB, TRBV5-8, TRBV6-1, TRBD1, TRBD2, TRBJ1-1, TRBJ1-2, TRBJ1-3, TRBJ1-4, TRBJ1-5, TRBJ1-6, TRBJ2-1, TRBJ2-2, TRBJ2-2P, TRBJ2-3, TRBJ2-4, TRBJ2-5, TRBJ2-6, TRBJ2-7, TRBC1, and TRBC2.

T-cell receptor delta locus (TRD) is a region on human chromosome 14 that contains genes for the TCR delta chains. For example, the TRD locus includes, e.g., TRDV (variable), TRDJ (joining), and TRDC (constant) genes. Exemplary genes encoding the T-cell receptor delta chains include, but are not limited to, TRDV1, TRDV2, TRDV3, TRDD1, TRDD2, TRDD3, TRDJ1, TRDJ2, TRDJ3, TRDJ4, and TRDC.

T-cell receptor gamma locus (TRG) is a region on human chromosome 7 that contains genes for the TCR gamma chains. For example, the TRG locus includes, e.g., TRGV (variable), TRGJ (joining), and TRGC (constant) genes. Exemplary genes encoding the T-cell receptor gamma chains include, but are not limited to, TRGV1, TRGV2, TRGV3, TRGV4, TRGV5, TRGV5P, TRGV6, TRGV7, TRGV8, TRGV9, TRGV10, TRGV11, TRGVA, TRGVB, TRGJ1, TRGJ2, TRGJP, TRGJP1, TRGJP2, TRGC1, and TRGC2.

In one embodiment, the target gene or gene product, or a fragment thereof, is selected from any of the genes or gene products described in Tables 2A-5B.

TABLE 2A
Exemplary genes with complete exonic coverage in an exemplary DNA-seq target capture reagent

| | | | | | | | | | |
|---|---|---|---|---|---|---|---|---|---|
| ABL1 | BTK | CTNNB | FAS | HIST1H1C | KDR | MYCN | PDK1 | | SUFU |
| | | | (TNFRSF6) | | | | | | |
| ACTB | BTLA | | | HIST1H1D | KEAP1 | MYD88 | PHF6 | | SUZ12 |
| AKT1 | c11orf30 | | FBXO11 | HIST1H1E | KIT | MYO18A | | | |
| | (EMSY) | | | | | | | | |
| AKT2 | CAD | CUX1 | FBXO31 | HIST1H2AC | KLHL6 | | | | TAF1 |
| AKT3 | CARD11 | CXCR4 | FBXW7 | HIST1H2AG | KMT2A | | PIK3CA | | TBL1XR1 |
| | | | | | (MLL) | | | | |
| ALK | CASP8 | | FGF10 | HIST1H2AL | ) | NCOR2 | PIK3CG | | |
| | CBFB | DAXX | | HIST1H2AM | KMT2C | NCSTN | PIK3R1 | RPTOR | TCF3 |
| | | | | | (MLL3) | | | | |
| AMER1 | CBL | DDR2 | FGF14 | HIST1H2BC | KRAS | NF1 | PIK3R2 | RUNX1 | TCL1A |
| (FAM123B | | | | | | | | | |
| or WTX) | | | | | | | | | |
| APC | CCND1 | DDX3X | FGF19 | HIST1H2BJ | LEF1 | NF2 | PIM1 | | TET2 |
| | CCND2 | | FGF23 | HIST1H2BK | LMO1 | NFE2L2 | PLCG2 | S1PR2 | TGFBR2 |
| APH1A | CCND3 | | FGF3 | HIST1H2BO | LRP1B | NFKBIA | PMS2 | | |
| AR | CCNE1 | DNM2 | FGF4 | HIST1H3B | LRRK2 | NKX2-1 | PNRC1 | SDHA | TLL2 |
| ARAF | CCT6B | DNMT3A | FGF6 | | MAF | NOD1 | POT1 | SDHB | TMEM30A |
| ARFRP1 | CD22 | DOT1L | | HNF1A | MAFB | NOTCH1 | PPP2R1A | SDHC | TMSB4XP8 |
| | | | | | | | | | (TMSL3) |
| ARHGAP26 | CD274 | DTX1 | FGFR1 | HRAS | MAGED1 | NOTCH2 | PRDM1 | SDHD | TNFAIP3 |
| (GRAF) | (PDL1) | | | | | | | | |
| ARID1A | CD36 | DUSP2 | FGFR2 | HSP90AA1 | MALT1 | | PRKAR1A | SERP2 | TNERSF11A |
| ARID2 | CD58 | DUSP9 | FGFR3 | ICK | MAP2K1 | | PRKDC | SETBP1 | TNFRSF14 |
| ASMTL | CD70 | EBF1 | FGFR4 | ID3 | MAP2K2 | NPM1 | PRSS8 | SETD2 | TNFRSF17 |
| ASXL1 | CD79A | ECT2L | FHIT | IDH1 | MAP2K4 | NRAS | PTCH1 | SF3B1 | TOP1 |
| ATM | CD79B | EED | FLCN | IDH2 | MAP3K1 | | PTEN | SGK1 | TP53 |
| ATR | CDC73 | EGFR | FLT1 | | MAP3K13 | NT5C2 | PTPN11 | SH2B3 | TP63 |
| ATRX | CDH1 | ELP2 | FLT3 | IGF1R | MAP3K14 | NTRK1 | PTPN2 | SMAD2 | TRAF2 |
| AURKA | CDK12 | EP300 | FLT4 | | MAP3K6 | NTRK2 | PTPN6 | SMAD4 | TRAF3 |
| | | | | | | | (SHP-1) | | |
| AURKB | CDK4 | EPHA3 | FLYWCH1 | IKBKE | MAP3K7 | NTRK3 | PTPRO | SMARCA1 | TRAF5 |
| AXIN1 | CDK6 | EPHA5 | FOXL2 | IKZF1 | MAPK1 | NUP93 | RAD21 | SMARCA4 | |
| AXL | CDK8 | EPHA7 | FOXO1 | IKZF2 | MCL1 | NUP98 | RAD50 | SMARCB1 | TSC1 |
| B2M | CDKN1B | EPHB1 | FOXO3 | IKZF3 | MDM2 | P2RY8 | RAD51 | | TSC2 |
| BAP1 | CDKN2A | ERBB2 | FOXP1 | IL7R | MDM4 | PAG1 | | SMC1A | TSHR |
| BARD1 | CDKN2B | ERBB3 | FRS2 | INHBA | MED12 | PAK3 | | SMC3 | TUSC3 |
| BCL10 | CDKN2C | ERBB4 | GADD45B | INPP4B | MEF2B | | | SMO | TYK2 |
| BCL11B | CEBPA | ERG | GATA1 | INPP5D | MEF2C | PALB2 | | SOCS1 | U2AF1 |
| | | | | (SHIP) | | | | | |
| BCL2 | CHD2 | ESR1 | GATA2 | IRF1 | MEN1 | | | SOCS2 | U2AF2 |
| BCL2L2 | CHEK1 | ETS1 | GATA3 | IRF4 | MET | | RAF1 | SOCS3 | VHL |
| BCL6 | CHEK2 | ETV6 | GID4 | IRF8 | MIB1 | | RARA | SOX10 | WDR90 |
| | | | (c17orf39) | | | | | | |
| BCL7A | | EXOSC6 | GNA11 | IRS2 | MITF | | RASGEF1A | SOX2 | WHSC1 |
| | | | | | | | | | (MMSET |
| | | | | | | | | | or NSD2) |
| BCOR | CIC | EZH2 | GNA12 | JAK1 | MKI67 | PASK | RB1 | SPEN | WISP3 |
| BCORL1 | CIITA | FAF1 | GNA13 | JAK2 | MLH1 | PAX5 | REL | SPOP | WT1 |
| BIRC3 | CKS1B | FAM46C | GNAQ | JAK3 | MPL | PBRM1 | RELN | SRC | XBP1 |
| BLM | CPS1 | FANCA | GNAS | JARID2 | MRE11A | PC | RET | SRSF2 | XPO1 |
| BRAF | CRBN | FANCC | GPR124 | JUN | MSH2 | PCBP1 | RHOA | STAG2 | |
| BRCA1 | CREBBP | FANCD2 | GRIN2A | KAT6A | MSH3 | PCLO | RICTOR | STAT3 | YY1AP1 |
| | | | | (MYST3) | | | | | |
| BRCA2 | CRKL | FANCE | GSK3B | KDM2B | MSH6 | PDCD1 | | STAT4 | ZMYM3 |
| BRD4 | CRLF2 | FANCF | GTSE1 | KDM4C | MTOR | PDCD11 | RNF43 | STAT5A | ZNF217 |
| BRIP1 | CSF1R | FANCG | HDAC1 | KDM5A | MUTYH | PDCD1LG2 | ROS1 | STAT5B | ZNF24 |
| (BACH1) | | | | | | (PDL2) | | | (ZSCAN3) |
| BRSK1 | CSF3R | | HDAC4 | KDM5C | MYC | PDGFRA | | STAT6 | ZNF703 |
| | CTCF | FANCL | HDAC7 | KDM6A | MYCL | PDGFRB | | STK11 | ZRSR2 |
| | | | | | (MYCL1) | | | | |
| BTG2 | CTNNA1 | | HGF | CALR | KMT2D | | | | |
| | | | | | (MLL2) | | | | |

TABLE 2B
Select DNA rearrangements

| | | | | | | | | | |
|---|---|---|---|---|---|---|---|---|---|
| ALK | BCL2 | BCL6 | BCR | BRAF | CCND1 | CRLF2 | EGFR | EPOR | ETV1 |
| ETV4 | ETV5 | ETV6 | EWSR1 | FGFR2 | IGH | IGK | IGL | JAK1 | JAK2 |
| KMT2A(MLL) | MYC | NTRK1 | PDGFRA | PDGFRB | RAF1 | RARA | RET | ROS1 | TMPRSS2 |
| TRG | | | | | | | | | |

TABLE 2C
Select RNA gene fusions

| | | | | | | | | | |
|---|---|---|---|---|---|---|---|---|---|
| ABI1 | ABL1 | ABL2 | ACSL6 | AFF1 | AFF4 | ALK | ARHGAP26 | ARHGEF12 | ARID1A |
| | | | | | | | (GRAF) | | |
| ARNT | ASXL1 | ATF1 | ATGS | ATIC | BCL10 | BCL11A | BCL11B | BCL2 | BCL3 |
| BCL6 | BCL7A | BCL9 | BCOR | BCR | BIRC3 | BRAF | BTG1 | CAMTA1 | CARS |
| CBFA2T3 | CBFB | CBL | CCND1 | CCND2 | CCND3 | CD274 | CDK6 | CDX2 | CHIC2 |
| | | | | | | (PD-L1) | | | |
| CHN1 | CIC | CIITA | CLP1 | CLTC | CLTCL1 | CNTRL | COL1A1 | CREB3L1 | CREB3L2 |
| | | | | | | (CEP110) | | | |
| CREBBP | CRLF2 | CSF1 | CTNNB1 | DDIT3 | DDX10 | DDX6 | DEK | DUSP22 | EGFR |
| EIF4A2 | ELF4 | ELL | ELN | EML4 | EP300 | EPOR | EPS15 | ERBB2 | ERG |
| ETS1 | ETV1 | ETV4 | ETV5 | ETV6 | EWSR1 | FCGR2B | FCRL4 | FEV | FGFR1 |
| FGFR1OP | FGFR2 | FGFR3 | FLI1 | FNBP1 | FOXO1 | FOXO3 | FOXO4 | FOXP1 | FSTL3 |
| FUS | GAS7 | GLI1 | GMPS | GPHN | HERPUD1 | HEY1 | HIP1 | HIST1H41 | HLF |
| HMGA1 | HMGA2 | HOXA11 | HOXA13 | HOXA3 | HOXA9 | HOXC11 | HOXC13 | HOXD11 | HOXD13 |
| HSP90AA1 | HSP90AB1 | IGH | IGK | IGL | IKZF1 | IL21R | IL3 | IRF4 | ITK |
| JAK1 | JAK2 | JAK3 | JAZF1 | KAT6A | KDSR | KIF5B | KMT2A | LASP1 | LCP1 |
| | | | | (MYST3) | | | (MLL) | | |
| LMO1 | LMO2 | LPP | LYL1 | MAF | MAFB | MALT1 | MDS2 | MECOM | MKL1 |
| MLF1 | MLLT1 | MLLT10 | MLLT3 | MLLT4 | MLLT6 | MN1 | MNX1 | MSI2 | MSN |
| | (ENL) | (AF10) | | (AF6) | | | | | |
| MUC1 | MYB | MYC | MYH11 | MYH9 | NACA | NBEAP1 | NCOA2 | NDRG1 | NF1 |
| | | | | | | (BCL8) | | | |
| NF2 | NFKB2 | NIN | NOTCH1 | NPM1 | NR4A3 | NSD1 | NTRK1 | NTRK2 | NTRK3 |
| NUMA1 | NUP214 | NUP98 | NUTM2A | OMD | P2RY8 | PAFAH1B2 | PAX3 | PAX5 | PAX7 |
| PBX1 | PCM1 | PCSK7 | PDCD1LG2 | PDE4DIP | PDGFB | PDGFRA | PDGFRB | PER1 | PHF1 |
| | | | (PD-L2) | | | | | | |
| PICALM | PIM1 | PLAG1 | PML | POU2AF1 | PPP1CB | PRDM1 | PRDM16 | PRRX1 | PSIP1 |
| PTCH1 | PTK7 | RABEP1 | RAF1 | RALGDS | RAP1GDS1 | RARA | RBM15 | RET | RHOH |
| RNF213 | ROS1 | RPL22 | RPN1 | RUNX1 | RUNX1T1 | RUNX2 | SEC31A | SEPT5 | SEPT6 |
| | | | | | (ETO) | | | | |
| SEPT9 | SET | SH3GL1 | SLC1A2 | SNX29 | SRSF3 | SS18 | SSX1 | SSX2 | SSX4 |
| | | | | (RUNDC2A) | | | | | |
| STAT6 | STL | SYK | TAF15 | TAL1 | TAL2 | TBL1XR1 | TCF3 | TCL1A | TEC |
| | | | | | | | (E2A) | (TCL1) | |
| TET1 | TFE3 | TFG | TFPT | TFRC | TLX1 | TLX3 | TMPRSS2 | TNFRSF11A | TOP1 |
| TP63 | TPM3 | TPM4 | TRIM24 | TRIP11 | TTL | TYK2 | USP6 | WHSC1 | or |
| | | | | | | | | (MMSET | |
| NSD2) | WHSC1L1 | YPEL5 | ZBTB16 | ZMYM2 | ZNF384 | ZNF52 | | | |

TABLE 3A
Exemplary genes with select introns covered in an exemplary DNA-seq target capture reagent

| | | | | | | | | | |
|---|---|---|---|---|---|---|---|---|---|
| ABL1 | ABL2 | ACVR1B | AKT1 | AKT2 | AKT3 | ALK | AMER1 | APC | AR |
| | | | | | | | (FAM123B) | | |
| ARAF | ARFRP1 | ARID1A | ARID1B | ARID2 | ASXL1 | ATM | ATR | ATRX | AURKA |
| AURKB | AXIN1 | AXL | BAP1 | BARD1 | BCL2 | BCL2L1 | BCL2L2 | BCL6 | BCOR |
| BCORL1 | BLM | BRAF | BRCA1 | BRCA2 | BRD4 | BRIP1 | BTG1 | BTK | C11orf30 |
| | | | | | | | | | (EMSY) |
| CARD11 | CBFB | CBL | CCND1 | CCND2 | CCND3 | CCNE1 | CD274 | CD79A | CD79B |
| | | | | | | | (PD-L1) | | |
| CDC73 | CDH1 | CDK12 | CDK4 | CDK6 | CDK8 | CDKN1A | CDKN1B | CDKN2A | CDKN2B |
| CDKN2C | CEBPA | CHD2 | CHD4 | CHEK1 | CHEK2 | CIC | CREBBP | CRKL | CRLF2 |
| CSF1R | CTCF | CTNNA1 | CTNNB1 | CUL3 | CYLD | DAXX | DDR2 | DICER1 | DNMT3A |
| DOT1L | EGFR | EP300 | EPHA3 | EPHA5 | EPHA7 | EPHB1 | ERBB2 | ERBB3 | ERBB4 |
| ERG | ERRFl1 | ESR1 | EZH2 | FAM46C | FANCA | FANCC | FANCD2 | FANCE | FANCF |
| FANCG | FANCL | FAS | FAT1 | FBXW7 | FGF10 | FGF14 | FGF19 | FGF23 | FGF3 |
| FGF4 | FGF6 | FGFR1 | FGFR2 | FGFR3 | FGFR4 | FH | FLCN | FLT1 | FLT3 |
| FLT4 | FOXL2 | FOXP1 | FRS2 | FUBP1 | GABRA6 | GATA1 | GATA2 | GATA3 | GATA4 |
| GATA6 | GID4 | GLl1 | GNA11 | GNA13 | GNAQ | GNAS | GPR124 | GRIN2A | GRM3 |
| | (C17orf39) | | | | | | | | |
| GSK3B | H3F3A | HGF | HNF1A | HRAS | HSD3B1 | HSP90AA1 | IDH1 | IDH2 | IGF1R |
| IGF2 | IKBKE | IKZF1 | IL7R | INHBA | INPP4B | IRF2 | IRF4 | IRS2 | JAK1 |
| JAK2 | JAK3 | JUN | KAT6A | KDM5A | KDM5C | KDM6A | KDR | KEAP1 | KEL |
| | | | (MYST3) | | | | | | |
| KIT | KLHL6 | KMT2A | KMT2C | KMT2D | KRAS | LMO1 | LRP1B | LYN | LZTR1 |
| | | (MLL) | (MLL3) | (MLL2) | | | | | |
| MAGI2 | MAP2K1 | MAP2K2 | MAP2K4 | MAP3K1 | MCL1 | MDM2 | MDM4 | MED12 | MEF2B |
| | (MEK1) | (MEK2) | | | | | | | |
| MEN1 | MET | MITF | MLH1 | MPL | MRE11A | MSH2 | MSH6 | MTOR | MUTYH |
| MYC | MYCL | MYCN | MYD88 | NF1 | NF2 | NFE2L2 | NFKBIA | NKX2-1 | NOTCH1 |
| | (MYCL1) | | | | | | | | |
| NOTCH2 | NOTCH3 | NPM1 | NRAS | NSD1 | NTRK1 | NTRK2 | NTRK3 | NUP93 | PAK3 |
| PALB2 | PARK2 | PAX5 | PBRM1 | PDCD1LG2 | PDGFRA | PDGFRB | PDK1 | PIK3C2B | PIK3CA |
| | | | | (PD-L2) | | | | | |
| PIK3CB | PIK3CG | PIK3R1 | PIK3R2 | PLCG2 | PMS2 | POLD1 | POLE | PPP2R1A | PRDM1 |
| PREX2 | PRKAR1A | PRKCI | PRKDC | PRSS8 | PTCH1 | PTEN | PTPN11 | QKI | RAC1 |
| RAD50 | RAD51 | RAF1 | RANBP2 | RARA | RB1 | RBM10 | RET | RICTOR | RNF43 |
| ROS1 | RPTOR | RUNX1 | RUNX1T1 | SDHA | SDHB | SDHC | SDHD | SETD2 | SF3B1 |
| SLIT2 | SMAD2 | SMAD3 | SMAD4 | SMARCA4 | SMARCB1 | SMO | SNCAIP | SOCS1 | SOX10 |
| SOX2 | SOX9 | SPEN | SPOP | SPTA1 | SRC | STAG2 | STAT3 | STAT4 | STK11 |
| SUFU | SYK | TAF1 | TBX3 | TERC | TERT | | TET2 | TGFBR2 | TNFAIP3 |
| | | | | | (Promoter | | | | |
| | | | | | only) | | | | |
| TNFRSF14 | TOP1 | TOP2A | TP53 | TSC1 | TSC2 | TSHR | U2AF1 | VEGFA | VHL |
| WISP3 | WT1 | XPO1 | ZBTB2 | ZNF217 | ZNF703 | | | | |

TABLE 3B
Select rearrangements

| | | | | | | | | | |
|---|---|---|---|---|---|---|---|---|---|
| ALK | BCL2 | BCR | BRAF | BRCA1 | BRCA2 | BRD4 | EGFR | ETV1 | ETV4 |
| ETV5 | ETV6 | FGFR1 | FGFR2 | FGFR3 | KIT | MSH2 | MYB | MYC | NOTCH2 |
| NTRK1 | NTRK2 | PDGFRA | RAF1 | RARA | RET | ROS1 | TMPRSS2 | | |

TABLE 4A
Exemplary genes targeted in an exemplary
RNA-seq target capture reagent

| | | | |
|---|---|---|---|
| BRCA1 | CRKL | MDM2 | SMO |
| BRCA2 | EGFR | MET | TP53 |
| CCND1 | ERBB2 | MYC | VEGFA |
| CD274 (PD-L1) | ERRFI1 | MYCN | |
| CDH1 | FGFR1 | NF1 | |
| CDK4 | FGFR2 | PDCD1LG2 (PD-L2) | |
| CDK6 | FOXL2 | PTEN | |
| CDKN2A | KRAS | PTPN11 | |

TABLE 4B
Select Exons

| | | | | | | | | | |
|---|---|---|---|---|---|---|---|---|---|
| ABL1 | AKT1 | ALK | ARAF | BRAF | BTK | CTNNB1 | DDR2 | ESR1 | EZH2 |
| FGFR3 | FLT3 | GNA11 | GNAQ | GNAS | HRAS | IDH1 | IDH2 | JAK2 | JAK3 |
| KIT | MAP2K1(MEK1) | MAP2K2(MEK2) | MPL | MTOR | MYD88 | NPM1 | NRAS | PDGFRA | PDGFRB |
| PIK3CA | RAFI | RET | TERT | | | | | | |

TABLE 4C
Select rearrangements

| | | |
|---|---|---|
| ALK | FGFR3 | RET |
| EGFR | PDGFRA | ROS1 |

TABLE 5A
Additional exemplary genes with complete exonic coverage in an exemplary DNA-seq target capture reagent

| | | | | | | | | | |
|---|---|---|---|---|---|---|---|---|---|
| ABL1 | ACVR1B | AKT1 | AKT2 | AKT3 | ALK | ALOX12B | AMER1 | APC | AR |
| | | | | | | | (FAM123B) | | |
| ARAF | ARFRP1 | ARID1A | ASXL1 | ATM | ATR | ATRX | AURKA | AURKB | AXIN1 |
| AXL | BAP1 | BARD1 | BCL2 | BCL2L1 | BCL2L2 | BCL6 | BCOR | BCORL1 | BRAF |
| BRCA1 | BRCA2 | BRD4 | BRIP1 | BTG1 | BTG2 | BTK | C11orf30 | CALR | CARD11 |
| | | | | | | | (EMSY) | | |
| CASP8 | CBFB | CBL | CCND1 | CCND2 | CCND3 | CCNE1 | CD22 | CD274 | CD70 |
| | | | | | | | | (PD-L1) | |
| CD79A | CD79B | CDC73 | CDH1 | CDK12 | CDK4 | CDK6 | CDK8 | CDKN1A | CDKN1B |
| CDKN2A | CDKN2B | CDKN2C | CEBPA | CHEK1 | CHEK2 | CIC | CREBBP | CRKL | CSF1R |
| CSF3R | CTCF | CTNNA1 | CTNNB1 | CUL3 | CUL4A | CXCR4 | CYP17A1 | DAXX | DDR1 |
| DDR2 | DIS3 | DNMT3A | DOT1L | EED | EGFR | EP300 | EPHA3 | EPHB1 | EPHB4 |
| ERBB2 | ERBB3 | ERBB4 | ERCC4 | ERG | ERRFI1 | ESR1 | EZH2 | FAM46C | FANCA |
| FANCC | FANCG | FANCL | FAS | FBXW7 | FGF10 | FGF12 | FGF14 | FGF19 | FGF23 |
| FGF3 | FGF4 | FGF6 | FGFR1 | FGFR2 | FGFR3 | FGFR4 | FH | FLCN | FLT1 |
| FLT3 | FOXL2 | FUBP1 | GABRA6 | GATA3 | GATA4 | GATA6 | GID4 | GNA11 | GNA13 |
| | | | | | | | (C17orf39) | | |
| GNAQ | GNAS | GRM3 | GSK3B | H3F3A | HDAC1 | HGF | HNF1A | HRAS | HSD3B1 |
| ID3 | IDH1 | IDH2 | IGF1R | IKBKE | IKZF1 | INPP4B | IRF2 | IRF4 | IRS2 |
| JAK1 | JAK2 | JAK3 | JUN | KDMSA | KDM5C | KDM6A | KDR | KEAP1 | KEL |
| KIT | KLHL6 | KMT2A | KMT2D | KRAS | LTK | LYN | MAF | MAP2K1 | MAP2K2 |
| | | (MLL) | (MLL2) | | | | | (MEK1) | (MEK2) |
| MAP2K4 | MAP3K1 | MAP3K13 | MAPK1 | MCL1 | MDM2 | MDM4 | MED12 | MEF2B | MEN1 |
| MERTK | MET | MITF | MKNK1 | MLH1 | MPL | MRE11A | MSH2 | MSH3 | MSH6 |
| MST1R | MTAP | MTOR | MUTYH | MYC | MYCL | MYCN | MYD88 | NBN | NF1 |
| | | | | | (MYCL1) | | | | |
| NF2 | NFE2L2 | NFKBIA | NKX2-1 | NOTCH1 | NOTCH2 | NOTCH3 | NPM1 | NRAS | NT5C2 |
| NTRK1 | NTRK2 | NTRK3 | P2RY8 | PALB2 | PARK2 | PARP1 | PARP2 | PARP3 | PAX5 |
| PBRM1 | PDCD1 | PDCD1LG2 | PDGFRA | PDGFRB | PDK1 | PIK3C2B | PIK3C2G | PIK3CA | PIK3CB |
| | (PD-1) | (PD-L2) | | | | | | | |
| PIK3R1 | PIM1 | PMS2 | POLD1 | POLE | PPARG | PPP2R1A | PPP2R2A | PRDM1 | PRKAR1A |
| PRKCI | PTCH1 | PTEN | PTPN11 | PTPRO | QKI | RAC1 | RAD21 | RAD51 | RAD51B |
| RAD51C | RAD51D | RAD52 | RAD54L | RAF1 | RARA | RB1 | RBM10 | REL | RET |
| RICTOR | RNF43 | ROS1 | RPTOR | SDHA | SDHB | SDHC | SDHD | SETD2 | SF3B1 |
| SGK1 | SMAD2 | SMAD4 | SMARCA4 | SMARCB1 | SMO | SNCAIP | SOCS1 | SOX2 | SOX9 |
| SPEN | SPOP | SRC | STAG2 | STAT3 | STK11 | SUFU | SYK | TBX3 | TEK |
| TET2 | TGFBR2 | TIPARP | TNFAIP3 | TNFRSF14 | TP53 | TSC1 | TSC2 | TYRO3 | U2AF1 |
| VEGFA | VHL | WHSC1 | WHSC1L1 | WT1 | XPO1 | XRCC2 | ZNF217 | ZNF703 | |
| | | (MMSET) | | | | | | | |

TABLE 5B
Select rearrangements

| | | | | | | | | | |
|---|---|---|---|---|---|---|---|---|---|
| ALK | BCL2 | BCR | BRAF | BRCA1 | BRCA2 | CD74 | EGFR | ETV4 | ETV5 |
| ETV6 | EWSR1 | EZR | FGFR1 | FGFR2 | FGFR3 | KIT | KMT2A(MLL) | MSH2 | MYB |
| MYC | NOTCH2 | NTRK1 | NTRK2 | NUTM1 | PDGFRA | RAF1 | RARA | RET | ROS1 |
| RSPO2 | SDC4 | SLC34A2 | TERC | TERT (promoter only) | TMPRSS2 | | | | |

Additional exemplary genes are described, e.g., in Tables 1-11 of International Application Publication No. WO2012/092426, the content of which is incorporated by reference in its entirety.

Applications of the foregoing methods include, but are not limited to, using a library of oligonucleotides containing all known sequence variants (or a subset thereof) of a particular gene or genes for sequencing in medical specimens.

### Type of Alterations

The methods described herein can be used in combination with, or as part of, a method for evaluating genomic alterations, as described herein.

Various types of alterations (e.g., somatic alterations) can be evaluated and used for the analysis of genomic alterations. For example, genomic alterations associated with cancer and/or tumor mutational burden can be analyzed. In some embodiments, the methods described herein are useful for analyzing samples with low tumor content and/or low amounts of tumor nucleic acids.

### Somatic Alterations

In certain embodiments, the alteration evaluated in accordance with a method described herein is a somatic alteration.

In certain embodiments, the alteration (e.g., somatic alteration) is a coding short variant, e.g., a base substitution or an indel (insertion or deletion). In certain embodiments, the alteration (e.g., somatic alteration) is a point mutation. In other embodiments, the alteration (e.g., somatic alteration) is other than a rearrangement, e.g., other than a translocation. In certain embodiments, the alteration (e.g., somatic alteration) is a splice variant,

In certain embodiments, the alteration (e.g., somatic alteration) is a silent mutation, e.g., a synonymous alteration. In other embodiments, the alteration (e.g., somatic alteration) is a non-synonymous single nucleotide variant (SNV). In other embodiments, the alteration (e.g., somatic alteration) is a passenger mutation, e.g., an alteration that has no detectable effect on the fitness of a clone of cells. In certain embodiments, the alteration (e.g., somatic alteration) is a variant of unknown significance (VUS), e.g., an alteration, the pathogenicity of which can neither be confirmed nor ruled out. In certain embodiments, the alteration (e.g., somatic alteration) has not been identified as being associated with a cancer phenotype,

In certain embodiments, the alteration (e.g., somatic alteration) is not associated with, or is not known to be associated with, an effect on cell division, growth or survival. In other embodiments, the alteration (e.g., somatic alteration) is associated with an effect on cell division, growth or survival.

In certain embodiments, an increased level of a somatic alteration is an increased level of one or more classes or types of a somatic alteration (e.g., a rearrangement, a point mutation, an indel, or any combination thereof). In certain embodiments, an increased level of a somatic alteration is an increased level of one class or type of a somatic alteration (e.g., a rearrangement only, a point mutation only, or an indel only). In certain embodiments, an increased level of a somatic alteration is an increased level of a somatic alteration at a position (e.g., a nucleotide positions, e.g., at one or more nucleotide positions), or at a region, (e.g., at a nucleotide region, e.g., at one or more nucleotide regions). In certain embodiments, an increased level of a somatic alteration is an increased level of a somatic alteration (e.g., a somatic alteration described herein).

### Functional Alterations

In certain embodiments, the alteration (e.g., a somatic alteration) is a functional alteration in a subgenomic interval. In other embodiments, the alteration (e.g., a somatic alteration) is not a known functional alteration in a subgenomic interval. For example, when tumor mutational burden is evaluated, the number of alterations (e.g., somatic alterations) can exclude one or more functional alterations.

In some embodiments, the functional alteration is an alteration that, compared with a reference sequence, e.g., a wild-type or unmutated sequence, has an effect on cell division, growth or survival, e.g., promotes cell division, growth or survival. In certain embodiments, the functional alteration is identified as such by inclusion in a database of functional alterations, e.g., the COSMIC database (cancer.sanger.ac.uk/cosmic; Forbes et al. Nucl. Acids Res. 2015; 43 (D1): D805-D811). In other embodiments, the functional alteration is an alteration with known functional status, e.g., occurring as a known somatic alteration in the COSMIC database. In certain embodiments, the functional alteration is an alteration with a likely functional status, e.g., a truncation in a tumor suppressor gene. In certain embodiments, the functional alteration is a driver mutation, e.g., an alteration that gives a selective advantage to a clone in its microenvironment, e.g., by increasing cell survival or reproduction. In other embodiments, the functional alteration is an alteration capable of causing clonal expansions. In certain embodiments, the functional alteration is an alteration capable of causing one, two, three, four, five, or all of the following: (a) self-sufficiency in a growth signal; (b) decreased, e.g., insensitivity, to an antigrowth signal; (c) decreased apoptosis; (d) increased replicative potential; (e) sustained angiogenesis; or (f) tissue invasion or metastasis.

In certain embodiments, the functional alteration is not a passenger mutation, e.g., is not an alteration that has no detectable effect on the fitness of a clone of cells. In certain embodiments, the functional alteration is not a variant of unknown significance (VUS), e.g., is not an alteration, the pathogenicity of which can neither be confirmed nor ruled out.

In certain embodiments, a plurality (e.g., about 10%, 20%, 30%, 40%, 50%, 60%, 70%, 80%, 90%, or more) of functional alterations in a gene described in Tables 2A-5B are excluded. In certain embodiments, all functional alterations in a gene described in Tables 2A-5B are excluded. In certain embodiments, a plurality of functional alterations in a plurality of genes described in Tables 2A-5B is excluded. In certain embodiments, all functional alterations in all genes described in Tables 2A-5B are excluded.

### Germline Alterations

In certain embodiments, the alteration is a germline alteration. In other embodiments, the alteration is not a germline alteration. In certain embodiments, the alteration is not identical or similar to, e.g., is distinguishable from, a germline alteration. For example, when tumor mutational burden is evaluated, the number of alterations can exclude the number of germline alterations.

In certain embodiments, the germline alteration is a single nucleotide polymorphism (SNP), a base substitution, an indel (e.g., an insertion or a deletion), or a silent alteration (e.g., synonymous alteration).

In certain embodiments, the germline alteration is identified by use of a method that does not use a comparison with a matched normal sequence. In other embodiments, the germline alteration is identified by a method comprising the use of an SGZ algorithm. In certain embodiments, the germline alteration is identified as such by inclusion in a database of germline alterations, e.g., the dbSNP database (www.ncbi.nlm.nih.gov/SNP/index.html; Sherry et al. Nucleic Acids Res. 2001; 29(1): 308-311). In other embodiments, the germline alteration is identified as such by inclusion in two or more counts of the ExAC database (exac.broadinstitute.org; Exome Aggregation Consortium et al. “Analysis of protein-coding genetic variation in 60,706 humans,” bioRxiv preprint. Oct. 30, 2015). In some embodiments, the germline alteration is identified as such by inclusion in the 1000 Genome Project database (www.1000genomes.org; McVean et al. Nature. 2012; 491, 56-65). In some embodiments, the germline alteration is identified as such by inclusion in the ESP database (Exome Variant Server, NHLBI GO Exome Sequencing Project (ESP), Seattle, WA (evs.gs.washington.edu/EVS/).

### Samples

The methods described herein can be used to evaluate tumor fraction in various types of samples from a number of different sources.

In some embodiments, the sample comprises a nucleic acid, e.g., DNA, RNA, or both. In certain embodiments, the sample comprises one or more nucleic acids from a tumor. In certain embodiments, the sample further comprises one or more non-nucleic acid components from the tumor, e.g., a cell, protein, carbohydrate, or lipid. In certain embodiments, the sample further comprises one or more nucleic acids from a non-tumor cell or tissue.

In certain embodiments, the sample is acquired from a liquid biopsy. In certain embodiments, the sample is not acquired from a tissue biopsy. In certain embodiment, the sample is a liquid sample. In certain embodiments, the sample is free, or essentially free, of solids. In some embodiments, a liquid biopsy comprises blood, plasma, cerebrospinal fluid, sputum, stool, urine, or saliva,

In certain embodiments, the sample is acquired from a subject having a solid tumor, a hematological cancer, or a metastatic form thereof. In certain embodiments, the sample is obtained from a subject having a cancer, or at risk of having a cancer. In certain embodiments, the sample is obtained from a subject who has not received a therapy to treat a cancer, is receiving a therapy to treat a cancer, or has received a therapy to treat a cancer, as described herein.

In some embodiments, the sample comprises one or more nucleic acids, e.g., DNA, RNA, or both, from a premalignant or malignant cell, a cell from a solid tumor, a soft tissue tumor or a metastatic lesion, a cell from a hematological cancer, a histologically normal cell, a circulating tumor cells (CTCs), or a combination thereof. In some embodiments, the sample comprises one or more cells chosen from a premalignant or malignant cell, a cell from a solid tumor, a soft tissue tumor or a metastatic lesion, a cell from a hematological cancer, a histologically normal cell, a circulating tumor cell (CTC), or a combination thereof.

In certain embodiments, the sample comprises cell-free DNA (cfDNA). In certain embodiments, the sample comprises circulating tumor DNA (ctDNA). In certain embodiments, the sample comprises blood, serum, or plasma. In certain embodiments, the sample comprises cerebral spinal fluid (CSF). In certain embodiments, the sample comprises pleural effusion. In certain embodiments, the sample comprises ascites. In certain embodiments, the sample comprises urine. In certain embodiments, the sample comprises a resection, a needle biopsy, a fine needle aspirate, or a cytology smear. In certain embodiments, the sample is a formalin-fixed paraffin-embedded (FFPE) sample.

A variety of tissue can be the source of the samples used in the present methods. Genomic or subgenomic nucleic acid (e.g., DNA or RNA) can be isolated from a subject's sample (e.g., a sample comprising tumor cells, a blood sample, a blood constituent sample, a sample comprising cell-free DNA (cfDNA), a sample comprising circulating tumor DNA (ctDNA), a sample comprising circulating tumor cells (CTCs), or any normal control (e.g., a normal adjacent tissue (NAT)).

In some embodiments, the sample comprises a nucleic acid, e.g., DNA, RNA, or both, e.g., from a tumor. The nucleic acid can be a DNA or RNA. In certain embodiments, the sample further comprises a non-nucleic acid component, e.g., a cell, protein, carbohydrate, or lipid, e.g., from the tumor. In certain embodiments, the sample further comprises a nucleic acid from a normal cell or tissue.

In certain embodiments, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. In certain embodiments, the sample is a blood sample. In certain embodiments, the tissue sample is a blood constituent sample. In certain embodiments, the sample is a cfDNA sample. In certain embodiments, the sample is a ctDNA sample. In certain embodiments, the sample is a CTC sample. In other embodiments, the tissue sample is a bone marrow aspirate (BMA) sample. The isolating step can include flow-sorting of individual chromosomes; and/or micro-dissecting a subject's sample (e.g., a sample described herein).

In other embodiments, the sample comprises one or more premalignant or malignant cells. In certain embodiments, the sample is acquired from a solid tumor, a soft tissue tumor, or a metastatic lesion. In certain embodiments, the sample is acquired from a hematologic malignancy or premaligancy. In other embodiments, the sample comprises a tissue or cells from a surgical margin. In certain embodiments, the sample comprises tumor-infiltrating lymphocytes. The sample can be histologically normal tissue. In an embodiment, the sample comprises one or more non-malignant cells.

In certain embodiments, the FFPE sample has one, two or all of the following properties: (a) has a surface area of about 10 mm² or greater, about 25 mm² or greater, or about 50 mm² or greater; (b) has a sample volume of about 0.1 mm³ or greater, about 0.2 mm³ or greater, about 0.3 mm³ or greater, about 0.4 mm³ or greater, about 0.5 mm³ or greater, about 0.6 mm³ or greater, about 0.7 mm³ or greater, about 0.8 mm³ or greater, about 0.9 mm³ or greater, about 1 mm³ or greater, about 2 mm³ or greater, about 3 mm³ or greater, about 4 mm³ or greater, or about 5 mm³ or greater; (c) has a cellularity of about 50% or more, about 60% or more, about 70% or more, about 80% or more, or about 90% or more; and/or (d) has a count of nucleated cells of about 10,000 cells or more, about 20,000 cells or more, about 30,000 cells or more, about 40,000 cells or more, or about 50,000 cells or more.

In one embodiment, the method further includes acquiring a sample, e.g., a sample described herein. The sample can be acquired directly or indirectly. In an embodiment, the sample is acquired, e.g., by isolation or purification, from a sample that comprises cfDNA. In an embodiment, the sample is acquired, e.g., by isolation or purification, from a sample that comprises ctDNA. In an embodiment, the sample is acquired, e.g., by isolation or purification, from a sample that comprises both a malignant cell and a non-malignant cell (e.g., tumor-infiltrating lymphocyte). In an embodiment, the sample is acquired, e.g., by isolation or purification, from a sample that comprises CTCs.

In other embodiments, the method includes evaluating a sample, e.g., a histologically normal sample, e.g., from a surgical margin, using the methods described herein. In some embodiments, samples obtained from histologically normal tissues (e.g., otherwise histologically normal tissue margins) may still have an alteration as described herein. The methods may thus further include re-classifying a sample based on the presence of the detected alteration. In an embodiment, multiple samples, e.g., from different subjects, are processed simultaneously.

In an embodiment, the method includes isolating nucleic acids from a sample to provide an isolated nucleic acid sample. In an embodiment, the method includes isolating nucleic acids from a control to provide an isolated control nucleic acid sample. In an embodiment, a method further comprises rejecting a sample with no detectable nucleic acid.

In an embodiment, the method further comprises determining if a primary control is available and if so isolating a control nucleic acid (e.g., DNA) from said primary control. In an embodiment, the method further comprises determining if NAT is present in said sample (e.g., where no primary control sample is available). In an embodiment, a method further comprises acquiring a sub-sample enriched for non-tumor cells, e.g., by macrodissecting non-tumor tissue from said NAT in a sample not accompanied by a primary control. In an embodiment, a method further comprises determining that no primary control and no NAT is available and marking said sample for analysis without a matched control,

In an embodiment, a method further comprises acquiring a value for nucleic acid yield in said sample and comparing the acquired value to a reference criterion, e.g., wherein if said acquired value is less than said reference criterion, then amplifying the nucleic acid prior to library construction. In an embodiment, a method further comprises acquiring a value for the size of nucleic acid fragments in said sample and comparing the acquired value to a reference criterion, e.g., a size, e.g., average size, of at least 300, 600, or 900 bps. A parameter described herein can be adjusted or selected in response to this determination.

In certain embodiments, the method includes isolating nucleic acids from an aged sample, e.g., an aged FFPE sample. The aged sample can be, for example, years old, e.g., 1 year, 2 years, 3 years, 4 years, 5 years, 10 years, 15 years, 20 years, 25 years, 50 years, 75 years, or 100 years old or older.

Nucleic acids can be obtained from samples of various sizes. For example, nucleic acids can be isolated from a sample from 5 to 200 μm, or larger. For example, the sample can measure 5 μm, 10 μm, 20 μm, 30 μm, 40 μm, 50 μm, 70 μm, 100 μm, 110 μm, 120 μm, 150 μm or 200 μm or larger.

Protocols for DNA isolation from a sample are known in the art, e.g., as provided in Example 1 of International Patent Application Publication No. WO 2012/092426. Additional methods to isolate nucleic acids (e.g., DNA) from formaldehyde- or paraformaldehyde-fixed, paraffin-embedded (FFPE) tissues are disclosed, e.g., in Cronin M. et al., (2004) Am J Pathol. 164 (1): 35-42; Masuda N. et al., (1999) Nucleic Acids Res. 27 (22): 4436-4443; Specht K. et al., (2001) Am J Pathol. 158 (2): 419-429, Ambion RecoverAll™ Total Nucleic Acid Isolation Protocol (Ambion, Cat. No. AM1975, September 2008), Maxwell® 16 FFPE Plus LEV DNA Purification Kit Technical Manual (Promega Literature #TM349, February 2011), E.Z.N.A.® FFPE DNA Kit Handbook (OMEGA bio-tek, Norcross, GA, product numbers D3399-00, D3399-01, and D3399-02; June 2009), and QIAamp® DNA FFPE Tissue Handbook (Qiagen, Cat. No. 37625, October 2007). RecoverAll™ Total Nucleic Acid Isolation Kit uses xylene at elevated temperatures to solubilize paraffin-embedded samples and a glass-fiber filter to capture nucleic acids. Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA. Protocols for DNA isolation from blood are disclosed, e.g., in the Maxwell® 16 LEV Blood DNA Kit and Maxwell® 16 Buccal Swab LEV DNA Purification Kit Technical Manual (Promega Literature #TM333, Jan. 1, 2011).

Protocols for RNA isolation are disclosed, e.g., in the Maxwell® 16 Total RNA Purification Kit Technical Bulletin (Promega Literature #TB351, August 2009).

The isolated nucleic acids (e.g., genomic DNA) can be fragmented or sheared by practicing routine techniques. For example, genomic DNA can be fragmented by physical shearing methods, enzymatic cleavage methods, chemical cleavage methods, and other methods well known to those skilled in the art. The nucleic acid library can contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library is a portion of the genome, e.g., where the complexity of the genome is reduced by design. In some embodiments, any selected portion of the genome can be used with a method described herein. In certain embodiments, the entire exome or a subset thereof is isolated.

In certain embodiments, the method further includes isolating nucleic acids from the sample to provide a library (e.g., a nucleic acid library as described herein). In certain embodiments, the sample includes whole genomic, subgenomic fragments, or both. The isolated nucleic acids can be used to prepare nucleic acid libraries. Protocols for isolating and preparing libraries from whole genomic or subgenomic fragments are known in the art (e.g., Illumina's genomic DNA sample preparation kit). In certain embodiments, the genomic or subgenomic DNA fragment is isolated from a subject's sample (e.g., a sample described herein). In one embodiment, the sample is a preserved specimen, e.g., embedded in a matrix, e.g., an FFPE block or a frozen sample. In certain embodiments, the isolating step includes flow-sorting of individual chromosomes; and/or microdissecting the sample. In certain embodiments, the amount of nucleic acid used to generate the nucleic acid library is less than 5 micrograms, less than 1 microgram, or less than 500 ng, less than 200 ng, less than 100 ng, less than 50 ng, less than 10 ng, less than 5 ng, or less than 1 ng.

In still other embodiments, the nucleic acids used to generate the library include RNA or cDNA derived from RNA. In some embodiments, the RNA includes total cellular RNA. In other embodiments, certain abundant RNA sequences (e.g., ribosomal RNAs) have been depleted. In some embodiments, the poly(A)-tailed mRNA fraction in the total RNA preparation has been enriched. In some embodiments, the cDNA is produced by random-primed cDNA synthesis methods. In other embodiments, the cDNA synthesis is initiated at the poly(A) tail of mature mRNAs by priming by oligo (dT)-containing oligonucleotides. Methods for depletion, poly(A) enrichment, and cDNA synthesis are well known to those skilled in the art.

In other embodiments, the nucleic acids are fragmented or sheared by a physical or enzymatic method, and optionally, ligated to synthetic adapters, size-selected (e.g., by preparative gel electrophoresis) and amplified (e.g., by PCR). Alternative methods for DNA shearing are known in the art, e.g., as described in Example 4 in International Patent Application Publication No. WO 2012/092426. For example, alternative DNA shearing methods can be more automatable and/or more efficient (e.g., with degraded FFPE samples). Alternatives to DNA shearing methods can also be used to avoid a ligation step during library preparation.

In other embodiments, the isolated DNA (e.g., the genomic DNA) is fragmented or sheared. In some embodiments, the library includes less than 50% of genomic DNA, such as a subfraction of genomic DNA that is a reduced representation or a defined portion of a genome, e.g., that has been subfractionated by other means. In other embodiments, the library includes all or substantially all genomic DNA.

In other embodiments, the fragmented and adapter-ligated group of nucleic acids is used without explicit size selection or amplification prior to hybrid selection. In some embodiments, the nucleic acid is amplified by a specific or non-specific nucleic acid amplification method that is well known to those skilled in the art. In some embodiments, the nucleic acid is amplified, e.g., by a whole-genome amplification method such as random-primed strand-displacement amplification,

The methods described herein can be performed using a small amount of nucleic acids, e.g., when the amount of source DNA or RNA is limiting (e.g., even after whole-genome amplification). In one embodiment, the nucleic acid comprises less than about 5 μg, 4 μg, 3 μg, 2 μg, 1 μg, 0.8 μg, 0.7 μg, 0.6 μg, 0.5 μg, or 400 ng, 300 ng, 200 ng, 100 ng, 50 ng, 10 ng, 5 ng, 1 ng, or less of nucleic acid sample. For example, one can typically begin with 50-100 ng of genomic DNA. One can start with less, however, if one amplifies the genomic DNA (e.g., using PCR) before the hybridization step, e.g., solution hybridization. Thus it is possible, but not essential, to amplify the genomic DNA before hybridization, e.g., solution hybridization.

In an embodiment, the sample comprises DNA, RNA (or cDNA derived from RNA), or both, from a non-cancer cell or a non-malignant cell, e.g., a tumor-infiltrating lymphocyte. In an embodiment, the sample comprises DNA, RNA (or cDNA derived from RNA), or both, from a non-cancer cell or a non-malignant cell, e.g., a tumor-infiltrating lymphocyte, and does not comprise, or is essentially free of, DNA, RNA (or cDNA derived from RNA), or both, from a cancer cell or a malignant cell.

In an embodiment, the sample comprises DNA, RNA (or cDNA derived from RNA) from a cancer cell or a malignant cell. In an embodiment, the sample comprises DNA, RNA (or cDNA derived from RNA) from a cancer cell or a malignant cell, and does not comprise, or is essentially free of, DNA, RNA (or cDNA derived from RNA), or both, from a non-cancer cell or a non-malignant cell, e.g., a tumor-infiltrating lymphocyte.

In an embodiment, the sample comprises DNA, RNA (or cDNA derived from RNA), or both, from a non-cancer cell or a non-malignant cell, e.g., a tumor-infiltrating lymphocyte, and DNA, RNA (or cDNA derived from RNA), or both, from a cancer cell or a malignant cell.

In certain embodiments, the sample is acquired from a subject having a cancer. Exemplary cancers include, but are not limited to, B cell cancer, e.g., multiple myeloma, melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypercosinophilia, chronic eosinophilic leukemia, neuroendocrine cancers, carcinoid tumors, and the like.

In an embodiment, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm. Premaligancy, as used herein, refers to a tissue that is not yet malignant but is poised to become malignant.

In some embodiments, a sample described herein is also referred to as a specimen. In some embodiments, the sample is a tissue sample, blood sample or bone marrow sample.

In some embodiments, the blood sample comprises cell-free DNA (cfDNA). In some embodiments, cfDNA comprises DNA from healthy tissue, e.g., non-diseased cells, or tumor tissue, e.g., tumor cells. In some embodiments cfDNA from tumor tissue comprises circulating tumor DNA (ctDNA). In some embodiments, ctDNA samples are obtained, e.g., collected, from a patient with a solid tumor, e.g., lung cancer, breast cancer or colon cancer.

In some embodiments, the sample, e.g., specimen, is a formalin-fixed paraffin embedded (FFPE) specimen. In some embodiments, the FPPE specimen includes, but is not limited to specimens chosen from: core-needle biopsies, fine-needle aspirates, or effusion cytologies. In some embodiments, the sample comprises an FPPE block and one original hematoxylin and cosin (H&E) stained slide. In some embodiments, the sample comprises unstained slides (e.g., positively charged, unbaked and 4-5 microns thick; e.g., 1, 2, 3, 4, 5, 6, 7, 8, 9, 10 or more such slides) and one or more H&E stained slides.

In some embodiments, the sample comprises an FPPE block or unstained slides, e.g., 1, 2, 3, 4, 5, 6, 7, 8, 9, 10, 11, 12, 13, 14, 15, 16 or more unstained slides and one or more H&E slide. In some embodiments, the sample comprises tissue that is formalin-fixed and embedded into a paraffin block, e.g., using a standard fixation method, e.g. as described herein.

In some embodiments, the sample comprises a surface area of at least 1-30 mm², e.g., about 5-25 mm². In some embodiments, the sample comprises a surface area of at least 1, 2, 3, 4, 5, 6, 7, 8, 9, or 10 mm², e.g., 5 mm². In some embodiments, the sample comprises a surface area of at least 5 mm². In some embodiments, the sample comprises a surface area of about 20, 21, 22, 23, 24, 25, 26, 27, 28, 29 or 30 mm², e.g., 25 mm². In some embodiments, the sample comprises a surface area of 25 mm².

In some embodiments, the sample comprises a surface volume of at least 1-5 mm³, e.g., about 2 mm³. In some embodiments, a surface volume of about 2 mm³ comprises a sample having a surface area of about 25 mm² at a depth of about 80 microns, e.g., at least or more than 80 microns.

In some embodiments, the sample comprises a tumor content, e.g., comprising tumor nuclei. In some embodiments, the sample comprises a tumor content with at least 5-50%, 10-40%, 15-25%, or 20-30% tumor nuclei. In some embodiments, the sample comprises a tumor content of at least 20% tumor nuclei. In some embodiments, the sample comprises a tumor content of about 30% tumor nuclei. In some embodiments, percent tumor nuclei is determined, e.g., calculated, by dividing the number of tumor cells by the total number of all cells with nuclei. In some embodiments, when the sample is a liver sample, e.g., comprising hepatocytes, higher tumor content may be required. In some embodiments, hepatocytes have nuclei with twice, e.g., double, the DNA content of other, e.g., non-hepatocyte, somatic nuclei. In some embodiments, sensitivity of detection of an alteration, e.g., as described herein, depends on tumor content of the sample, e.g., a lower tumor content can result in lower sensitivity of detection.

In some embodiments, DNA is extracted from nucleated cells from the sample. In some embodiments, a sample has a low nucleated cellularity, e.g., when the sample is comprised mainly of erythrocytes, lesional cells that contain excessive cytoplasm, or tissue with fibrosis. In some embodiments, a sample with low nucleated cellularity may require more, e.g., greater, tissue volume, e.g., more than 2 mm³, for DNA extraction.

In some embodiments, the FPPE sample, e.g., specimen, is prepared using a standard fixation method to preserve nucleic acid integrity. In some embodiments, the standard fixation method comprises using 10% neutral-buffered formalin, e.g., for 6-72 hours. In some embodiments, the method does not include fixatives such as Bouins, B5, AZF of Holland's. In some embodiments, the method dose not comprise decalcification. In some embodiments, the method includes decalcification. In embodiments, decalcification is performed with EDTA. In some embodiments, strong acids, e.g., hydrochloric acid, sulfuric acid or picric acid, are not used for decalcification.

In some embodiments, the sample comprises an FPPE block or unstained slides, e.g., 1, 2, 3, 4, 5, 6, 7, 8, 9, 10, 11, 12, 13, 14, 15, 16 or more unstained slides and one or more H&E slides. In some embodiments, the sample comprises tissue that is formalin-fixed and embedded into a paraffin block, e.g., using a standard fixation method, e.g. as described herein.

In some embodiments, the sample comprises peripheral whole blood or bone marrow aspirate. In some embodiments, the sample, e.g., lesion tissue, comprises at least 20% nucleated elements. In some embodiments, the peripheral whole blood sample or bone marrow aspirate sample is collected at a volume of about 2.5 ml. In some embodiments, the blood sample is shipped, e.g., at ambient temperature, e.g., 43-99° F. or 6-37° C., on the same day as collection. In some embodiments, the blood sample is not frozen or refrigerated.

In some embodiments, the sample comprises isolated, e.g., extracted, nucleic acid, e.g., DNA or RNA. In some embodiments, the isolated nucleic acid comprises DNA or RNA, e.g., in nuclease-free water.

In some embodiments, the sample comprises a blood sample, e.g., peripheral whole blood sample. In some embodiments, the peripheral whole blood sample is collected in, e.g., two tubes, e.g., with about 8.5 ml blood per tube. In some embodiments, the peripheral whole blood sample is collected by venipuncture, e.g., according to CLSI H3-A6. In some embodiments, the blood is immediately mixed, e.g., by gentle inversion, for, e.g., about 8-10 times. In some embodiments, inversion is performed by a complete, e.g., full, 180° turn, e.g., of the wrist. In some embodiments, the blood sample is shipped, e.g., at ambient temperature, e.g., 43-99° F. or 6-37° C. on the same day as collection. In some embodiments, the blood sample is not frozen or refrigerated. In some embodiments, the collected blood sample is kept, e.g., stored, at 43-99° F. or 6-37° C.

### Subjects

In some embodiments, the sample is obtained, e.g., collected, from a subject, e.g., patient, with a condition or disease, e.g., a hyperproliferative disease (e.g., as described herein) or a non-cancer indication. In some embodiments, the disease is a hyperproliferative disease. In some embodiments, the hyperproliferative disease is a cancer, e.g., a solid tumor or a hematological cancer. In some embodiments, the cancer is a solid tumor. In some embodiments, the cancer is a hematological cancer, e.g. a leukemia or lymphoma.

In some embodiments, the subject has a cancer. In some embodiments, the subject has been, or is being treated, for cancer. In some embodiments, the subject is in need of being monitored for cancer progression or regression, e.g., after being treated with a cancer therapy. In some embodiments, the subject is in need of being monitored for relapse of cancer. In some embodiments, the subject is at risk of having a cancer. In some embodiments, the subject has not been treated with a cancer therapy. In some embodiments, the subject has a genetic predisposition to a cancer (e.g., having a mutation that increases his or her baseline risk for developing a cancer). In some embodiments, the subject has been exposed to an environment (e.g., radiation or chemical) that increases his or her risk for developing a cancer. In some embodiments, the subject is in need of being monitored for development of a cancer.

In some embodiments, the patient has been previously treated with a targeted therapy, e.g., one or more targeted therapies. In some embodiments, for a patient who has been previously treated with a targeted therapy, a post-targeted therapy sample, e.g., specimen is obtained, e.g., collected. In some embodiments, the post-targeted therapy sample is a sample obtained, e.g., collected, after the completion of the targeted therapy.

In some embodiments, the patient has not been previously treated with a targeted therapy. In some embodiments, for a patient who has not been previously treated with a targeted therapy, the sample comprises a resection, e.g., an original resection, or a recurrence, e.g., disease recurrence post-therapy, e.g., non-targeted therapy. In some embodiments, the sample is or is part of a primary tumor or a metastasis, e.g., metastasis biopsy. In some embodiments, the sample is obtained from a site, e.g., tumor site, with the highest percent of tumor, e.g., tumor cells, as compared to adjacent sites, e.g., adjacent sites with tumor cells. In some embodiments, the sample is obtained from a site, e.g., tumor site, with the largest tumor focus as compared to adjacent sites, e.g., adjacent sites with tumor cells.

In some embodiments, the disease is chosen from: non-small cell lung cancer (NSCLC), melanoma, breast cancer, colorectal cancer (CRC), or ovarian cancer. In some embodiments, an NSCLC described herein includes NSCLC having, e.g., an EGFR alteration (e.g., exon 19 deletion or exon 21 L858R alteration), ALK rearrangement, or BRAF V600E. In some embodiments, a melanoma described herein includes melanoma having a BRAF alteration, e.g., V600E and/or V600K. In some embodiments, a breast cancer described herein includes breast cancer having an ERBB2 (HER2) amplification. In some embodiments, a colorectal cancer described herein includes a colorectal cancer having wild-type KRAS, e.g., absence of mutations in codon 12 and/or 13, or absence of mutations in codons 2, 3, and/or 4. In some embodiments, a colorectal cancer described herein includes a colorectal cancer having wild-type NRAS, e.g., absence of mutations in codons 2, 3, and/or 4. In some embodiments, a colorectal cancer described herein includes a colorectal cancer having a wild-type KRAS, e.g., as described herein, and a wild-type NRAS, e.g., as described herein. In some embodiments, an ovarian cancer described herein includes an ovarian cancer having a BRCA1 and/or BRCA2 alteration.

### Target Capture Reagents

Methods described herein provide for optimized sequencing of a large number of genes and gene products from samples, e.g., from a cancer described herein, from one or more subjects by the appropriate selection of target capture reagents, e.g., target capture reagents for use in solution hybridization, for the selection of target nucleic acid molecules to be sequenced.

Any combination of two, three, four, five, or more pluralities of target capture reagents can be used, for example, a combination of first and second pluralities of target capture reagents; first and third pluralities of target capture reagents; first and fourth pluralities of target capture reagents; first and fifth pluralities of target capture reagents; second and third pluralities of target capture reagents; second and fourth pluralities of target capture reagents; second and fifth pluralities of target capture reagents; third and fourth pluralities of target capture reagents; third and fifth pluralities of target capture reagents; fourth and fifth pluralities of target capture reagents; first, second and third pluralities of target capture reagents; first, second and fourth pluralities of target capture reagents; first, second and fifth pluralities of target capture reagents; first, second, third, and fourth pluralities of target capture reagents; first, second, third, fourth and fifth pluralities of target capture reagents, and so on.

In some embodiments, the method comprises:
(a) acquiring a library comprising a plurality of nucleic acid molecules (e.g., target nucleic acid molecules) from a sample, e.g., a plurality of tumor nucleic acid molecules from a sample, e.g., a sample described herein;
(b) contacting the library with two, three, or more pluralities of target capture reagents to provide selected nucleic acid molecules (e.g., a library catch);
(c) acquiring a read for a subject interval from a nucleic acid molecule, e.g., a tumor nucleic acid molecule from said library or library catch, e.g., by a method comprising sequencing, e.g., with a next-generation sequencing method;
(d) aligning said read by an alignment method, e.g., an alignment method described herein; and
(e) assigning a nucleotide value (e.g., calling a mutation, e.g., with a Bayesian method or a method described herein) from said read for a nucleotide position.

In some embodiments, the level of sequencing depth as used herein (e.g., X-fold level of sequencing depth) refers to the number of reads (e.g., unique reads), after detection and removal of duplicate reads, e.g., PCR duplicate reads. In other embodiments, duplicate reads are evaluated, e.g., to support detection of copy number alteration (CNAs).

In one embodiment, the target capture reagent selects a subject interval containing one or more rearrangements, e.g., an intron containing a genomic rearrangement. In such embodiments, the target capture reagent is designed such that repetitive sequences are masked to increase the selection efficiency. In those embodiments where the rearrangement has a known juncture sequence, complementary target capture reagents can be designed to the juncture sequence to increase the selection efficiency.

In some embodiments, the method comprises the use of target capture reagents designed to capture two or more different target categories, each category having a different design strategy. In some embodiments, the method (e.g., hybrid capture method) and composition disclosed herein capture a subset of target sequences (e.g., target nucleic acid molecules) and provide homogenous coverage of the target sequence, while minimizing coverage outside of that subset. In one embodiment, the target sequences include the entire exome out of genomic DNA, or a selected subset thereof. In another embodiment, the target sequences include a large chromosomal region, e.g., a whole chromosome arm. The methods and compositions disclosed herein provide different target capture reagents for achieving different sequencing depths and patterns of coverage for complex target nucleic acid sequences (e.g., nucleic acid libraries).

In an embodiment, the method comprises providing selected nucleic acid molecules of one or a plurality of nucleic acid libraries (e.g., a library catch). For example, the method comprises:
providing one or a plurality of libraries (e.g., one or a plurality of nucleic acid libraries) comprising a plurality of nucleic acid molecules, e.g., target nucleic acid nucleic acid molecules (e.g., including a plurality of tumor nucleic acid molecules and/or reference nucleic acid molecules);
contacting the one or a plurality of libraries, e.g., in a solution-based reaction, with two, three, or more pluralities of target capture reagents (e.g., oligonucleotide target capture reagents) to form a hybridization mixture comprising a plurality of target capture reagent/nucleic acid molecule hybrids;
separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture,
thereby providing a library catch (e.g., a selected or enriched subgroup of nucleic acid molecules from the one or a plurality of libraries).

In one embodiment, each of the first, second, or third plurality of target capture reagents has a unique recovery efficiency. In some embodiments, at least two or three pluralities of target capture reagents have recovery efficiency values that differ.

In certain embodiments, the value for recovery efficiency is modified by one or more of: differential representation of different target capture reagents, differential overlap of target capture reagent subsets, differential target capture reagent parameters, mixing of different target capture reagents, and/or using different types of target capture reagents. For example, a variation in recovery efficiency (e.g., relative sequence coverage of each target capture reagent/target category) can be adjusted, e.g., within a plurality of target capture reagents and/or among different pluralities of target capture reagents, by altering one or more of:
(i) Differential representation of different target capture reagents—The target capture reagent design to capture a given target (e.g., a target nucleic acid molecule) can be included in more/fewer number of copies to enhance/reduce relative target sequencing depths;
(ii) Differential overlap of target capture reagent subsets—The target capture reagent design to capture a given target (e.g., a target nucleic acid molecule) can include a longer or shorter overlap between neighboring target capture reagents to enhance/reduce relative target sequencing depths;
(iii) Differential target capture reagent parameters—The target capture reagent design to capture a given target (e.g., a target nucleic acid molecule) can include sequence modifications/shorter length to reduce capture efficiency and lower the relative target sequencing depths;
(iv) Mixing of different target capture reagents-Target capture reagents that are designed to capture different target sets can be mixed at different molar ratios to enhance/reduce relative target sequencing depths;
(v) Using different types of oligonucleotide target capture reagents—In certain embodiments, the target capture reagent can include:
(a) one or more chemically (e.g., non-enzymatically) synthesized (e.g., individually synthesized) target capture reagents,
(b) one or more target capture reagents synthesized in an array,
(c) one or more enzymatically prepared, e.g., in vitro transcribed, target capture reagents;
(d) any combination of (a), (b) and/or (c),
(e) one or more DNA oligonucleotides (e.g., a naturally or non-naturally occurring DNA oligonucleotide),
(f) one or more RNA oligonucleotides (e.g., a naturally or non-naturally occurring RNA oligonucleotide),
(g) a combination of (e) and (f), or
(h) a combination of any of the above.

The different oligonucleotide combinations can be mixed at different ratios, e.g., a ratio chosen from 1:1, 1:2, 1:3, 1:4, 1:5, 1:10, 1:20, 1:50; 1:100, 1:1000, or the like. In one embodiment, the ratio of chemically-synthesized target capture reagent to array-generated target capture reagent is chosen from 1:5, 1:10, or 1:20. The DNA or RNA oligonucleotides can be naturally- or non-naturally-occurring. In certain embodiments, the target capture reagents include one or more non-naturally-occurring nucleotides to, e.g., increase melting temperature. Exemplary non-naturally occurring oligonucleotides include modified DNA or RNA nucleotides. Exemplary modified nucleotides (e.g., modified RNA or DNA nucleotides) include, but are not limited to, a locked nucleic acid (LNA), wherein the ribose moiety of an LNA nucleotide is modified with an extra bridge connecting the 2′ oxygen and 4′ carbon; peptide nucleic acid (PNA), e.g., a PNA composed of repeating N-(2-aminoethyl)-glycine units linked by peptide bonds; a DNA or RNA oligonucleotide modified to capture low GC regions; a bicyclic nucleic acid (BNA); a crosslinked oligonucleotide; a modified 5-methyl deoxycytidine; and 2,6-diaminopurine. Other modified DNA and RNA nucleotides are known in the art.

In certain embodiments, a substantially uniform or homogeneous coverage of a target sequence (e.g., a target nucleic acid molecule) is obtained. For example, within each target capture reagent/target category, uniformity of coverage can be optimized by modifying target capture reagent parameters, for example, by one or more of:
(i) Increasing/decreasing target capture reagent representation or overlap can be used to enhance/reduce coverage of targets (e.g., target nucleic acid molecules), which are under/over-covered relative to other targets in the same category;
(ii) For low coverage, hard to capture target sequences (e.g., high GC content sequences), expand the region being targeted with the target capture reagents to cover, e.g., adjacent sequences (e.g., less GC-rich adjacent sequences);
(iii) Modifying a target capture reagent sequence can be used to reduce secondary structure of the target capture reagent and enhance its recovery efficiency;
(iv) Modifying a target capture reagent length can be used to equalize melting hybridization kinetics of different target capture reagents within the same category. Target capture reagent length can be modified directly (by producing target capture reagents with varying lengths) or indirectly (by producing target capture reagents of consistent length, and replacing the target capture reagent ends with arbitrary sequence);
(v) Modifying target capture reagents of different orientation for the same target region (i.e. forward and reverse strand) may have different binding efficiencies. The target capture reagent with either orientation providing optimal coverage for each target may be selected;
(vi) Modifying the amount of a binding entity, e.g., a capture tag (e.g. biotin), present on each target capture reagent may affect its binding efficiency. Increasing/decreasing the tag level of target capture reagents targeting a specific target may be used to enhance/reduce the relative target coverage;
(vii) Modifying the type of nucleotide used for different target capture reagents can be used to affect binding affinity to the target, and enhance/reduce the relative target coverage; or
(viii) Using modified oligonucleotide target capture reagents, e.g., having more stable base pairing can be used to equalize melting hybridization kinetics between areas of low or normal GC content relative to high GC content,

In an embodiment, the method comprises the use of a plurality of target capture reagents that includes a target capture reagent that selects a tumor nucleic acid molecule, e.g., a nucleic acid molecule comprising a subject interval from a tumor cell. The tumor nucleic acid molecule can be any nucleotide sequence present in a tumor cell, e.g., a mutated, a wild-type, a reference or an intron nucleotide sequence, as described herein, that is present in a tumor or cancer cell. In one embodiment, the tumor nucleic acid molecule includes an alteration (e.g., one or more mutations) that appears at a low frequency, e.g., about 5% or less of the cells from the sample harbor the alteration in their genome. In other embodiments, the tumor nucleic acid molecule includes an alteration (e.g., one or more mutations) that appears at a frequency of about 10% of the cells from the sample. In other embodiments, the tumor nucleic acid molecule includes a subgenomic interval from an intron sequence, e.g., an intron sequence as described herein, a reference sequence that is present in a tumor cell.

In other embodiments, the method comprises amplifying the library catch (e.g., by PCR), In other embodiments, the library catch is not amplified.

In another aspect, the invention features target capture reagents described herein and combinations of individual plurality of target capture reagents described herein. The target capture reagents can be part of a kit which can optionally comprise instructions, standards, buffers or enzymes or other reagents.

### Design and Construction of Target Capture Reagents

In some embodiments, a target capture reagent is a molecule, which can bind to and thereby allow capture of a target molecule. For example, a target capture reagent can be a bait, e.g., a nucleic acid molecule, e.g., a DNA or RNA molecule, which can hybridize to (e.g., be complementary to), and thereby allow capture of a target nucleic acid. In some embodiments, the target capture reagent, e.g., bait, is a capture oligonucleotide. In certain embodiments, the target nucleic acid is a genomic DNA molecule. In other embodiments, the target nucleic acid is an RNA molecule or a cDNA molecule derived from an RNA molecule. In one embodiment, the target capture reagent is a DNA molecule. In one embodiment, the target capture reagent is an RNA molecule. In one embodiment, the target capture reagent is suitable for solution phase hybridization. In one embodiment, the target capture reagent is suitable for solid phase hybridization. In one embodiment, the target capture reagent is suitable for both solution phase and solid phase hybridization.

Typically, DNA molecules are used as target capture reagent sequences, although RNA molecules can also be used. In some embodiments, a DNA molecule target capture reagent can be single stranded DNA (ssDNA) or double-stranded DNA (dsDNA).

In some embodiments, an RNA-DNA duplex is more stable than a DNA-DNA duplex, and therefore provides for potentially better capture of nucleic acids. RNA target capture reagents can be made as described elsewhere herein, using methods known in the art including, but not limited to, de novo chemical synthesis and transcription of DNA molecules using a DNA-dependent RNA polymerase. In one embodiment, the target capture reagent sequence is produced using a known nucleic acid amplification method, such as PCR, e.g., using human DNA or pooled human DNA samples as the template. The oligonucleotides can then be converted to RNA target capture reagents. In one embodiment, in vitro transcription is used, for example, based on adding an RNA polymerase promoter sequence to one end of the oligonucleotide. In one embodiment, the RNA polymerase promoter sequence is added at the end of the target capture reagent by amplifying or re-amplifying the target capture reagent sequence, e.g., using PCR or another nucleic acid amplification method, e.g., by tailing one primer of each target-specific primer pairs with an RNA promoter sequence. In one embodiment, the RNA polymerase is a T7 polymerase, a SP6 polymerase, or a T3 polymerase. In one embodiment, RNA target capture reagent is labeled with a tag, e.g., an affinity tag. In one embodiment, RNA target capture reagent is made by in vitro transcription, e.g., using biotinylated UTP. In another embodiment, RNA target capture reagent is produced without biotin and then biotin is crosslinked to the RNA molecule using a method well known in the art, such as psoralen crosslinking. In one embodiment, the RNA target capture reagent is an RNase-resistant RNA molecule, which can be made, e.g., by using modified nucleotides during transcription to produce a RNA molecule that resists RNase degradation. In one embodiment, the RNA target capture reagent corresponds to only one strand of the double-stranded DNA target. Typically, such RNA target capture reagents are not self-complementary and are more effective as hybridization drivers.

The target capture reagents can be designed from reference sequences, such that the target capture reagents are optimal for selecting targets of the reference sequences. In some embodiments, target capture reagent sequences are designed using a mixed base (e.g., degeneracy). For example, the mixed base(s) can be included in the target capture reagent sequence at the position(s) of a common SNP or mutation, to optimize the target capture reagent sequences to catch both alleles (e.g., SNP and non-SNP; mutant and non-mutant). In some embodiments, all known sequence variations (or a subset thereof) can be targeted with multiple oligonucleotide target capture reagents, rather than by using mixed degenerate oligonucleotides.

In certain embodiments, the target capture reagent includes an oligonucleotide (or a plurality of oligonucleotides) between about 100 nucleotides and 300 nucleotides in length. Typically, the target capture reagent includes an oligonucleotide (or a plurality of oligonucleotides) between about 130 nucleotides and 230 nucleotides, or about 150 and 200 nucleotides, in length. In other embodiments, the target capture reagent includes an oligonucleotide (or a plurality of oligonucleotides) between about 300 nucleotides and 1000 nucleotides in length.

In some embodiments, the target nucleic acid molecule-specific sequences in the oligonucleotide are between about 40 and 1000 nucleotides, about 70 and 300 nucleotides, about 100 and 200 nucleotides in length, typically between about 120 and 170 nucleotides in length.

In some embodiments, the target capture reagent includes a binding entity. The binding entity can be an affinity tag. In some embodiments, the affinity tag is a biotin molecule or a hapten. In certain embodiments, the binding entity allows for separation of the target capture reagent/nucleic acid molecule hybrids from the hybridization mixture by binding to a partner, such as an avidin molecule, or an antibody that binds to the hapten or an antigen-binding fragment thereof.

In other embodiments, the oligonucleotides in the target capture reagent contain forward and reverse complement sequences for the same target nucleic acid molecule sequence whereby the oligonucleotides with reverse-complemented nucleic acid molecule-specific sequences also carry reverse complement universal tails. This can lead to RNA transcripts that are the same strand, i.e., not complementary to each other.

In other embodiments, the target capture reagent includes oligonucleotides that contain degenerate or mixed bases at one or more positions. In still other embodiments, the target capture reagent includes multiple or substantially all known sequence variants present in a population of a single species or community of organisms. In one embodiment, the target capture reagent includes multiple or substantially all known sequence variants present in a human population.

In other embodiments, the target capture reagent includes cDNA sequences or is derived from cDNA sequences. In other embodiments, the target capture reagent includes amplification products (e.g., PCR products) that are amplified from genomic DNA, cDNA or cloned DNA.

In other embodiments, the target capture reagent includes RNA molecules. In some embodiments, the set includes chemically, enzymatically modified, or in vitro transcribed RNA molecules, including but not limited to, those that are more stable and resistant to RNase.

In yet other embodiments, the target capture reagents are produced by a method described in US 2010/0029498 and Gnirke, A. et al. (2009) Nat Biotechnol. 27 (2): 182-189, incorporated herein by reference. For example, biotinylated RNA target capture reagents can be produced by obtaining a pool of synthetic long oligonucleotides, originally synthesized on a microarray, and amplifying the oligonucleotides to produce the target capture reagent sequences. In some embodiments, the target capture reagents are produced by adding an RNA polymerase promoter sequence at one end of the target capture reagent sequences, and synthesizing RNA sequences using RNA polymerase. In one embodiment, libraries of synthetic oligodeoxynucleotides can be obtained from commercial suppliers, such as Agilent Technologies, Inc., and amplified using a known nucleic acid amplification method.

Accordingly, a method of making the aforesaid target capture reagent is provided. The method includes, for example, selecting one or more target capture reagents, e.g., target-specific bait oligonucleotide sequences (e.g., one or more mutation capturing, reference or control oligonucleotide sequences as described herein); obtaining a pool of target capture reagents, e.g., target-specific bait oligonucleotide sequences (e.g., synthesizing the pool of target-specific bait oligonucleotide sequences, e.g., by microarray synthesis); and optionally, amplifying the target capture reagents, e.g., target-specific bait oligonucleotide sequences.

In other embodiments, the method further includes amplifying (e.g., by PCR) the oligonucleotides using one or more biotinylated primers. In some embodiments, the oligonucleotides include a universal sequence at the end of each oligonucleotide attached to the microarray. The methods can further include removing the universal sequences from the oligonucleotides. Such methods can also include removing the complementary strand of the oligonucleotides, annealing the oligonucleotides, and extending the oligonucleotides. In some of these embodiments, the method for amplifying (e.g., by PCR) the oligonucleotides uses one or more biotinylated primers. In some embodiments, the method further includes size selecting the amplified oligonucleotides,

In one embodiment, an RNA target capture reagent is made. The methods include producing a set of target capture reagent sequences according to the methods described herein, adding an RNA polymerase promoter sequence at one end of the target capture reagent sequences, and synthesizing RNA sequences using RNA polymerase. The RNA polymerase can be chosen from a T7 RNA polymerase, an SP6 RNA polymerase, or a T3 RNA polymerase. In other embodiments, the RNA polymerase promoter sequence is added at the ends of the target capture reagent sequences by amplifying (e.g., by PCR) the target capture reagent sequences. In embodiments where the target capture reagent sequences are amplified by PCR with specific primer pairs out of genomic DNA or cDNA, adding an RNA promoter sequence to 5′ end of one of the two specific primers in each pair will lead to a PCR product that can be transcribed into an RNA target capture reagent using a standard method.

In other embodiments, target capture reagents can be produced using human DNA or pooled human DNA samples as the template. In such embodiments, the oligonucleotides are amplified by polymerase chain reaction (PCR). In other embodiments, the amplified oligonucleotides are reamplified by rolling circle amplification or hyperbranched rolling circle amplification. The same methods also can be used to produce target capture reagent sequences using human DNA or pooled human DNA samples as the template. The same methods can also be used to produce target capture reagent sequences using a sub-fraction of a genome obtained by other methods, including but not limited to restriction digestion, pulsed-field gel electrophoresis, flow-sorting, CsCl density gradient centrifugation, selective kinetic re-association, microdissection of chromosome preparations, and other fractionation methods known to those skilled in the art.

In certain embodiments, the number of target capture reagents (e.g., baits) in the plurality of target capture reagents is less than 1,000. In other embodiments, the number of target capture reagents (e.g., baits) in the plurality target capture reagent is greater than 1,000, greater than 5,000, greater than 10,000, greater than 20,000, greater than 50,000, greater than 100,000, or greater than 500,000.

The length of the target capture reagent sequence can be between about 70 nucleotides and 1000 nucleotides. In one embodiment, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

Each target capture reagent sequence can include a target-specific (e.g., a nucleic acid molecule-specific) target capture reagent sequence and universal tails on one or both ends. As used herein, the term “target capture reagent sequence” can refer to the target-specific target capture reagent sequence or the entire oligonucleotide including the target-specific “target capture reagent sequence” and other nucleotides of the oligonucleotide. The target-specific sequences in the target capture reagents are between about 40 nucleotides and 1000 nucleotides in length. In one embodiment, the target-specific sequence is between about 70 nucleotides and 300 nucleotides in length. In another embodiment, the target-specific sequence is between about 100 nucleotides and 200 nucleotides in length. In yet another embodiment, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In one embodiment, the target capture reagent is an oligomer (e.g., comprised of RNA oligomers, DNA oligomers, or a combination thereof) about 50 to 200 nucleotides in length (e.g., about 50, 60, 80, 90, 100, 110, 120, 130, 140, 150, 160, 170, 190, or 200 nucleotides in length). In one embodiment, each target capture reagent oligomer includes about 120 to 170, or typically, about 120 nucleotides, which are a target-specific target capture reagent sequence. The target capture reagent can comprise additional non-target-specific nucleotide sequences at one or both ends. The additional nucleotide sequences can be used, e.g., for PCR amplification or as a target capture reagent identifier. In certain embodiments, the target capture reagent additionally comprises a binding entity as described herein (e.g., an affinity tag such as a biotin molecule), The binding entity, e.g., biotin molecule, can be attached to the target capture reagent, e.g., at the 5′-end, 3′-end, or internally (e.g., by incorporating a biotinylated nucleotide), of the target capture reagent. In one embodiment, the biotin molecule is attached at 5′-end of the target capture reagent.

In one exemplary embodiment, the target capture reagent is an oligonucleotide about 150 nucleotides in length, of which 120 nucleotides are target-specific “target capture reagent sequence”. The other 30 nucleotides (e.g., 15 nucleotides on each end) are universal arbitrary tails used for PCR amplification. The tails can be any sequence selected by the user. For example, the pool of synthetic oligonucleotides can include oligonucleotides of the sequence of 5′-ATCGCACCAGCGTGTN₁₂₀CACTGCGGCTCCTCA-3′ (SEQ ID NO: 1) with N₁₂₀ indicating the target-specific target capture reagent sequences.

The target capture reagent sequences described herein can be used for selection of exons and short target sequences. In one embodiment, the target capture reagent is between about 100 nucleotides and 300 nucleotides in length. In another embodiment, the target capture reagent is between about 130 nucleotides and 230 nucleotides in length. In yet another embodiment, the target capture reagent is between about 150 nucleotides and 200 nucleotides in length. The target-specific sequences in the target capture reagents, e.g., for selection of exons and short target sequences, are between about 40 nucleotides and 1000 nucleotides in length. In one embodiment, the target-specific sequence is between about 70 nucleotides and 300 nucleotides in length. In another embodiment, the target-specific sequence is between about 100 nucleotides and 200 nucleotides in length. In yet another embodiment, the target-specific sequence is between about 120 nucleotides and 170 nucleotides in length.

In some embodiments, long oligonucleotides can minimize the number of oligonucleotides necessary to capture the target sequences. For example, one oligonucleotide can be used per exon. It is known in the art that the mean and median lengths of the protein-coding exons in the human genome are about 164 and 120 base pairs, respective. Longer target capture reagent sequences can be more specific and capture better than shorter ones. As a result, the success rate per oligonucleotide target capture reagent sequence is higher than with short oligonucleotides. In one embodiment, the minimum target capture reagent-covered sequence is the size of one target capture reagent (e.g., 120-170 bases), e.g., for capturing exon-sized targets. In determining the length of the target capture reagent sequences, one also can take into consideration that unnecessarily long target capture reagents catch more unwanted DNA directly adjacent to the target. Longer oligonucleotide target capture reagents can also be more tolerant to polymorphisms in the targeted region in the DNA samples than shorter ones. Typically, the target capture reagent sequences are derived from a reference genome sequence. If the target sequence in the actual DNA sample deviates from the reference sequence, for example if it contains a single nucleotide polymorphism (SNP), it can hybridize less efficiently to the target capture reagent and may therefore be under-represented or completely absent in the sequences hybridized to the target capture reagent sequences. Allelic drop-outs due to SNPs can be less likely with the longer synthetic target capture reagent molecules for the reason that a single mismatch in, e.g., 120 to 170 bases can have less of an effect on hybrid stability than a single mismatch in, 20 or 70 bases, which are the typical target capture reagent or primer lengths in multiplex amplification and microarray capture, respectively.

For selection of targets that are long compared to the length of the capture target capture reagents, such as genomic regions, target capture reagent sequence lengths are typically in the same size range as the target capture reagents for short targets mentioned above, except that there is no need to limit the maximum size of target capture reagent sequences for the sole purpose of minimizing targeting of adjacent sequences. Alternatively, oligonucleotides can be tiled across a much wider window (typically 600 bases). This method can be used to capture DNA fragments that are much larger (e.g., about 500 bases) than a typical exon. As a result, much more unwanted flanking non-target sequences are selected.

### Synthesis of Target Capture Reagents

The target capture reagents can be, for example, any type of oligonucleotide, e.g., DNA or RNA. The DNA or RNA target capture reagents (“oligo target capture reagents”) can be synthesized individually, or can be synthesized in an array, as a DNA or RNA target capture reagent (e.g., “array baits”). An oligo target capture reagent, whether provided in an array format, or as an isolated oligo, is typically single stranded. The target capture reagent can additionally comprise a binding entity as described herein (e.g., an affinity tag such as a biotin molecule). The binding entity, e.g., biotin molecule, can be attached to the target capture reagent, e.g., at 5′ or 3′-end of the target capture reagent, typically, at 5′-end of the target capture reagent. Target capture reagents can be synthesized by a method described in the art, e.g., as described in International Patent Application Publication No. WO 2012/092426, or International Patent Application Publication No. WO 2015/021080, the entire contents of which are herein incorporated by reference.

### Hybridization Conditions

The methods featured in the invention include the step of contacting the library (e.g., the nucleic acid library) with a plurality of target capture reagents to provide a selected library catch. The contacting step can be effected in solution hybridization. In certain embodiments, the method includes repeating the hybridization step by one or more additional rounds of solution hybridization. In some embodiments, the method further includes subjecting the library catch to one or more additional rounds of solution hybridization with the same or different collection of target capture reagents. Hybridization methods that can be adapted for use in the methods herein are described in the art, e.g., as described in International Patent Application Publication No. WO 2012/092426.

Additional embodiments or features of the present invention are as follows:

In certain embodiments, the method comprises determining the presence or absence of an alteration associated, e.g., positively or negatively, with a cancerous phenotype (e.g., at least 10, 20, 30, 50 or more of the alterations in the genes or gene products described herein) in the sample. In other embodiments, the method comprises determining genomic signatures, e.g., continuous/complex biomarkers (e.g., the level of tumor mutational burden). In other embodiments, the method comprises determining one or more genomic signatures, e.g., continuous/complex biomarkers, e.g., the level of microsatellite instability, or the presence or absence of heterozygosity (LOH). The method includes contacting the nucleic acids in the sample in a solution-based reaction according to any of the methods and target capture reagents described herein to obtain a library catch; and sequencing (e.g., by next-generation sequencing) all or a subset of the library catch, thereby determining the presence or absence of the alteration in the genes or gene products described herein.

In certain embodiments, the target capture reagent includes an oligonucleotide (or a plurality of oligonucleotides) between about 100 nucleotides and 300 nucleotides in length. Typically, the target capture reagent includes an oligonucleotide (or a plurality of oligonucleotides) between about 130 nucleotides and 230 nucleotides, or about 150 and 200 nucleotides, in length. In other embodiments, the target capture reagent includes an oligonucleotide (or a plurality of oligonucleotides) between about 300 nucleotides and 1000 nucleotides in length.

In other embodiments, the target capture reagents include cDNA sequences or are derived from cDNAs sequences. In one embodiment, the cDNA is prepared from an RNA sequence, e.g., a tumor- or cancer cell-derived RNA, e.g., an RNA obtained from a tumor-FFPE sample, a blood sample, or a bone marrow aspirate sample. In other embodiments, the target capture reagent includes amplification products (e.g., PCR products) that are amplified from genomic DNA, cDNA or cloned DNA.

In certain embodiments, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single individual. In some embodiments, a library can comprise nucleic acid molecules from more than one subject (e.g., 2, 3, 4, 5, 6, 7, 8, 9, 10, 20, 30 or more subjects), e.g., two or more libraries from different subjects can be combined to form a library having nucleic acid molecules from more than one subject. In one embodiment, the subject is a human having, or at risk of having, a cancer or tumor.

In some embodiments, the method comprises the step of contacting one or a plurality of libraries (e.g., one or a plurality of nucleic acid libraries) with a plurality of target capture reagents to provide a selected subgroup of nucleic acids, e.g., a library catch. In one embodiment, the contacting step is effected in a solid support, e.g., an array. Suitable solid supports for hybridization are described in, e.g., Albert, T. J. et al. (2007) Nat. Methods 4(11): 903-5; Hodges, E. et al. (2007) Nat. Genet. 39(12): 1522-7; and Okou, D. T. et al. (2007) Nat. Methods 4(11): 907-9, the contents of which are hereby incorporated by reference. In other embodiments, the contacting step is effected in solution hybridization. In certain embodiments, the method includes repeating the hybridization step by one or more additional rounds of hybridization. In some embodiments, the method further includes subjecting the library catch to one or more additional rounds of hybridization with the same or different collection of target capture reagents.

In yet other embodiments, the method further includes the step of subjecting the library catch to genotyping, thereby identifying the genotype of the selected nucleic acids.

In certain embodiments, the method further includes one or more of:
i) fingerprinting the sample;
ii) quantifying the abundance of a gene or gene product (e.g., a gene or gene product as described herein) in the sample (e.g., quantifying the relative abundance of a transcript in the sample);
iii) identifying the sample as belonging to a particular subject (e.g., a normal control or a cancer patient);
iv) identifying a genetic trait in the sample (e.g., one or more subject's genetic make-up (e.g., ethnicity, race, familial traits));
v) determining the ploidy in the nucleic acid sample; determining a loss of heterozygosity in the sample;
vi) determining the presence or absence of an alteration described herein, e.g., a nucleotide substitution, copy number alteration, indel, or rearrangement, in the sample;
vii) determining the level of tumor mutational burden and/or microsatellite instability (and/or other complex biomarker) in the sample; or
viii) determining the level of tumor/normal cellular admixture in the sample.

The different oligonucleotide combinations can be mixed at different ratios, e.g., a ratio chosen from 1:1, 1:2, 1:3, 1:4, 1:5, 1:10, 1:20, 1:50; 1:100, 1:1000, or the like. In one embodiment, the ratio of chemically-synthesized target capture reagents (e.g., baits) to array-generated target capture reagents (e.g., baits) is chosen from 1:5, 1:10, or 1:20. The DNA or RNA oligonucleotides can be naturally- or non-naturally-occurring. In certain embodiments, the target capture reagents (e.g., baits) include one or more non-naturally-occurring nucleotides to, e.g., increase melting temperature. Exemplary non-naturally occurring oligonucleotides include modified DNA or RNA nucleotides. An exemplary modified RNA nucleotide is a locked nucleic acid (LNA), wherein the ribose moiety of an LNA nucleotide is modified with an extra bridge connecting the 2′ oxygen and 4′ carbon (Kaur, H; Arora, A; Wengel, J; Maiti, S; Arora, A.; Wengel, J.; Maiti, S. (2006). “Thermodynamic, Counterion, and Hydration Effects for the Incorporation of Locked Nucleic Acid Nucleotides into DNA Duplexes”. Biochemistry 45 (23): 7347-55). Other modified exemplary DNA and RNA nucleotides include, but are not limited to, peptide nucleic acid (PNA) composed of repeating N-(2-aminoethyl)-glycine units linked by peptide bonds (Egholm, M. et al. (1993) Nature 365 (6446): 566-8); a DNA or RNA oligonucleotide modified to capture low GC regions; a bicyclic nucleic acid (BNA) or a crosslinked oligonucleotide; a modified 5-methyl deoxycytidine; and 2,6-diaminopurine. Other modified DNA and RNA nucleotides are known in the art.

In an embodiment, a method further comprises acquiring a library wherein the size of said nucleic acid fragments in the library are less than or equal to a reference value, and said library is made without a fragmentation step between DNA isolation and making the library.

In an embodiment, a method further comprises acquiring nucleic acid fragments and if the size of said nucleic acid fragments are equal to or greater than a reference value and are fragmented and then such nucleic acid fragments are made into a library.

In an embodiment, a method further comprises labeling each of a plurality of library nucleic acid molecules, e.g., by addition of an identifiable distinct nucleic acid sequence (a barcode), to each of a plurality of nucleic acid molecules.

In an embodiment, a method further comprises attaching a primer to each of a plurality of library nucleic acid molecules.

In an embodiment, a method further comprises providing a plurality of target capture reagents and selecting a plurality of target capture reagents, said selection being responsive to: 1) a patient characteristic, e.g., age, stage of tumor, prior treatment, or resistance; 2) tumor type; 3) a characteristic of the sample; 4) a characteristic of a control sample; 5) presence or type of control; 6) a characteristic of the isolated tumor (or control) nucleic acid sample; 7) a library characteristic; 8) a mutation known to be associated with the type of tumor in the sample; 9) a mutation not known to be associated with the type of tumor in the sample; 10) the ability to sequence (or hybridize to or recover) a sequence or identify a mutation, e.g., the difficulty associated with sequence having a high GC region or a rearrangement; or 11) the genes being sequenced.

In an embodiment, a method further comprises responsive, e.g., to a determination of a low number of tumor cells in said sample, selecting a target capture reagent, or plurality of target capture reagents, giving relatively highly efficient capture of nucleic acid molecules of a first gene as compared with nucleic acid molecules of a second gene, e.g., wherein a mutation in the first gene is associated the tumor phenotype for the tumor type of the sample, optionally wherein a mutation in the second gene is not associated with the tumor phenotype for the tumor type of the sample.

In an embodiment, the method further comprises acquiring a value for a library catch characteristic, e.g., the nucleic acid concentration, and comparing the acquired value with a reference criterion for the characteristic.

In an embodiment, a method further comprises selecting a library with a value for a library characteristic that meets the reference criterion for library quantitation.

### Sequencing

The methods and systems described herein can be used in combination with, or as part of, a method or system for sequencing nucleic acids.

In some embodiments, nucleic acid molecules from a library are isolated, e.g., using solution hybridization, thereby providing a library catch. The library catch, or a subgroup thereof, can be sequenced. Accordingly, the methods described herein can further include analyzing the library catch. In some embodiments, the library catch is analyzed by a sequencing method, e.g., a next-generation sequencing method as described herein. In some embodiments, the method includes isolating a library catch by solution hybridization, and subjecting the library catch to nucleic acid sequencing. In certain embodiments, the library catch is re-sequenced.

Any method of sequencing known in the art can be used. Sequencing of nucleic acids, e.g., isolated by solution hybridization, are typically carried out using next-generation sequencing (NGS). Sequencing methods suitable for use herein are described in the art, e.g., as described in International Patent Application Publication No, WO 2012/092426.

In an embodiment, at least 10, 20, 30, 40, 50, 60, 70, 80, or 90% of the reads acquired or analyzed are for subject intervals from genes described herein, e.g., genes from Tables 2A-5B. In an embodiment, at least 0.01, 0.02, 0.03, 0.04, 0.05, 0.1, 0.2, 0.3, 0.4, 0.5, 0.6, 0.7, 0.8, 0.9, 1.0, 2.0, 5.0, 10, 15, or 30 megabases, e.g., genomic bases, are sequenced. In an embodiment, the method comprises acquiring a nucleotide sequence read obtained from a sample described herein. In an embodiment, the reads are provided by an NGS sequencing method.

The methods disclosed herein can be used to detect alterations present in the genome, whole exome or transcriptome of a subject, and can be applied to DNA and RNA sequencing, e.g., targeted DNA and/or RNA sequencing. In some embodiments, a transcript of a gene described herein is sequenced. In other embodiments, the method includes detection of a change (e.g., an increase or decrease) in the level of a gene or gene product, e.g., a change in expression of a gene or gene product described herein. The methods can, optionally, include a step of enriching a sample for a target RNA. In other embodiments, the methods include the step of depleting the sample of certain high abundance RNAs, e.g., ribosomal or globin RNAs. The RNA sequencing methods can be used, alone or in combination with the DNA sequencing methods described herein. In one embodiment, the method includes performing a DNA sequencing step and an RNA sequencing step. The methods can be performed in any order. For example, the method can include confirming by RNA sequencing the expression of an alteration described herein, e.g., confirming expression of a mutation or a fusion detected by the DNA sequencing methods of the invention. In other embodiments, the method includes performing an RNA sequencing step, followed by a DNA sequencing step.

### Alignment

Methods disclosed herein can integrate the use of multiple, individually tuned, alignment methods or algorithms to optimize performance in sequencing methods, particularly in methods that rely on massively parallel sequencing of a large number of diverse genetic events in a large number of diverse genes, e.g., methods of analyzing samples, e.g., from a cancer described herein.

In some embodiments, the alignment method used to analyze reads is not individually customized or tuned to each of a number of variants in different genes. In some embodiments, a multiple alignment method that is individually customized or tuned to at least a subset of a number of variants in different genes is used to analyze reads. In some embodiments, a multiple alignment method that is individually customized or tuned to each of a number of variants in different genes is used to analyze reads. In some embodiments, tuning can be a function of (one or more of) the gene (or other subject interval) being sequenced, the tumor type in the sample, the variant being sequenced, or a characteristic of the sample or the subject. The selection or use of alignment conditions that are individually tuned to a number of subject intervals to be sequenced allows optimization of speed, sensitivity and specificity. The method is particularly effective when the alignments of reads for a relatively large number of diverse subject intervals are optimized,

In some embodiments, a read from each of X unique subject intervals is aligned with a unique alignment method, wherein unique subject interval (e.g., subject interval or expressed subject interval) means different from the other X−1 subject intervals, and wherein the unique alignment method means different from the other X−1 alignment method, and X is at least 2.

In an embodiment, subject intervals from at least X genes, e.g. at least X genes from Tables 2A-5B, are aligned with a unique alignment method, and X is equal to 2, 3, 4, 5, 10, 15, 20, 30, 40, 50, 60, 70, 80, 90, 100, 200, 300, 400, 500, or greater.

In an embodiment, a method comprises selecting or using an alignment method for analyzing, e.g., aligning, a read, wherein said alignment method is a function of, is selected responsive to, or is optimized for, one or more or all of:
(i) tumor type, e.g., the tumor type in said sample;
(ii) the gene, or type of gene, in which said subject interval (e.g., subject interval or expressed subject interval) being sequenced is located, e.g., a gene or type of gene characterized by a variant or type of variant, e.g., a mutation, or by a mutation of a frequency;
(iii) the site (e.g., nucleotide position) being analyzed;
(iv) the type of variant, e.g., a substitution, within the subject interval (e.g., subject interval or expressed subject interval) being evaluated;
(v) the type of sample, e.g., a sample described herein; and
(vi) sequence in or near said subject interval being evaluated, e.g., the expected propensity for misalignment for said subject interval (e.g., subject interval or expressed subject interval), e.g., the presence of repeated sequences in or near said subject interval (e.g., subject interval or expressed subject interval).

As referred to elsewhere herein, in some embodiments, a method is particularly effective when the alignment of reads for a relatively large number of subject intervals is optimized. Thus, in an embodiment, at least X unique alignment methods are used to analyze reads for at least X unique subject intervals, wherein unique means different from the other X−1, and X is equal to 2, 3, 4, 5, 10, 15, 20, 30, 50, 100, 200, 300, 400, 500, 600, 700, 800, 900, 1,000, or greater.

In an embodiment, subject intervals from at least X genes from Tables 2A-5B, are analyzed, and X is equal to 2, 3, 4, 5, 10, 15, 20, 30, 40, 50, 60, 70, 80, 90, 100, 200, 300, 400, 500, or greater.

In an embodiment, a unique alignment method is applied to subject intervals in each of at least 3, 5, 10, 20, 40, 50, 60, 70, 80, 90, 100, 200, 300, 400, or 500 different genes.

In an embodiment, a nucleotide position in at least 20, 40, 60, 80, 100, 120, 140, 160 or 180, 200, 300, 400, or 500 genes, e.g., genes from Tables 2A-5B, is assigned a nucleotide value. In an embodiment, a unique alignment method is applied to subject intervals in each of at least 10, 20, 30, 40, or 50% of said genes analyzed.

Methods disclosed herein allow for the rapid and efficient alignment of troublesome reads, e.g., a read having a rearrangement. Thus, in an embodiment where a read for a subject interval (e.g., a subject interval or an expressed subject interval) comprises a nucleotide position with a rearrangement, e.g., a translocation, the method can comprise using an alignment method that is appropriately tuned and that includes:
selecting a rearrangement reference sequence for alignment with a read, wherein said rearrangement reference sequence aligns with a rearrangement (in some embodiments, the reference sequence is not identical to the genomic rearrangement); and
comparing, e.g., aligning, a read with said rearrangement reference sequence.

In some embodiments, a different method, e.g., another method is used to align troublesome reads. These methods are particularly effective when the alignment of reads for a relatively large number of diverse subject intervals is optimized. By way of example, a method of analyzing a sample can comprise:
performing a comparison, e.g., an alignment comparison, of a read under a first set of parameters (e.g., a first mapping algorithm or with a first reference sequence), and
determining if said read meets a first alignment criterion (e.g., the read can be aligned with said first reference sequence, e.g., with less than a number of mismatches);
if said read fails to meet the first alignment criterion, performing a second alignment comparison under a second set of parameters, (e.g., a second mapping algorithm or with a second reference sequence); and,
optionally, determining if said read meets said second criterion (e.g., the read can be aligned with said second reference sequence with less than a predefined number of mismatches),
wherein said second set of parameters comprises use of a set of parameters, e.g., said second reference sequence, which, compared with said first set of parameters, is more likely to result in an alignment with a read for a variant, e.g., a rearrangement, e.g., an insertion, deletion, or translocation.

In embodiments, an alignment method from the section entitled “Alignment” herein is combined with a mutation calling method from the section entitled “Mutation Calling” herein and/or a target capture reagent from the section entitled “Target Capture Reagents” herein and/or the section entitled “Design and Construction of Target Capture Reagents” herein). The method can be applied to a set of subject intervals from the section entitled “Gene Selection” herein and/or a sample from the section entitled “Sample” herein from a subject from the section “Subject” herein. The terms “individual,” “patient,” and “subject” are used synonymously, and refers to an animal including a human.

Alignment is typically the process of matching a read with a location, e.g., a genomic location. Misalignment (e.g., the placement of base-pairs from a short read on incorrect locations in the genome)., e.g., misalignment due to sequence context (e.g., presence of repetitive sequence) of reads around an actual cancer mutation can lead to reduction in sensitivity of mutation detection, as reads of the alternate allele may be shifted off the main pile-up of alternate allele reads. If the problematic sequence context occurs where no actual mutation is present, misalignment may introduce artifactual reads of “mutated” alleles by placing actual reads of reference genome bases onto the wrong location. Because mutation-calling algorithms for multiplied multigene analysis should be sensitive to even low-abundance mutations, these misalignments may increase false positive discovery rates/reduce specificity.

As discussed herein, reduced sensitivity for actual mutations may be addressed by evaluating the quality of alignments (manually or in an automated fashion) around expected mutation sites in the genes being analyzed. The sites to be evaluated can be obtained from databases of cancer mutations (e.g. COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment processes may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE),

Reduced specificity (increased false positive rate) in the evaluated gene regions due to misalignment can be assessed by manual or automated examination of all mutation calls in samples sequenced. Those regions found to be prone to spurious mutation calls due to misalignment can be subjected to same alignment remedies as above. In cases where no algorithmic remedy is found possible, “mutations” from the problem regions can be classified or screened out from the test panel.

Methods disclosed herein allow the use of multiple, individually tuned, alignment methods or algorithms to optimize performance in the sequencing of subject intervals associated with rearrangements, e.g., indels, particularly in methods that rely on massively parallel sequencing of a large number of diverse genetic events in a large number of diverse genes, e.g., from samples. In some embodiments, a multiple alignment method that is individually customized or tuned to each of a number of rearrangements in different genes is used to analyze reads. In some embodiments, tuning can be a function of one or more of the subject intervals (e.g., one or more of the genes) being sequenced, the tumor type associated with the sample, the variant being sequenced, or a characteristic of the sample or the subject. This selection or use of alignment conditions finely tuned to a number of subject intervals to be sequenced allows optimization of speed, sensitivity and specificity. The method is particularly effective when the alignment of reads for a relatively large number of diverse subject intervals is optimized. In embodiments, the method includes the use of an alignment method optimized for rearrangements and others optimized for subject intervals not associated with rearrangements.

In some embodiments, an alignment selector is used. “Alignment selector,” as used herein, refers to a parameter that allows or directs the selection of an alignment method, e.g., an alignment algorithm or parameter, that can optimize the sequencing of a subject interval. An alignment selector can be specific to, or selected as a function, e.g., of one or more of the following:

1. The sequence context, e.g., sequence context, of a subject interval (e.g., the nucleotide position to be evaluated) that is associated with a propensity for misalignment of reads for said subject interval. E.g., the existence of a sequence element in or near the subject interval to be evaluated that is repeated elsewhere in the genome can cause misalignment and thereby reduce performance. Performance can be enhanced by selecting an algorithm or an algorithm parameter that minimizes misalignment. In this case the value for the alignment selector can be a function of the sequence context, e.g., the presence or absence of a sequence of length that is repeated at least a number of times in the genome (or in the portion of the genome being analyzed).
2. The tumor type being analyzed. E.g., a specific tumor type can be characterized by increased rate of deletions. Thus, performance can be enhanced by selecting an algorithm or algorithm parameter that is more sensitive to indels. In this case the value for the alignment selector can be a function of the tumor type, e.g., an identifier for the tumor type. In an embodiment the value is the identity of the tumor type, e.g., a solid tumor or a hematologic malignancy (or premaligancy).
3. The gene, or type of gene, being analyzed, e.g., a gene, or type of gene, can be analyzed. Oncogenes, by way of example, are often characterized by substitutions or in-frame indels. Thus, performance can be enhanced by selecting an algorithm or algorithm parameter that is particularly sensitive to these variants and specific against others. Tumor suppressors are often characterized by frame-shift indels. Thus, performance can be enhanced by selecting an algorithm or algorithm parameter that is particularly sensitive to these variants. Thus, performance can be enhanced by selecting an algorithm or algorithm parameter matched with the subject interval. In this case the value for the alignment selector can be a function of the gene or gene type, e.g., an identifier for gene or gene type. In an embodiment the value is the identity of the gene.
4. The site (e.g., nucleotide position) being analyzed. In this case the value for the alignment selector can be a function of the site or the type of site, e.g., an identifier for the site or site type. In an embodiment the value is the identity of the site. (E.g., if the gene containing the site is highly homologous with another gene, normal/fast short read alignment algorithms (e.g., BWA) may have difficulty distinguishing between the two genes, potentially necessitating more intensive alignment methods (Smith-Waterman) or even assembly (ARACHNE). Similarly, if the gene sequence contains low-complexity regions (e.g., AAAAAA), more intensive alignment methods may be necessary.
5. The variant, or type of variant, associated with the subject interval being evaluated. E.g., a substitution, insertion, deletion, translocation or other rearrangement. Thus, performance can be enhanced by selecting an algorithm or algorithm parameter that is more sensitive to the specific variant type. In this case the value for the alignment selector can be a function of the type of variant, e.g., an identifier for the type of variant. In an embodiment the value is the identity of the type of variant, e.g., a substitution.
6. The type of sample, e.g., a sample described herein. Sample type/quality can affect error (spurious observation of non-reference sequence) rate. Thus, performance can be enhanced by selecting an algorithm or algorithm parameter that accurately models the true error rate in the sample. In this case the value for the alignment selector can be a function of the type of sample, e.g., an identifier for the sample type. In an embodiment, the value is the identity of the sample type.

Generally, the accurate detection of indel mutations is an exercise in alignment, as the spurious indel rate on the sequencing platforms disabled herein is relatively low (thus, even a handful of observations of correctly aligned indels can be strong evidence of mutation). Accurate alignment in the presence of indels can be difficult however (especially as indel length increases). In addition to the general issues associated with alignment, e.g., of substitutions, the indel itself can cause problems with alignment. (For instance, a deletion of 2 bp of a dinucleotide repeat cannot be readily definitively placed.) Both sensitivity and specificity can be reduced by incorrect placement of shorter (<15 bp) apparent indel-containing reads. Larger indels (getting closer in magnitude to the length of individual reads, e.g., reads of 36 bp) can cause failure to align the read at all, making detection of the indel impossible in the standard set of aligned reads.

Databases of cancer mutations can be used to address these problems and improve performance. To reduce false positive indel discovery (improve specificity), regions around commonly expected indels can be examined for problematic alignments due to sequence context and addressed similarly to substitutions above. To improve sensitivity of indel detection, several different processes of using information on the indels expected in cancer can be used. E.g., short-reads contained expected indels can be simulated and alignment attempted. The alignments can be studied and problematic indel regions can have alignment parameters adjusted, for instance by reducing gap open/extend penalties or by aligning partial reads (e.g. the first or second half of a read).

Alternatively, initial alignment can be attempted not just with the normal reference genome, but also with alternate versions of the genome, containing each of the known or likely cancer indel mutations. In this process, reads of indels that initially failed to align or aligned incorrectly are placed successfully on the alternate (mutated) version of the genome.

In this way, indel alignment (and thus calling) can be optimized for the expected cancer genes/sites. As used herein, a sequence alignment algorithm embodies a computational method or process used to identify from where in the genome a read sequence (e.g., a short-read sequence, e.g., from next-generation sequencing) most likely originated by assessing the similarity between the read sequence and a reference sequence. A variety of algorithms can be applied to the sequence alignment problem. Some algorithms are relatively slow, but allow relatively high specificity. These include, e.g., dynamic programming-based algorithms. Dynamic programming is a method for solving complex problems by breaking them down into simpler steps. Other processes are relatively more efficient, but are typically not as thorough. These include, e.g., heuristic algorithms and probabilistic methods designed for large-scale database search.

Alignment parameters are used in alignment algorithms to adjust performance of an algorithm, e.g., to produce an optimal global or local alignment between a read sequence and a reference sequence. Alignment parameters can give weights for match, mismatch, and indels. For example, lower weights allow alignments with more mismatches and indels.

Sequence context, e.g., presence of repetitive sequences (e.g., tandem repeats, interspersed repeats), low-complexity regions, indels, pseudogenes, or paralogs can affect the alignment specificity (e.g., cause misalignment). As used herein, misalignment refers to the placement of base-pairs from the short read on incorrect locations in the genome.

The sensitivity of alignment can be increased when an alignment algorithm is selected or an alignment parameter is adjusted based on tumor type, e.g., a tumor type that tends to have a particular mutation or mutation type.

The sensitivity of alignment can be increased when an alignment algorithm is selected or an alignment parameter is adjusted based on a particular gene type (e.g., oncogene, tumor suppressor gene). Mutations in different types of cancer-associated genes can have different impacts on cancer phenotype. For example, mutant oncogene alleles are typically dominant. Mutant tumor suppressor alleles are typically recessive, which means that in most cases both alleles of a tumor suppressor genes must be affected before an effect is manifested.

The sensitivity of alignment can be adjusted (e.g., increased) when an alignment algorithm is selected or an alignment parameter is adjusted based on mutation type (e.g., single nucleotide polymorphism, indel (insertion or deletion), inversion, translocation, tandem repeat).

The sensitivity of alignment can be adjusted (e.g., increased) when an alignment algorithm is selected or an alignment parameter is adjusted based on mutation site (e.g., a mutation hotspot). A mutation hotspot refers to a site in the genome where mutations occur up to 100 times more frequently than the normal mutation rate.

The sensitivity/specificity of alignment can be adjusted (e.g., increased) when an alignment algorithm is selected or an alignment parameter is adjusted based on sample type (e.g., cfDNA sample, ctDNA sample, FFPE sample, or CTC sample).

In some embodiments, NGS reads can be aligned to a known reference sequence or assembled de novo. For example, the NGS reads can be aligned to a reference sequence (e.g., a wild-type sequence). Methods of sequence alignment for NGS are described e.g., in Trapnell C. and Salzberg S. L. Nature Biotech., 2009, 27:455-457. Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data.

Optimization of alignment is described in the art, e.g., as set out in International Patent Application Publication No. WO 2012/092426.

### Mutation Calling

Methods disclosed herein can integrate the use of customized or tuned mutation calling parameters to optimize performance in sequencing methods, particularly in methods that rely on massively parallel sequencing of a large number of diverse genetic events in a large number of diverse genes, e.g., from samples, e.g., from a cancer described herein.

In some embodiments, mutation calling for each of a number of subject intervals is not individually customized or fine-tuned. In some embodiments, mutation calling for at least a subset of a number of subject intervals is, individually, customized or fine-tuned. In some embodiments, mutation calling for each of a number of subject intervals is, individually, customized or fine-tuned. The customization or tuning can be based on one or more of the factors described herein, e.g., the type of cancer in a sample, the gene in which the subject interval to be sequenced is located, or the variant to be sequenced. This selection or use of alignment conditions finely tuned to a number of subject intervals to be sequenced allows optimization of speed, sensitivity and specificity. The method is particularly effective when the alignment of reads for a relatively large number of diverse subject intervals is optimized.

In some embodiments, a nucleotide value is assigned for a nucleotide position in each of X unique subject intervals is assigned by a unique calling method, wherein unique subject interval (means different from the other X−1 subject intervals (e.g., subgenomic intervals, expressed subgenomic intervals, or both), and wherein the unique calling method means different from the other X−1 calling method, and X is at least 2. The calling methods can differ, and thereby be unique, e.g., by relying on different Bayesian prior values.

In an embodiment, assigning said nucleotide value is a function of a value which is or represents the prior (e.g., literature) expectation of observing a read showing a variant, e.g., a mutation, at said nucleotide position in a tumor of type.

In an embodiment, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique (as opposed to the value for the other assignments) value which is or represents the prior (e.g., literature) expectation of observing a read showing a variant, e.g., a mutation, at said nucleotide position in a tumor of type.

In an embodiment, assigning said nucleotide value is a function of a set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone).

In an embodiment, the mutation calling method described herein can include the following:
acquiring, for a nucleotide position in each of said X subject intervals:
   (i) a first value which is or represents the prior (e.g., literature) expectation of observing a read showing a variant, e.g., a mutation, at said nucleotide position in a tumor of type X; and
   (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone);
responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

In an embodiment, the method comprises one or more or all of:
(i) assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is based on a unique (as opposed to the other assignments) first and/or second values;
(ii) the assignment of method of (i), wherein at least 10, 20, 30, 40, 50, 60, 70, 80, 90, 100, 200, 300, 400, or 500 of the assignments are made with first values which are a function of a probability of a variant being present in less than 5, 10, or 20%, e.g., of the cells in a tumor type;
(iii) assigning a nucleotide value (e.g., calling a mutation) for at least X nucleotide positions, each of which of which being associated with a variant having a unique (as opposed to the other X−1 assignments) probability of being present in a tumor of type, e.g., the tumor type of said sample, wherein, optionally, each of said of X assignments is based on a unique (as opposed to the other X−1 assignments) first and/or second value (wherein X=2, 3, 5, 10, 20, 40, 50, 60, 70, 80, 90, 100, 200, 300, 400, or 500);
(iv) assigning a nucleotide value (e.g., calling a mutation) at a first and a second nucleotide position, wherein the likelihood of a first variant at said first nucleotide position being present in a tumor of type (e.g., the tumor type of said sample) is at least 2, 5, 10, 20, 30, or 40 times greater than the likelihood of a second variant at said second nucleotide position being present, wherein, optionally, each assignment is based on a unique (as opposed to the other assignments) first and/or second value;
(v) assigning a nucleotide value to a plurality of nucleotide positions (e.g., calling mutations), wherein said plurality comprises an assignment for variants falling into one or more, e.g., at least 3, 4, 5, 6, 7, or all, of the following probability percentage ranges: less than or equal to 0.01; greater than 0.01 and less than or equal to 0.02; greater than 0.02 and less than or equal to 0.03; greater than 0.03 and less than or equal to 0.04; greater than 0.04 and less than or equal to 0.05; greater than 0.05 and less than or equal to 0.1; greater than 0.1 and less than or equal to 0.2; greater than 0.2 and less than or equal to 0.5; greater than 0.5 and less than or equal to 1.0; greater than 1.0 and less than or equal to 2.0; greater than 2.0 and less than or equal to 5.0; greater than 5.0 and less than or equal to 10.0; greater than 10.0 and less than or equal to 20.0; greater than 20.0 and less than or equal to 50.0; and greater than 50 and less than or equal to 100.0%,
wherein, a probability range is the range of probabilities that a variant at a nucleotide position will be present in a tumor type (e.g., the tumor type of said sample) or the probability that a variant at a nucleotide position will be present in the recited percentage (%) of the cells in a sample, a library from the sample, or library catch from that library, for a preselected type (e.g., the tumor type of said sample), and
wherein, optionally, each assignment is based on a unique first and/or second value (e.g., unique as opposed to the other assignments in a recited probability range or unique as opposed to the first and/or second values for one or more or all of the other listed probability ranges).
(vi) assigning a nucleotide value (e.g., calling a mutation) for at least 1, 2, 3, 5, 10, 20, 40, 50, 60, 70, 80, 90, 100, 200, 300, 400, 500, 600, 700, 800, 900, or 1,000 nucleotide positions each, independently, having a variant present in less than 50, 40, 25, 20, 15, 10, 5, 4, 3, 2, 1, 0.5, 0.4, 0.3, 0.2, or 0.1% of the DNA in said sample, wherein, optionally, each assignment is based on a unique (as opposed to the other assignments) first and/or second value;
(vii) assigning a nucleotide value (e.g., calling a mutation) at a first and a second nucleotide position, wherein the likelihood of a variant at the first position in the DNA of said sample is at least 2, 5, 10, 20, 30, or 40 times greater than the likelihood of a variant at said second nucleotide position in the DNA of said sample, wherein, optionally, each assignment is based on a unique (as opposed to the other assignments) first and/or second value;
(viii) assigning a nucleotide value (e.g., calling a mutation) in one or more or all of the following:
(1) at least 1, 2, 3, 4 or 5 nucleotide positions having a variant present in less than 1% of the cells in said sample, of the nucleic acids in a library from said sample, or the nucleic acid in a library catch from that library;
(2) at least 1, 2, 3, 4 or 5 nucleotide positions having a variant present in 1-2% of the cells in said sample, of the nucleic acid in a library from said sample, or the nucleic acid in a library catch from that library;
(3) at least 1, 2, 3, 4 or 5 nucleotide positions having a variant present in greater than 2% and less than or equal to 3% of the cells in said sample, of the nucleic acid in a library from said sample, or the nucleic acid in a library catch from that library
(4) at least 1, 2, 3, 4 or 5 nucleotide positions having a variant present in greater than 3% and less than or equal to 4% of the cells in said sample, of the nucleic acid in a library from said sample, or the nucleic acid in a library catch from that library;
(5) at least 1, 2, 3, 4 or 5 nucleotide positions having a variant present in greater than 4% and less than or equal to 5% of the cells in said sample, of the nucleic acid in a library from said sample, or the nucleic acid in a library catch from that library;
(6) at least 1, 2, 3, 4 or 5 nucleotide positions having a variant present in greater than 5% and less than or equal to 10% of the cells in said sample, of the nucleic acid in a library from said sample, or the nucleic acid in a library catch from that library;
(7) at least 1, 2, 3, 4 or 5 nucleotide positions having a variant present in greater than 10% and less than or equal to 20% of the cells in said sample, of the nucleic acid in a library from said sample, or the nucleic acid in a library catch from that library;
(8) at least 1, 2 3, 4 or 5 nucleotide positions having a variant present in greater than 20% and less than or equal to 40% of the cells in said sample, of the nucleic acid in a library from said sample, or the nucleic acid in a library catch from that library;
(9) at least 1, 2 3, 4 or 5 nucleotide positions having a variant present at greater than 40% and less than or equal to 50% of the cells in said sample, of the nucleic acid in a library from said sample, or the nucleic acid in a library catch from that library; or
(10) at least 1, 2 3, 4 or 5 nucleotide positions having a variant present in greater than 50% and less than or equal to 100% of the cells in said sample, of the nucleic acid in a library from said sample, or the nucleic acid in a library catch from that library;
wherein, optionally, each assignment is based on a unique first and/or second value (e.g., unique as opposed to the other assignments in the recited range (e.g., the range in (1) of less than 1%) or unique as opposed to a first and/or second values for a determination in one or more or all of the other listed ranges); or
(ix) assigning a nucleotide value (e.g., calling a mutation) at each of X nucleotide positions, each nucleotide position, independently, having a likelihood (of a variant being present in the DNA of said sample) that is unique as compared with the likelihood for a variant at the other X−1 nucleotide positions, wherein X is equal to or greater than 1, 2, 3, 5, 10, 20, 40, 50, 60, 70, 80, 90, 100, 200, 300, 400, 500, 600, 700, 800, 900, or 1,000, and wherein each assignment is based on a unique (as opposed to the other assignments) first and/or second value.

In some embodiments, a “threshold value” is used to evaluate reads, and select from the reads a value for a nucleotide position, e.g., calling a mutation at a specific position in a gene. In some embodiments, a threshold value for each of a number of subject intervals is customized or fine-tuned. The customization or tuning can be based on one or more of the factors described herein, e.g., the type of cancer in a sample, the gene in which the subject interval (subgenomic interval or expressed subgenomic interval) to be sequenced is located, or the variant to be sequenced. This provides for calling that is finely tuned to each of a number of subject intervals to be sequenced. In some embodiments, the method is particularly effective when a relatively large number of diverse subgenomic intervals are analyzed.

Thus, in another embodiment, the method comprises the following mutation calling method:
acquiring, for each of said X subject intervals, a threshold value, wherein each of said acquired X threshold values is unique as compared with the other X−1 threshold values, thereby providing X unique threshold values;
for each of said X subject intervals, comparing an observed value which is a function of the number of reads having a nucleotide value at a nucleotide position with its unique threshold value, thereby applying to each of said X subject intervals its unique threshold value; and optionally, responsive to the result of said comparison, assigning a nucleotide value to a nucleotide position,
wherein X is equal to or greater than 2.

In an embodiment, the method includes assigning a nucleotide value to at least 2, 3, 5, 10, 20, 40, 50, 60, 70, 80, 90, 100, 200, 300, 400, 500, 600, 700, 800, 900, or 1,000 nucleotide positions, each having, independently, a first value that is a function of a probability that is less than 0.5, 0.4, 0.25, 0.15, 0.10, 0.05, 0.04, 0.03, 0.02, or 0.01.

In an embodiment, the method includes assigning a nucleotide value to at each of at least X nucleotide positions, each independently having a first value that is unique as compared with the other X−1 first values, and wherein each of said X first values is a function of a probability that is less than 0.5, 0.4, 0.25, 0.15, 0.10, 0.05, 0.04, 0.03, 0.02, or 0.01, wherein X is equal to or greater than 1, 2, 3, 5, 10, 20, 40, 50, 60, 70, 80, 90, 100, 200, 300, 400, 500, 600, 700, 800, 900, or 1,000.

In an embodiment, a nucleotide position in at least 20, 40, 60, 80, 100, 120, 140, 160 or 180, 200, 300, 400, or 500 genes, e.g., genes from Tables 2A-5B, is assigned a nucleotide value. In an embodiment unique first and/or second values are applied to subject intervals in each of at least 10, 20, 30, 40, or 50% of said genes analyzed.

Embodiments of the method can be applied where threshold values for a relatively large number of subject intervals are optimized, as is seen, e.g., from the following embodiments.

In an embodiment, a unique threshold value is applied to subject intervals, e.g., subgenomic intervals or expressed subgenomic intervals, in each of at least 3, 5, 10, 20, 40, 50, 60, 70, 80, 90, 100, 200, 300, 400, 500, 600, 700, 800, 900, or 1,000 different genes.

In an embodiment, a nucleotide position in at least 20, 40, 60, 80, 100, 120, 140, 160 or 180, 200, 300, 400, or 500 genes, e.g., genes from Tables 2A-5B, is assigned a nucleotide value. In an embodiment a unique threshold value is applied to a subgenomic interval in each of at least 10, 20, 30, 40, or 50% of said genes analyzed.

In an embodiment, a nucleotide position in at least 5, 10, 20, 30, or 40 genes from Tables 2A-5B is assigned a nucleotide value. In an embodiment a unique threshold value is applied to a subject interval (e.g., a subgenomic interval or an expressed subgenomic interval) in each of at least 10, 20, 30, 40, or 50% of said genes analyzed.

Elements of that module can be included in methods of analyzing a tumor. In embodiments, an alignment method from the section entitled “Mutation Calling” is combined with an alignment method from the section entitled “Alignment” herein and/or target capture reagents from the section entitled “Target Capture Reagents” herein and/or the sections entitled “Design and Construction of Target Capture Reagents” and “Competition of Target Capture Reagents” herein). The method can be applied to a set of subject intervals from the section entitled “Gene Selection” herein and/or a sample from the section entitled “Sample” herein from a subject from the section “Subject” herein.

Base calling refers to the raw output of a sequencing device. Mutation calling refers to the process of selecting a nucleotide value, e.g., A, G, T, or C, for a nucleotide position being sequenced. Typically, the sequencing reads (or base calling) for a position will provide more than one value, e.g., some reads will give a T and some will give a G. Mutation calling is the process of assigning a nucleotide value, e.g., one of those values to the sequence. Although it is referred to as “mutation” calling it can be applied to assign a nucleotide value to any nucleotide position, e.g., positions corresponding to mutant alleles, wild-type alleles, alleles that have not been characterized as either mutant or wild-type, or to positions not characterized by variability. Methods for mutation calling can include one or more of the following: making independent calls based on the information at each position in the reference sequence (e.g., examining the sequence reads; examining the base calls and quality scores; determining the probability of observed bases and quality scores given a potential genotype; and assigning genotypes (e.g., using Bayes rule)); removing false positives (e.g., using depth thresholds to reject SNPs with read depth much lower or higher than expected; local realignment to remove false positives due to small indels); and performing linkage disequilibrium (LD)/imputation based analysis to refine the calls.

Equations to calculate the genotype likelihood associated with a specific genotype and position are described, e.g., in Li H. and Durbin R. Bioinformatics, 2010; 26(5): 589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described, e.g., in Browning B. L. and Yu Z. Am. J. Hum. Genet. 2009, 85(6): 847-61. Examples of low-coverage SNP calling methods are described, e.g., in Li Y. et al., Annu. Rev. Genomics Hum. Genet. 2009, 10:387-406.

After alignment, detection of substitutions can be performed using a calling method, e.g., Bayesian mutation calling method; which is applied to each base in each of the subject intervals, e.g., exons of the gene to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation,

Methods have been developed that address limited deviations from frequencies of 50% or 100% for the analysis of cancer DNA. (e.g., SNVMix—Bioinformatics. 2010 Mar. 15; 26(6): 730-736.) Methods disclosed herein however allow consideration of the possibility of the presence of a mutant allele in anywhere between 1% and 100% of the sample DNA, and especially at levels lower than 50%. This process is particularly important for the detection of mutations in low-purity FFPE samples of natural (multi-clonal) tumor DNA.

An advantage of a Bayesian mutation-detection process is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ˜1e-6. The likelihood of specific mutations at many sites in a typical multigenic cancer genome panel can be orders of magnitude higher. These likelihoods can be derived from public databases of cancer mutations (e.g., COSMIC). Indel calling is a process of finding bases in the sequencing data that differ from the reference sequence by insertion or deletion, typically including an associated confidence score or statistical evidence metric.

Methods of indel calling can include the steps of identifying candidate indels, determining genotype likelihood through local re-alignment, and performing LD-based genotype inference and calling. Typically, a Bayesian process is used to obtain potential indel candidates, and then these candidates are tested together with the reference sequence in a Bayesian framework.

Algorithms to generate candidate indels are described, e.g., in McKenna A. et al., Genome Res. 2010; 20(9):1297-303; Ye K. et al., Bioinformatics, 2009; 25(21):2865-71; Lunter G. and Goodson M. Genome Res. 2011; 21(6):936-9; and Li H. et al., Bioinformatics 2009, Bioinformatics 25(16):2078-9.

Methods for generating indel calls and individual-level genotype likelihoods include, e.g., the Dindel algorithm (Albers C. A. et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

In an embodiment, at least 10, 20, 30, 40, 50, 60, 70, 80, or 90% of the mutation calls made in the method are for subject intervals from genes or gene products described herein, e.g., genes or gene products from Tables 2A-5B. In an embodiment, at least 10, 20, 30, 40, 50, 60, 70, 80, or 90% of the unique threshold values described herein are for subject intervals from genes or gene products described herein, e.g., genes or gene products from Tables 2A-5B. In an embodiment, at least 10, 20, 30, 40, 50, 60, 70, 80, or 90% of the mutation calls annotated, or reported to a third party, are for subject intervals from genes or gene products described herein, e.g., genes or gene products from Tables 2A-5B.

In an embodiment, the assigned value for a nucleotide position is transmitted to a third party, optionally, with explanatory annotation. In an embodiment, the assigned value for a nucleotide position is not transmitted to a third party. In an embodiment, the assigned value for a plurality of nucleotide position is transmitted to a third party, optionally, with explanatory annotations, and the assigned value for a second plurality of nucleotide position is not transmitted to a third party.

In an embodiment, the method comprises assigning one or more reads to a subject, e.g., by barcode deconvolution.

In an embodiment, the method comprises assigning one or more reads as a tumor read or a control read, e.g., by barcode deconvolution. In an embodiment, the method comprises mapping, e.g., by alignment with a reference sequence, each of said one or more reads. In an embodiment, the method comprises memorializing a called mutation.

In an embodiment, the method comprises annotating a called mutation, e.g., annotating a called mutation with an indication of mutation structure, e.g., a missense mutation, or function, e.g., a disease phenotype. In an embodiment, the method comprises acquiring nucleotide sequence reads for tumor and control nucleic acid. In an embodiment, the method comprises calling a nucleotide value, e.g., a variant, e.g., a mutation, for each of the subject intervals (e.g., subgenomic intervals, expressed subgenomic intervals, or both), e.g., with a Bayesian calling method or a non-Bayesian calling method. In an embodiment, the method comprises evaluating a plurality of reads that include at least one SNP. In an embodiment, the method comprises determining an SNP allele ratio in the sample and/or control read.

In some embodiments, the method further comprises building a database of sequencing/alignment artifacts for the targeted subgenomic regions. In an embodiment, the database can be used to filter out spurious mutation calls and improve specificity. In an embodiment the database is built by sequencing unrelated samples or cell-lines and recording non-reference allele events that appear more frequently than expected due to random sequencing error alone in 1 or more of these normal samples. This process may classify germline variation as artifact, but that is acceptable in a method concerned with somatic mutations. This misclassification of germline variation as artifact may be ameliorated if desired by filtering this database for known germline variations (removing common variants) and for artifacts that appear in only 1 individual (removing rarer variations).

Optimization of mutation calling is described in the art, e.g., as set out in International Patent Application Publication No. WO 2012/092426.

### SGZ Algorithm

Various types of alterations, e.g., somatic alterations and germline mutations, can be detected by a method (e.g., a sequencing, alignment, or mutation calling method) described herein. In certain embodiments, a germline mutation is further identified by a method using the SGZ (somatic-germline-zygosity) algorithm. See, for example U.S. Pat. No. 9,792,403 and Sun et al., A computational process to distinguish somatic vs. germline origin of genomic alteration from deep sequencing of cancer specimens without a matched normal, PLOS Computational Biology (February 2018).

In clinical practice, matched normal controls are not commonly obtained. In some embodiments, although well-characterized genomic alterations do not require normal tissue for interpretation, at least some alterations will be unknown in whether they are germline or somatic, in the absence of a matched normal control. SGZ is a computational method for predicting somatic versus germline origin and homozygous versus heterozygous or sub-clonal state of variants identified from next-generation sequencing of cancer specimens.

The SGZ method does not require a matched normal control, allowing for broad application in a clinical setting. SGZ predicts the somatic vs. germline status of each alteration identified by modeling the alteration's allele frequency (AF), taking into account the tumor content, tumor ploidy, and the local copy number. Accuracy of the prediction depends on the depth of sequencing and copy number model fit, which can be achieved by sequencing to high depth, covering cancer-related genes and genome-wide single nucleotide polymorphisms (SNPs). Calls are made using a statistic based on read depth and local variability of SNP AF.

In some embodiments, the method further comprises characterizing a variant, e.g., a mutation, in a tissue (e.g., a tumor) or a sample, from a subject, e.g., a human, e.g., a cancer patient, comprising:
a) acquiring:
i) a sequence coverage input (SCI), which comprises, for each of a plurality of selected subject intervals, e.g., exons, a value for normalized sequence coverage at the selected subject intervals;
ii) an SNP allele frequency input (SAFI), which comprises, for each of a plurality of selected germline SNPs, a value for the allele frequency, in the tumor or sample;
iii) a variant allele frequency input (VAFI), which comprises the allele frequency for said variant, e.g., mutation, in the tumor or sample;
b) acquiring values, as a function of SCI and SAFI, for:
C, for each of a plurality of genomic segments, wherein C is a genomic segment total copy number;
M, for each of a plurality of genomic segments, wherein M is a genomic segment minor allele copy number; and
p, wherein p is sample purity; and
c) acquiring one or both of:
i) a value for variant type, e.g. mutation type, e.g., g, which is indicative of the variant, e.g., a mutation, being somatic, a subclonal somatic variant, germline, or not-distinguishable, and is a function of VAFI, p, C, and M;
ii) an indication of the zygosity of the variant, e.g., mutation, in the tumor or sample, as function of C and M.

In an embodiment, the analysis can be performed without the need for analyzing non-tumor tissue from the subject. In an embodiment, the analysis is performed without analyzing non-tumor tissue from the subject, e.g., non-tumor tissue from the same subject is not sequenced.

In an embodiment, the SCI comprises values that are a function, e.g., the log of the ratio, of the number of reads for a subject interval, e.g., from the sample, and the number or reads for a control, e.g., a process-matched control. In an embodiment, the SCI comprises values, e.g., log r values, for at least 10, 25, 50, 100, 150, 200, 250, 300, 350, 400, 450, 500, 1,000, 2,000, 3,000, 4,000, 5,000, 6,000, 7,000, 8,000, 9,000, or 10,000, subject intervals, e.g., exons. In an embodiment, the SCI comprises values, e.g., log r values, for at least 100 subject intervals, e.g., exons. In an embodiment, the SCI comprises values, e.g., log r values, for 1,000 to 10,000, 2,000 to 9,000, 3,000 to 8,000, 3,000 to 7,000, 3,000 to 6,000, or 4,000 to 5,000, subject intervals, e.g., exons. In an embodiment, the SCI comprises values, e.g., log r values, for subject intervals, e.g., exons, from at least 10, 25, 50, 100, 150, 200, 250, 300, 350, 400, 450, 500, 1,000, 2,000, 3,000, or 4,000, genes.

In an embodiment, at least one, a plurality, or substantially all of the values comprised in the SCI are corrected for correlation with GC content.

In an embodiment, a subject interval, e.g., an exon, from the sample has at least 10, 20, 30, 40, 50, 100, 150, 200, 250, 300, 350, 400, 450, 500, 600, 700, 800, 900, or 1,000 reads. In an embodiment, a plurality, e.g., at least 10, 25, 50, 100, 150, 200, 250, 300, 350, 400, 450, 500, 1,000, 2,000, 3,000, 4,000, 5,000, 6,000, 7,000, 8,000, 9,000, or 10,000, subject intervals, e.g., exons, from the sample has a number of reads. In an embodiment, the number of reads is at least 10, 20, 30, 40, 50, 100, 150, 200, 250, 300, 350, 400, 450, 500, 600, 700, 800, 900, or 1,000. In an embodiment, the plurality of germline SNPs comprise at least 10, 25, 50, 100, 150, 200, 250, 300, 350, 400, 450, 500, 1,000, 2,000, 3,000, 4,000, 5000, 6000, 7000, 8000, 9000, 10,000, or 15,000 germline SNPs.

In an embodiment, the plurality of germline SNPs comprises at least 100 germline SNPs. In an embodiment, the plurality of germline SNPs comprises 500 to 5,000, 1,000 to 4,000, or 2,000 to 3,000 germline SNPs. In an embodiment, the allele frequency is a minor allele frequency. In an embodiment, the allele frequency is an alternative allele, e.g., an allele other than a standard allele in a human genome reference database.

In an embodiment, the method comprises characterizing a plurality of variants, e.g., mutants, in the sample. In an embodiment, the method comprises characterizing at least 2, 3, 4, 5, 6, 7, 8 9, 10, 25, 50, 100, 150, 200, 250, 300, 350, 400, 450, or 500 variants, e.g., mutants. In an embodiment, the method comprises characterizing variants, e.g., mutants, in at least 2, 3, 4, 5, 6, 7, 89, 10, 25, 50, 100, 150, 200, 250, 300, 350, 400, 450, or 500 different genes.

In an embodiment, the method comprises acquiring a VAFI for at least 2, 3, 4, 5, 6, 7, 8 9, 10, 25, 50, 100, 150, 200, 250, 300, 350, 400, 450, or 500 variants, e.g., mutants. In an embodiment, the method comprises performing one, two or all, of steps a), b), and c) for at least 2, 3, 4, 5, 6, 7, 8 9, 10, 25, 50, 100, 150, 200, 250, 300, 350, 400, 450, or 500 variants, e.g., mutants. In an embodiment, values of C, M, and p are, have, or can be obtained by, fitting a genome-wide copy number model to one or both of the SCI and the SAFI. In an embodiment, values of C, M, and p fit a plurality of genome-wide copy number model inputs of the SCI and the SAFI, In an embodiment, a genomic segment comprises a plurality of subject intervals, e.g., exons, e.g., subject intervals which have been assigned a SCI value.

In an embodiment, a genomic segment comprises at least 10, 20, 30, 40, 50, 60, 70, 80, 90, 100, 125, 150, 175, 200, 225, 250, 275, 300, 400, or 500 subject intervals, e.g., exons. In an embodiment, a genomic segment comprises 10 to 1,000, 20 to 900, 30 to 700, 40 to 600, 50 to 500, 60 to 400, 70 to 300, 80 to 200, 80 to 150, or 80 to 120, 90 to 110, or about 100, subject intervals, e.g., exons. In an embodiment, a genomic segment comprises between 100 and 10,000, 100 and 5,000, 100 and 4,000, 100 and 3,000, 100 and 2,000, or 100 and 1,000, subject intervals, e.g., exons. In an embodiment, a genomic segment comprises 10 to 1,000, 20 to 900, 30 to 700, 40 to 600, 50 to 500, 60 to 400, 70 to 300, 80 to 200, 80 to 150, or 80 to 120, 90 to 110, or about 100 genomic SNPs, which have been assigned a SAFI value. In an embodiment, a genomic segment comprises between 100 and 10,000, 100 and 5,000, 100 and 4,000, 100 and 3,000, 100 and 2,000, or 100 and 1,000, genomic SNPs which have been assigned a SAFI value.

In an embodiment, each of a plurality of genomic segments are characterized by having one or both of:

a measure of normalized sequence coverage, e.g., log r, that differ by no more than a preselected amount, e.g., the values for log 2 r for subject intervals, e.g., exons, within the boundaries of the genomic segment differ by no more than a reference value, or are substantially constant; and SNP allele frequencies for germline SNPs that differ by no more than a preselected amount, e.g., the values for germline SNP allele frequencies for subject intervals, e.g., exons, within the boundaries of the genomic segment differ by no more than a reference value, or are substantially constant,

In an embodiment, the number of subject intervals, e.g., exons, that are contained in, or are combined to form, a genomic segment is at least 2, 5, 10, 15, 20, 50, or 100 times the number of genomic segments. In an embodiment, the number of subject intervals, e.g., exons, is at least 3, 4, 5, 6, 7, 8, 9, 10, 11, 12, 13, 14, or 15 times the number of genomic segments.

In an embodiment, a boundary for a genomic segment is provided. In an embodiment, the method comprises assembling sequences for subject intervals, e.g., exons, into genetic segments.

In an embodiment, the method comprises assembling sequences for subject intervals, with a method described herein, e.g., a method comprising a circular binary segmentation (CBS), an HMM based method, a Wavelet based method, or a Cluster along Chromosomes method.

In an embodiment, fitting the genome-wide copy number model to the SCI comprises using the equation of:

$$log {Ratio}_{i} = {log}_{2} \frac{{pC}_{i} + 2 ( 1 - p )}{p ψ + 2 ( 1 - p )} ,$$

In an embodiment, ψ=(ΣᵢIᵢCᵢ)/ΣᵢIᵢ, let Iᵢ be the length of a genomic segment.

In an embodiment, fitting the genome-wide copy number model to the SAFI comprises using the equation of:

$$AF = \frac{pM + 1 ( 1 - p )}{pC + 2 ( 1 - p )} ,$$

where AF is allele frequency.

In an embodiment, the fitting comprises using Gibbs sampling. In an embodiment, fitting comprises using e.g., Markov chain Monte Carlo (MCMC) algorithm, e.g., ASCAT (Allele-Specific Copy Number Analysis of Tumors), OncoSNP, or PICNIC (Predicting Integral Copy Numbers In Cancer). In an embodiment, fitting comprises using Metropolis-Hastings MCMC. In an embodiment, fitting comprises using a non-Bayesian process, e.g., a frequentist process, e.g., using least squares fitting.

In an embodiment, g is determined by determining the fit of values for VAFI, p, C, and M to a model for somatic/germline status. In an embodiment, the method comprises acquiring an indication of heterozygosity for said variant, e.g., mutation. In an embodiment, sample purity (p) is global purity, e.g., is the same for all genomic segments.

In an embodiment, the value of g is acquired by:

$$AF = \frac{pM + g ( 1 - p )}{pC + 2 ( 1 - p )} ,$$

where Ar is allele frequency,

In an embodiment, a value of g that is close to 0, e.g., does not differ significantly from 0, indicates the variant is a somatic variant. In an embodiment, a value of g that is 0, or close to 0, e.g., within a distance from 0, e.g., a value of g of less than 0.4, indicates the variant is a somatic variant. In an embodiment, a value of g that is close to 1, e.g., does not differ significantly from 1, indicates the variant is a germline variant. In an embodiment, a value of g that is 1, or close to 1, e.g., within a distance from 1, e.g., a value of g of more than 0.6, indicates the variant is a germline variant. In an embodiment, a value of g is less than 1 but more than 0, e.g., if it is less than 1 by an amount and more than 0 by an amount, e.g., if g is between 0.4 and 0.6, it indicates an indistinguishable result.

In an embodiment, a value of g that is significantly less than 0, is indicative of a subclonal somatic variant.

In an embodiment, the value of g is acquired by:

$$AF = \frac{pM ′ + g ( 1 - p )}{pC + 2 ( 1 - p )} ,$$

where AF is allele frequency, and M′=C−M (e.g., when M is a non-minor allele frequency), e.g., the variant is a germline polymorphism if g=1 and the variant is a somatic mutation if g=0.

In an embodiment, the somatic/germline status is determined, e.g., when the sample purity is below about 40%, e.g., between about 10% and 30%, e.g., between about 10% and 20%, or between about 20% and 30%.

In an embodiment, when: a value of M equal to 0 not equal to C is indicative of absence of the variant, e.g., mutation, e.g., not existent in the tumor; a non-zero value of M equal to Cis indicative of homozygosity of the variant, e.g., mutation, e.g., with loss of heterozygosity (LOH); a value of M equal to 0 equal to C indicates a homozygous deletion of the variant, e.g., mutation, e.g., not existent in the tumor; and a non-zero value of M not equal to C is indicative of heterozygosity of the variant, e.g., mutation.

In an embodiment, the method comprises acquiring an indication of zygosity for said variant, e.g., mutation. In an embodiment, the mutation status is determined as homozygous (e.g., LOH) if M=C≠0. In an embodiment, the mutation status is determined as homozygous deletion if M=C=0. In an embodiment, the mutation status is determined as heterozygous is 0<M<C. In an embodiment, the mutation is absent from the tumor if M=0 and C≠0. In an embodiment, the zygosity is determined, e.g., when the sample purity is greater than about 80%, e.g., between about 90% and 100%, e.g., between about 90% and 95%, or between about 95% and 100%.

In an embodiment, the control is a sample of euploid (e.g., diploid) tissue from a subject other than the subject from which the sample is from, or a sample of mixed euploid (e.g., diploid) tissues from one or more (e.g., at least 2, 3, 4, or 5) subjects other than the subject from which the sample is from. In an embodiment, the method comprises sequencing each of the selected subject intervals and each of the selected germline SNPs, e.g., by next generation sequencing (NGS). In an embodiment, the sequence coverage prior to normalization is at least about 10×, 20×, 30×, 50×, 100×, 250×, 500×, 750×, 800×, 900×, 1,000×, 1,500×, 2,000×, 2,500×, 3,000×, 3,500×, 4,000×, 4,500×, 5,000×, 5,500×, 6,000×, 6,500×, 7,000×, 7,500×, 8,000×, 8,500×, 9,000×, 9,500×, or 10,000× the depth of the sequencing.

In an embodiment, the subject has received an anti-cancer therapy. In an embodiment the subject has received an anti-cancer therapy and is resistant to the therapy or exhibits disease progression. In an embodiment the subject has received an anti-cancer therapy which is selected from: a therapeutic agent that has been approved by the FDA, EMA, or other regulatory agency; or a therapeutic agent that has been not been approved by the FDA, EMA, or other regulatory agency. In an embodiment the subject has received an anti-cancer therapy in the course of a clinical trial, e.g., a Phase I, Phase II, or Phase III clinical trial (or in an ex-US equivalent of such a trial). In an embodiment the variant is positively associated with the type of tumor present in the subject, e.g., with occurrence of, or resistance to treatment. In an embodiment the variant is not positively associated with the type of tumor present in the subject. In an embodiment the variant is positively associated with a tumor other than the type of tumor present in the subject. In an embodiment the variant is a variant that is not positively associated with the type of tumor present in the subject.

In an embodiment, the method can memorialize, e.g., in a database, e.g., a machine readable database, provide a report containing, or transmit, a descriptor for one or more of: the presence, absence, or frequency, of other mutations in the tumor, e.g., other mutations associated with the tumor type in the sample, other mutations not associated with the tumor type in the sample, or other mutations associated with a tumor other than the tumor type in the sample; the characterization of the variant; the allele or gene; or the tumor type, e.g., the name of the type of tumor, whether the tumor is primary or secondary; a subject characteristic; or therapeutic alternatives, recommendations, or choices.

In an embodiment, a descriptor relating to the characterization of the variant comprises a descriptor for zygosity or germline vs somatic status. In an embodiment, a descriptor relating to a subject characteristic comprises a descriptor for one or more of: the subject's identity; one or more of the subject's, age, gender, weight, or other similar characteristic, occupation; the subject's medical history, e.g., occurrence of the tumor or of other disorders; the subject's family medical history, e.g., relatives who share or do not share the variant; or the subject's prior treatment history, e.g., the treatment received, response to a previously administered anti-cancer therapy, e.g., disease resistance, responsiveness, or progression.

The SGZ algorithm is also described in Sun et al. PLOS Comput Biol. 2018; 14(2):e1005965; Sun et al. Cancer Research 2014; 74(19S):1893-1893; International Application Publication No. WO2014/183078, U.S. Pat. No. 9,792,403, and U.S. Application Publication No. 2014/0336996, the contents of which are incorporated by reference in their entirety.

### Tumor Mutational Burden

The methods described herein can be used in combination with, or as part of, a method for evaluating tumor mutational burden (TMB).

In certain embodiments, the method comprises providing a sequence of a set of subgenomic intervals from a sample (e.g., a sample described herein); and determining a value for the mutational burden, wherein the value is a function of the number of alterations in the set of subgenomic intervals. In certain embodiments, the set of subgenomic intervals are from a set of genes, for example, a set of genes that does not include the entire genome or exome. In certain embodiments, the set of subgenomic intervals is a set of coding subgenomic intervals. In other embodiments, the set of subgenomic intervals contains one or more coding subgenomic intervals and one or more non-coding subgenomic intervals. In certain embodiments, the value for the mutational burden is a function of the number of alterations (e.g., somatic alterations) in the set of subgenomic intervals. In certain embodiments, the number of alterations excludes the number of functional alterations, germline alterations, or both.

The methods described herein can also include, e.g., one or more of: acquiring a library comprising a plurality of tumor nucleic acid molecules from the sample; contacting the library with target capture reagents to provide selected tumor nucleic acid molecules by hybridization, thereby providing a library catch; acquiring a read for a subgenomic interval comprising an alteration from the tumor nucleic acid molecule from the library catch; aligning the read by an alignment method; assigning a nucleotide value from the read for a nucleotide position; and selecting a set of subgenomic intervals from a set of the assigned nucleotide positions, wherein the set of subgenomic intervals are from a set of genes.

In certain embodiments, the mutational burden is measured in a sample from a subject, e.g., a subject described herein. In certain embodiments, the mutational burden is expressed as a percentile, e.g., among the mutational burdens in samples from a reference population. In certain embodiments, the reference population includes patients having the same type of cancer as the subject. In other embodiments, the reference population includes patients who are receiving, or have received, the same type of therapy, as the subject. In certain embodiments, the mutational burden obtained by a method described herein, e.g., by evaluating the level of an alteration (e.g., a somatic alteration) in a set of genes set forth in Tables 1A-4B, correlates with the whole genome or exome mutational burden.

The terms “mutational burden,” “mutation burden,” “mutation load,” and “mutational load” are used interchangeably herein. In the context of a tumor, a mutational load is also referred to herein as “tumor mutational burden,” “tumor mutation burden,” or “TMB.” Without wishing to be bound by theory, it is believed that in some embodiments, TMB can be considered as a type of genomic signature, e.g., a continuous/complex biomarker.

As used herein, the term “mutation burden” or “mutational burden” refers to the level, e.g., number, of an alteration (e.g., one or more alterations, e.g., one or more somatic alterations) per a predefined unit (e.g., per megabase) in a set of genes (e.g., in the coding regions of the set of genes). Mutational burden can be measured, e.g., on a whole genome or exome basis, or on the basis of a subset of genome or exome. In certain embodiments, the mutational burden measured on the basis of a subset of genome or exome can be extrapolated to determine a whole genome or exome mutational burden.

In an embodiment, the method comprises:
a) providing a sequence, e.g., a nucleotide sequence, of a set of subject intervals (e.g., coding subject intervals) from the sample, wherein the set of subject intervals are from a set of genes; and
b) determining a value for the mutational burden, wherein the value is a function of the number of an alteration (e.g., one or more alterations), e.g., a somatic alteration (e.g., one or more somatic alterations), in the set of subject intervals.

In certain embodiments, the number of an alteration excludes a functional alteration in a subject interval. In other embodiments, the number of an alteration excludes a germline alteration in a subject interval. In certain embodiments, the number of an alteration excludes a functional alteration in a subject interval and a germline alteration in a subject interval.

In certain embodiments, the set of subject intervals comprises coding subject intervals. In other embodiments, the set of subject intervals comprises non-coding subject intervals. In certain embodiments, the set of subject intervals comprises coding subject intervals. In other embodiments, the set of subject intervals comprises one or more coding subject intervals and one or more non-coding subject intervals. In certain embodiments, about 5% or more, about 10% or more, about 20% or more, about 30% or more, about 40% or more, about 50% or more, about 60% or more, about 70% or more, about 80% or more, about 90% or more, or about 95% or more, of the subject intervals in the set of subject intervals are coding subject intervals. In other embodiments, about 90% or less, about 80% or less, about 70% or less, about 60% or less, about 50% or less, about 40% or less, about 30% or less, about 20% or less, about 10% or less, or about 5% or less, of the subject intervals in the set of subject intervals are non-coding subject intervals.

In other embodiments, the set of subject intervals does not comprise the entire genome or the entire exome. In other embodiments, the set of coding subject intervals does not comprise the entire exome.

In certain embodiments, the set of genes does not comprise the entire genome or the entire exome. In other embodiments, the set of genes comprises or consists of one or more genes set forth in Tables 2A-5B.

In certain embodiments, the value is expressed as a function of the set of genes. In certain embodiments, the value is expressed as a function of the coding regions of the set of genes. In other embodiments, the value is expressed as a function of the non-coding regions of the set of genes. In certain embodiments, the value is expressed as a function of the exons of the set of genes. In other embodiments, the value is expressed as a function of the introns of the set of genes.

In certain embodiments, the value is expressed as a function of the set of genes sequenced. In certain embodiments, the value is expressed as a function of the coding regions of the set of genes sequenced. In other embodiments, the value is expressed as a function of the non-coding regions of the set of genes sequenced. In certain embodiments, the value is expressed as a function of the exons of the set of genes sequenced. In other embodiments, the value is expressed as a function of the introns of the set of genes sequenced.

In certain embodiments, the value is expressed as a function of the number of an alteration (e.g., a somatic alteration) in a number of positions of the set of genes. In certain embodiments, the value is expressed as a function of the number of an alteration (e.g., a somatic alteration) in a number of positions of the coding regions of the set of genes. In other embodiments, the value is expressed as a function of the number of an alteration (e.g., a somatic alteration) in a number of positions of the non-coding regions of the set of genes. In certain embodiments, the value is expressed as a function of the number of an alteration (e.g., a somatic alteration) in a number of positions of the exons of the set of genes. In other embodiments, the value is expressed as a function of the number of an alteration (e.g., a somatic alteration) in a number of positions of the introns of the set of genes.

In certain embodiments, the value is expressed as a function of the number of an alteration (e.g., a somatic alteration) in a number of positions of the set of genes sequenced. In certain embodiments, the value is expressed as a function of the number of an alteration (e.g., a somatic alteration) in a number of positions of the coding regions of the set of genes sequenced. In other embodiments, the value is expressed as a function of the number of an alteration (e.g., a somatic alteration) in a number of positions of the non-coding regions of the set of genes sequenced. In certain embodiments, the value is expressed as a function of the number of an alteration (e.g., a somatic alteration) in a number of positions of the exons of the set of genes sequenced. In other embodiments, the value is expressed as a function of the number of an alteration (e.g., a somatic alteration) in a number of positions of the introns of the set of genes sequenced.

In certain embodiments, the value is expressed as a function of the number of an alteration (e.g., a somatic alteration) per a unit, e.g., as a function of the number of a somatic alteration per megabase.

In certain embodiments, the value is expressed as a function of the number of an alteration (e.g., a somatic alteration) per megabase in the set of genes. In certain embodiments, the value is expressed as a function of the number of an alteration (e.g., a somatic alteration) per megabase in the coding regions of the set of genes. In other embodiments, the value is expressed as a function of the number of an alteration (e.g., a somatic alteration) per megabase in the non-coding regions of the set of genes. In certain embodiments, the value is expressed as a function of the number of an alteration (e.g., a somatic alteration) per megabase in the exons of the set of genes. In other embodiments, the value is expressed as a function of the number of an alteration (e.g., somatic alteration) per megabase in the introns of the set of genes.

In certain embodiments, the value is expressed as a function of the number of an alteration (e.g., a somatic alteration) per megabase in the set of genes sequenced. In certain embodiments, the value is expressed as a function of the number of an alteration (e.g., a somatic alteration) per megabase in the coding regions of the set of genes sequenced. In other embodiments, the value is expressed as a function of the number of an alteration (e.g., a somatic alteration) per megabase in the non-coding regions of the set of genes sequenced. In certain embodiments, the value is expressed as a function of the number of an alteration (e.g., a somatic alteration) per megabase in the exons of the set of genes sequenced. In other embodiments, the value is expressed as a function of the number of an alteration (e.g., a somatic alteration) per megabase in the introns of the set of genes sequenced.

In certain embodiments, the mutational burden is extrapolated to a larger portion of the genome, e.g., to the exome or the entire genome, e.g., to obtain the total mutational burden. In other embodiments, the mutational burden is extrapolated to a larger portion of the exome, e.g., to the entire exome.

In certain embodiments, the sample is from a subject. In certain embodiments, the subject has a disorder, e.g., a cancer. In other embodiments, the subject is receiving, or has received, a therapy, e.g., an immunotherapy.

In certain embodiments, the mutational burden is expressed as a percentile, e.g., among the mutational burdens in samples from a reference population. In certain embodiments, the reference population includes patients having the same type of cancer as the subject. In other embodiments, the reference population includes patients who are receiving, or have received, the same type of therapy, as the subject.

In certain embodiments, the method comprises:
(i) acquiring a library comprising a plurality of tumor nucleic acid molecules from the sample;
(ii) contacting the library with a target capture reagent to provide selected tumor nucleic acid molecules, wherein said target capture reagent hybridizes with the tumor nucleic acid molecule, thereby providing a library catch;
(iii) acquiring a read for a subject interval comprising an alteration (e.g., a somatic alteration) from a tumor nucleic acid molecule from said library catch, e.g., by a next-generation sequencing method;
(iv) aligning said read by an alignment method;
(v) assigning a nucleotide value from said read for a nucleotide position;
(vi) selecting a set of subject intervals (e.g., coding subject intervals) from a set of the assigned nucleotide positions, wherein the set of subject intervals are from a set of genes; and
(vii) determining a value for the mutational burden, wherein the value is a function of the number of an alteration (e.g., one or more alterations), e.g., a somatic alteration (e.g., one or more somatic alterations), in the set of subject intervals.

In certain embodiments, the number of an alteration (e.g., a somatic alteration) excludes a functional alteration in a subject interval. In other embodiments, the number of an alteration excludes a germline alteration in a subject interval. In certain embodiments, the number of an alteration (e.g., a somatic alteration) excludes a functional alteration in a subject interval and a germline alteration in a subject interval.

Other methods for evaluating tumor mutational burden are described in International Application Publication No. WO2017/151524, the content of which is incorporated by reference in its entirety.

### Applications

Methods disclosed herein allow integration of a number of optimized elements including optimized target capture reagent (e.g., bait)-based selection, optimized alignment, and optimized mutation calling, as applied, e.g., to cancer related segments of the genome. Methods described herein provide for NGS-based analysis of tumors that can be optimized on a cancer-by-cancer, gene-by-gene and site-by-site basis. This can be applied e.g., to the genes/sites and tumor types described herein. The methods optimize levels of sensitivity and specificity for mutation detection with a given sequencing technology. Cancer by cancer, gene by gene, and site by site optimization provides very high levels of sensitivity/specificity (e.g., >99% for both) that are essential for a clinical product,

Without wishing to be bound by theory, it is believed that in some embodiments, the methods described herein can be applied to general sequencing applications which would benefit from increased sensitivity in detection of selected genomic regions. For example, those applications include, but are not limited to, hereditary cancer panels with increased coverage based upon prevalence, other whole exome sequencing (WES) tests targeted to specific disease pathways, and prenatal testing with enrichment for candidate actionable focal events.

In some embodiments, the method further comprises selecting a treatment responsive to the evaluation of a genomic alteration, e.g., a somatic alteration. In some embodiments, the method can further comprise selecting a treatment responsive to the evaluation of mutational burden, e.g., an increased or decreased level of mutational burden. In some embodiments, the method further comprises administering a treatment responsive to the evaluation of a genomic alteration. In some embodiments, the treatment may comprise a cancer treatment or tumor therapy. In some embodiments, the cancer treatment or tumor therapy may comprise, for example, chemotherapy, radiation therapy, a targeted immunotherapy, or surgery. In some embodiments, the cancer treatment or tumor therapy may comprise, for example, a targeted immunotherapy selected based on a result of a genomic profiling assay (e.g., a comprehensive genomic profiling (CGP) test, a gene expression profiling test, a cancer hotspot panel test, a DNA methylation test, a DNA fragmentation test, an RNA fragmentation test, or any combination thereof).

In some embodiments, the method further comprises classifying the sample or the subject from which the sample was derived responsive to the evaluation of a genomic alteration. In some embodiments, the method further comprises determining clinical trial eligibility for a subject from which a sample is obtained.

In some embodiments, the method further comprises one or more of the steps of obtaining the sample from the subject, extracting nucleic acid molecules (e.g., a mixture of tumor nucleic acid molecules and non-tumor nucleic acid molecules) from the sample, ligating adapters to the nucleic acid molecules extracted from the sample, amplifying the nucleic acid molecules (e.g., using the polymerase chain reaction (PCR) or an isothermal amplification technique), capturing nucleic acid molecules from the amplified nucleic acid molecules (e.g., wherein the captured nucleic acid molecules are captured from the amplified nucleic acid molecules by hybridization to one or more bait molecules, and wherein the one or more bait molecules comprise one or more nucleic acid molecules that each comprising a region that is complementary to a region of a captured nucleic acid molecule), sequencing the nucleic acid molecules extracted from the sample, (or library proxies derived therefrom) using, e.g., a next-generation sequencing technique and/or a next-generation sequencer, generating, displaying, transmitting, and/or delivering a report, e.g., an electronic, web-based, or paper report, to the patient or to another person or entity, a caregiver, a healthcare provider, a physician, an oncologist, an electronic medical record system, a hospital, clinic, third-party payor, insurance company or government office. In some embodiments, the report comprises output from the method described herein. In some embodiments, the report is transmitted via a computer network or peer-to-peer connection.

Methods described herein provide for clinical and regulatory grade comprehensive genomic profiling analysis and interpretation of genomic aberrations for a comprehensive set of plausibly actionable genes (which may typically range from 50 to 500 genes) using next-generation sequencing technologies from routine, real-world samples in order to inform optimal treatment and disease management decisions.

Methods described herein provide one-stop-shopping for oncologists/pathologists to send a sample and receive a comprehensive genomic profiling analysis and description of the genomic and other molecular changes for a tumor, in order to inform optimal treatment and disease management decisions.

Methods described herein provide a robust, real-world clinical oncology diagnostic tool that takes standard available samples and in one test provides a comprehensive genomic and other molecular aberration analysis to provide the oncologist with a comprehensive description of what aberrations may be driving the tumor and could be useful for informing the oncologists treatment decisions.

Methods described herein provide for a comprehensive genomic profiling analysis of a patient's cancer genome, e.g., by next-generation sequencing (NGS), with clinical grade quality. Methods include the most relevant genes and potential alterations and include one or more of the analysis of mutations (e.g., indels or base substitutions), copy number, rearrangements, e.g., translocations, expression, and epigenetic markers. The output of the genetic analysis can be contextualized with descriptive reporting of actionable results. Methods connect the use with an up to date set of relevant scientific and medical knowledge.

In some embodiments, the method analyzes a sample derived from a human body for the purpose of providing information for the diagnosis, prevention or treatment of any disease (e.g., cancer) or impairment of, or the assessment of the health of, human beings. In some embodiments, the method is performed in accordance with the guidelines provided by Clinical Laboratory Improvement Amendment (CLIA) and/or the College of American Pathologists (CAP). In some embodiments, the method is performed in a CLIA and/or CAP certified facility. In some embodiments, the method is performed in accordance with the guidelines provided by the Food and Drug Administration (FDA), the European Medicines Agency (EMA), Quality System Regulation (QSR), European Commission (CE), e.g., CE in vitro diagnostics (CE-IVD), Chinese Food and Drug Administration (CFDA) or other regulatory agency. In some embodiments, the method is performed in a FDA, QSR, CE or CFDA certified facility. In some embodiments, the method is performed in a QSR certified facility. In some embodiments, the method analyzes a clinical grade sample, e.g., a sample suitable for clinical practice, trials, or management of patient care. In some embodiments, the sample comprises a retrospective sample and/or a prospective sample. In some embodiments, a retrospective sample comprises a sample analyzed before or after a treatment has been administered or is a research sample. In some embodiments, a prospective sample comprises a sample from a subject that has not been treated with a treatment. In some embodiments, use of a method described herein to analyze a prospective sample can result in a prediction of the outcome of a therapy on the subject from which the sample was obtained, e.g., derived.

In some embodiments, the method is used as a diagnostic, e.g., as described herein. In some embodiments, the method is used in or with a companion diagnostic. In some embodiments, the method is used as a complementary diagnostic.

In some embodiments, the validity of the method is established (e.g., under CLIA regulations) by determination of one or more (e.g., two, three, four, five, or all) of accuracy, precision, sensitivity, specificity, reportable range, or reference interval. In certain embodiments, accuracy is determined by the coverage and quality (e.g., Phred scores), e.g., for known variants (e.g., SNP, indel) in targeted regions. In certain embodiments, precision is determined by the sequence replication and coverage distribution between different operators and instruments, e.g., for known variants. In certain embodiments, specificity is determined by the false positive rate, degree with which a false variant is identified at a specific coverage threshold, e.g., in several samples with well characterized targets. In certain embodiments, sensitivity is determined by the likelihood test that detects known variant, e.g., in several samples with well characterized targets. In certain embodiments, reportable range is determined by the intron buffer and exon region of one or more genes, e.g., with repeat regions, indels, or allele drop outs. In certain embodiments, reference interval is determined by sequence variation background measurement, e.g., in an unaffected population.

In some embodiments, the method is performed in a setting (e.g., under CAP regulations) that includes consideration for one or more (e.g., two, three, four, five, or all) of validated sample extraction, library preparation, barcoding, pooling, target enrichment, or bioinformatics (e.g., how precise and sensitive variants are called).

Methods described herein provide for increasing both the quality and efficiency of patient care. This includes applications where a tumor is of a rare or poorly studied type such that there is no standard of care or the patient is refractory to established lines of therapy and a rational basis for selection of further therapy or for clinical trial participation could be useful. E.g., the methods allow, at any point of therapy, selection where the oncologist would benefit by having the full “molecular image” and/or “molecular sub-diagnosis” available to inform decision making. The results can be used to determine whether a patient may be eligible to enroll in a clinical trial.

Methods described herein can comprise providing a report, e.g., in electronic, web-based, or paper form, to the patient or to another person or entity, e.g., a caregiver, e.g., a physician, e.g., an oncologist, a hospital, clinic, third-party payor, insurance company or government office. The report can comprise output from the method, e.g., the identification of nucleotide values, the indication of the presence or absence of an alteration, mutation, or wild-type sequence, e.g., for subject intervals associated with a tumor of the type of the sample. The report can also comprise information on the level of tumor mutational burden. The report can also comprise information on one or more other genomic signatures, e.g., continuous/complex biomarkers, e.g., the level of microsatellite instability, or the presence or absence of heterozygosity (LOH). The report can also comprise information on the role of a sequence, e.g., an alteration, mutation, or wild-type sequence, in disease. Such information can include information on prognosis, resistance, or potential or suggested therapeutic options. The report can comprise information on the likely effectiveness of a therapeutic option, the acceptability of a therapeutic option, or the advisability of applying the therapeutic option to a patient, e.g., a patient having a sequence, alteration identified in the test, and in embodiments, identified in the report. E.g., the report can include information, or a recommendation on, the administration of a drug, e.g., the administration at a dosage or in a treatment regimen, e.g., in combination with other drugs, to the patient. In an embodiment, not all mutations identified in the method are identified in the report. E.g., the report can be limited to mutations in genes having a level of correlation with the occurrence, prognosis, stage, or susceptibility of the cancer to treatment, e.g., with a therapeutic option. Methods featured herein allow for delivery of the report, e.g., to an entity described herein, within 7, 14, or 21 days from receipt of the sample by the entity practicing the method. Thus, methods featured in the invention allow a quick turnaround time, e.g., within 7, 14 or 21 days of receipt of sample.

Methods described herein can also be used to evaluate a histologically normal sample, e.g., samples from surgical margins. If one or more alterations as described herein is detected, the tissue can be re-classified, e.g., as malignant or premalignant, and/or the course of treatment can be modified.

In some embodiments, the methods described herein are useful in non-cancer applications, e.g., in forensic applications (e.g., identification as alternative to, or in addition to, use of dental records), paternity testing, and disease diagnosis and prognosis, e.g., for an infectious disease, an autoimmune disorder, cystic fibrosis, Huntington's Disease, Alzheimer's Disease, among others. For example, identification of genetic alterations by the methods described herein can indicate the presence or risk of an individual for developing a particular disorder.

### Systems

In another aspect, the invention features a system for evaluating genomic alterations in a sample, e.g., in accordance with a method described herein. The system includes at least one processor operatively connected to a memory, the at least one processor when executing is configured to perform a method of analyzing a sample as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Other features and advantages of the invention will be apparent from the detailed description, drawings, and from the claims.

### OTHER EMBODIMENTS

Alternatively, or in combination with the methods described herein, in some embodiments, the method further comprises one or more (e.g., 2, 3, 4, 5, 6, 7, or all) of (a)-(h):
(a) providing nucleic acid molecules (e.g., cfDNA) from a sample (e.g., a blood sample), e.g., using a plurality of target capture reagents described herein;
(b) attaching adapters comprising barcodes that comprises a plurality of different barcode sequences to the nucleic acid molecules, thereby generating tagged parent nucleic acid molecules;
(c) amplifying the tagged parent nucleic acid molecules to produce amplified tagged progeny nucleic acid molecules;
(d) sequencing the amplified tagged progeny nucleic acid molecules to produce a plurality of sequence reads from each of the tagged parent nucleic acid molecules, wherein each sequence read of the plurality of sequence reads comprises a barcode sequence and a sequence derived from a nucleic acid molecule;
(e) mapping sequence reads of the plurality of sequence reads to one or more reference sequences;
(f) grouping the sequence reads mapped in e) into families based at least on barcode sequences of the sequence reads, each of the families comprising sequence reads comprising the same barcode sequence, whereby each of the families comprises sequence reads amplified from the same tagged parent nucleic acid molecule;
(g) at each of a plurality of subject intervals in the one or more reference sequences, collapsing sequence reads in each family to yield a mutation call for each family at the subject interval; or
(h) detecting, at one or more subject intervals, one or more genomic aberrations, e.g., an indel, copy number variation, transversion, translocation, inversion, deletion, aneuploidy, partial aneuploidy, polyploidy, chromosomal instability, chromosomal structure alteration, gene fusion, chromosome fusion, gene truncation, gene amplification, gene duplication, chromosomal lesion, DNA lesion, abnormal change in nucleic acid chemical modification, abnormal change in epigenetic pattern, abnormal change in nucleic acid methylation, or a combination thereof.

Alternatively, or in combination with the methods described herein, in some embodiments, the method further comprises one or more (e.g., 2, 3, 4, 5, 6, 7, 8, or all) of (a)-(i), e.g., to quantify a genomic alteration (e.g., a single nucleotide variant):
(a) providing nucleic acid molecules (e.g., cfDNA) from a sample (e.g., a blood sample), e.g., using a plurality of target capture reagents described herein;
(b) attaching adapters comprising barcodes that comprises distinct barcode sequences to said nucleic acid molecules to generate tagged parent nucleic acid molecules;
(c) amplifying the tagged parent nucleic acid molecules to produce amplified tagged progeny nucleic acid molecules;
(d) sequencing the amplified tagged progeny nucleic acid molecules to produce a plurality of sequence reads from each parent nucleic acid molecules, wherein each sequence read comprises a barcode sequence and a sequence derived from the nucleic acid molecules;
(e) grouping the plurality of sequence reads produced from each tagged parent nucleic acid molecule into families based on (i) the barcode sequence and (ii) one or more of: sequence information at a beginning of the sequence derived from the nuclei acid, sequence information at an end of the sequence derived from the nucleic acid, or length of the sequence read, wherein each family comprises sequence reads of tagged progeny nucleic acid molecules amplified from a unique nucleic acid molecule among the tagged parent nucleic acid molecules;
(f) comparing the sequence reads grouped within each family to each other to determine consensus sequences for each family, wherein each of the consensus sequences corresponds to a unique nucleic acid molecule among the tagged parent nucleic acid molecules;
(g) providing one or more reference sequences comprising one or more subject intervals;
(h) identifying consensus sequences that map to a given subject interval of said one or more subject intervals; or
(i) determining a number of consensus sequences that map to the given subject interval that comprises a genomic alteration, thereby quantifying the genomic alteration in the sample.

Alternatively, or in combination with the methods described herein, in some embodiments, the method further comprises one or more (e.g., 2, 3, 4, 5, 6, 7, or all) of (a)-(h):
(a) providing nucleic acid molecules (e.g., cfDNA) from a sample (e.g., a blood sample), e.g., using a plurality of target capture reagents described herein;
(b) converting the plurality of nucleic acid molecules into a plurality of tagged parent nucleic acid molecules, wherein each of the tagged parent nucleic acid molecules comprises (i) a sequence from a nucleic acid molecule of the plurality of nucleic acid molecules, and (ii) an identifier sequence comprising one or more barcodes;
(c) amplifying the plurality of tagged parent nucleic acid molecules to produce a corresponding plurality of amplified progeny nucleic acid molecules;
(d) sequencing the plurality of amplified progeny nucleic acid molecules to produce a set of sequence reads;
(e) mapping sequence reads of the set of sequence reads to one or more reference sequences;
(f) grouping the sequence reads into families, each of the families comprising sequence reads comprising the same identifier sequence and having the same start and stop positions, wherein each of the families comprises sequence reads amplified from the same tagged parent nucleic acid molecule;
(g) at each subject interval of a plurality of subject intervals in the one or more reference sequences, collapsing sequence reads in each family to yield a mutation call for each family at the subject interval; or
(h) determining a frequency of one or more mutations called at the subject interval from among the families.

Alternatively, or in combination with the methods described herein, in some embodiments, the method further comprises one or more (e.g., 2, 3, 4, 5, or all) of (a)-(f), e.g., to detect copy number variation:
(a) providing nucleic acid molecules (e.g., cfDNA) from a sample (e.g., a blood sample), e.g., using a plurality of target capture reagents described herein;
(b) sequencing the nucleic acid molecules, wherein each of the nucleic acid molecules generates a plurality of sequence reads;
(c) filtering out reads that fail to meet a set accuracy, quality score, or mapping score threshold;
(d) mapping the plurality of sequence reads to a reference sequence;
(e) quantifying mapped reads or unique sequence reads in a plurality of regions of the reference sequence; and
(f) determining copy number variation in one or more of the plurality of predefined regions by: i) normalizing a number of reads in the plurality of regions to each other, or a number of unique sequence reads in the plurality of regions to each other; and/or ii) processing a number of reads in the plurality of regions or a number of unique sequence reads in the plurality of regions with numbers obtained from a control sample.

Alternatively, or in combination with the methods described herein, in some embodiments, the method further comprises one or more (e.g., 2, 3, 4, 5, 6, 7, or all) of (a)-(h), e.g., to detect copy number variation:
(a) providing nucleic acid molecules (e.g., cfDNA) from a sample (e.g., a blood sample), e.g., using a plurality of target capture reagents described herein;
(b) sequencing the nucleic acid molecules, wherein each of the nucleic acid molecules generates a plurality of sequence reads;
(c) filtering out reads that fail to meet a set accuracy, quality score, or mapping score threshold;
(d) mapping sequence reads derived from the sequencing onto a reference sequence;
(e) determining unique sequence reads corresponding to the nucleic acid molecules from among the sequence reads;
(f) identifying a subset of mapped unique sequence reads that include a variant as compared to the reference sequence at each mappable base position;
(g) for each mappable base position, determining a ratio of (a) a number of mapped unique sequence reads that include a variant as compared to the reference sequence, to (b) a number of total unique sequence reads for each mappable base position; and
(h) processing the ratio with a similarly derived number from a reference sample.

Alternatively, or in combination with the methods described herein, in some embodiments, the method further comprises one or more (e.g., 2, 3, 4, 5, 6, 7, or all) of (a)-(h):
(a) tagging double-stranded DNA molecules (e.g., cfDNA) in a sample (e.g., a blood sample) from a subject with a set of duplex tags, wherein the set of duplex tags comprises a plurality of different molecular barcodes, wherein each duplex tag of the set of duplex tags differently tags complementary strands of a double-stranded DNA molecule of the double-stranded DNA molecules in the sample to provide tagged strands, and wherein the tagging is performed with at least a 10× excess of duplex tags as compared to the double-stranded DNA molecules, which excess of duplex tags is sufficient to tag at least 20% of the double-stranded DNA molecules in the sample from the subject;
(b) for each genetic locus in a set of one or more genetic loci in a reference genome, selectively enriching the tagged strands for a subset of the tagged strands that maps to the genetic locus, to provide enriched tagged strands, e.g., using a plurality of target capture reagents described herein;
(c) sequencing at least a portion of the enriched tagged strands to generate a plurality of raw sequence reads from the sample from the subject;
(d) grouping the plurality of raw sequence reads into a plurality of families, each family comprising raw sequence reads generated from a same parent polynucleotide, which grouping is based on (i) molecular barcodes associated with the parent polynucleotides and (ii) information from beginning and/or end portions of the raw sequences of the parent polynucleotides;
(e) collapsing the plurality of raw sequence reads grouped into the plurality of families into a plurality of consensus sequence reads, each consensus sequence read of the plurality of consensus sequence reads (i) comprising a plurality of consensus bases for each genetic locus in the set of one or more genetic loci and (ii) being representative of single strands of the double-stranded DNA molecules;
(f) for each genetic locus in the set of one or more genetic loci, determining a first quantitative measure of the enriched tagged strands that map to the genetic locus for which complementary strands are detected in the plurality of consensus sequence reads;
(g) for each genetic locus in the set of one or more genetic loci, determining a second quantitative measure of the enriched tagged strands that map to the genetic locus for which only one strand among complementary strands is detected in the plurality of consensus sequence reads; or
(h) for each genetic locus in the set of one or more genetic loci, determining a third quantitative measure of the enriched tagged strands that map to the genetic locus for which neither complementary strand is detected in the plurality of consensus sequence reads, wherein the third quantitative measure is calculated based at least in part on the first and second quantitative measures, thereby detecting the double-stranded DNA molecules in the sample from the subject.

Alternatively, or in combination with the methods described herein, in some embodiments, the method further comprises one or both of (a)-(b), e.g., for enriching for multiple genomic regions:
(a) bringing a predetermined amount of nucleic acid from a sample in contact with a plurality of target capture reagents described herein comprising:
(i) a first plurality of target capture reagents that selectively hybridizes to a first set of genomic regions of the nucleic acid from the sample, which first plurality of target capture reagents is provided at a first concentration that is less than a saturation point of the first plurality of target capture reagents, and
(ii) a second plurality of target capture reagents that selectively hybridizes to a second set of genomic regions of the nucleic acid from the sample, which second plurality of target capture reagents is provided at a second concentration that is at or above a saturation point of the second plurality of target capture reagents; and
(b) enriching the nucleic acid from the sample for the first set of genomic regions and the second set of genomic regions, thereby producing an enriched nucleic acid.

Alternatively, or in combination with the methods described herein, in some embodiments, the method further comprises one or more (e.g., 2, 3, 4, or all) of (a)-(e):
(a) providing a plurality of target capture reagent mixtures, wherein each of the plurality of target capture reagent mixtures comprises a first plurality of target capture reagents that selectively hybridizes to a first set of genomic regions and a second plurality target capture reagents that selectively hybridizes to a second set of genomic regions,
wherein the first plurality of target capture reagents is at different concentrations across the plurality of target capture reagent mixtures and the second plurality of target capture reagents is at the same concentration across the plurality of target capture reagent mixtures;
(b) contacting each of the plurality of target capture reagent mixtures with a sample (e.g., a blood sample) to capture nucleic acids from the sample with the first plurality of target capture reagents and the second plurality of target capture reagents, wherein the second plurality of target capture reagents in each target capture reagent mixture is provided at a first concentration that is at or above a saturation point of the second plurality of target capture reagents, wherein nucleic acids from the sample are captured by the first plurality of target capture reagents and the second plurality of target capture reagents;
(c) sequencing a portion of the nucleic acids captured with each target capture reagent mixture to produce sets of sequence reads within an allocated number of sequence reads;
(d) determining the read depth of sequence reads for the first plurality of target capture reagents and the second plurality of target capture reagents for each target capture reagent mixture; or
(e) identifying at least one target capture reagent mixture that provides read depths for the second set of genomic regions;
wherein the read depths for the second set of genomic regions provides a sensitivity of detecting of a genetic variant of at least 0.0001% minor allele frequency (MAF).

Other embodiments are described in U.S. Pat. Nos. 9,598,731, 9,834,822, 9,840,743, 9,902,992, 9,920,366, and 9,850,523, the contents of which are incorporated by reference in their entity.

In embodiments of a method described herein a step or parameter in the method is used to modify a downstream step or parameter in the method.

In an embodiment, a characteristic of the sample is used to modify a downstream step or parameter in one or more or all of: isolation of nucleic acid from said sample; library construction; design or selection of target capture reagents (e.g., baits); hybridization conditions; sequencing; read mapping; selection of a mutation calling method; mutation calling; or mutation annotation.

In an embodiment, a characteristic of an isolated tumor, or control, nucleic acid is used to modify a downstream step or parameter in one or more or all of: isolation of nucleic acid from said sample; library construction; design or selection of target capture reagents (e.g., baits); hybridization conditions; sequencing; read mapping; selection of a mutation calling method; mutation calling; or mutation annotation.

In an embodiment, a characteristic of a library is used to modify a downstream step or parameter in one or more or all of: re-isolation of nucleic acid from said sample; subsequent library construction; design or selection of target capture reagents (e.g., baits); hybridization conditions; sequencing; read mapping; selection of a mutation calling method; mutation calling; or mutation annotation.

In an embodiment, a characteristic of a library catch is used to modify a downstream step or parameter in one or more or all of: re-isolation of nucleic acid from said sample; subsequent library construction; design or selection of target capture reagents (e.g., baits); hybridization conditions; sequencing; read mapping; selection of a mutation calling method; mutation calling; or mutation annotation.

In an embodiment, a characteristic of the sequencing method is used to modify a downstream step or parameter in one or more or all of: re-isolation of nucleic acid from said sample; subsequent library construction; design or selection of target capture reagents (e.g., baits); subsequent determination of hybridization conditions subsequent sequencing; read mapping; selection of a mutation calling method; mutation calling; or mutation annotation.

In an embodiment, characteristic of the collection of mapped reads is used to modify a downstream step or parameter in one or more or all of: re-isolation of nucleic acid from said sample; subsequent library construction; design or selection of target capture reagents (e.g., baits); subsequent determination of hybridization conditions subsequent sequencing; subsequent read mapping; selection of a mutation calling method; mutation calling; or mutation annotation.

In an embodiment, the method comprises acquiring a value for a sample characteristic, e.g., acquiring a value: for the proportion of tumor cells in said sample; for the cellularity of said sample; or from an image of the sample. In embodiments, the method includes, responsive to said acquired value for a sample characteristic, selecting a parameter for: isolation of nucleic acid from a sample, library construction; design or selection of target capture reagents (e.g., baits); target capture reagent (e.g., bait)/library nucleic acid molecule hybridization; sequencing; or mutation calling.

In an embodiment, the method further comprising acquiring a value for the amount of tumor tissue present in said sample, comparing said acquired value with a reference criterion, and if said reference criterion is met, accepting said sample, e.g., accepting said sample if said sample contains greater than 30, 40 or 50% tumor cells. In an embodiment, a method further comprises acquiring a sub-sample enriched for tumor cells, e.g., by macrodissecting tumor tissue from said sample, from a sample that fails to meet the reference criterion.

In an embodiment, the method further comprising acquiring a value for the amount of tumor nucleic acids (e.g., DNA) present in said sample, comparing said acquired value with a reference criterion, and if said reference criterion is met, accepting said sample. In an embodiment, the method further comprises acquiring a sub-sample enriched for tumor nucleic acids, e.g., by macrodissecting tumor tissue from said sample, from a sample that fails to meet the reference criterion.

In an embodiment, a method further comprises providing an association of a tumor type, a gene, and a genetic alteration (a TGA) for a subject. In an embodiment, a method further comprises providing a database having a plurality of elements, wherein each element comprises a TGA.

In an embodiment, a method further comprises characterizing a TGA of a subject comprising: determining if said TGA is present in a database, e.g., a database of validated TGAs; associating information for the TGA from the database with said TGA (annotating) from said subject; and optionally, determining if a second or subsequent TGA for said subject is present in said database and if so associating information for the second or subsequent TGA from the database with said second TGA present in said patient. In an embodiment, the method further comprises memorializing the presence or absence of a TGA, and optionally an associated annotation, of a subject to form a report. In an embodiment, a method further comprises transmitting said report to a recipient party.

In an embodiment, a method further comprises characterizing a TGA of a subject comprising: determining if said TGA is present in a database, e.g., a database of validated TGAs; or determining if a TGA not in said database has a known clinically relevant gene or alteration and if so providing an entry for said TGA in said database. In an embodiment, the method further comprises memorializing the presence or absence of a mutation found in the DNA of the sample from a subject to form a report.

### Exemplary Embodiment

The following embodiments are exemplary and are not intended to limit the scope of the invention.

Embodiment 1. A method of determining a tumor fraction of a sample from a subject, the method comprising:
acquiring a value for a target variable associated with a subgenomic interval in the sample;
determining, from the target variable, a certainty metric;
accessing a determined relationship between a stored certainty metric and a stored tumor fraction; and
determining, with reference to the certainty metric and the determined relationship, the tumor fraction of the sample.

Embodiment 2. The method of embodiment 1, wherein the subgenomic interval comprises at least one nucleotide.

Embodiment 3. The method of embodiment 2, wherein the at least one nucleotide is associated with a single nucleotide polymorphism (SNP).

Embodiment 4. The method of any of embodiments 1-3, wherein the subgenomic interval comprises two or more nucleotides.

Embodiment 5. The method of any of embodiments 1-4, wherein the subgenomic interval comprises one or more nucleotides of a gene described herein.

Embodiment 6. The method of any of embodiments 1-5, wherein the certainty metric is one of a deviation from an expected log 2ratio for the subgenomic interval or a deviation from an expected allele fraction for the subgenomic interval.

Embodiment 7. The method of any of embodiments 1-6, wherein a plurality of values for the target variable, e.g., at a plurality of subgenomic intervals, are acquired.

Embodiment 8. The method of embodiment 7, wherein the plurality of subgenomic intervals comprises 2, 5, 10, 20, 30, 40, 50, 60, 70, 80, 90, 100, 150, 200, 250, 300, or more subgenomic intervals.

Embodiment 9. The method of any of embodiments 1-8, wherein the target variable comprises a comparison of the abundance of alleles associated with the subgenomic interval in the sample.

Embodiment 10. The method of any of embodiments 1-9, wherein the comparison is between the abundance of one allele and the abundance of all alleles.

Embodiment 11. The method of any of embodiments 1-9, wherein the comparison is between the abundance of one allele and the abundance of an alternative allele.

Embodiment 12. The method of any of embodiments 1-11, wherein the target variable comprises an allele fraction, or a comparison (e.g., ratio) of the abundance of a maternal or paternal allele relative to the abundance of maternal and paternal alleles.

Embodiment 13. The method of embodiment 12, wherein the maternal allele is more abundant than the paternal allele in the sample.

Embodiment 14. The method of embodiment 12, wherein the paternal allele is more abundant than the maternal allele in the sample.

Embodiment 15. The method of any of embodiments 1-14, wherein the value for the target variable is between 0 and 0.5, between 0 and 1, or between 0.5 and 1.

Embodiment 16. The method of any of embodiments 1-15, wherein the target variable comprises a comparison (e.g., ratio) of the difference in the abundance of a maternal allele and a paternal allele relative to the abundance of the maternal or paternal allele.

Embodiment 17. The method of embodiment 16, wherein the maternal allele is more abundant than the paternal allele in the sample.

Embodiment 18. The method of embodiment 16, wherein the paternal allele is more abundant than the maternal allele in the sample.

Embodiment 19. The method of any of embodiments 1-18, wherein the target variable comprises a comparison of the abundance of alleles at a subgenomic interval in the sample and the abundance of alleles at the subgenomic interval in a reference sample.

Embodiment 20. The method of embodiment 19, wherein the reference sample is obtained from a healthy subject, or a subject who does not have the cancer, or is not at risk of having the cancer.

Embodiment 21. The method of any of embodiments 19 or 20, wherein the target variable comprises a comparison (e.g., ratio) of the abundance of a maternal allele and a paternal allele in the sample relative to the abundance of the maternal allele and the paternal allele in the reference sample.

Embodiment 22. The method of any of embodiments 19 or 20, wherein the target variable comprises a comparison (e.g., ratio) of the difference in the abundance of a maternal allele and a paternal allele in the sample and the abundance of the maternal allele and the paternal allele in the reference sample, relative to the abundance of the maternal allele and the paternal allele in the reference sample.

Embodiment 23. The method of any of embodiments 1-22, wherein the subgenomic interval is heterozygous (in terms of the alleles associated with the subgenomic interval).

Embodiment 24. The method of any of embodiments 1-22, wherein the subgenomic interval is homozygous, semizygous, or hemizygous (in terms of the alleles associated with the subgenomic interval).

Embodiment 25. The method of any of embodiments 1-24, wherein at least one allele associated with the subgenomic interval is involved in copy number alteration, e.g., is amplified, in the sample.

Embodiment 26. The method of any of embodiments 1-25, wherein the certainty metric is a deviation metric, e.g., a deviation metric described herein, or any p-moment or a combination thereof.

Embodiment 27. The method of embodiment 26, wherein the deviation metric measures the deviation of a value for the target variable from a reference value, e.g., an expected value described herein.

Embodiment 28. The method of any of embodiments 26-27, wherein the deviation metric measures the deviation of a ratio of the abundance of a maternal or paternal allele, relative to the abundance of maternal and paternal alleles, from an expected ratio (e.g., 0.5).

Embodiment 29. The method of any of embodiments 26-28, wherein the deviation metric measures the deviation of a ratio of the difference in the abundance of a maternal allele and a paternal allele, relative to the abundance of the maternal or paternal allele, from an expected ratio (e.g., 0).

Embodiment 30. The method of any of embodiments 26-29, wherein the deviation metric measures the deviation of a ratio of the abundance of a maternal allele and a paternal allele in the sample, relative to the abundance of the maternal allele and the paternal allele in the reference sample, from an expected ratio (e.g., 0).

Embodiment 31. The method of embodiment 30, wherein the ratio comprises a log ratio, e.g., a log 2 ratio.

Embodiment 32. The method of any of embodiments 26-31, wherein the deviation metric measures the deviation of a ratio of the difference in the abundance of a maternal allele and a paternal allele in the sample and the abundance of the maternal allele and the paternal allele in the reference sample, relative to the abundance of the maternal allele and the paternal allele in the reference sample, from an expected ratio (e.g., 0).

Embodiment 33. The method of any of embodiments 26-32, wherein the deviation metric comprises a root mean squared (p=2-moment) deviation metric, or any combination of p-moment variation metrics.

Embodiment 34. The method of any of embodiments 26-32, wherein the deviation metric comprises a log 2ratio metric.

Embodiment 35. The method of any of embodiments 26-32, wherein the deviation metric comprises a root mean squared (p=2-moment) deviation metric, or any combination of p-moment variation metrics.

Embodiment 36. The method of any of embodiments 1-25, wherein the certainty metric does not measure the deviation of a value for the target variable from a reference value, e.g., an expected value.

Embodiment 37. The method of any of embodiments 1-25 or 36, wherein the certainty metric is an entropy metric, e.g., a metric that inherently measures relative certainty of the target variable, e.g., an entropy metric described herein, or any p-moment or a combination thereof

Embodiment 38. The method of embodiment 37, wherein the entropy metric measures the certainty of a ratio of the abundance of a maternal or paternal allele relative to the abundance of maternal and paternal alleles.

Embodiment 39. The method of any of embodiments 37-38, wherein the entropy metric measures the certainty of a ratio of the abundance of a maternal allele and a paternal allele in the sample relative to the abundance of the maternal allele and the paternal allele in the reference sample.

Embodiment 40. The method of embodiment 39, wherein the ratio comprises a log ratio, e.g., a log₂ ratio.

Embodiment 41. The method of any of embodiments 1-40, further comprising

sequencing the sample, e.g., by next-generation sequencing (NGS), e.g., to determine the abundance of an allele at the subgenomic interval.

Embodiment 42. The method of any of embodiments 1-41, wherein the certainty metric is a function of allele coverage at the subgenomic interval, e.g., when sequencing is used to determine the abundance of the allele.

Embodiment 43. The method of any of embodiments 1-41, further comprising

performing array hybridization on the sample, e.g., to determine the abundance of an allele at the genomic locus.

Embodiment 44. The method of embodiment 43, wherein the certainty metric is a function of allele intensity at the subgenomic interval, e.g., when array hybridization is used to determine the abundance of the allele.

Embodiment 45. The method of any of embodiments 1-44, wherein the subgenomic interval is selected based on its expected allele fraction.

Embodiment 46. The method of embodiment 45, wherein the expected allele fraction is a 0.50 allele fraction in a subset of individuals in a healthy population.

Embodiment 47. The method of embodiment 45, wherein the expected allele fraction is other than 0, 0.50, or 1, in a subject having abnormal cell growth.

Embodiment 48. The method of any of embodiments 1-47, wherein the subgenomic interval is selected based on its respective allele location, and wherein the respective allele location is expected to have an allele fraction other than 0.50 in a subject having a particular disease ontology,

Embodiment 49. The method of embodiment 48, wherein the particular disease ontology is one of a cancer condition or a precancer condition.

Embodiment 50. The method of any of embodiments 1-49, further comprising:
accessing a training dataset of information obtained from clinical specimens (or cell-lines, or in silico simulated sample sets), the information including a plurality of relationships between stored certainty metrics and stored tumor fractions from a subject population; and
applying a machine learning process to the training dataset to determine the determined relationship between the stored certainty metrics and the stored tumor fractions.

Embodiment 51. A computer system comprising:
a database configured to store a determined relationship between a stored certainty metric and a stored tumor fraction;
a processor; and
a memory communicatively coupled to the processor and including instructions that when executed by the processor cause the processor to:
acquire a value for a target variable at a subgenomic interval in the sample;
determine, from the target variable, a certainty metric;
access, in the database, the determined relationship between the stored certainty metric and the stored tumor fraction; and
determine, with reference to the certainty metric and the determined relationship, the tumor fraction of the sample.

Embodiment 52. The computer system of embodiment 51, wherein the memory further includes instructions that when executed by the processor cause the processor to:
access a training dataset of information obtained from clinical specimens (or cell-lines, or in silico simulated sample sets), the information including a plurality of relationships between stored certainty metrics and corresponding stored tumor fractions, the plurality of relationships having been determined from a subject population; and
apply a machine learning process to the training dataset to determine the determined relationship between the stored certainty metrics and corresponding stored tumor fractions.

Embodiment 53. A method of treating a disease in a subject, the method comprising:
responsive to an estimation of tumor fraction, administering an effective amount of a therapy to the subject, thereby treating the disease,
wherein, the estimation of tumor fraction comprises:
acquiring a value for a target variable at a subgenomic interval in a sample from the subject;
determining, from the target variable, a certainty metric;
accessing a determined relationship between a stored certainty metric and a stored tumor fraction; and
determining, with reference to the certainty metric and the determined relationship, the tumor fraction of the sample.

Embodiment 54. A method of evaluating a disease in a subject, the method comprising:
acquiring a first value for a target variable at a subgenomic interval in a first sample from the subject;
determining, from the target variable, a first certainty metric;
accessing a determined relationship between a stored certainty metric and a stored tumor fraction; and
determining, with reference to the first certainty metric and the determined relationship, a tumor fraction of the first sample;
acquiring a second value for the target variable at the subgenomic interval in a second sample from the subject;
determining, from the target variable, a second certainty metric;
determining, with reference to the second certainty metric and the determined relationship, the tumor fraction of the second sample; and
comparing the tumor fraction of the first sample to the tumor fraction of the second sample, thereby evaluating the disease in the subject.

Embodiment 55. The method of embodiment 54, wherein the first sample is taken at a first time point, and wherein the second sample is taken at a second time point.

Embodiment 56. The method of embodiment 55, wherein the first time point is before the subject has been administered a therapy, and wherein the second time point is after the subject has been administered the therapy.

Embodiment 57. A method of evaluating a subject, the method comprising:
acquiring a value for a target variable at a subgenomic interval in a sample from the subject;
determining, from the target variable, a certainty metric;
accessing a determined relationship between a stored certainty metric and a stored tumor fraction; and
determining, with reference to the certainty metric and the determined relationship, the tumor fraction of the sample, thereby evaluating the subject.

Embodiment 58. A method of evaluating a therapy, the method comprising:
acquiring a value for a target variable at a subgenomic interval in a sample from a subject who has been administered a therapy;
determining, from the target variable, a certainty metric;
accessing a determined relationship between a stored certainty metric and a stored tumor fraction; and
determining, with reference to the certainty metric and the determined relationship, a tumor fraction of the sample, thereby evaluating the efficacy of the administered therapy.

Embodiment 59. A method of providing a report, the method comprising:
acquiring a value for a target variable at a subgenomic interval in a sample from a subject;
determining, from the target variable, a certainty metric;
accessing a determined relationship between a stored certainty metric and a stored tumor fraction; and
determining, with reference to the certainty metric and the determined relationship, a tumor fraction of the sample; and
recording the tumor fraction in a report.

Embodiment 60. A method of evaluating a biopsy from a subject, the method comprising:
acquiring a value for a target variable at a subgenomic interval in a biopsy from the subject;
determining, from the target variable, a certainty metric;
accessing a determined relationship between a stored certainty metric and a stored tumor fraction; and
determining, with reference to the certainty metric and the determined relationship, a tumor fraction of the biopsy, thereby evaluating the biopsy.

Embodiment 61. The system or method of any of embodiments 1-60, wherein the subject has a cancer, or is at risk of having a cancer, or may have a cancer,

Embodiment 62. The system or method of embodiment 61, wherein the cancer is a solid tumor.

Embodiment 63. The system or method of embodiment 61, wherein the cancer is a hematological cancer, e.g., a leukemia or a lymphoma,

Embodiment 64. The system or method of any of embodiments 1-63, wherein the sample is a liquid sample, e.g., a blood or serum sample,

Embodiment 65. The system or method of any of embodiments 1-63, wherein the sample is a solid sample, e.g., an FFPE sample.

Embodiment 66. The system or method of any of embodiments 1-63, wherein the sample comprises cell-free DNA (cfDNA) or circulating tumor DNA (ctDNA).

Embodiment 67. The system or method of any of embodiments 1-66, wherein the subject is undergoing monitoring for at least one disease.

Embodiment 68. The system or method of any of embodiments 1-67, wherein the subject is undergoing diagnosis for at least one disease.

Embodiment 69. The system or method of any of embodiments 1-68, wherein the subject has an expected tumor fraction of less than or equal to 0.30.

Embodiment 70. The system or method of any of embodiments 1-69, further comprising determining a treatment for the subject based on the tumor fraction of the sample from the subject.

Embodiment 71. The system or method of embodiment 70, further comprising administering the treatment to the subject.

Embodiment 72. A method of discovering tumor content in a subject, the method comprising:
acquiring a value for a target variable at a subgenomic interval in a biopsy from the subject;
determining, from the target variable, a certainty metric;
accessing a determined relationship between a stored certainty metric and a stored tumor fraction; and
determining, with reference to the certainty metric and the determined relationship, a sample tumor fraction of the sample, thereby discovering tumor content in the subject,
   Interactions with Others

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### EQUIVALENTS

Those skilled in the art will recognize or be able to ascertain using no more than routine experimentation, many equivalents to the specific embodiments of the invention described herein. Such equivalents are intended to be encompassed by the following claims.

### EXAMPLES

### Example 1—Comparison of Tumor Fraction Estimated Using MSAF and the Entropy for a Probability Distribution of Allele Frequencies

Maximum somatic allele frequency (MSAF) and allele fraction (AF) was determined for a cultures of HCC1954 and HCC1143 cell cultures across SNP loci within the TP53 subgenomic interval using methods generally described in Clark et al., Analytical Validation of a Hybrid Capture-Based Next-Generation Sequencing Clinical Assay for Genomic Profiling of Cell-Free Circulating Tumor DNA, J. Molecular Diagnostics, vol. 20, pp. 686-702 (2018). MSAF was used as a proxy for tumor fraction of each sample. To obtain different tumor fractions (i.e., MSAF), the cell lines were serially diluted with paired normal DNA. A probability distribution function (PDF) for all allele frequencies was determined for each sample cell culture, and a corresponding entropy for each PDF was determined.

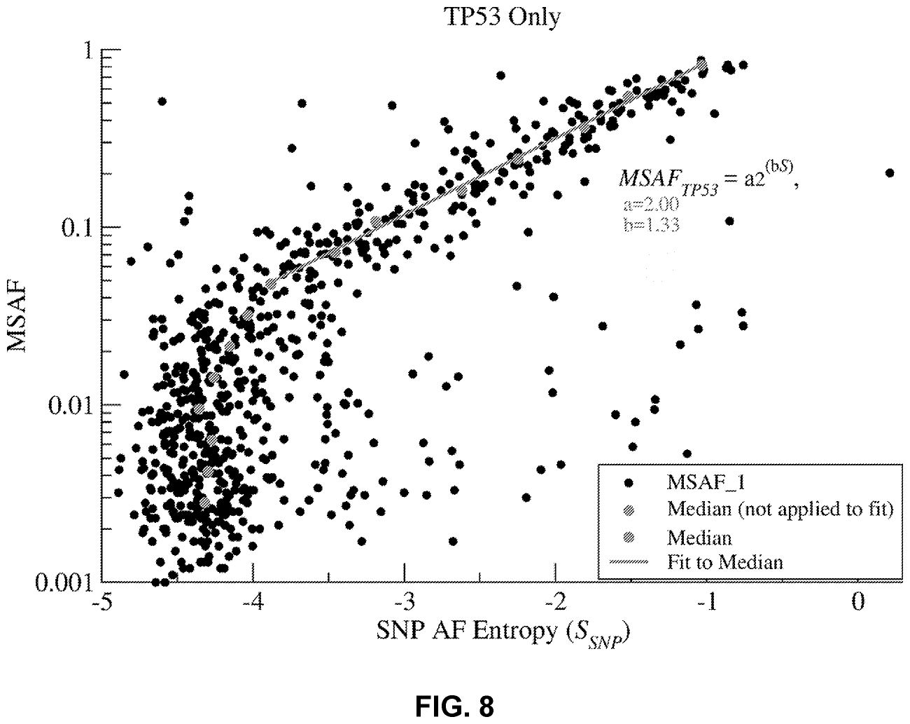

Tumor fraction (as represented by the MSAF proxy) was plotted against the determined entropy for each cell, as shown in FIG. 8. A linear relationship was determined between entropy of the probability distribution function and log of the tumor fraction for tumor fraction above 0.05%.

### Example 2—Simulation Data for Determining Tumor Fractions

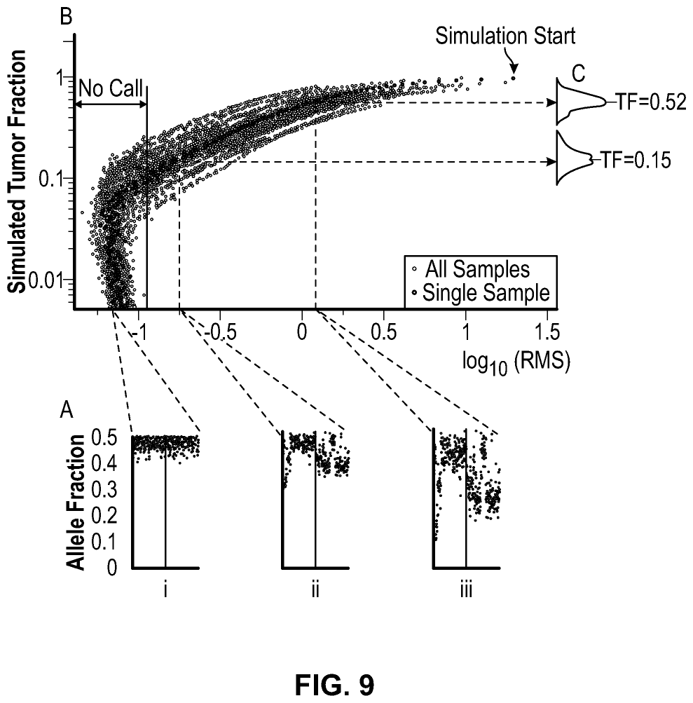

FIG. 9 provides an example of simulation data for determining tumor fraction according to the tumor fraction estimator (TFE) method illustrated in FIG. 1. Panel A of FIG. 9 provides plots of genomically-sorted minor allele frequencies as found, for example, on chromosome 6 and 7 for samples comprising: (i) low-tumor content, (ii) moderate tumor content, and (iii) high tumor content. Panel B provides a plot of simulated tumor fraction as a function of the log of root mean square deviation in allele frequencies (log₁₀(RMS)). The grey dots represent the data for all 56 tumor samples diluted in-silico. The set of black dots is an example of a single sample dilution with its in-silico normal. Panel C shows the distribution of expected tumor fraction values for each calculated RMS value. The grey density plots show the distribution of TF found about the RMS value for the allele frequency plots shown in panel A(ii) and (iii). The allele frequency plot shown in panel A(i) produces no estimate of tumor fraction.

### Example 3—Circulating Cell-Free DNA Yield and Circulating-Tumor DNA Quantity from Liquid Biopsies of 12,139 Cancer Patients

The amounts of circulating cell-free DNA (cfDNA) and circulating tumor DNA (ctDNA) present in peripheral blood liquid biopsies can vary due to pre-analytic/analytic variables. In this study, the impact of patient age, sex, stage, and tumor type on cfDNA yield, ctDNA fraction, and estimated ctDNA quantity was examined for a large cohort of clinical liquid biopsy samples. A retrospective analysis was performed of 12,139 consecutive samples received for liquid biopsy clinical testing. Significant differences in both cfDNA yield and estimated ctDNA quantity were observed based on the underlying tumor type that initiated the liquid biopsy analysis and the stage of the patient (p<0.001). In addition, significant differences in ctDNA quantity were present based on both the patient age and sex (p<0.001), Importantly, a significantly higher success rate of issuing a clinically useful report was observed for patients with higher levels of cfDNA yield and ctDNA quantity (p<0.001). The quantity of ctDNA detected varied significantly based on patient age, sex, stage, and tumor type, which could offer an explanation for why certain liquid biopsy specimens are more likely to fail to provide clinically meaningful results. In addition, the results of this study could impact future clinical decisions on the blood sample volumes required to enable successful liquid biopsy testing.

Introduction: With the growth of predictive biomarkers gaining approval as companion diagnostics for cancer therapy, testing of patients for clinically-actionable biomarkers has become standard of care. While most predictive biomarkers for solid tumors uses a tissue biopsy from the patient as a sample type, an adequate tissue sample is sometimes difficult to obtain either because the health status of the patient will not allow the patient to undergo a tissue biopsy, and/or because it is not technically feasible. For these patients, liquid biopsies are an attractive alternative specimen type for testing due to the relatively non-invasive nature of obtaining a liquid biopsy. In addition, although a tissue based specimen type is still the only choice for most companion diagnostics, for early detection and surveillance a liquid biopsy process could be the more favored methodology. However, it is important to note that for early detection, the sensitivity of liquid biopsies to screen for cancers may be affected by clinical factors such as age, sex, stage, and tumor type.

Generally, cell-free DNA (cfDNA) is extracted from the liquid biopsy, which can be from whole blood, urine, or cerebrospinal fluid. cfDNA is comprised of fragments of DNA that are released from normal and cancerous cells during apoptosis and necrosis. The fraction of cfDNA derived from tumor tissue is termed circulating tumor DNA (ctDNA), and these DNA fragments contain the same genomic alterations as the tumor cells. Sequencing of ctDNA has many clinical utilities including early detection of cancer in patients, monitoring progression and re-occurrence in cancer patients, and evaluation of biomarkers that can enable patients to be eligible for targeted therapies.

The amount of cfDNA quantified can depend on a variety of pre-analytic and analytic factors. Low levels of cfDNA is normally detected in healthy individuals and increased levels of cfDNA accumulate when individuals experience cellular stress. Higher levels of cfDNA is observed in older patients, patients with cancer, stroke, autoimmune disease, sepsis, and even after exercise. Specifically, in cancer patients, levels of cfDNA in peripheral blood tend to correlate with tumor burden and metastasis. Multiple quality control measures are needed to ensure that a clinical report utilizing cfDNA as a specimen type delivers sensitive and specific results. When using cfDNA from the plasma of whole blood as a specimen type for comprehensive genomic profiling (CGP), occasionally, an insufficient amount of cfDNA or ctDNA is available for sequencing, resulting in a failed specimen. In this study, the impact of patient age, sex, stage of disease, and tumor type on cfDNA yield, ctDNA fraction, and estimated ctDNA quantity from a large cohort of clinical samples submitted for CGP testing was evaluated.

### Materials and Methods

Patient cohort: A retrospective analysis of 12,139 consecutively received samples for liquid biopsy assay testing. Age, sex, stage of disease, and cancer diagnosis data were extracted from the clinical documents accompanying the sample. All cancer diagnoses were grouped into one of 14 tumor types and, as a secondary analysis, certain tumor groups were subdivided into subgroups where it was hypothesized that there could be potentially significant differences in cfDNA yield due to biological differences between those subgroups. The age of the patient was grouped into <50, 50-59, 60-69, 70-79, and ≥80 years old.

Liquid Biopsy assay: The liquid biopsy assay was performed in a Clinical Laboratory Improvement Amendments (CLIA)-certified and College of American Pathologists (CAP)-accredited laboratory. The liquid biopsy assay was previously described (Clark, et al. (2018), “Analytical Validation of a Hybrid Capture-Based Next-Generation Sequencing Clinical Assay for Genomic Profiling of Cell-Free Circulating Tumor DNA”, J. Molec. Diagnostics 20(5):686-702). The liquid biopsy assay used a next generation sequencing platform and a hybrid capture methodology that detects base substitutions, insertions/deletions, and copy number alterations in 70 commonly altered oncogenes and select gene rearrangements and reports microsatellite instability (MSI) status.

Briefly, two tubes of whole blood (8.5 mL per tube) were collected from each patient by venipuncture according to CLSI H3-A6. The whole blood was transported at room temperature (was not frozen before processing) and less than 14 days elapsed between time blood is drawn and processed for all samples. Once received, the plasma was separated from the whole blood by centrifugation and visual inspection was performed to assess for hemolysis. If the sample passed visual inspection for hemolysis, cfDNA was extracted, and isolated cfDNA was quantified using the 4200 TapeStation (Agilent Technologies, Santa Clara, CA).

A minimum of 25 ng of extracted cfDNA was required for a sample to undergo sequencing per the assay specifications. Upon completion of sequencing, quality control (QC) criteria assessing the sequencing data were utilized to determine whether a sample should result in a Pass report, Fail report, or Qualified report. Pass reports were issued for samples that met all QC metrics; Fail reports were issued for samples that did not meet critical QC metrics to allow for the sensitivity and specificity that is specified on our package insert for the assay; and Qualified reports were issued for samples where QC metrics were not met, but genomic alterations (GA) were found. For these reports, the sensitivity is likely decreased, but a Qualified report was issued because the specificity is not decreased. Two important QC metrics assessed on the sequencing data include sequence coverage and contamination.

For the purposes of this study, in determining whether a GA detected versus a GA not detected clinical report resulted, only pathogenic and likely pathogenic GA were considered, and variants of unknown significance were not considered.

Determination of composite tumor fraction (cTF): A composite tumor fraction methodology, as described elsewhere herein, was designed to measure the ctDNA fraction of the total circulating cfDNA, across a broad range of tumor content values. This method leverages two complementary metrics which are a proprietary tumor fraction estimator (TFE) and the maximum somatic allele frequency (MSAF) method.

The TFE is based on a measure of tumor aneuploidy that incorporates observed deviations in, for example, SNP coverage across the genome for a given sample. Calculated values for this metric are calibrated against a training set based on samples with well-defined tumor fractions to generate an estimate of the tumor fraction. The MSAF method estimates the fraction of ctDNA versus that of all sources of cfDNA in plasma, including circulating germline DNA from patients. The MSAF is determined by determining the allele fraction for all known somatic, likely somatic, and variant of unknown significance substitution alterations detected at >2000× median unique coverage by non-PCR duplicate read pairs, excluding certain common and rare germline variants. A select list of variants associated with clonal hematopoiesis are not considered while determining MSAF.

The composite tumor fraction measurement defaults to the TFE's value when available. When lack of tumor aneuploidy limits the TRE's ability to return an informative estimate of ctDNA fraction, MSAF is used to generate a ctDNA fraction value. In this case, only variants with allele fraction at or below THE's limit of detection are used as input for MSAF. When the TFE's estimate is unavailable or impacted by failed quality control measures, the composite tumor fraction is generated from MSAF as previously described. In effect, the MSAF process is typically used at low ctDNA fractions due to its higher reliability at lower ctDNA fractions, and the TFE process is used at high ctDNA fractions due to its higher reliability at higher ctDNA fractions,

Estimated ctDNA Quantity: ctDNA quantity was estimated by multiplying the cfDNA yield by the calculated composite tumor fraction.

Data Analysis: The data were examined to determine if there was a significant difference in cfDNA yield and estimated ctDNA quantity based on sex, age, stage of disease, tumor type, resulting Pass/Fail/Qualified reports, and clinical reports with no GA reported. In addition, the ctDNA fraction and its correlation with sex, age, stage of disease, and tumor type was examined. Univariate analysis to include one variable (sex, age, tumor type, stage, Pass/Qualified/Fail report, or no GA reported) at a time was performed to evaluate its impact on outcomes (cfDNA, ctDNA, or ctDNA fraction). Because the cfDNA, ctDNA and ctDNA fraction are not normally distributed even after box-cox transformation (Shapiro-Wilk test P value<0.001), the univariate analysis was implemented with a non-parametric statistical test, Kruskal-Wallis Rank Sum test or Wilcoxon Rank Sum test, which was applied to test if the outcome distributions are the same among multiple or two covariate groups, respectively. P<0.05 was considered significant in this analysis.

In addition, multivariate regression analysis was performed on the clinical factors of patient age, sex, stage, and tumor type. Because the cfDNA, ctDNA fraction, ctDNA, and their log-transformations are not normally distributed and many outliers are observed, general linear regression models can't be fit. Instead, quantile regression models were fit for the median of cfDNA, ctDNA fraction, and ctDNA with all interested variables to implement the multivariate analysis. ANOVA was further applied to test if each interested factor (patient age, sex, stage, and tumor type) was significantly associated with the median of cfDNA, ctDNA fraction, and ctDNA by adjusting other factors.

### Results:

Patient cohort: A total of 12,139 clinical liquid biopsy samples were analyzed including 52.6% (6,391/12,139) females and 47.2% (5,734/12,139) males. Sex status was not available for 14 patients. Of the 12,139 samples, it was possible to make a determination of the stage of the disease in 79.2% (9618) of the patients. Almost all (91.8%, 8833/9618) of the samples with known stage of disease were stage 4 disease. The cohort characteristics are summarized in Table 6.

TABLE 6
Median and mean circulating cell-free DNA yields based on
sex, age, stage of disease, and tumor type, Pass/Qualified/Fail
report, and genomic alterations (GA) detected report

| | Sample | Median | Mean | P- | ***P- |
|---|---|---|---|---|---|
| | Count | (ng) | (ng) | value | value |
| Overall | 12139 | 92 | 252 | | |
| Sex | | | | *0.307 | 0.380 |
| Female | 6391 | 91 | 251 | | |
| Male | 5734 | 94 | 253 | | |
| Age | | | | **0.119 | 0.510 |
| <50 years old | 1370 | 89 | 292 | | |
| 50-59 years old | 2252 | 97 | 276 | | |
| 60-69 years old | 3721 | 94 | 258 | | |
| 70-79 years old | 3300 | 93 | 233 | | |
| ≥80 years old | 1496 | 87 | 207 | | |
| Stage | | | | **<0.001 | <0.001 |
| 1 | 100 | 69 | 129 | | |
| 2 | 157 | 63 | 131 | | |
| 3 | 528 | 72 | 147 | | |
| 4 | 8833 | 96 | 263 | | |
| Tumor type | | | | **<0.001 | <0.001 |
| Hematologic Malignancy | 42 | 108 | 482 | | |
| Neuroendocrine Tumors | 289 | 89 | 323 | | |
| Prostate Carcinoma | 1148 | 86 | 323 | | |
| Breast Carcinoma | 1732 | 94 | 303 | | |
| Unknown/Unspecified/ | 685 | 107 | 296 | | |
| Low Count | | | | | |
| GI Carcinoma | 2970 | 121 | 290 | | |
| H&N Tumors | 119 | 73 | 245 | | |
| Skin Tumors | 173 | 76 | 238 | | |
| Endocrine Tumors | 71 | 97 | 229 | | |
| Soft Tissue Tumors | 183 | 82 | 208 | | |
| GU Carcinoma | 249 | 90 | 198 | | |
| NSCLC | 3866 | 81 | 182 | | |
| GYN Carcinoma | 549 | 81 | 181 | | |
| CNS Tumors | 63 | 51 | 118 | | |
| Pass/Qualified/Fail | | | | **<0.001 | |
| Pass | 9933 | 109 | 279 | | |
| Qualified | 1285 | 43 | 162 | | |
| Fail | 774 | 40 | 97 | | |
| GA | | | | *<0.001 | |
| GA Detected | 9375 | 104 | 286 | | |
| GA Not Detected | 2617 | 67 | 143 | | |

*Wilcoxon Rank Sum test
**Kruskal-Wallis Rank Sum test
***Multivariate regression analysis on sex, age, stage, tumor type

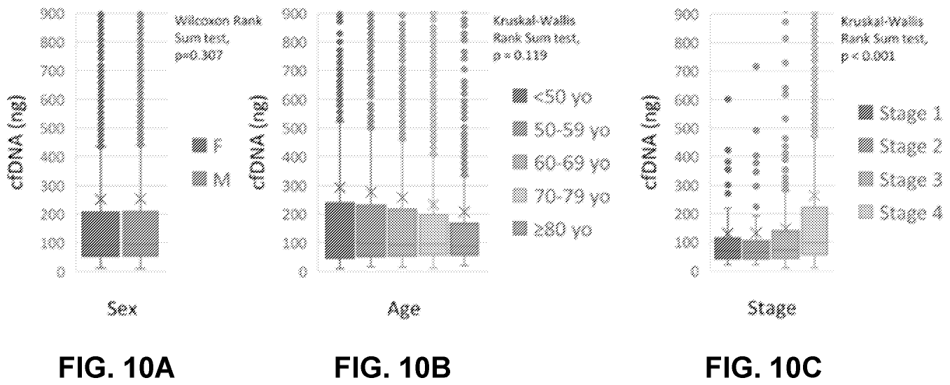

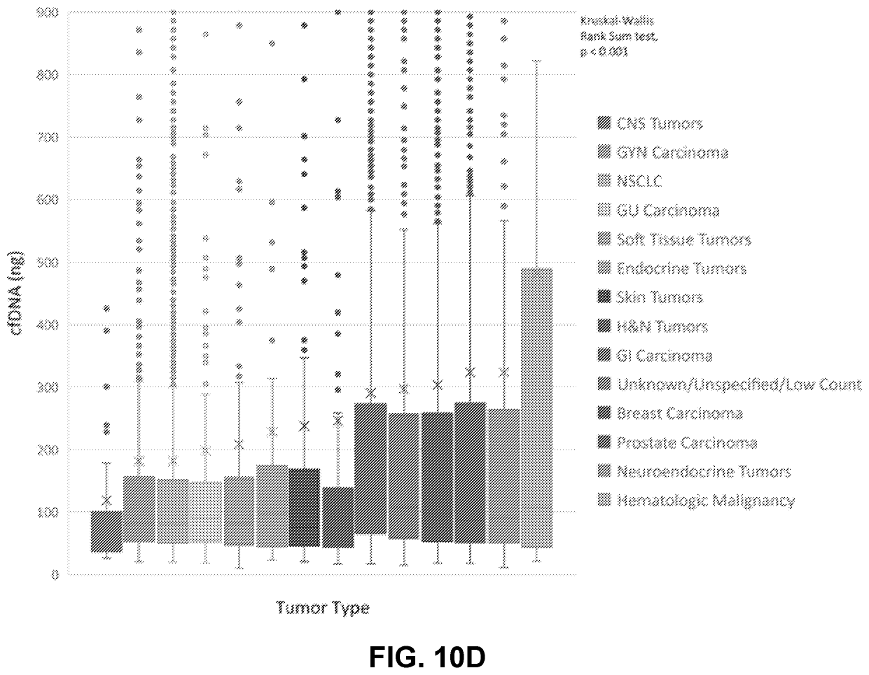

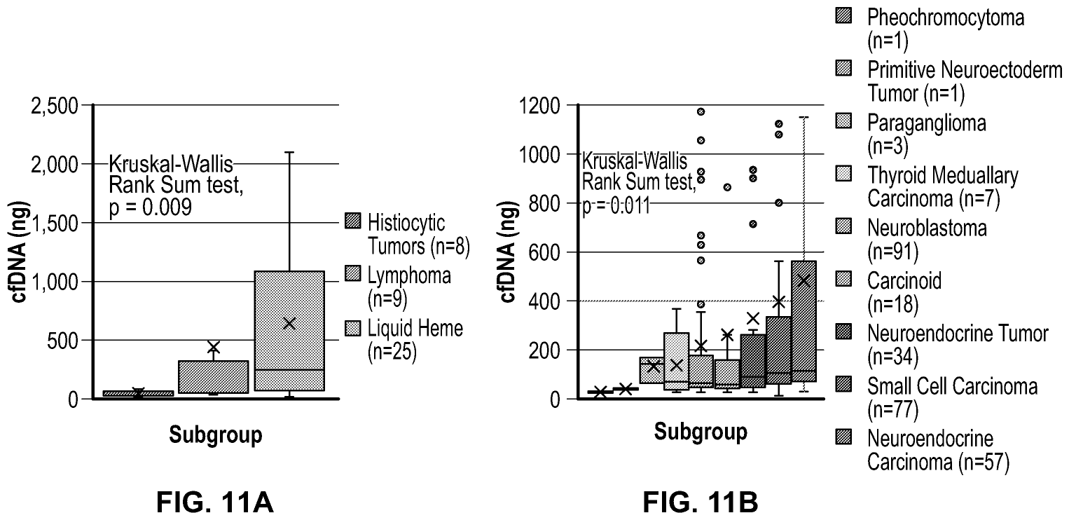

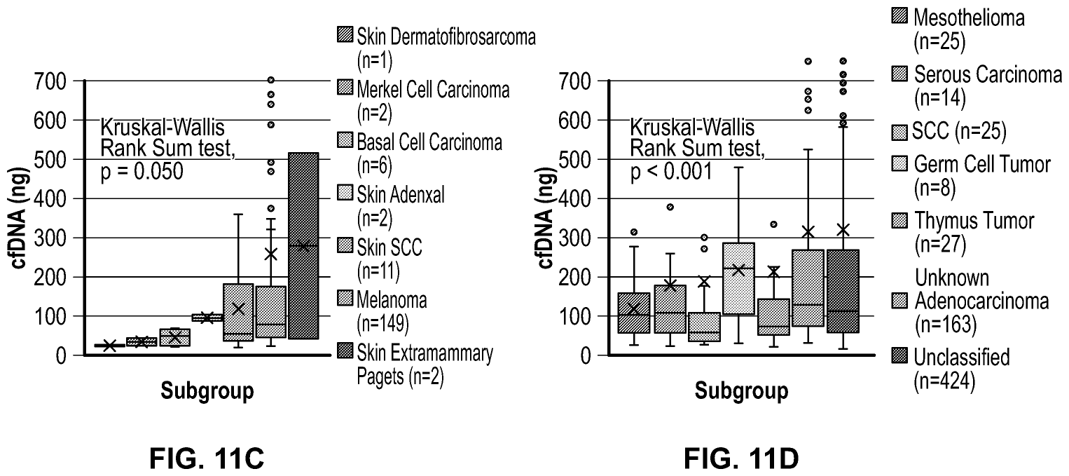

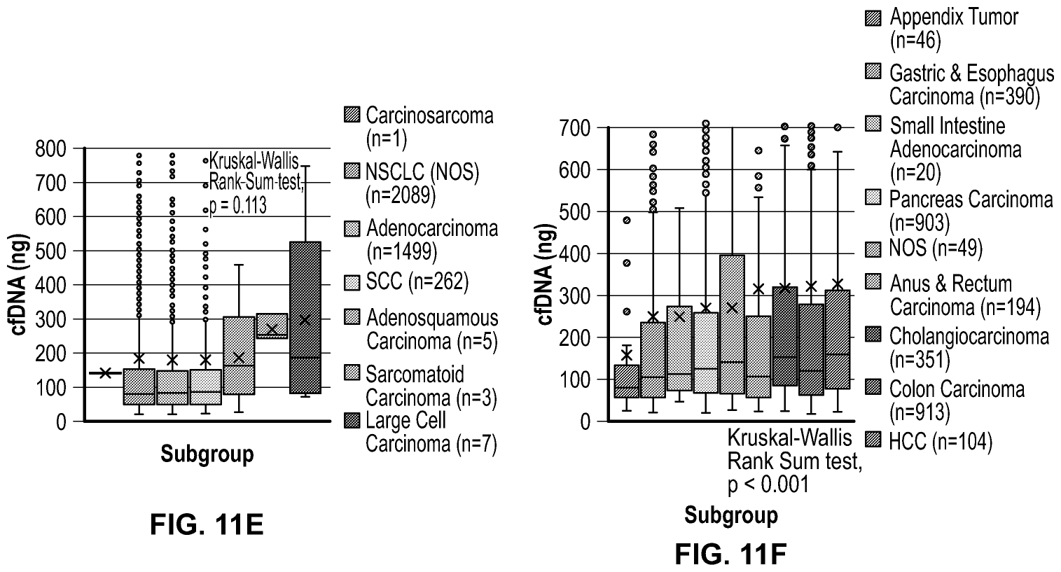

cfDNA yield Based on Sex, Age, Stage, and Tumor Type: cfDNA yield is highly correlated with tumor type and stage of disease. No significant differences in cfDNA yield was observed between samples based on sex (Wilcoxon Rank Sum test, p=0.307; multivariate analysis: ANOVA, p=0.380) (Table 6, FIG. 10A). A downward trend was observed in the average cfDNA yield as the age of patients increased, although no significance was found (Kruskal-Wallis Rank Sum test, p=0.119; multivariate analysis: ANOVA, p=0.510) (Table 6, FIG. 10B). The average amount of cfDNA yield increased significantly with increasing stage of disease of the patient (Kruskal-Wallis Rank Sum test, p<0.001; multivariate analysis: ANOVA, p<0.001) (Table 6, FIG. 10C). Average cfDNA yield differed significantly based on the patient's tumor type (Kruskal-Wallis Rank Sum test, p<0.001; multivariate analysis: ANOVA, p<0.001) (Table 6, FIG. 10D). Certain tumor types such as hematologic malignancy, neuroendocrine tumor, prostate carcinoma, breast carcinoma, and gastrointestinal (GI) carcinoma had above average cfDNA yields while head and neck tumors, skin tumors, endocrine tumors, soft tissue tumors, genitourinary (GU) tumors other than prostate carcinoma, non-small cell lung cancer (NSCLC), gynecologic (GYN) tumors, and central nervous system tumors (CNS) had below average cfDNA yield (FIG. 10D). Of note, CNS tumors had the lowest cfDNA yield. In addition, when hematologic malignancies, neuroendocrine tumors, skin tumors, unknown/unspecified, NSCLC, gastrointestinal cancer were divided into subgroups, a significant difference between the subgroups of those tumor types was observed with the exception of skin tumors and NSCLC (Kruskal-Wallis Rank Sum test [only subgroups where n>5 was included in statistical analysis], p=0.009, p=0.011, p=0.050, p<0.001, p=0.113, p<0.001, respectively) (FIGS. 11A-F).

FIGS. 10A-D provide non-limiting examples of box-whisker plots of circulating cell-free DNA (cfDNA) yield based on patient's sex, age, stage, and tumor type. FIG. 10A: Plot of cfDNA yield versus sex. No significant differences in cfDNA yield was observed between samples based on sex (Wilcoxon Rank Sum test, p=0.307). FIG. 10B: Plot of cfDNA yield versus age. A downward trend was observed in the average cfDNA yield as the age of patients increased, although no significance was found (Kruskal-Wallis Rank Sum test, p=0.119). FIG. 10C: Plot of cfDNA yield versus disease stage. The average amount of cfDNA yield increased significantly with increasing stage of disease of the patient (Kruskal-Wallis Rank Sum test, p<0.001). FIG. 10D: Plot of cfDNA yield versus tumor type. Average cfDNA yield differed significantly based on the patient's tumor type with the highest cfDNA yield in hematologic malignancies and central nervous system tumors having the lowest cfDNA yield (Kruskal-Wallis Rank Sum test, p<0.001). Tumor types arranged with increasing mean. Not all outliers are represented, and x denotes mean yield.

FIGS. 11A-F provide non-limiting examples of box-whisker plots of circulating cell-free DNA (cfDNA) yield based on subgroups of select tumor types. FIG. 11A: Plot of cfDNA yield versys hematologic subgroup. Significant differences in cfDNA yield was observed between the subgroups of hematologic malignancies (Kruskal-Wallis Rank Sum test, p=0.009). FIG. 11B: Plot of cfDNA yield versus neuroendocrine subgroup. Significant differences in cfDNA yield was observed between the subgroups of neuroendocrine tumors (Kruskal-Wallis Rank Sum test, p=0.011). FIG. 11C: Plot of cfDNA yield versys skin subgroup. No significant differences in cfDNA yield was observed between the subgroups of skin tumors (Kruskal-Wallis Rank Sum test, p=0.050). FIG. 11D: Plot of cfDNA yield for unknown/unclassified/low count subgroups. Significant differences in cfDNA yield was observed between the subgroups of unknown/unclassified/low count (Kruskal-Wallis Rank Sum test, p<0.001). FIG. 11E: Plot of cfDNA yield versus non-small cell lung cancer (NSCLC) subgroup. No significant differences in cfDNA yield was observed between the subgroups of non-small cell lung cancers (Kruskal-Wallis Rank Sum test, p=0.113). FIG. 11F: Plot of cfDNA yield versus gastrointestinal subgroup. Significant differences in cfDNA yield was observed between the subgroups of gastrointestinal cancer (Kruskal-Wallis Rank Sum test, p<0.001). Tumor type subgroups arranged with increasing mean and only subgroups where n>5 was included in statistical analysis. Not all outliers are represented, and x denotes mean yield.

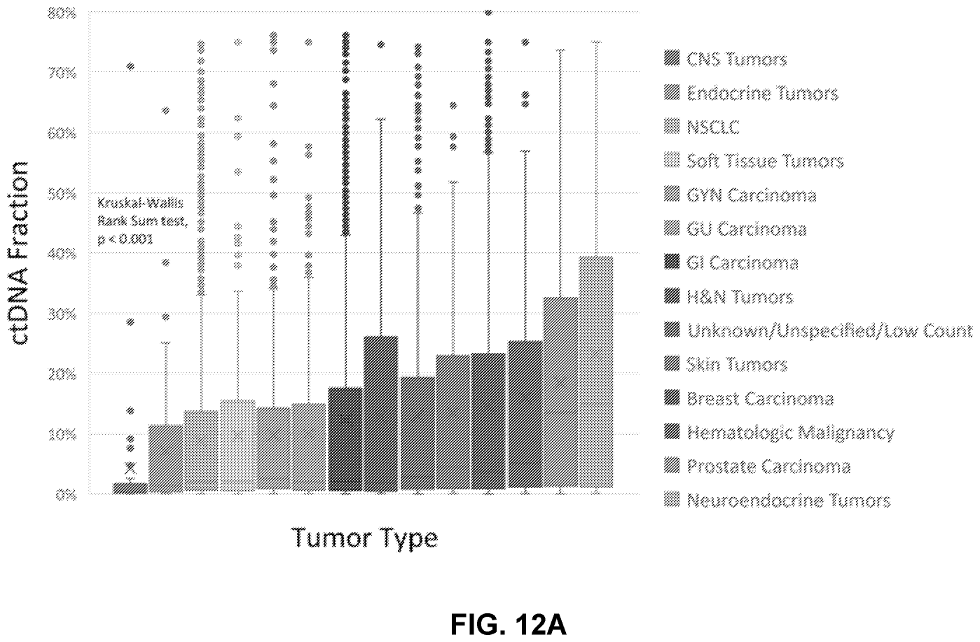

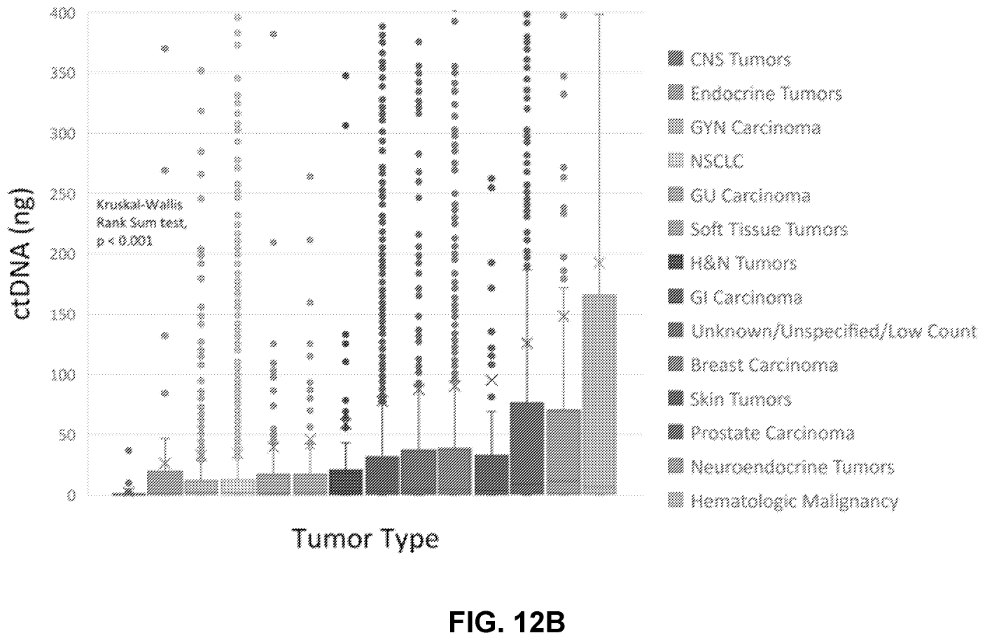

ctDNA fraction Based on Sex, Age, Stage, and Tumor Type: Overall, ctDNA fraction was low with a median of 2.6% and mean of 12.2%. ctDNA fraction was highly correlated with sex, age, stage, and tumor type (Table 7). A significant difference was found in ctDNA fraction based on gender of patients (Wilcoxon Rank Sum test, p<0.001; multivariate analysis; ANOVA, p<0.001) (Table 7). Significant differences were observed between the different age groups with a downward trend in the ctDNA fraction as the age of the patients increased (Kruskal-Wallis Rank Sum test, p<0.001, multivariate analysis: ANOVA, p<0.001) (Table 7). The ctDNA fraction increased significantly with increasing stage of disease of the patient (Kruskal-Wallis Rank Sum test, p<0.001; multivariate analysis: ANOVA, p<0.001) (Table 7). Significant differences were discovered between ctDNA fraction based on tumor types (Kruskal-Wallis Rank Sum test, p<0.001; multivariate analysis: ANOVA, p<0.001) (Table 7, FIG. 12A). Many of the tumor types that had higher cfDNA levels also had higher than average ctDNA fraction, namely, neuroendocrine carcinoma, prostate carcinoma, hematologic malignancies, breast carcinoma, and GI carcinoma.

TABLE 7
Median and mean circulating tumor DNA fraction based
on sex, age, stage of disease, and tumor type

| | Sample | | | P- | ***P- |
|---|---|---|---|---|---|
| Column1 | Count | Median | Mean | value | value |
| Overall | 10336 | 2.6% | 12.2% | | |
| Sex | | | | *<0.001 | <0.001 |
| Female | 5437 | 2.3% | 11.3% | | |
| Male | 4887 | 3.0% | 13.1% | | |
| Age | | | | **<0.001 | <0.001 |
| <50 years old | 995 | 4.9% | 16.5% | | |
| 50-59 years old | 1847 | 3.3% | 13.7% | | |
| 60-69 years old | 3213 | 2.5% | 12.2% | | |
| 70-79 years old | 2929 | 2.4% | 10.8% | | |
| ≥80 years old | 1352 | 2.2% | 9.9% | | |
| Stage | | | | **<0.001 | <0.001 |
| 1 | 75 | 1.0% | 5.9% | | |
| 2 | 121 | 1.1% | 6.3% | | |
| 3 | 426 | 1.5% | 7.5% | | |
| 4 | 7633 | 2.9% | 12.7% | | |
| Tumor type | | | | **<0.001 | <0.001 |
| Neuroendocrine | 31 | 15.0% | 23.2% | | |
| Tumors | | | | | |
| Prostate Carcinoma | 236 | 13.5% | 18.5% | | |
| Hematologic | 987 | 5.2% | 16.0% | | |
| Malignancy | | | | | |
| Breast Carcinoma | 1509 | 3.6% | 14.5% | | |
| Skin Tumors | 565 | 4.6% | 13.4% | | |
| Unknown/Unspecified/ | 2515 | 2.9% | 13.0% | | |
| Low Count | | | | | |
| H&N Tumors | 97 | 1.8% | 12.8% | | |
| GI Carcinoma | 144 | 2.2% | 12.4% | | |
| GU Carcinoma | 45 | 2.0% | 10.0% | | |
| GYN Carcinoma | 137 | 2.6% | 9.8% | | |
| Soft Tissue Tumors | 208 | 2.1% | 9.8% | | |
| NSCLC | 3342 | 2.0% | 8.9% | | |
| Endocrine Tumors | 484 | 1.5% | 7.1% | | |
| CNS Tumors | 36 | 0.3% | 4.3% | | |

*Wilcoxon Rank Sum test
**Kruskal-Wallis Rank Sum test
***Multivariate regression analysis on sex, age, stage, tumor type

FIGS. 12A-B provide non-limiting examples of box-whisker plots of circulating tumor DNA (ctDNA) fraction and estimated ctDNA quantity based on tumor type. FIG. 12A: Plot of ctDNA fraction versus tumor type. Significant differences were discovered between ctDNA fraction based on tumor types (Kruskal-Wallis Rank Sum test, p<0.001). Most of the ctDNA fraction was below 20% meaning that the majority of the cfDNA was shed from normal cells and not tumor cells. FIG. 12B: Plot of ctDNA yield versus tumor type. Average estimated ctDNA quantity differed significantly based on the patient's tumor type (Kruskal-Wallis Rank Sum test, p<0.001). It was observed that similar tumor types had higher estimated ctDNA quantity as seen in analysis with cfDNA yield. Tumor types arranged with increasing mean. Not all outliers are represented, and x denotes mean yield.

Estimated ctDNA quantity Based on Sex, Age, Stage, and Tumor Type: Estimated ctDNA quantity was highly correlated with sex, age, stage, and tumor type of the patients. Higher ctDNA quantity was observed in samples from male vs female in the overall cohort (Wilcoxon Rank Sum test, p<0.001; multivariate analysis: ANOVA, p=0.015) (Table 8). Significant differences were observed between the different age groups with a downward trend in the ctDNA quantity as the age of patients increased (Kruskal-Wallis Rank Sum test, p<0.001; multivariate analysis: ANOVA, p<0.001) (Table 8). The average amount of ctDNA quantity increased significantly with increasing stage of disease of the patient (Kruskal-Wallis Rank Sum test, p<0.001; multivariate analysis: ANOVA, p<0.001) (Table 8). Average ctDNA quantity also differed significantly based on the patient's tumor type (Kruskal-Wallis Rank Sum test, p<0.001; multivariate analysis: ANOVA, p<0.001) (Table 8, FIG. 12B). Particular tumor types were observed to have higher estimated ctDNA quantity as seen in analysis with cfDNA yield. Specifically, hematologic malignancy, neuroendocrine tumor, prostate carcinoma, skin tumors, breast carcinoma, GI carcinoma had above average ctDNA yields (Table 8, FIG. 12B). Similar to cfDNA yields, CNS tumors have the lowest ctDNA quantity; however, even though skin tumors had higher average ctDNA quantity then many of the other tumor types, most cases had relatively lower ctDNA quantity and the higher average ctDNA quantity was influenced by outliers with extremely high ctDNA quantity (FIG. 12B).

TABLE 8
Median and mean estimated circulating tumor DNA quantity based
on sex, age, stage of disease, tumor type, Pass/Qualified/Fail
report and genomic alterations (GA) detected report.

| | Sample | Median | Mean | P- | ***P- |
|---|---|---|---|---|---|
| Column1 | Count | (ng) | (ng) | value | value |
| Overall | 10336 | 2.8 | 68.8 | | |
| Sex | | | | *<0.001 | 0.015 |
| Female | 5437 | 2.6 | 63.2 | | |
| Male | 4887 | 3.1 | 75 | | |
| Age | | | | **<0.001 | <0.001 |
| <50 years old | 995 | 6.3 | 107.2 | | |
| 50-59 years old | 1847 | 4.1 | 81.1 | | |
| 60-69 years old | 3213 | 2.7 | 70.2 | | |
| 70-79 years old | 2929 | 2.5 | 54.8 | | |
| ≥80 years old | 1352 | 2.1 | 50.8 | | |
| Stage | | | | **<0.001 | <0.001 |
| 1 | 75 | 0.79 | 16.4 | | |
| 2 | 121 | 0.82 | 26.3 | | |
| 3 | 426 | 1.2 | 16.5 | | |
| 4 | 7633 | 3.24 | 73.4 | | |
| Tumor type | | | | **<0.001 | <0.001 |
| Hematologic Malignancy | 31 | 6.7 | 192.4 | | |
| Neuroendocrine Tumors | 236 | 11.5 | 148.2 | | |
| Prostate Carcinoma | 987 | 8.8 | 125.6 | | |
| Skin Tumors | 1509 | 4.1 | 95.4 | | |
| Breast Carcinoma | 565 | 4.6 | 90.4 | | |
| Unknown/Unspecified/ | 2515 | 4.3 | 87.4 | | |
| Low Count | | | | | |
| GI Carcinoma | 97 | 2.9 | 77.6 | | |
| H&N Tumors | 144 | 1.9 | 58.9 | | |
| Soft Tissue Tumors | 45 | 2.2 | 45.8 | | |
| GU Carcinoma | 137 | 1.7 | 39.5 | | |
| NSCLC | 208 | 1.8 | 34 | | |
| GYN Carcinoma | 3342 | 2.6 | 32.9 | | |
| Endocrine Tumors | 484 | 1.4 | 26.1 | | |
| CNS Tumors | 36 | 0.3 | 2.1 | | |
| Pass/Qualified/Fail | | | | **<0.001 | |
| Pass | 9092 | 3.3 | 71.2 | | |
| Qualified | 1203 | 1.2 | 52.7 | | |
| Fail | 41 | 0.8 | 9.3 | | |
| GA | | | | *<0.001 | |
| GA Detected | 9011 | 3.9 | 76.7 | | |
| GA Not Detected | 1325 | 0.7 | 14.8 | | |

*Wilcoxon Rank Sum test
**Kruskal-Wallis Rank Sum test
***Multivariate regression analysis on sex, age, stage, tumor type

Numbers of Samples Passing Quality Control: Of the 12,139 samples received, 1.2% (147/12,139) were under the minimum amount of cfDNA required for the liquid biopsy assay and were not advanced for further processing (Supplemental FIG. 1). Next, of the 11,992 samples that were advanced for further processing, 82.8% (9933) of the samples were issued a Pass report, 10.7% (1285) of the samples were issued a Qualified report, and 6.5% (774) samples were issued a Fail report. The two QC metrics that are most important in determining whether a sample results in a Pass report, Qualified report, and Fail report are sequence coverage and contamination as mentioned above. Of the Pass and Qualified reports, most (83.3%, 9345/11218) of the reports issued had a least one GA identified.

Correlation of cfDNA yield and estimated ctDNA quantity with Pass/Fail/Qualified Status and GA detection: Overall, a significant positive correlation was observed between the clinical usefulness of the clinical reports issued and both the cfDNA yield and estimated ctDNA quantity. Specifically, a significant difference was observed in the average amount of cfDNA and whether the bioinformatic quality control check segmented a case as a Pass, Fail, or Qualified report with Pass reports having the highest average cfDNA yield and Fail reports having the lowest cfDNA yield (Kruskal-Wallis Rank Sum test, p<0.001) (Table 6). A significant difference was also observed in the average amount of cfDNA and whether a GA not detected clinical report was issued with GA not detected clinical reports having a lower average cfDNA yield and reports with a GA detected having a higher average cfDNA yield (Wilcoxon Rank Sum test, p<0.001) (Table 6). These trends were also the same for estimated ctDNA quantity based on Pass/Qualified/Fail report and GA detection status (Kruskal-Wallis Rank Sum test, p<0.001 and Wilcoxon Rank Sum test, p<0.001, respectively) (Table 8).

Discussion: Liquid biopsy processes involve sensitive analysis of circulating material to inform the care of cancer patients. Such highly sensitive methods require adequate material in order to perform reliably. For example, it has been reported that sensitivity diminishes dramatically with some assays when lower amounts of DNA input are used. For this reason, it is essential to understand the clinical factors that influence liquid biopsy sample quantity.

In a large cohort of clinical samples, the cfDNA yield and ctDNA quantity were found to be impacted by the underlying tumor type that initiated the liquid biopsy analysis. In addition, a decrease cfDNA yield and ctDNA quantity was observed as the patients' age increased, although significance was only found for ctDNA quantity. Lastly, the data for the large cohort used reaffirmed that patients with higher stage of disease have higher levels of cfDNA and ctDNA, To the best of our knowledge, this is the largest study in the literature that examined cfDNA yields in a variety of tumor types and examined its correlation with patient sex, age, stage, and tumor type. While cfDNA yields based on these parameters are not well characterized in the literature, more literature is available examining ctDNA levels. A study by Bettegowada, et al. (2014) “Detection of Circulating Tumor DNA in Early- and Late-Stage Human Malignancies”, Sci Transl Med 6: 224ra24, examined the ctDNA yield of 640 patients with various cancer tumor types. Two important findings in that study were patients with metastatic disease have higher rates of detectable ctDNA and variable rates of detectable ctDNA are present depending on tumor type. In that study, gliomas had the lowest rate of detectable ctDNA in the liquid biopsy. Similarly, this study highlights differences in ctDNA quantity based on tumor type and shows CNS tumors had the lowest average ctDNA yield. The present study indicates that different tumor types have different rates of shedding ctDNA into the bloodstream. Specifically, in CNS tumors, the most likely biological reason for shedding the least amount of ctDNA is due to the presence of the blood-brain barrier. This supports using cerebrospinal fluid (CSF) as a sample type instead of whole blood, when feasible. Neuroendocrine tumor, prostate carcinoma, hematologic malignancy, breast carcinoma, and GI carcinoma were found to have higher levels of both cfDNA and ctDNA, indicating these tumor types are promising candidates for a blood-based liquid biopsy. These data suggest that patient age, stage, and tumor type might influence the sample type to submit for CGP when multiple sample types are available. For example, in a younger patient with a higher stage neuroendocrine tumor, a liquid biopsy might be preferred to a tissue biopsy; however, in an older patient with a lower stage CNS tumor, a tissue biopsy or CSF sample might be preferred.

A significant difference in cfDNA yield based on sex of patient was not observed, but a significant higher level of ctDNA quantity and ctDNA fraction in males was observed. It was originally hypothesized that the difference in ctDNA yield based on sex is largely attributed to tumor specific biological mechanisms. However, with multivariate regression analysis to control for tumor type as well as patient age and stage, a significant difference of cfDNA yield based on sex was still not observed, and a significantly higher level of ctDNA quantity and ctDNA fraction based on sex was still observed. This could be due to intrinsic biologic mechanisms of how normal and tumor cells shed DNA into the bloodstream of female and male cancer patients. Future research to examine whether biological differences are present between female and male cancer patients to cause differences in the ctDNA quantity and ctDNA fraction that is shed into the bloodstream is warranted.

Consistent with the ctDNA quantity, the fraction of ctDNA in the cfDNA also differed based on age, sex, stage, and tumor type. Also, the fraction of ctDNA was low, with half of the specimens having a ctDNA fraction under 2.6%, indicating the majority of detected cfDNA was shed from normal cells.

The clinical impact of having variable rates of cfDNA and ctDNA levels was evaluated by examining how these levels correlated with whether a specimen resulted in a Pass, Qualified, or Fail report based on a bioinformatics pipeline and whether a GA was detected in the sample. It was found that not only was cfDNA and ctDNA level highly correlated with whether a sample was likely to result in a Pass, Qualified, or Fail report, it was also highly correlated with whether a GA was identified on resulting clinical report. Here, the sex, age, stage, and tumor type of the patient were found to have important implications of whether a useful clinical report is generated from a peripheral blood liquid biopsy. This information could offer an explanation on why certain liquid biopsy specimens are more likely to fail sequencing or generate a clinically useful report.

Two limitations existed in the present study. The first was that as a large referral laboratory, applicant had limited clinical information on the patient cohort; but this is also one of the major strengths of the present study. Due to the same collection methodology, sample processing, and liquid biopsy assay used for all the samples in this cohort, the pre-analytic/analytic variables are constant in this data set. However, examples of clinical information that was not available include whether the sample was a pre/post therapy sample, the disease status at the time of blood draw, and location of metastases (e.g., bone involvement vs. no bone involvement). It is well known that metastatic solid tumors that involve bone will have higher levels of cfDNA, ctDNA, and circulating tumor cells; and had this information been available, further analysis could have been used to control for this potential confounding variable. Additional clinical information would have been valuable to shed even more light on selection of sample type to use in various tumor types. In addition, the way estimated ctDNA levels were determined is based on ctDNA fraction, and hence if a ctDNA fraction could not be determined based on MSAF or TFE, the ctDNA levels for those samples couldn't be estimated.

Conclusion: In a large clinical cohort, ctDNA quantity varied significantly based on the patient's sex, age, stage, and tumor type. This information could offer an explanation on why certain liquid biopsy specimens are more likely to fail sequencing or generate a clinically useful report. In addition, this could impact future clinical decisions on the blood sample volumes required for successful ctDNA levels to facilitate liquid biopsy testing.

1. A method of identifying a genomic sequence of interest as germline or somatic, the method comprising:
providing a plurality of nucleic acid molecules obtained from a sample from a subject, wherein the plurality of nucleic acid molecules comprises a mixture of tumor nucleic acid molecules and non-tumor nucleic acid molecules;
optionally, ligating one or more adapters onto one or more nucleic acid molecules from the plurality of nucleic acid molecules;
amplifying nucleic acid molecules from the plurality of nucleic acid molecules;
capturing nucleic acid molecules from the amplified nucleic acid molecules, wherein the captured nucleic acid molecules are captured from the amplified nucleic acid molecules by hybridization to one or more bait molecules;
sequencing, by a sequencer, the captured nucleic acid molecules to obtain a plurality of sequence reads corresponding to one or more genomic loci within a subgenomic interval in the sample;
receiving, at one or more processors, a plurality of values, each value indicative of an allele fraction at a corresponding locus within the subgenomic interval in the sample;
determining, by the one or more processors, a certainty metric value indicative of a dispersion of the plurality of values;
determining, by the one or more processors, a first estimate of the tumor fraction of the sample, the first estimate based on the certainty metric value for the sample and a predetermined relationship between one or more stored certainty metric values and one or more stored tumor fraction values;
determining, by the one or more processors, whether a value associated with the first estimate is greater than a first threshold;
based on a determination that the value associated with the first estimate is greater than the first threshold, outputting, by the one or more processors, the first estimate as the tumor fraction of the sample; and
based on a determination that the value associated with the first estimate is less than or equal to the first threshold:
determining, by the one or more processors, a second estimate of the tumor fraction of the sample based on an allele frequency determination; and
outputting, by the one or more processors, the second estimate as the tumor fraction of the sample.

2.-19. (canceled)

20. The method of claim 1, wherein the tumor fraction is a value indicative of a ratio of circulating tumor DNA (ctDNA) to total cell-free DNA (cfDNA) in the sample.

21. The method of claim 1, wherein the first threshold is indicative of a minimum detectable quantity for the tumor fraction of the sample.

22. The method of claim 1, wherein determining whether the value associated with the first estimate of the tumor fraction of the sample is greater than the first threshold comprises determining whether the first estimate is greater than a defined tumor fraction threshold.

23. The method of claim 1, wherein determining whether the value associated with the first estimate of the tumor fraction of the sample is greater than a first threshold comprises determining whether a statistical lower bound associated with the first estimate is greater than 0.

24. The method of claim 1, wherein determining the second estimate of the tumor fraction of the sample based on the allele frequency determination comprises:
determining whether a quality metric for the plurality of values is greater than a second threshold;
based on a determination that the quality metric for the plurality of values is greater than the second threshold, determining the second estimate for the tumor fraction of the sample based on a first determination of somatic allele frequency, and
based on a determination that the quality metric for the plurality of values is less than or equal to the second threshold, determining the second estimate for the tumor fraction of the sample based on a second determination of somatic allele frequency.

25. The method of claim 23, wherein the quality metric for the plurality of values is indicative of an average sequence coverage for the sample, an allele coverage at each of the loci corresponding to the plurality of values, a degree of nucleic acid contamination in the sample, a number of single nucleotide polymorphism (SNP) loci within the loci corresponding to the plurality of values, or any combination thereof.

26. The method of claim 25, wherein the quality metric for the plurality of values is indicative of a minimum average sequence coverage for the sample, a minimum allele coverage at each of the loci corresponding to the plurality of values, a maximum degree of nucleic acid contamination in the sample, a minimum number of single nucleotide polymorphism (SNP) loci within the loci corresponding to the plurality of values, or any combination thereof.

27. The method of claim 24, wherein the second threshold comprises a specified lower limit of the quality metric.

28. The method of claim 24, wherein the first determination of somatic allele frequency comprises a determination of variant allele frequencies associated with the plurality of values after excluding variant alleles that are present at an allele frequency greater than an upper bound for the first estimate of the tumor fraction of the sample, and the second estimate of the tumor fraction of the sample is set equal to a maximum variant allele frequency detected.

29. The method of claim 24, wherein the second determination of somatic allele frequency comprises a determination of variant allele frequencies for all variant alleles associated with the plurality of values, and the second estimate of the tumor fraction of the sample is set equal to a maximum variant allele frequency detected.

30. The method of claim 24, wherein the first determination of somatic allele frequency and the second determination of somatic allele frequency further comprise removing variant allele frequencies from the determination that correspond to germline variants, clonal hematopoiesis of indeterminate potential (CHIP) variants, and sequencing artifact variants, prior to determining the second estimate of the tumor fraction of the sample.

31. The method of claim 30, wherein the first determination of somatic allele frequency and the second determination of somatic allele frequency further comprise using a variant allele frequency for a rearrangement as the second estimate of the tumor fraction of the sample if rearrangements are detected in the sample.

32. (canceled)

33. (canceled)

34. The method of claim 1, wherein the certainty metric value for the sample is indicative of a deviation of each of the plurality of values from a corresponding expected value.

35.-38. (canceled)

39. The method of claim 34, wherein each value within the plurality of values is a ratio of the difference in abundance between a maternal allele and a paternal allele, relative to an abundance of the maternal allele or the paternal allele at the corresponding locus, and the expected value comprises the expected ratio of the difference in abundance between a maternal allele and a paternal allele, relative to an abundance of the maternal allele or the paternal allele, wherein the expected value is the expected ratio for a non-tumorous sample.

40.-41. (canceled)

42. The method of claim 1, further comprising determining a probability distribution function for the plurality of values; wherein the certainty metric value for the sample is determined using the probability distribution function.

43.-46. (canceled)

47. A method of determining a tumor fraction of a sample from a subject, comprising:
receiving, at one or more processors, a plurality of values, each value indicative of a difference between an allele coverage of a locus in a tumor sample and an allele coverage of the same locus in a non-tumor sample at a plurality of loci within a sub genomic interval;
determining, by the one or more processors, a certainty metric value indicative of a dispersion of the plurality of values;
determining, by the one or more processors, a first estimate of the tumor fraction of the sample, the first estimate based on the certainty metric value for the sample and a predetermined relationship between one or more stored certainty metric values and one or more stored tumor fraction values;
determining, by the one or more processors, whether a value associated with the first estimate is greater than a first threshold;
based on a determination that the value associated with the first estimate is greater than the first threshold, outputting, by the one or more processors, the first estimate as the tumor fraction of the sample; and
based on a determination that the value associated with the first estimate of the tumor fraction is less than or equal to the first threshold:
determining, by the one or more processors, a second estimate of the tumor fraction of the sample based on an allele frequency determination; and
outputting, by the one or more processors, the second estimate as the tumor fraction of the sample.

48. The method of claim 47, wherein the tumor fraction is a value indicative of the ratio of circulating tumor DNA (ctDNA) to total cell-free DNA (cfDNA) in the sample.

49. The method of claim 47, wherein the first threshold is indicative of a minimum detectable quantity for the tumor fraction of the sample.

50.-110. (canceled)

111. A computer system comprising:
a processor; and
a memory communicatively coupled to the processor, configured to store:
a predetermined relationship between one or more stored certainty metric values and one or more associated stored tumor fraction values; and
instructions that, when executed by the processor cause the processor to:
receive a plurality of values, each value indicative of: (i) an allele fraction at a corresponding locus within a subgenomic interval in a sample, or (ii) a difference between an allele coverage of a locus in the sample and an allele coverage of the same locus in a non-tumor sample at a plurality of loci within a subgenomic interval;
calculate a certainty metric value indicative of a dispersion for the plurality of values;
calculate a first estimate of the tumor fraction of the sample, the first estimate based on the certainty metric value and the stored predetermined relationship;
determine whether a value associated with the first estimate is greater than a first threshold;
based on a determination that the value associated with the first estimate is greater than the first threshold, output the first estimate as the tumor fraction of the sample; and
based on a determination that the value associated with the first estimate is less than or equal to the first threshold:
calculate a second estimate of the tumor fraction of the sample based on an allele frequency determination; and
output the second estimate as the tumor fraction of the sample.

112.-132. (canceled)


SEQUENCE LISTING (1 sequence)

SEQ ID NO: 1 - DNA, 150 bp, Artificial Sequence
  Synthetic Construct
  misc_feature 16, 17, 18, 19, 20, 21, 22, 23, 24, 25, 26, 27, 28, 29,30, 31, 32, 33, 34, 35, 36, 37, 38, 39, 40, 41, 42, 43, 44,45, 46, 47, 48, 49, 50, 51, 52, 53, 54, 55, 56, 57, 58,59, 60, 61, 62, 63, 64, 65, 66, 67, 68, 69, 70, 71, 72,73: n = A,T,C or G
  misc_feature 74, 75, 76, 77, 78, 79, 80, 81, 82, 83, 84, 85, 86, 87,88, 89, 90, 91, 92, 93, 94, 95, 96, 97, 98, 99, 100, 101, 102,103, 104, 105, 106, 107, 108, 109, 110, 111, 112, 113, 114,115, 116, 117, 118, 119, 120, 121, 122, 123, 124, 125: n = A,T,C or G
  misc_feature 126, 127, 128, 129, 130, 131, 132, 133, 134, 135: n = A,T,C or G
atcgcaccagcgtgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactgcggctcctca